SEQUENCE LISTING (470 sequences)

SEQ ID NO: 1 - protein, 21 aa, Unknown
  Description of Unknown Malate dehydrogenase peptide
MLSALAKPVGAALARSFSTSA

SEQ ID NO: 2 - DNA, 63 bp, Ipomoea batatas
atgttcaggcactcttctcgactcctagctcgcgccaccacaatggggtggcgtcgccccttc

SEQ ID NO: 3 - DNA, 63 bp, Artificial Sequence
  Description of Artificial Sequence Synthetic oligonucleotide
ttcagatccgcgcttgtccgatcctccgcctcggcgaagcagtcgcttctccgccgcagcttc

SEQ ID NO: 4 - DNA, 63 bp, Artificial Sequence
  Description of Artificial Sequence Synthetic oligonucleotide
atgaaactatttcttttactagttatctctgcttcaatgctaattgatggcttagttaatgct

SEQ ID NO: 5 - DNA, 63 bp, Artificial Sequence
  Description of Artificial Sequence Synthetic oligonucleotide
ttcagatccgcgcttgtccgatcctccgcctcggcgaagcagtcgcttctccgccgcagcttt

SEQ ID NO: 6 - DNA, 63 bp, Brassica napus; identical to SEQ ID NO: 5
  CDS (1)..(63)

SEQ ID NO: 7 - protein, 21 aa, Brassica napus
FRSALVRSSASAKQSLLRRSF

SEQ ID NO: 8 - DNA, 63 bp, Artificial Sequence
  Description of Artificial Sequence Synthetic oligonucleotide
ttcagatccgcgcttgtccgatcctccgcctcggcgaagcagtcgcttctccgccgcatttcg

SEQ ID NO: 9 - protein, 21 aa, Artificial Sequence
  Description of Artificial Sequence Synthetic peptide
FRSALVRSSASAKQSLLRRIS

SEQ ID NO: 10 - DNA, 63 bp, Artificial Sequence; identical to SEQ ID NO: 5
  Description of Artificial Sequence Synthetic oligonucleotide

SEQ ID NO: 11 - protein, 21 aa, Artificial Sequence
  Description of Artificial Sequence Synthetic peptide
FRSALVRSSASAKQSLLRRSF

SEQ ID NO: 12 - DNA, 63 bp, Artificial Sequence
  Description of Artificial Sequence Synthetic oligonucleotide
ttcagatccgcgcttgtccgatcctccgcctcggcgaagcagtcgcttctccgccgccacttc

SEQ ID NO: 13 - protein, 21 aa, Artificial Sequence
  Description of Artificial Sequence Synthetic peptide
FRSALVRSSASAKQSLLRRHF

SEQ ID NO: 14 - DNA, 63 bp, Artificial Sequence
  Description of Artificial Sequence Synthetic oligonucleotide
atggccgcacgcctcgcgctggtggcggcgctcctgtgcgccggtgccacggccgccgcggcg

SEQ ID NO: 15 - DNA, 63 bp, Nicotiana tabacum
  CDS (1)..(63)
ggaaagctaagtacacttttatttgctctggtcctctatgtcatagccgcaggagctaatgca

SEQ ID NO: 16 - DNA, 63 bp, Nicotiana tabacum
cctttcgattcatgtgaaaataaacgagaccaggagatacagtatcggcgtcctcgattacgt

SEQ ID NO: 17 - RNA, 63 nt, Nicotiana tabacum
ggaaagcuaaguacacuuuuauuugcucugguccucuaugucauagccgcaggagcuaaugca

SEQ ID NO: 18 - protein, 21 aa, Nicotiana tabacum
GKLSTLLFALVLYVIAAGANA

SEQ ID NO: 19 - DNA, 63 bp, Brassica napus; identical to SEQ ID NO: 3
  CDS (1)..(63)

SEQ ID NO: 20 - DNA, 63 bp, Brassica napus
gaagctgcggcggagaagcgactgcttcgccgaggcggaggatcggacaagcgcggatctgaa

SEQ ID NO: 21 - DNA, 63 bp, Artificial Sequence
  Description of Artificial Sequence Synthetic oligonucleotide
aaagctgcggcggagaagcgactgcttcgccgaggcggaggatcggacaagcgcggatctgaa

SEQ ID NO: 22 - DNA, 63 bp, Artificial Sequence
  Description of Artificial Sequence Synthetic oligonucleotide
ggaaagctaagtacacttttatttgccctggtcctctatgtcatagccgcaggagctaatgca

SEQ ID NO: 23 - protein, 21 aa, Artificial Sequence
  Description of Artificial Sequence Synthetic peptide
GKLSTLLFALVLYVIAAGANA

SEQ ID NO: 24 - DNA, 63 bp, Artificial Sequence
  Description of Artificial Sequence Synthetic oligonucleotide
tgcattagctcctgcggctatgacatagaggaccagggcaaataaaagtgtacttagctttcc

SEQ ID NO: 25 - DNA, 255 bp, Arabidopsis thaliana
  CDS (1)..(255)
atgacaaagcgtgagtataattctcaacccgagatgttagaaggtgcaaaatcaataggtgccggagctgctacaattgcttcagcgggagctgctatcggtattggaaacgtattcagttctttgattcattctgtggcgcgaaatccatcattggctaaacaatcatttggttatgccattttgggctttgctctaaccgaagctattgcattgtttgccccaatgatggcctttttgatcttattcgtattc

SEQ ID NO: 26 - protein, 85 aa, Arabidopsis thaliana
MTKREYNSQPEMLEGAKSIGAGAATIASAGAAIGIGNVFSSLIHSVARNPSLAKQSFGYAILGFALTEAIALFAPMMAFLILFVF

SEQ ID NO: 27 - DNA, 255 bp, Artificial Sequence
  Description of Artificial Sequence Synthetic polynucleotide
atgacaaagcgtgagtataattctcaacccgagatgttagaaggtgcaaaatcaataggtgccggagctgctacaattgcttcagcgggagctgctatcggtattggaaacgtattcagttctttgattcattctgtggcgcgaaatccatcattggctaaacaatcatttggttatgccattttgggttttgctctaaccgaagctattgcattgtttgccccaatgatggcctttttgatcttattcgtattc

SEQ ID NO: 28 - protein, 85 aa, Artificial Sequence; identical to SEQ ID NO: 26
  Description of Artificial Sequence Synthetic polypeptide

SEQ ID NO: 29 - DNA, 318 bp, Arabidopsis thaliana
gaatacgaataagatcaaaaaggccatcattggggcaaacaatgcaatagcttcggttagagcaaagcccaaaatggcataaccaaatgattgtttagccaatgatggatttcgcgccacagaatgaatcaaagaactgaatacgtttccaataccgatagcagctcccgctgaagcaattgtagcagctccggcacctattgattttgcaccttctaacatctcgggttgagaattatactcacgctttgttccggcacctattgattttgcaccttctaacatctcgggttgagaattatactcacgctttgtcat

SEQ ID NO: 30 - DNA, 255 bp, Artificial Sequence
  Description of Artificial Sequence Synthetic polynucleotide
gaatacgaataagatcaaaaaggccatcattggggcaaacaatgcaatagcttcggttagagcaaaacccaaaatggcataaccaaatgattgtttagccaatgatggatttcgcgccacagaatgaatcaaagaactgaatacgtttccaataccgatagcagctcccgctgaagcaattgtagcagctccggcacctattgattttgcaccttctaacatctcgggttgagaattatactcacgctttgtcat

SEQ ID NO: 31 - DNA, 63 bp, Arabidopsis thaliana
  CDS (1)..(63)
atgaagatcagacttagcataaccatcatacttttatcatacacagtggctacggtggccgga

SEQ ID NO: 32 - protein, 21 aa, Arabidopsis thaliana
MKIRLSITIILLSYTVATVAG

SEQ ID NO: 33 - DNA, 63 bp, Artificial Sequence
  Description of Artificial Sequence Synthetic oligonucleotide
atgaagatcagacttagcctaaccatcatacttttatcatacacagtggctacggtggccgga

SEQ ID NO: 34 - protein, 21 aa, Artificial Sequence
  Description of Artificial Sequence Synthetic peptide
MKIRLSLTIILLSYTVATVAG

SEQ ID NO: 35 - DNA, 63 bp, Arabidopsis thaliana
tccggccaccgtagccactgtgtatgataaaagtatgatggttatgctaagtctgatcttcat

SEQ ID NO: 36 - DNA, 63 bp, Artificial Sequence
  Description of Artificial Sequence Synthetic oligonucleotide
tccggccaccgtagccactgtgtatgataaaagtatgatggttaggctaagtctgatcttcat

SEQ ID NO: 37 - DNA, 63 bp, Mesobuthus martensii; identical to SEQ ID NO: 4
  CDS (1)..(63)

SEQ ID NO: 38 - protein, 21 aa, Mesobuthus martensii
MKLFLLLVISASMLIDGLVNA

SEQ ID NO: 39 - DNA, 63 bp, Artificial Sequence
  Description of Artificial Sequence Synthetic oligonucleotide
atgaaactatttcttttactagttatctctgcttcaatactaattgatggcttagttaatgct

SEQ ID NO: 40 - protein, 21 aa, Artificial Sequence
  Description of Artificial Sequence Synthetic peptide
MKLFLLLVISASILIDGLVNA

SEQ ID NO: 41 - DNA, 63 bp, Mesobuthus martensii
agcattaactaagccatcaattagcattgaagcagagataactagtaaaagaaatagtttcat

SEQ ID NO: 42 - DNA, 63 bp, Artificial Sequence
  Description of Artificial Sequence Synthetic oligonucleotide
agcattaactaagccatcaattagtattgaagcagagataactagtaaaagaaatagtttcat

SEQ ID NO: 43 - DNA, 63 bp, Saccharomyces cerevisiae
  CDS (1)..(63)
gccgttcatgtttactctgggttgccttggtggggaactatcgcggccaccaccatcctcatt

SEQ ID NO: 44 - protein, 21 aa, Saccharomyces cerevisiae
AVHVYSGLPWWGTIAATTILI

SEQ ID NO: 45 - DNA, 63 bp, Artificial Sequence
  Description of Artificial Sequence Synthetic oligonucleotide
gccgttcatgtttactctgggttgccttggtgggcaactatcgcggccaccaccatcctcatt

SEQ ID NO: 46 - protein, 21 aa, Artificial Sequence
  Description of Artificial Sequence Synthetic peptide
AVHVYSGLPWWATIAATTILI

SEQ ID NO: 47 - DNA, 63 bp, Saccharomyces cerevisiae
aatgaggatggtggtggccgcgatagttccccaccaaggcaacccagagtaaacatgaacggc

SEQ ID NO: 48 - DNA, 63 bp, Artificial Sequence
  Description of Artificial Sequence Synthetic oligonucleotide
aatgaggatggtggtggccgcgatagttgcccaccaaggcaacccagagtaaacatgaacggc

SEQ ID NO: 49 - DNA, 63 bp, Ipomoea batatas; identical to SEQ ID NO: 2
  CDS (1)..(63)

SEQ ID NO: 50 - protein, 21 aa, Ipomoea batatas
MFRHSSRLLARATTMGWRRPF

SEQ ID NO: 51 - DNA, 63 bp, Artificial Sequence
  Description of Artificial Sequence Synthetic oligonucleotide
atgttcaggcactcttctcgattcctagctcgcgccaccacaatggggtggcgtcgccccttc

SEQ ID NO: 52 - protein, 21 aa, Artificial Sequence
  Description of Artificial Sequence Synthetic peptide
MFRHSSRFLARATTMGWRRPF

SEQ ID NO: 53 - DNA, 63 bp, Ipomoea batatas
gaaggggcgacgccaccccattgtggtggcgcgagctaggagtcgagaagagtgcctgaacat

SEQ ID NO: 54 - DNA, 63 bp, Artificial Sequence
  Description of Artificial Sequence Synthetic oligonucleotide
gaaggggcgacgccaccccattgtggtggcgcgagctaggaatcgagaagagtgcctgaacat

SEQ ID NO: 55 - DNA, 63 bp, Triticum aestivum; identical to SEQ ID NO: 14
  CDS (1)..(63)

SEQ ID NO: 56 - protein, 21 aa, Triticum aestivum
MAARLALVAALLCAGATAAAA

SEQ ID NO: 57 - DNA, 63 bp, Artificial Sequence
  Description of Artificial Sequence Synthetic oligonucleotide
atggccgcacgcctcgcgctgttggcggcgctcctgtgcgccggtgccacggccgccgcggcg

SEQ ID NO: 58 - protein, 21 aa, Artificial Sequence
  Description of Artificial Sequence Synthetic peptide
MAARLALLAALLCAGATAAAA

SEQ ID NO: 59 - DNA, 63 bp, Triticum aestivum
cgccgcggcggccgtggcaccggcgcacaggagcgccgccaccagcgcgaggcgtgcggccat

SEQ ID NO: 60 - DNA, 63 bp, Artificial Sequence
  Description of Artificial Sequence Synthetic oligonucleotide
cgccgcggcggccgtggcaccggcgcacaggagcgccgccaacagcgcgaggcgtgcggccat

SEQ ID NO: 61 - DNA, 63 bp, Polistes dominulus
  CDS (1)..(63)
atgaaaattagttgcttaatttgtctcgtaattgttcttacgatcattcatttgtctcaagct

SEQ ID NO: 62 - protein, 21 aa, Polistes dominulus
MKISCLICLVIVLTIIHLSQA

SEQ ID NO: 63 - DNA, 63 bp, Artificial Sequence
  Description of Artificial Sequence Synthetic oligonucleotide
atgaaaattagtagcttaatttgtctcgtaattgttcttacgatcattcatttgtctcaagct

SEQ ID NO: 64 - protein, 21 aa, Artificial Sequence
  Description of Artificial Sequence Synthetic peptide
MKISSLICLVIVLTIIHLSQA

SEQ ID NO: 65 - DNA, 63 bp, Polistes dominulus
agcttgagacaaatgaatgatcgtaagaacaattacgagacaaattaagcaactaattttcat

SEQ ID NO: 66 - DNA, 63 bp, Artificial Sequence
  Description of Artificial Sequence Synthetic oligonucleotide
agcttgagacaaatgaatgatcgtaagaacaattacgagacaaattaagctactaattttcat

SEQ ID NO: 67 - DNA, 63 bp, Artificial Sequence
  Description of Artificial Sequence Synthetic oligonucleotide
atgaaaattagttgcttaattcgtctcgtaattgttcttacgatcattcatttgtctcaagct

SEQ ID NO: 68 - protein, 21 aa, Artificial Sequence
  Description of Artificial Sequence Synthetic peptide
MKISCLIRLVIVLTIIHLSQA

SEQ ID NO: 69 - DNA, 63 bp, Artificial Sequence
  Description of Artificial Sequence Synthetic oligonucleotide
agcttgagacaaatgaatgatcgtaagaacaattacgagacgaattaagcaactaattttcat

SEQ ID NO: 70 - DNA, 63 bp, Arabidopsis thaliana
  CDS (1)..(63)
ttcagatctatgctcgtccgatcttctgcctccgcgaagcaggcggttatccgccgtagcttc

SEQ ID NO: 71 - protein, 21 aa, Arabidopsis thaliana
FRSMLVRSSASAKQAVIRRSF

SEQ ID NO: 72 - DNA, 63 bp, Artificial Sequence
  Description of Artificial Sequence Synthetic oligonucleotide
ttcagatctatgctcgtccgatcttctgcctccgcgaatcaggcggttatccgccgtagcttc

SEQ ID NO: 73 - protein, 21 aa, Artificial Sequence
  Description of Artificial Sequence Synthetic peptide
FRSMLVRSSASANQAVIRRSF

SEQ ID NO: 74 - DNA, 63 bp, Arabidopsis thaliana
gaagctacggcggataaccgcctgcttcgcggaggcagaagatcggacgagcatagatctgaa

SEQ ID NO: 75 - DNA, 63 bp, Artificial Sequence
  Description of Artificial Sequence Synthetic oligonucleotide
gaagctacggcggataaccgcctgattcgcggaggcagaagatcggacgagcatagatctgaa

SEQ ID NO: 76 - DNA, 63 bp, Saccharomyces cerevisiae
  CDS (1)..(63)
atgcaaaaaattttcagaccattccaattaacgagaggctttacctcttccgtaaaaaacttc

SEQ ID NO: 77 - protein, 21 aa, Saccharomyces cerevisiae
MQKIFRPFQLTRGFTSSVKNF

SEQ ID NO: 78 - DNA, 63 bp, Artificial Sequence
  Description of Artificial Sequence Synthetic oligonucleotide
atgcaagaaattttcagaccattccaattaacgagaggctttacctcttccgtaaaaaacttc

SEQ ID NO: 79 - protein, 21 aa, Artificial Sequence
  Description of Artificial Sequence Synthetic peptide
MQEIFRPFQLTRGFTSSVKNF

SEQ ID NO: 80 - DNA, 63 bp, Saccharomyces cerevisiae
gaagttttttacggaagaggtaaagcctctcgttaattggaatggtctgaaaattttttgcat

SEQ ID NO: 81 - DNA, 63 bp, Artificial Sequence
  Description of Artificial Sequence Synthetic oligonucleotide
gaagttttttacggaagaggtaaagcctctcgttaattggaatggtctgaaaatttcttgcat

SEQ ID NO: 82 - DNA, 63 bp, Homo sapiens
  CDS (1)..(63)
ctgcccgccgcgctgctccgccgcccgggacttggccgcctcgtccgccacgcccgtgcctat

SEQ ID NO: 83 - protein, 21 aa, Homo sapiens
LPAALLRRPGLGRLVRHARAY

SEQ ID NO: 84 - DNA, 63 bp, Artificial Sequence
  Description of Artificial Sequence Synthetic oligonucleotide
ctgcccgccgcgctgctccgccgcccgggacttggccgcctcgtccgccaggcccgtgcctat

SEQ ID NO: 85 - protein, 21 aa, Artificial Sequence
  Description of Artificial Sequence Synthetic peptide
LPAALLRRPGLGRLVRQARAY

SEQ ID NO: 86 - DNA, 63 bp, Homo sapiens
ataggcacgggcgtggcggacgaggcggccaagtcccgggcggcggagcagcgcggcgggcag

SEQ ID NO: 87 - DNA, 63 bp, Artificial Sequence
  Description of Artificial Sequence Synthetic oligonucleotide
ataggcacgggcctggcggacgaggcggccaagtcccgggcggcggagcagcgcggcgggcag

SEQ ID NO: 88 - DNA, 63 bp, Artificial Sequence
  Description of Artificial Sequence Synthetic oligonucleotide
atgttcaggcactcttctcggctcctagctcgggccaccacaatggggtggcgtcgccccttc

SEQ ID NO: 89 - protein, 21 aa, Artificial Sequence
  Description of Artificial Sequence Synthetic peptide
MFRHSSRLLARATTMGWRRPF

SEQ ID NO: 90 - DNA, 63 bp, Artificial Sequence
  Description of Artificial Sequence Synthetic oligonucleotide
gaaggggcgacgccaccccattgtggtggcccgagctaggagccgagaagagtgcctgaacat

SEQ ID NO: 91 - DNA, 63 bp, Hordeum vulgare
  CDS (1)..(63)
atggcggcacgcctgatgctggtggcggcgctcctgtgcgcggcgacggccatggccacggcg

SEQ ID NO: 92 - protein, 21 aa, Hordeum vulgare
MAARLMLVAALLCAATAMATA

SEQ ID NO: 93 - DNA, 63 bp, Artificial Sequence
  Description of Artificial Sequence Synthetic oligonucleotide
atggcggcacgcctgatgctggtggcggcgctcctgtgtgcggcgacggcgatggccacggcg

SEQ ID NO: 94 - protein, 21 aa, Artificial Sequence
  Description of Artificial Sequence Synthetic peptide
MAARLMLVAALLCAATAMATA

SEQ ID NO: 95 - DNA, 63 bp, Hordeum vulgare
cgccgtggccatggccgtcgccgcgcacaggagcgccgccaccagcatcaggcgtgccgccat

SEQ ID NO: 96 - DNA, 63 bp, Artificial Sequence
  Description of Artificial Sequence Synthetic oligonucleotide
cgccgtggccatcgccgtcgccgcacacaggagcgccgccaccagcatcaggcgtgccgccat

SEQ ID NO: 97 - DNA, 63 bp, Artificial Sequence
  Description of Artificial Sequence Synthetic oligonucleotide
atggcggcacgactgatgctggtggcggcgctcttgtgcgcggcgacggccatggccacggcg

SEQ ID NO: 98 - DNA, 63 bp, Artificial Sequence
  Description of Artificial Sequence Synthetic oligonucleotide
cgccgtggccatggccgtcgccgcgcacaagagcgccgccaccagcatcagtcgtgccgccat

SEQ ID NO: 99 - DNA, 63 bp, Bos taurus
  CDS (1)..(63)
gtatcccgtctgagctcgcgtctgcaagctctggcctcggccccatgccgttcgctcagttgt

SEQ ID NO: 100 - protein, 21 aa, Bos taurus
VSRLSSRLQALASAPCRSLSC

SEQ ID NO: 101 - DNA, 63 bp, Artificial Sequence
  Description of Artificial Sequence Synthetic oligonucleotide
gtatcccgtctgagctctcgactgcaagctctggcctcggccccatgccgttcgctcagttgt

SEQ ID NO: 102 - protein, 21 aa, Artificial Sequence
  Description of Artificial Sequence Synthetic peptide
VSRLSSRLQALASAPCRSLSC

SEQ ID NO: 103 - DNA, 63 bp, Bos taurus
acaactgagcgaacggcatggggccgaggccagagcttgcagacgcgagctcagacgggatac

SEQ ID NO: 104 - DNA, 63 bp, Artificial Sequence
  Description of Artificial Sequence Synthetic oligonucleotide
acaactgagcgaacggcatggggccgaggccagagcttgcagtcgagagctcagacgggatac

SEQ ID NO: 105 - DNA, 63 bp, Glycine max
  CDS (1)..(63)
gcttctctggcgttgctccgcagaaccacgctctcccactcgcacgtgcgtgcacgagccttc

SEQ ID NO: 106 - protein, 21 aa, Glycine max
ASLALLRRTTLSHSHVRARAF

SEQ ID NO: 107 - DNA, 63 bp, Artificial Sequence
  Description of Artificial Sequence Synthetic oligonucleotide
gcttctctggcgttgctccgcagaaccacgctctcccactcgcacgtacgtgcacgagctttc

SEQ ID NO: 108 - protein, 21 aa, Artificial Sequence
  Description of Artificial Sequence Synthetic peptide
ASLALLRRTTLSHSHVRARAF

SEQ ID NO: 109 - DNA, 63 bp, Glycine max
  CDS (1)..(63)
gaaggctcgtgcacgcacgtgcgagtgggagagcgtggttctgcggagcaacgccagagaagc

SEQ ID NO: 110 - DNA, 63 bp, Artificial Sequence
  Description of Artificial Sequence Synthetic oligonucleotide
gaaagctcgtgcacgtacgtgcgagtgggagagcgtggttctgcggagcaacgccagagaagc

SEQ ID NO: 111 - DNA, 63 bp, Camellia sinensis
  CDS (1)..(63)
atggcagcaatggtggacgccaagcctgcagcttcagtgcaaggcactccccttttggctacg

SEQ ID NO: 112 - protein, 21 aa, Camellia sinensis
MAAMVDAKPAASVQGTPLLAT

SEQ ID NO: 113 - DNA, 63 bp, Artificial Sequence
  Description of Artificial Sequence Synthetic oligonucleotide
atggcagcaatggtggacgccaagcctgcagcttcagtgcaggggactccccttttggctacg

SEQ ID NO: 114 - protein, 21 aa, Artificial Sequence
  Description of Artificial Sequence Synthetic peptide
MAAMVDAKPAASVQGTPLLAT

SEQ ID NO: 115 - DNA, 63 bp, Camellia sinensis
cgtagccaaaaggggagtgccttgcactgaagctgcaggcttggcgtccaccattgctgccat

SEQ ID NO: 116 - DNA, 63 bp, Artificial Sequence
  Description of Artificial Sequence Synthetic oligonucleotide
cgtagccaaaaggggagtcccctgcactgaagctgcaggcttggcgtccaccattgctgccat

SEQ ID NO: 117 - DNA, 63 bp, Artificial Sequence
  Description of Artificial Sequence Synthetic oligonucleotide
ttcagatctatgctagtccgatcttctgcctccgcgaagcaggcggttattcgccgtagcttc

SEQ ID NO: 118 - protein, 21 aa, Artificial Sequence
  Description of Artificial Sequence Synthetic peptide
FRSMLVRSSASAKQAVIRRSF

SEQ ID NO: 119 - DNA, 63 bp, Artificial Sequence
  Description of Artificial Sequence Synthetic oligonucleotide
gaagctacggcgaataaccgcctgcttcgcggaggcagaagatcggactagcatagatctgaa

SEQ ID NO: 120 - DNA, 63 bp, Artificial Sequence
  Description of Artificial Sequence Synthetic oligonucleotide
ttcagatctatgctcgtccgatcctctgcctcagcgaagcaggcggttatccgccgtagcttc

SEQ ID NO: 121 - DNA, 63 bp, Artificial Sequence
  Description of Artificial Sequence Synthetic oligonucleotide
gaagctacggcggataaccgcctgcttcgctgaggcagaggatcggacgagcatagatctgaa

SEQ ID NO: 122 - DNA, 63 bp, Artificial Sequence
  Description of Artificial Sequence Synthetic oligonucleotide
ttcagatctatgctcgtccgatcttctgcctccgcgaagcaggcggttatacgccgcagcttc

SEQ ID NO: 123 - DNA, 63 bp, Artificial Sequence
  Description of Artificial Sequence Synthetic oligonucleotide
gaagctgcggcgtataaccgcctgcttcgcggaggcagaagatcggacgagcatagatctgaa

SEQ ID NO: 124 - DNA, 63 bp, Arabidopsis thaliana
  CDS (1)..(63)
ttccgatcaatgattgttcgatctgcttccccagtgaagcagggtcttctccgcagaggattc

SEQ ID NO: 125 - protein, 21 aa, Arabidopsis thaliana
FRSMIVRSASPVKQGLLRRGF

SEQ ID NO: 126 - DNA, 63 bp, Artificial Sequence
  Description of Artificial Sequence Synthetic oligonucleotide
ttccgatcgatgattgtgcgatctgcttccccagtgaagcagggtcttctccgcagaggattc

SEQ ID NO: 127 - protein, 21 aa, Artificial Sequence
  Description of Artificial Sequence Synthetic peptide
FRSMIVRSASPVKQGLLRRGF

SEQ ID NO: 128 - DNA, 63 bp, Arabidopsis thaliana
gaatcctctgcggagaagaccctgcttcactggggaagcagatcgaacaatcattgatcggaa

SEQ ID NO: 129 - DNA, 63 bp, Artificial Sequence
  Description of Artificial Sequence Synthetic oligonucleotide
gaatcctctgcggagaagaccctgcttcactggggaagcagatcgcacaatcatcgatcggaa

SEQ ID NO: 130 - DNA, 63 bp, Artificial Sequence
  Description of Artificial Sequence Synthetic oligonucleotide
ttccgatcaatgattgttcgatctgcctccccagtgaagcagggccttctccgcagaggattc

SEQ ID NO: 131 - DNA, 63 bp, Artificial Sequence
  Description of Artificial Sequence Synthetic oligonucleotide
gaatcctctgcggagaaggccctgcttcactggggaggcagatcgaacaatcattgatcggaa

SEQ ID NO: 132 - DNA, 63 bp, Artificial Sequence
  Description of Artificial Sequence Synthetic oligonucleotide
ttccgatcaatgattgttcgatctgcttccccagtcaagcagggtcttctccgaagaggattc

SEQ ID NO: 133 - DNA, 63 bp, Artificial Sequence
  Description of Artificial Sequence Synthetic oligonucleotide
gaatcctcttcggagaagaccctgcttgactggggaagcagatcgaacaatcattgatcggaa

SEQ ID NO: 134 - DNA, 63 bp, Rattus norvegicus
  CDS (1)..(63)
ctgcccgccgcattgcttcgtcacccaggtctgcgccgtctggtgctccaggcgcgtacgtac

SEQ ID NO: 135 - protein, 21 aa, Rattus norvegicus
LPAALLRHPGLRRLVLQARTY

SEQ ID NO: 136 - DNA, 63 bp, Artificial Sequence
  Description of Artificial Sequence Synthetic oligonucleotide
ctgcccgccgcattgcttcgtcacccaggtctgcgccgtctggtactacaggcgcgtacgtac

SEQ ID NO: 137 - protein, 21 aa, Artificial Sequence
  Description of Artificial Sequence Synthetic peptide
LPAALLRHPGLRRLVLQARTY

SEQ ID NO: 138 - DNA, 63 bp, Rattus norvegicus
gtacgtacgcgcctggagcaccagacggcgcagacctgggtgacgaagcaatgcggcgggcag

SEQ ID NO: 139 - DNA, 63 bp, Artificial Sequence
  Description of Artificial Sequence Synthetic oligonucleotide
gtacgtacgcgcctgtagtaccagacggcgcagacctgggtgacgaagcaatgcggcgggcag

SEQ ID NO: 140 - DNA, 63 bp, Saccharomyces cerevisiae
  CDS (1)..(63)
ttacgttcaattattggaaagagtgcatcaagatcattgaatttcgtcgctaagcgttcatat

SEQ ID NO: 141 - protein, 21 aa, Saccharomyces cerevisiae
LRSIIGKSASRSLNFVAKRSY

SEQ ID NO: 142 - DNA, 63 bp, Artificial Sequence
  Description of Artificial Sequence Synthetic oligonucleotide
ttacgttccattattggaaagagtgcatcaagatcattgaatttcgtcgctaagcgttcgtat

SEQ ID NO: 143 - protein, 21 aa, Artificial Sequence
  Description of Artificial Sequence Synthetic peptide
LRSIIGKSASRSLNFVAKRSY

SEQ ID NO: 144 - DNA, 63 bp, Saccharomyces cerevisiae
atatgaacgcttagcgacgaaattcaatgatcttgatgcactctttccaataattgaacgtaa

SEQ ID NO: 145 - DNA, 63 bp, Artificial Sequence
  Description of Artificial Sequence Synthetic oligonucleotide
atacgaacgcttagcgacgaaattcaatgatcttgatgcactctttccaataatggaacgtaa

SEQ ID NO: 146 - DNA, 63 bp, Phaseolus vulgaris
  CDS (1)..(63)
atggcttcctccaagttactctccctagccctcttccttgtgcttctcacacacgcaaactca

SEQ ID NO: 147 - protein, 21 aa, Phaseolus vulgaris
MASSKLLSLALFLVLLTHANS

SEQ ID NO: 148 - DNA, 63 bp, Artificial Sequence
  Description of Artificial Sequence Synthetic oligonucleotide
atggcttcctcaaagttactgtccctagccctcttccttgtgcttctcacacacgcaaactca

SEQ ID NO: 149 - protein, 21 aa, Artificial Sequence
  Description of Artificial Sequence Synthetic peptide
MASSKLLSLALFLVLLTHANS

SEQ ID NO: 150 - DNA, 63 bp, Phaseolus vulgaris
tgagtttgcgtgtgtgagaagcacaaggaagagggctagggagagtaacttggaggaagccat

SEQ ID NO: 151 - DNA, 63 bp, Artificial Sequence
  Description of Artificial Sequence Synthetic oligonucleotide
tgagtttgcgtgtgtgagaagcacaaggaagagggctagggacagtaactttgaggaagccat

SEQ ID NO: 152 - DNA, 63 bp, Artificial Sequence
  Description of Artificial Sequence Synthetic oligonucleotide
atggcttcctctaagttactctccctagccctcttccttgtgcttctcacacacgctaactca

SEQ ID NO: 153 - DNA, 63 bp, Artificial Sequence
  Description of Artificial Sequence Synthetic oligonucleotide
tgagttagcgtgtgtgagaagcacaaggaagagggctagggagagtaacttagaggaagccat

SEQ ID NO: 154 - DNA, 63 bp, Artificial Sequence
  Description of Artificial Sequence Synthetic oligonucleotide
atggcttcctccaagttactctccctagctctcttcctcgtgcttctcacacacgcaaactca

SEQ ID NO: 155 - DNA, 63 bp, Artificial Sequence
  Description of Artificial Sequence Synthetic oligonucleotide
tgagtttgcgtgtgtgagaagcacgaggaagagagctagggagagtaacttggaggaagccat

SEQ ID NO: 156 - DNA, 63 bp, Artificial Sequence
  Description of Artificial Sequence Synthetic oligonucleotide
tttagatctgcgcttgtccgatcctccgcctcggcgaagcagtcgcttctccgccgcagcttc

SEQ ID NO: 157 - DNA, 63 bp, Artificial Sequence
  Description of Artificial Sequence Synthetic oligonucleotide
gaagctgcggcggagaagcgactgcttcgccgaggcggaggatcggacaagcgcagatctaaa

SEQ ID NO: 158 - DNA, 63 bp, Artificial Sequence
  Description of Artificial Sequence Synthetic oligonucleotide
atggcggcacgcctgatgctggtggcggcgctcttgtgcgcggcgacggccatggccaccgcg

SEQ ID NO: 159 - DNA, 63 bp, Artificial Sequence
  Description of Artificial Sequence Synthetic oligonucleotide
cgcggtggccatggccgtcgccgcgcacaagagcgccgccaccagcatcaggcgtgccgccat

SEQ ID NO: 160 - DNA, 63 bp, Artificial Sequence
  Description of Artificial Sequence Synthetic oligonucleotide
atggccgcacgcctcgcgctggtagcggcgctcctgtgcgccggtgctacggccgccgcggcg

SEQ ID NO: 161 - protein, 21 aa, Artificial Sequence
  Description of Artificial Sequence Synthetic peptide
MAARLALVAALLCAGATAAAA

SEQ ID NO: 162 - DNA, 63 bp, Artificial Sequence
  Description of Artificial Sequence Synthetic oligonucleotide
cgccgcggcggccgtagcaccggcgcacaggagcgccgctaccagcgcgaggcgtgcggccat

SEQ ID NO: 163 - DNA, 63 bp, Spinacia oleracea
  CDS (1)..(63)
atggcttgttcaatgctgaatggtgttgataaactcgccttaatcagtgggaaaaccccaaat

SEQ ID NO: 164 - protein, 21 aa, Spinacia oleracea
MACSMLNGVDKLALISGKTPN

SEQ ID NO: 165 - DNA, 63 bp, Artificial Sequence
  Description of Artificial Sequence Synthetic oligonucleotide
atggcttgttctatgctgaatggtgttgataaactcgccttaatcagtggaaaaaccccaaat

SEQ ID NO: 166 - protein, 21 aa, Artificial Sequence
  Description of Artificial Sequence Synthetic peptide
MACSMLNGVDKLALISGKTPN

SEQ ID NO: 167 - DNA, 63 bp, Spinacia oleracea
atttggggttttcccactgattaaggcgagtttatcaacaccattcagcattgaacaagccat

SEQ ID NO: 168 - DNA, 63 bp, Artificial Sequence
  Description of Artificial Sequence Synthetic oligonucleotide
atttggggtttttccactgattaaggcgagtttatcaacaccattcagcatagaacaagccat

SEQ ID NO: 169 - DNA, 63 bp, Artificial Sequence
  Description of Artificial Sequence Synthetic oligonucleotide
atgcaaaaaattttcagaccatttcaattaacgagaggctttacctcttccgtaaagaacttc

SEQ ID NO: 170 - protein, 21 aa, Artificial Sequence
  Description of Artificial Sequence Synthetic peptide
MQKIFRPFQLTRGFTSSVKNF

SEQ ID NO: 171 - DNA, 63 bp, Artificial Sequence
  Description of Artificial Sequence Synthetic oligonucleotide
gaagttctttacggaagaggtaaagcctctcgttaattgaaatggtctgaaaattttttgcat

SEQ ID NO: 172 - DNA, 63 bp, Artificial Sequence
  Description of Artificial Sequence Synthetic oligonucleotide
gcttctctggcgttgctccgcagaaccacgctctcccattctcacgtgcgtgcacgagccttc

SEQ ID NO: 173 - DNA, 63 bp, Artificial Sequence
  Description of Artificial Sequence Synthetic oligonucleotide
gaaggctcgtgcacgcacgtgagaatgggagagcgtggttctgcggagcaacgccagagaagc

SEQ ID NO: 174 - DNA, 63 bp, Artificial Sequence
  Description of Artificial Sequence Synthetic oligonucleotide
ttcagatctatgctcgtccgatcttctgcgtccgcgaagcaggcggtgatccgccgtagcttc

SEQ ID NO: 175 - DNA, 63 bp, Artificial Sequence
  Description of Artificial Sequence Synthetic oligonucleotide
gaagctacggcggatcaccgcctgcttcgcggacgcagaagatcggacgagcatagatctgaa

SEQ ID NO: 176 - DNA, 63 bp, Artificial Sequence
  Description of Artificial Sequence Synthetic oligonucleotide
ttcagatctatgctcgtccgatcttctgcctccgcgaaacaggcggttatccgccgcagcttc

SEQ ID NO: 177 - DNA, 63 bp, Artificial Sequence
  Description of Artificial Sequence Synthetic oligonucleotide
gaagctgcggcggataaccgcctgtttcgcggaggcagaagatcggacgagcatagatctgaa

SEQ ID NO: 178 - DNA, 63 bp, Artificial Sequence
  Description of Artificial Sequence Synthetic oligonucleotide
ctgcccgccgcattgctacgtcacccaggcctgcgccgtctggtgctccaggcgcgtacgtac

SEQ ID NO: 179 - DNA, 63 bp, Artificial Sequence
  Description of Artificial Sequence Synthetic oligonucleotide
gtacgtacgcgcctggagcaccagacggcgcaggcctgggtgacgtagcaatgcggcgggcag

SEQ ID NO: 180 - DNA, 63 bp, Artificial Sequence
  Description of Artificial Sequence Synthetic oligonucleotide
ttacgttcaattattggaaagagtgcgtcgagatcattgaatttcgtcgctaagcgttcatat

SEQ ID NO: 181 - DNA, 63 bp, Artificial Sequence
  Description of Artificial Sequence Synthetic oligonucleotide
atatgaacgcttagcgacgaaattcaatgatctcgacgcactctttccaataattgaacgtaa

SEQ ID NO: 182 - DNA, 63 bp, Artificial Sequence
  Description of Artificial Sequence Synthetic oligonucleotide
cttcccgccgcgctgctccgccgcccgggacttggccgcctcgtccgccatgcccgtgcctat

SEQ ID NO: 183 - protein, 21 aa, Artificial Sequence
  Description of Artificial Sequence Synthetic peptide
LPAALLRRPGLGRLVRHARAY

SEQ ID NO: 184 - DNA, 63 bp, Artificial Sequence
  Description of Artificial Sequence Synthetic oligonucleotide
ataggcacgggcatggcggacgaggcggccaagtcccgggcggcggagcagcgcggcgggaag

SEQ ID NO: 185 - DNA, 63 bp, Artificial Sequence
  Description of Artificial Sequence Synthetic oligonucleotide
atggcttcctccaagttactctcgctggccctcttccttgtgcttctcacacacgcaaactca

SEQ ID NO: 186 - DNA, 63 bp, Artificial Sequence
  Description of Artificial Sequence Synthetic oligonucleotide
tgagtttgcgtgtgtgagaagcacaaggaagagggccagcgagagtaacttggaggaagccat

SEQ ID NO: 187 - DNA, 63 bp, Artificial Sequence
  Description of Artificial Sequence Synthetic oligonucleotide
atgaaaattagttgcttaatttgcctggtaattgttcttacgatcattcatttgtctcaagct

SEQ ID NO: 188 - protein, 21 aa, Artificial Sequence
  Description of Artificial Sequence Synthetic peptide
MKISCLICLVIVLTIIHLSQA

SEQ ID NO: 189 - DNA, 63 bp, Artificial Sequence
  Description of Artificial Sequence Synthetic oligonucleotide
ttcagatccgctctcgtccgatcctccgcctcggcgaagcagtcgcttctccgccgcagcttc

SEQ ID NO: 190 - DNA, 63 bp, Artificial Sequence
  Description of Artificial Sequence Synthetic oligonucleotide
gaagctgcggcggagaagcgactgcttcgccgaggcggaggatcggacgagagcggatctgaa

SEQ ID NO: 191 - DNA, 63 bp, Artificial Sequence
  Description of Artificial Sequence Synthetic oligonucleotide
atgaagatcagacttagcatcaccatcatacttttatcatacacagttgctacggtggccgga

SEQ ID NO: 192 - protein, 21 aa, Artificial Sequence
  Description of Artificial Sequence Synthetic peptide
MKIRLSITIILLSYTVATVAG

SEQ ID NO: 193 - DNA, 63 bp, Artificial Sequence
  Description of Artificial Sequence Synthetic oligonucleotide
tccggccaccgtagcaactgtgtatgataaaagtatgatggtgatgctaagtctgatcttcat

SEQ ID NO: 194 - DNA, 63 bp, Artificial Sequence
  Description of Artificial Sequence Synthetic oligonucleotide
atgaagatcagacttagcataaccatcatccttttatcttacacagtggctacggtggccgga

SEQ ID NO: 195 - DNA, 63 bp, Artificial Sequence
  Description of Artificial Sequence Synthetic oligonucleotide
tccggccaccgtagccactgtgtaagataaaaggatgatggttatgctaagtctgatcttcat

SEQ ID NO: 196 - DNA, 63 bp, Artificial Sequence
  Description of Artificial Sequence Synthetic oligonucleotide
atgaagatcagacttagcataaccatcatactgttatcttacacagtggctacggtggccgga

SEQ ID NO: 197 - DNA, 63 bp, Artificial Sequence
  Description of Artificial Sequence Synthetic oligonucleotide
tccggccaccgtagccactgtgtaagataacagtatgatggttatgctaagtctgatcttcat

SEQ ID NO: 198 - DNA, 63 bp, Artificial Sequence
  Description of Artificial Sequence Synthetic oligonucleotide
ggaaagctaagtacacttttatttgctctggttctctatgtcatagccgcaggagcaaatgca

SEQ ID NO: 199 - DNA, 63 bp, Artificial Sequence
  Description of Artificial Sequence Synthetic oligonucleotide
tgcatttgctcctgcggctatgacatagagaaccagagcaaataaaagtgtacttagctttcc

SEQ ID NO: 200 - DNA, 63 bp, Artificial Sequence
  Description of Artificial Sequence Synthetic oligonucleotide
atggcggcacgcctgatgctcgtggcggcgctcctgtgcgctgcgacggccatggccacggcg

SEQ ID NO: 201 - DNA, 63 bp, Artificial Sequence
  Description of Artificial Sequence Synthetic oligonucleotide
cgccgtggccatggccgtcgcagcgcacaggagcgccgccacgagcatcaggcgtgccgccat

SEQ ID NO: 202 - DNA, 63 bp, Artificial Sequence
  Description of Artificial Sequence Synthetic oligonucleotide
atggcggcacgcctgatgcttgtggcggcgctcctgtgcgcggcgacggccatggccacggca

SEQ ID NO: 203 - DNA, 63 bp, Artificial Sequence
  Description of Artificial Sequence Synthetic oligonucleotide
tgccgtggccatggccgtcgccgcgcacaggagcgccgccacaagcatcaggcgtgccgccat

SEQ ID NO: 204 - DNA, 63 bp, Artificial Sequence
  Description of Artificial Sequence Synthetic oligonucleotide
atggcggcacgcctgatgctggtggcggcgctcctgtgcgcagcgacggccatggccacggcc

SEQ ID NO: 205 - DNA, 63 bp, Artificial Sequence
  Description of Artificial Sequence Synthetic oligonucleotide
ggccgtggccatggccgtcgctgcgcacaggagcgccgccaccagcatcaggcgtgccgccat

SEQ ID NO: 206 - DNA, 63 bp, Artificial Sequence
  Description of Artificial Sequence Synthetic oligonucleotide
atggcggcacgcctgatgctggtggcggcgctcctctgcgcggcgacggccatggccaccgcg

SEQ ID NO: 207 - DNA, 63 bp, Artificial Sequence
  Description of Artificial Sequence Synthetic oligonucleotide
cgcggtggccatggccgtcgccgcgcagaggagcgccgccaccagcatcaggcgtgccgccat

SEQ ID NO: 208 - DNA, 63 bp, Artificial Sequence
  Description of Artificial Sequence Synthetic oligonucleotide
atggcttgttcaatgcttaatggtgttgataaactcgccttaatcagtggaaaaaccccaaat

SEQ ID NO: 209 - DNA, 63 bp, Artificial Sequence
  Description of Artificial Sequence Synthetic oligonucleotide
atttggggtttttccactgattaaggcgagtttatcaacaccattaagcattgaacaagccat

SEQ ID NO: 210 - DNA, 63 bp, Artificial Sequence
  Description of Artificial Sequence Synthetic oligonucleotide
atgcaaaagattttcagaccattccaattaacgagaggctttacctcttccgtaaaaaatttc

SEQ ID NO: 211 - DNA, 63 bp, Artificial Sequence
  Description of Artificial Sequence Synthetic oligonucleotide
gaaattttttacggaagaggtaaagcctctcgttaattggaatggtctgaaaatcttttgcat

SEQ ID NO: 212 - DNA, 63 bp, Artificial Sequence
  Description of Artificial Sequence Synthetic oligonucleotide
atgcaaaaaatttttagaccattccaattgacgagaggctttacctcttccgtaaaaaacttc

SEQ ID NO: 213 - DNA, 63 bp, Artificial Sequence
  Description of Artificial Sequence Synthetic oligonucleotide
gaagttttttacggaagaggtaaagcctctcgtcaattggaatggtctaaaaattttttgcat

SEQ ID NO: 214 - DNA, 63 bp, Bos taurus
  CDS (1)..(63)
ctcccctccgcgttgctccgccgtccgggtttgggccgcctcgttcgccaggtccgcctctac

SEQ ID NO: 215 - protein, 21 aa, Bos taurus
LPSALLRRPGLGRLVRQVRLY

SEQ ID NO: 216 - DNA, 63 bp, Artificial Sequence
  Description of Artificial Sequence Synthetic oligonucleotide
ctcccctccgctttgctacgccgtccgggtttgggccgcctcgttcgccaggtccgcctctac

SEQ ID NO: 217 - protein, 21 aa, Artificial Sequence
  Description of Artificial Sequence Synthetic peptide
LPSALLRRPGLGRLVRQVRLY

SEQ ID NO: 218 - DNA, 63 bp, Bos taurus
gtagaggcggacctggcgaacgaggcggcccaaacccggacggcggagcaacgcggaggggag

SEQ ID NO: 219 - DNA, 63 bp, Artificial Sequence
  Description of Artificial Sequence Synthetic oligonucleotide
gtagaggcggacctggcgaacgaggcggcccaaacccggacggcgtagcaaagcggaggggag

SEQ ID NO: 220 - DNA, 63 bp, Artificial Sequence
  Description of Artificial Sequence Synthetic oligonucleotide
gcttccctggcgttgctccgcagaaccacgctctctcactcgcacgtgcgtgcacgagccttc

SEQ ID NO: 221 - protein, 21 aa, Glycine max
EGSCTHVRVGERGSAEQRQRS

SEQ ID NO: 222 - DNA, 63 bp, Artificial Sequence
  Description of Artificial Sequence Synthetic oligonucleotide
gaaggctcgtgcacgcacgtgcgagtgagagagcgtggttctgcggagcaacgccagggaagc

SEQ ID NO: 223 - protein, 21 aa, Artificial Sequence
  Description of Artificial Sequence Synthetic peptide
EGSCTHVRVRERGSAEQRQGS

SEQ ID NO: 224 - DNA, 63 bp, Artificial Sequence
  Description of Artificial Sequence Synthetic oligonucleotide
gcttccctggcgttgctccgcagaacgacgctctcccactcgcacgtgcgtgcacgagccttc

SEQ ID NO: 225 - DNA, 63 bp, Artificial Sequence
  Description of Artificial Sequence Synthetic oligonucleotide
gaaggctcgtgcacgcacgtgcgagtgggagagcgtcgttctgcggagcaacgccagggaagc

SEQ ID NO: 226 - DNA, 63 bp, Artificial Sequence
  Description of Artificial Sequence Synthetic oligonucleotide
gcttcactggcgttgctccgcagaaccacgctctcccactcgcacgtccgtgcacgagccttc

SEQ ID NO: 227 - DNA, 63 bp, Artificial Sequence
  Description of Artificial Sequence Synthetic oligonucleotide
gaaggctcgtgcacggacgtgcgagtgggagagcgtggttctgcggagcaacgccagtgaagc

SEQ ID NO: 228 - DNA, 63 bp, Artificial Sequence
  Description of Artificial Sequence Synthetic oligonucleotide
gcttctctggcgttgctccgcagaacaacgctctcccactcgcacgtacgtgcacgagccttc

SEQ ID NO: 229 - DNA, 63 bp, Artificial Sequence
  Description of Artificial Sequence Synthetic oligonucleotide
gaaggctcgtgcacgtacgtgcgagtgggagagcgttgttctgcggagcaacgccagagaagc

SEQ ID NO: 230 - DNA, 63 bp, Artificial Sequence
  Description of Artificial Sequence Synthetic oligonucleotide
gcatctttggcgttgctccgcagaaccacgctctcccactcgcacgtgcgtgcacgagccttc

SEQ ID NO: 231 - DNA, 63 bp, Artificial Sequence
  Description of Artificial Sequence Synthetic oligonucleotide
gaaggctcgtgcacgcacgtgcgagtgggagagcgtggttctgcggagcaacgccaaagatgc

SEQ ID NO: 232 - DNA, 63 bp, Artificial Sequence
  Description of Artificial Sequence Synthetic oligonucleotide
atggcagcaatggtggacgccaagcctgcagcatcagtacaaggcactccccttttggctacg

SEQ ID NO: 233 - DNA, 63 bp, Artificial Sequence
  Description of Artificial Sequence Synthetic oligonucleotide
cgtagccaaaaggggagtgccttgtactgatgctgcaggcttggcgtccaccattgctgccat

SEQ ID NO: 234 - DNA, 63 bp, Artificial Sequence
  Description of Artificial Sequence Synthetic oligonucleotide
ctgcccgccgcattgctgcgtcacccaggtctgcgccgtctggttctccaggcgcgtacgtac

SEQ ID NO: 235 - DNA, 63 bp, Artificial Sequence
  Description of Artificial Sequence Synthetic oligonucleotide
gtacgtacgcgcctggagaaccagacggcgcagacctgggtgacgcagcaatgcggcgggcag

SEQ ID NO: 236 - DNA, 63 bp, Artificial Sequence
  Description of Artificial Sequence Synthetic oligonucleotide
ctgcccgccgcattgcttcgtcaccctggtctgcgtcgtctggtgctccaggcgcgtacgtac

SEQ ID NO: 237 - DNA, 63 bp, Artificial Sequence
  Description of Artificial Sequence Synthetic oligonucleotide
gtacgtacgcgcctggagcaccagacgacgcagaccagggtgacgaagcaatgcggcgggcag

SEQ ID NO: 238 - DNA, 63 bp, Artificial Sequence
  Description of Artificial Sequence Synthetic oligonucleotide
ctgcccgccgcattgcttcgtcaccccggtctgcgccgtctggtgctccaggcacgtacgtac

SEQ ID NO: 239 - DNA, 63 bp, Artificial Sequence
  Description of Artificial Sequence Synthetic oligonucleotide
gtacgtacgtgcctggagcaccagacggcgcagaccggggtgacgaagcaatgcggcgggcag

SEQ ID NO: 240 - DNA, 63 bp, Artificial Sequence
  Description of Artificial Sequence Synthetic oligonucleotide
ctgcccgccgcattgcttcgtcacccaggactgcgccgtttggtgctccaggcgcgtacgtac

SEQ ID NO: 241 - DNA, 63 bp, Artificial Sequence
  Description of Artificial Sequence Synthetic oligonucleotide
gtacgtacgcgcctggagcaccaaacggcgcagtcctgggtgacgaagcaatgcggcgggcag

SEQ ID NO: 242 - DNA, 63 bp, Artificial Sequence
  Description of Artificial Sequence Synthetic oligonucleotide
ttacgttcaattattggaaagagtgcgtcaagatcattgaacttcgtcgctaagcgttcatat

SEQ ID NO: 243 - DNA, 63 bp, Artificial Sequence
  Description of Artificial Sequence Synthetic oligonucleotide
atatgaacgcttagcgacgaagttcaatgatcttgacgcactctttccaataattgaacgtaa

SEQ ID NO: 244 - DNA, 63 bp, Artificial Sequence
  Description of Artificial Sequence Synthetic oligonucleotide
ctgcccgctgcgctgctccgccgcccggggcttggccgcctcgtccgccacgcccgtgcctat

SEQ ID NO: 245 - DNA, 63 bp, Artificial Sequence
  Description of Artificial Sequence Synthetic oligonucleotide
ataggcacgggcgtggcggacgaggcggccaagccccgggcggcggagcagcgcagcgggcag

SEQ ID NO: 246 - DNA, 63 bp, Artificial Sequence
  Description of Artificial Sequence Synthetic oligonucleotide
atggcttcctccaagttactctccctagccctcttccttgtactgctcacacacgcaaactca

SEQ ID NO: 247 - DNA, 63 bp, Artificial Sequence
  Description of Artificial Sequence Synthetic oligonucleotide
tgagtttgcgtgtgtgagcagtacaaggaagagggctagggagagtaacttggaggaagccat

SEQ ID NO: 248 - DNA, 63 bp, Artificial Sequence
  Description of Artificial Sequence Synthetic oligonucleotide
atgaaaattagttgcttaatatgtctcgtaattgtacttacgatcattcatttgtctcaagct

SEQ ID NO: 249 - DNA, 63 bp, Artificial Sequence
  Description of Artificial Sequence Synthetic oligonucleotide
agcttgagacaaatgaatgatcgtaagtacaattacgagacatattaagcaactaattttcat

SEQ ID NO: 250 - DNA, 63 bp, Artificial Sequence
  Description of Artificial Sequence Synthetic oligonucleotide
ttcagatccgcccttgtccgatcctccgcctcggcgaagcagtcgctgctccgccgcagcttc

SEQ ID NO: 251 - DNA, 63 bp, Artificial Sequence
  Description of Artificial Sequence Synthetic oligonucleotide
gaagctgcggcggagcagcgactgcttcgccgaggcggaggatcggacaagggcggatctgaa

SEQ ID NO: 252 - DNA, 63 bp, Artificial Sequence
  Description of Artificial Sequence Synthetic oligonucleotide
ttcagatccgcgcttgtccggtcctccgcatcggcgaagcagtcgcttctccgccgcagcttc

SEQ ID NO: 253 - DNA, 63 bp, Artificial Sequence
  Description of Artificial Sequence Synthetic oligonucleotide
gaagctgcggcggagaagcgactgcttcgccgatgcggaggaccggacaagcgcggatctgaa

SEQ ID NO: 254 - DNA, 63 bp, Artificial Sequence
  Description of Artificial Sequence Synthetic oligonucleotide
ttcagatccgcgcttgtccgatcctccgcctcggcgaagcagtcgctcctccgccggagcttc

SEQ ID NO: 255 - DNA, 63 bp, Artificial Sequence
  Description of Artificial Sequence Synthetic oligonucleotide
gaagctccggcggaggagcgactgcttcgccgaggcggaggatcggacaagcgcggatctgaa

SEQ ID NO: 256 - DNA, 63 bp, Artificial Sequence
  Description of Artificial Sequence Synthetic oligonucleotide
atgaagatcagactaagcataaccatcatacttttatcatacaccgtggctacggtggccgga

SEQ ID NO: 257 - DNA, 63 bp, Artificial Sequence; identical to SEQ ID NO: 256
  Description of Artificial Sequence Synthetic oligonucleotide

SEQ ID NO: 258 - DNA, 63 bp, Artificial Sequence
  Description of Artificial Sequence Synthetic oligonucleotide
atggcggcacgcctgatgctggtggcggcgctcctgtgcgcggcaactgccatggccacggcg

SEQ ID NO: 259 - DNA, 63 bp, Artificial Sequence
  Description of Artificial Sequence Synthetic oligonucleotide
cgccgtggccatggcagttgccgcgcacaggagcgccgccaccagcatcaggcgtgccgccat

SEQ ID NO: 260 - DNA, 63 bp, Artificial Sequence
  Description of Artificial Sequence Synthetic oligonucleotide
atggccgcacggctcgcgctagtggcggcgctcctgtgcgccggtgccacggccgccgcggcg

SEQ ID NO: 261 - DNA, 63 bp, Artificial Sequence
  Description of Artificial Sequence Synthetic oligonucleotide
cgccgcggcggccgtggcaccggcgcacaggagcgccgccactagcgcgagccgtgcggccat

SEQ ID NO: 262 - DNA, 63 bp, Artificial Sequence
  Description of Artificial Sequence Synthetic oligonucleotide
atggccgcacgactcgcgctggtggcggcgctcctgtgtgccggtgccacggccgccgcggcg

SEQ ID NO: 263 - DNA, 63 bp, Artificial Sequence
  Description of Artificial Sequence Synthetic oligonucleotide
cgccgcggcggccgtggcaccggcacacaggagcgccgccaccagcgcgagtcgtgcggccat

SEQ ID NO: 264 - DNA, 63 bp, Artificial Sequence
  Description of Artificial Sequence Synthetic oligonucleotide
atggccgcacgcctcgcgctggtggcggcgctcctgtgcgccggtgcgacggccgcggcggcg

SEQ ID NO: 265 - DNA, 63 bp, Artificial Sequence
  Description of Artificial Sequence Synthetic oligonucleotide
cgccgccgcggccgtcgcaccggcgcacaggagcgccgccaccagcgcgaggcgtgcggccat

SEQ ID NO: 266 - DNA, 63 bp, Artificial Sequence
  Description of Artificial Sequence Synthetic oligonucleotide
atgttcaggcattcttctcgactcttagctcgcgccaccacaatggggtggcgtcgccccttc

SEQ ID NO: 267 - DNA, 63 bp, Artificial Sequence
  Description of Artificial Sequence Synthetic oligonucleotide
gaaggggcgacgccaccccattgtggtggcgcgagctaagagtcgagaagaatgcctgaacat

SEQ ID NO: 268 - DNA, 63 bp, Artificial Sequence
  Description of Artificial Sequence Synthetic oligonucleotide
atgttcaggcactcttctcgactcctggctcgcgccaccacaatggggtggcgtcgccctttc

SEQ ID NO: 269 - DNA, 63 bp, Artificial Sequence
  Description of Artificial Sequence Synthetic oligonucleotide
gaaagggcgacgccaccccattgtggtggcgcgagccaggagtcgagaagagtgcctgaacat

SEQ ID NO: 270 - DNA, 63 bp, Artificial Sequence
  Description of Artificial Sequence Synthetic oligonucleotide
atgttcaggcactcttctcgactcctagctcgcgccactacaatggggtggcgtcgccccttt

SEQ ID NO: 271 - DNA, 63 bp, Artificial Sequence
  Description of Artificial Sequence Synthetic oligonucleotide
aaaggggcgacgccaccccattgtagtggcgcgagctaggagtcgagaagagtgcctgaacat

SEQ ID NO: 272 - DNA, 63 bp, Artificial Sequence
  Description of Artificial Sequence Synthetic oligonucleotide
atgttcaggcactcttctcgactcctagctcgcgccaccacgatgggatggcgtcgccccttc

SEQ ID NO: 273 - DNA, 63 bp, Artificial Sequence
  Description of Artificial Sequence Synthetic oligonucleotide
gaaggggcgacgccatcccatcgtggtggcgcgagctaggagtcgagaagagtgcctgaacat

SEQ ID NO: 274 - DNA, 63 bp, Artificial Sequence
  Description of Artificial Sequence Synthetic oligonucleotide
atgttcaggcactcttctcgactgctagctcgcgccaccactatggggtggcgtcgccccttc

SEQ ID NO: 275 - DNA, 64 bp, Artificial Sequence
  Description of Artificial Sequence Synthetic oligonucleotide
gaaggggcgacgccaccccatagtggtggcgcgagctagcagtcgagaagagtgcctgaacatc

SEQ ID NO: 276 - DNA, 63 bp, Artificial Sequence
  Description of Artificial Sequence Synthetic oligonucleotide
ggaaaactaagtacacttttatttgctctggtcctctatgtcatagctgcaggagctaatgca

SEQ ID NO: 277 - DNA, 63 bp, Artificial Sequence
  Description of Artificial Sequence Synthetic oligonucleotide
tgcattagctcctgcagctatgacatagaggaccagagcaaataaaagtgtacttagttttcc

SEQ ID NO: 278 - DNA, 63 bp, Artificial Sequence
  Description of Artificial Sequence Synthetic oligonucleotide
ggaaagctaagcacacttttatttgctctggtcctctatgtcatagccgctggagctaatgca

SEQ ID NO: 279 - DNA, 63 bp, Artificial Sequence
  Description of Artificial Sequence Synthetic oligonucleotide
tgcattagctccagcggctatgacatagaggaccagagcaaataaaagtgtgcttagctttcc

SEQ ID NO: 280 - DNA, 63 bp, Artificial Sequence
  Description of Artificial Sequence Synthetic oligonucleotide
atggcggcacgcctgatgctggtggcggcgctactgtgcgcggcgacggccatggccacggca

SEQ ID NO: 281 - DNA, 63 bp, Artificial Sequence
  Description of Artificial Sequence Synthetic oligonucleotide
tgccgtggccatggccgtcgccgcgcacagtagcgccgccaccagcatcaggcgtgccgccat

SEQ ID NO: 282 - DNA, 63 bp, Artificial Sequence
  Description of Artificial Sequence Synthetic oligonucleotide
atggctgcacgcctcgcgctggtggcggcgctcttgtgcgccggtgccacggccgccgcggcg

SEQ ID NO: 283 - DNA, 63 bp, Artificial Sequence
  Description of Artificial Sequence Synthetic oligonucleotide
cgccgcggcggccgtggcaccggcgcacaagagcgccgccaccagcgcgaggcgtgcagccat

SEQ ID NO: 284 - DNA, 63 bp, Artificial Sequence
  Description of Artificial Sequence Synthetic oligonucleotide
atggcttgttcaatgctgaatggtgttgataaactcgccttaatcagtgggaagactccaaat

SEQ ID NO: 285 - DNA, 63 bp, Artificial Sequence
  Description of Artificial Sequence Synthetic oligonucleotide
atttggagtcttcccactgattaaggcgagtttatcaacaccattcagcattgaacaagccat

SEQ ID NO: 286 - DNA, 63 bp, Artificial Sequence
  Description of Artificial Sequence Synthetic oligonucleotide
gcttctctggcgttgctccgccgaaccacgctctcccactcgcacgtgcgtgcccgagccttc

SEQ ID NO: 287 - DNA, 63 bp, Artificial Sequence
  Description of Artificial Sequence Synthetic oligonucleotide
gaaggctcgggcacgcacgtgcgagtgggagagcgtggttcggcggagcaacgccagagaagc

SEQ ID NO: 288 - DNA, 63 bp, Artificial Sequence
  Description of Artificial Sequence Synthetic oligonucleotide
atggcagccatggtggacgccaagcctgcagcttcagtgcaaggcactcccctgttggctacg

SEQ ID NO: 289 - DNA, 63 bp, Artificial Sequence
  Description of Artificial Sequence Synthetic oligonucleotide
cgtagccaacaggggagtgccttgcactgaagctgcaggcttggcgtccaccatggctgccat

SEQ ID NO: 290 - DNA, 63 bp, Artificial Sequence
  Description of Artificial Sequence Synthetic oligonucleotide
atggcagcaatggtggatgccaagcctgcagcttcagtgcaaggcactccccttttggctacc

SEQ ID NO: 291 - DNA, 63 bp, Artificial Sequence
  Description of Artificial Sequence Synthetic oligonucleotide
ggtagccaaaaggggagtgccttgcactgaagctgcaggcttggcatccaccattgctgccat

SEQ ID NO: 292 - DNA, 63 bp, Artificial Sequence
  Description of Artificial Sequence Synthetic oligonucleotide
atggcagcaatggtggacgccaagcctgcagcttccgtgcaaggaactccccttttggctacg

SEQ ID NO: 293 - DNA, 63 bp, Artificial Sequence
  Description of Artificial Sequence Synthetic oligonucleotide
cgtagccaaaaggggagttccttgcacggaagctgcaggcttggcgtccaccattgctgccat

SEQ ID NO: 294 - DNA, 63 bp, Artificial Sequence
  Description of Artificial Sequence Synthetic oligonucleotide
atggcagcaatggtggacgccaagcctgcagcgtctgtgcaaggcactccccttttggctacg

SEQ ID NO: 295 - DNA, 63 bp, Artificial Sequence
  Description of Artificial Sequence Synthetic oligonucleotide
cgtagccaaaaggggagtgccttgcacagacgctgcaggcttggcgtccaccattgctgccat

SEQ ID NO: 296 - DNA, 63 bp, Artificial Sequence
  Description of Artificial Sequence Synthetic oligonucleotide
ctgcccgccgcattgctacgtcacccaggtttgcgccgtctggtgctccaggcgcgtacgtac

SEQ ID NO: 297 - DNA, 63 bp, Artificial Sequence
  Description of Artificial Sequence Synthetic oligonucleotide
gtacgtacgcgcctggagcaccagacggcgcaaacctgggtgacgtagcaatgcggcgggcag

SEQ ID NO: 298 - DNA, 63 bp, Artificial Sequence
  Description of Artificial Sequence Synthetic oligonucleotide
ctgcccgccgcgctgctccgccgaccgggacttggccgcctcgtccgtcacgcccgtgcctat

SEQ ID NO: 299 - DNA, 63 bp, Artificial Sequence
  Description of Artificial Sequence Synthetic oligonucleotide
ataggcacgggcgtgacggacgaggcggccaagtcccggtcggcggagcagcgcggcgggcag

SEQ ID NO: 300 - DNA, 63 bp, Artificial Sequence
  Description of Artificial Sequence Synthetic oligonucleotide
ctgcctgccgcgctgctccgccgcccgggacttggccgcctcgttcgccacgcccgtgcctat

SEQ ID NO: 301 - DNA, 63 bp, Artificial Sequence
  Description of Artificial Sequence Synthetic oligonucleotide
ataggcacgggcgtggcgaacgaggcggccaagtcccgggcggcggagcagcgcggcaggcag

SEQ ID NO: 302 - DNA, 63 bp, Artificial Sequence
  Description of Artificial Sequence Synthetic oligonucleotide
atgaaactatttcttttactagttatctcggcttcaatgctaattgatggcttagttaatgca

SEQ ID NO: 303 - protein, 21 aa, Artificial Sequence
  Description of Artificial Sequence Synthetic peptide
MKLFLLLVISASMLIDGLVNA

SEQ ID NO: 304 - DNA, 63 bp, Artificial Sequence
  Description of Artificial Sequence Synthetic oligonucleotide
tgcattaactaagccatcaattagcattgaagccgagataactagtaaaagaaatagtttcat

SEQ ID NO: 305 - DNA, 63 bp, Artificial Sequence
  Description of Artificial Sequence Synthetic oligonucleotide
atggcttcctcaaagttactctctctagccctcttccttgtgcttctcacacacgcaaactca

SEQ ID NO: 306 - DNA, 63 bp, Artificial Sequence
  Description of Artificial Sequence Synthetic oligonucleotide
tgagtttgcgtgtgtgagaagcacaaggaagagggctagagagagtaactttgaggaagccat

SEQ ID NO: 307 - DNA, 63 bp, Artificial Sequence
  Description of Artificial Sequence Synthetic oligonucleotide
atgaaaataagttgcttaatttgtctcgtgattgttcttacgatcattcatttgtctcaagct

SEQ ID NO: 308 - DNA, 63 bp, Artificial Sequence
  Description of Artificial Sequence Synthetic oligonucleotide
agcttgagacaaatgaatgatcgtaagaacaatcacgagacaaattaagcaacttattttcat

SEQ ID NO: 309 - DNA, 63 bp, Artificial Sequence
  Description of Artificial Sequence Synthetic oligonucleotide
ttcagatccgcgcttgtcagatcctccgcctcggcgaagcagtcgcttctccgccgcagcttt

SEQ ID NO: 310 - DNA, 63 bp, Artificial Sequence
  Description of Artificial Sequence Synthetic oligonucleotide
aaagctgcggcggagaagcgactgcttcgccgaggcggaggatctgacaagcgcggatctgaa

SEQ ID NO: 311 - DNA, 63 bp, Artificial Sequence
  Description of Artificial Sequence Synthetic oligonucleotide
atgttcaggcactcttctcgacttctagctcgcgccaccacaatgggttggcgtcgccccttc

SEQ ID NO: 312 - DNA, 63 bp, Artificial Sequence
  Description of Artificial Sequence Synthetic oligonucleotide
gaaggggcgacgccaacccattgtggtggcgcgagctagaagtcgagaagagtgcctgaacat

SEQ ID NO: 313 - DNA, 63 bp, Artificial Sequence
  Description of Artificial Sequence Synthetic oligonucleotide
ggaaagctaagtacacttttatttgctctggtcctctatgtcatagccgcaggagccaatgct

SEQ ID NO: 314 - DNA, 63 bp, Artificial Sequence
  Description of Artificial Sequence Synthetic oligonucleotide
tgcattagctccggcggctatgacatagaggcccagagcaaataaaagtgtacttagctttcc

SEQ ID NO: 315 - DNA, 63 bp, Artificial Sequence
  Description of Artificial Sequence Synthetic oligonucleotide
ggcaagctaagtacacttttatttgctctggtcctctatgtcattgccgcaggagctaatgca

SEQ ID NO: 316 - DNA, 63 bp, Artificial Sequence
  Description of Artificial Sequence Synthetic oligonucleotide
tgcattagctcctgcggcaatgacatagaggaccagagcaaataaaagtgtacttagcttgcc

SEQ ID NO: 317 - DNA, 63 bp, Artificial Sequence
  Description of Artificial Sequence Synthetic oligonucleotide
atggcagcacgcctcgcgctggtggcggcgctcctgtgcgccggtgccacggccgccgcggct

SEQ ID NO: 318 - DNA, 63 bp, Artificial Sequence
  Description of Artificial Sequence Synthetic oligonucleotide
agccgcggcggccgtggcaccggcgcacaggagcgccgccaccagcgcgaggcgtgctgccat

SEQ ID NO: 319 - DNA, 63 bp, Artificial Sequence
  Description of Artificial Sequence Synthetic oligonucleotide
atggccgcacgcctcgctctggtggcggcgctcctgtgcgccggtgcaacggccgccgcggcg

SEQ ID NO: 320 - DNA, 63 bp, Artificial Sequence
  Description of Artificial Sequence Synthetic oligonucleotide
cgccgcggcggccgttgcaccggcgcacaggagcgccgccaccagagcgaggcgtgcggccat

SEQ ID NO: 321 - DNA, 63 bp, Artificial Sequence
  Description of Artificial Sequence Synthetic oligonucleotide
atggccgcacgcctcgcgcttgtggcggcgctcctgtgcgccggtgccacggccgccgctgcg

SEQ ID NO: 322 - DNA, 63 bp, Artificial Sequence
  Description of Artificial Sequence Synthetic oligonucleotide
cgcagcggcggccgtggcaccggcgcacaggagcgccgccacaagcgcgaggcgtgcggccat

SEQ ID NO: 323 - DNA, 63 bp, Artificial Sequence
  Description of Artificial Sequence Synthetic oligonucleotide
atggcttgctcaatgctgaatggtgttgataaactcgccttaatcagtgggaaaacccctaat

SEQ ID NO: 324 - DNA, 63 bp, Artificial Sequence
  Description of Artificial Sequence Synthetic oligonucleotide
attaggggttttcccactgattaaggcgagtttatcaacaccattcagcattgagcaagccat

SEQ ID NO: 325 - DNA, 63 bp, Artificial Sequence
  Description of Artificial Sequence Synthetic oligonucleotide
gtatcccgtctgagctctcgtctgcaagccctggcctcggccccatgccgttcgctcagttgt

SEQ ID NO: 326 - DNA, 63 bp, Artificial Sequence
  Description of Artificial Sequence Synthetic oligonucleotide
acaactgagcgaacggcatggggccgaggccagggcttgcagacgagagctcagacgggatac

SEQ ID NO: 327 - DNA, 63 bp, Artificial Sequence
  Description of Artificial Sequence Synthetic oligonucleotide
gtatcccgtctgagctcgcgtctgcaagctctggcctctgccccctgccgttcgctcagttgt

SEQ ID NO: 328 - DNA, 63 bp, Artificial Sequence
  Description of Artificial Sequence Synthetic oligonucleotide
acaactgagcgaacggcagggggcagaggccagagcttgcagacgcgagctcagacgggatac

SEQ ID NO: 329 - DNA, 63 bp, Artificial Sequence
  Description of Artificial Sequence Synthetic oligonucleotide
atggcagcaatggtggacgccaagcccgctgcttcagtgcaaggcactccccttttggctacg

SEQ ID NO: 330 - DNA, 63 bp, Artificial Sequence
  Description of Artificial Sequence Synthetic oligonucleotide
cgtagccaaaaggggagtgccttgcactgaagcagcgggcttggcgtccaccattgctgccat

SEQ ID NO: 331 - DNA, 63 bp, Artificial Sequence
  Description of Artificial Sequence Synthetic oligonucleotide
ttacgttccattattggaaagagtgcttcaagatcattgaatttcgtcgctaagcgttcatat

SEQ ID NO: 332 - DNA, 63 bp, Artificial Sequence
  Description of Artificial Sequence Synthetic oligonucleotide
atatgaacgcttagcgacgaaattcaatgatcttgaagcactctttccaataatggaacgtaa

SEQ ID NO: 333 - DNA, 63 bp, Artificial Sequence
  Description of Artificial Sequence Synthetic oligonucleotide
ttacgttcgattattggaaagagtgcatcaagatctttgaatttcgtcgctaagcgttcatat

SEQ ID NO: 334 - DNA, 63 bp, Artificial Sequence
  Description of Artificial Sequence Synthetic oligonucleotide
atatgaacgcttagcgacgaaattcaaagatcttgatgcactctttccaataatcgaacgtaa

SEQ ID NO: 335 - DNA, 63 bp, Artificial Sequence
  Description of Artificial Sequence Synthetic oligonucleotide
ttgcccgccgcgctgctccgccggccgggacttggccgcctcgtccgccacgcccgtgcctat

SEQ ID NO: 336 - DNA, 63 bp, Artificial Sequence
  Description of Artificial Sequence Synthetic oligonucleotide
ataggcacgggcgtggcggacgaggcggccaagtcccggccggcggagcagcgcggcgggcaa

SEQ ID NO: 337 - DNA, 63 bp, Artificial Sequence
  Description of Artificial Sequence Synthetic oligonucleotide
atgaaactatttcttttgctagttatctctgcttcaatgctaattgatggcttagttaacgct

SEQ ID NO: 338 - DNA, 63 bp, Artificial Sequence
  Description of Artificial Sequence Synthetic oligonucleotide
agcgttaactaagccatcaattagcattgaagcagagataactagcaaaagaaatagtttcat

SEQ ID NO: 339 - DNA, 63 bp, Artificial Sequence
  Description of Artificial Sequence Synthetic oligonucleotide
atggcttcctccaagctactctccctagcactcttccttgtgcttctcacacacgcaaactca

SEQ ID NO: 340 - DNA, 63 bp, Artificial Sequence
  Description of Artificial Sequence Synthetic oligonucleotide
tgagtttgcgtgtgtgagaagcacaaggaagagtgctagggagagtagcttggaggaagccat

SEQ ID NO: 341 - DNA, 63 bp, Artificial Sequence
  Description of Artificial Sequence Synthetic oligonucleotide
atgaagattagttgcttaatttgtctcgtaattgttcttacgatcattcatttgtctcaagcc

SEQ ID NO: 342 - DNA, 63 bp, Artificial Sequence
  Description of Artificial Sequence Synthetic oligonucleotide
ggcttgagacaaatgaatgatcgtaagaacaattacgagacaaattaagcaactaatcttcat

SEQ ID NO: 343 - DNA, 63 bp, Artificial Sequence
  Description of Artificial Sequence Synthetic oligonucleotide
atgaaaattagttgcttgatttgtctcgtaattgttcttacgatcatccatttgtctcaagct

SEQ ID NO: 344 - DNA, 63 bp, Artificial Sequence
  Description of Artificial Sequence Synthetic oligonucleotide
agcttgagacaaatggatgatcgtaagaacaattacgagacaaatcaagcaactaattttcat

SEQ ID NO: 345 - DNA, 63 bp, Artificial Sequence
  Description of Artificial Sequence Synthetic oligonucleotide
atgaaaattagttgcttaatctgtctcgtaattgttcttacgatcattcatttgtctcaggct

SEQ ID NO: 346 - DNA, 63 bp, Artificial Sequence
  Description of Artificial Sequence Synthetic oligonucleotide
agcctgagacaaatgaatgatcgtaagaacaattacgagacagattaagcaactaattttcat

SEQ ID NO: 347 - DNA, 63 bp, Petunia sp.
  CDS (1)..(63)
gggaggcatgattatcatctatcaccaccaccagcaccaaagccagctgatcacactggccaa

SEQ ID NO: 348 - protein, 21 aa, Petunia sp.
GRHDYHLSPPPAPKPADHTGQ

SEQ ID NO: 349 - DNA, 63 bp, Artificial Sequence
  Description of Artificial Sequence Synthetic oligonucleotide
ggcaggcatgattatcatctatcacccccaccagcaccaaagccagctgatcacactggccaa

SEQ ID NO: 350 - protein, 21 aa, Artificial Sequence
  Description of Artificial Sequence Synthetic peptide
GRHDYHLSPPPAPKPADHTGQ

SEQ ID NO: 351 - DNA, 63 bp, Petunia sp.
ttggccagtgtgatcagctggctttggtgctggtggtggtgatagatgataatcatgcctccc

SEQ ID NO: 352 - DNA, 63 bp, Artificial Sequence
  Description of Artificial Sequence Synthetic oligonucleotide
ttggccagtgtgatcagctggctttggtgctggtgggggtgatagatgataatcatgcctgcc

SEQ ID NO: 353 - DNA, 63 bp, Artificial Sequence
  Description of Artificial Sequence Synthetic oligonucleotide
gtcagatctatgcttgtccgatcttctgcctccgcgaagcaggcggttatccgccgtagcttc

SEQ ID NO: 354 - protein, 21 aa, Artificial Sequence
  Description of Artificial Sequence Synthetic peptide
VRSMLVRSSASAKQAVIRRSF

SEQ ID NO: 355 - DNA, 63 bp, Artificial Sequence
  Description of Artificial Sequence Synthetic oligonucleotide
gaagctacggcggataaccgcctgcttcgcggaggcagaagatcggacaagcatagatctgac

SEQ ID NO: 356 - DNA, 63 bp, Artificial Sequence
  Description of Artificial Sequence Synthetic oligonucleotide
atgcaaaaaattttcagaccattccaaataacgagaggctttacctcttccttaaaaaacttc

SEQ ID NO: 357 - protein, 21 aa, Artificial Sequence
  Description of Artificial Sequence Synthetic peptide
MQKIFRPFQITRGFTSSLKNF

SEQ ID NO: 358 - DNA, 63 bp, Artificial Sequence
  Description of Artificial Sequence Synthetic oligonucleotide
gaagttttttaaggaagaggtaaagcctctcgttatttggaatggtctgaaaattttttgcat

SEQ ID NO: 359 - DNA, 63 bp, Artificial Sequence
  Description of Artificial Sequence Synthetic oligonucleotide
atggcagcaatggtggacgccaagcctgcagcgtcagcgcaaggcactccccttttggctacg

SEQ ID NO: 360 - protein, 21 aa, Artificial Sequence
  Description of Artificial Sequence Synthetic peptide
MAAMVDAKPAASAQGTPLLAT

SEQ ID NO: 361 - DNA, 63 bp, Artificial Sequence
  Description of Artificial Sequence Synthetic oligonucleotide
cgtagccaaaaggggagtgccttgcgctgacgctgcaggcttggcgtccaccattgctgccat

SEQ ID NO: 362 - DNA, 63 bp, Artificial Sequence
  Description of Artificial Sequence Synthetic oligonucleotide
ggaaagctaactacacttttatttgctctggtcctctatgtcatagtcgcaggagctaatgca

SEQ ID NO: 363 - protein, 21 aa, Artificial Sequence
  Description of Artificial Sequence Synthetic peptide
GKLTTLLFALVLYVIVAGANA

SEQ ID NO: 364 - DNA, 63 bp, Artificial Sequence
  Description of Artificial Sequence Synthetic oligonucleotide
tgcattagctcctgcgactatgacatagaggaccagagcaaataaaagtgtagttagctttcc

SEQ ID NO: 365 - DNA, 63 bp, Artificial Sequence
  Description of Artificial Sequence Synthetic oligonucleotide
ggaaagctaagtacacttttatttgctctggtcgtctatgtcatagccgcaggagctaatgcc

SEQ ID NO: 366 - protein, 21 aa, Artificial Sequence
  Description of Artificial Sequence Synthetic peptide
GKLSTLLFALVVYVIAAGANA

SEQ ID NO: 367 - DNA, 63 bp, Artificial Sequence
  Description of Artificial Sequence Synthetic oligonucleotide
ggcattagctcctgcggctatgacatagacgaccagagcaaataaaagtgtacttagctttcc

SEQ ID NO: 368 - DNA, 63 bp, Artificial Sequence
  Description of Artificial Sequence Synthetic oligonucleotide
atgttcaggcactcttgtcgactcctagctcgcgccaccacaatgggggggcgtcgccccttc

SEQ ID NO: 369 - protein, 21 aa, Artificial Sequence
  Description of Artificial Sequence Synthetic peptide
MFRHSCRLLARATTMGGRRPF

SEQ ID NO: 370 - DNA, 63 bp, Artificial Sequence
  Description of Artificial Sequence Synthetic oligonucleotide
gaaggggcgacgcccccccattgtggtggcgcgagctaggagtcgacaagagtgcctgaacat

SEQ ID NO: 371 - DNA, 51 bp, Arabidopsis thaliana
  CDS (1)..(51)
gcatctgctctcgctcttaagagactcctatcatcctccatcgctccacgt

SEQ ID NO: 372 - protein, 17 aa, Arabidopsis thaliana
ASALALKRLLSSSIAPR

SEQ ID NO: 373 - DNA, 51 bp, Artificial Sequence
  Description of Artificial Sequence Synthetic oligonucleotide
gcatctgctctcgctcttaagagactcctatcgtcctccatcgctccgcgt

SEQ ID NO: 374 - protein, 17 aa, Artificial Sequence
  Description of Artificial Sequence Synthetic peptide
ASALALKRLLSSSIAPR

SEQ ID NO: 375 - DNA, 51 bp, Arabidopsis thaliana
acgtggagcgatggaggatgataggagtctcttaagagcgagagcagatgc

SEQ ID NO: 376 - DNA, 51 bp, Artificial Sequence
  Description of Artificial Sequence Synthetic oligonucleotide
acgcggagcgatggaggacgataggagtctcttaagagcgagagcagatgc

SEQ ID NO: 377 - DNA, 51 bp, Solanum tuberosum
  CDS (1)..(51)
tggagagtggctcgatctgcggcgtcgactttccgccgtacgcggcggtta

SEQ ID NO: 378 - protein, 17 aa, Solanum tuberosum
WRVARSAASTFRRTRRL

SEQ ID NO: 379 - DNA, 51 bp, Artificial Sequence
  Description of Artificial Sequence Synthetic oligonucleotide
tggagagtggcgcgatcagcggcgtcgactttccgccgtacgcggcggtta

SEQ ID NO: 380 - protein, 17 aa, Artificial Sequence
  Description of Artificial Sequence Synthetic peptide
WRVARSAASTFRRTRRL

SEQ ID NO: 381 - DNA, 51 bp, Solanum tuberosum
taaccgccgcgtacggcggaaagtcgacgccgcagatcgagccactctcca

SEQ ID NO: 382 - DNA, 51 bp, Artificial Sequence
  Description of Artificial Sequence Synthetic oligonucleotide
gaattcttctcgatcctccaaacgtcatcttctgcagtctcttatgtgtta

SEQ ID NO: 383 - DNA, 93 bp, Solanum tuberosum
  CDS (1)..(93)
atggctatggcaatagctcttcggaggctttccgctacagttgacaaaccggttaagagtctctacaatggtggctctctctattacatgtca

SEQ ID NO: 384 - protein, 31 aa, Solanum tuberosum
MAMAIALRRLSATVDKPVKSLYNGGSLYYMS

SEQ ID NO: 385 - DNA, 93 bp, Artificial Sequence
  Description of Artificial Sequence Synthetic oligonucleotide
atggctatggcaatagctctgcggaggctttccgctacagttgacaaaccggttaagagtctctacaatggtggctatctctattacatgtca

SEQ ID NO: 386 - protein, 31 aa, Artificial Sequence
  Description of Artificial Sequence Synthetic polypeptide
MAMAIALRRLSATVDKPVKSLYNGGYLYYMS

SEQ ID NO: 387 - DNA, 93 bp, Solanum tuberosum
tgacatgtaatagagagagccaccattgtagagactcttaaccggtttgtcaactgtagcggaaagcctccgaagagctattgccatagccat

SEQ ID NO: 388 - DNA, 93 bp, Artificial Sequence
  Description of Artificial Sequence Synthetic oligonucleotide
tgacatgtaatagagatagccaccattgtagagactcttaaccggtttgtcaactgtagcggaaagcctccgcagagctattgccatagccat

SEQ ID NO: 389 - DNA, 51 bp, Artificial Sequence
  Description of Artificial Sequence Synthetic oligonucleotide
tggagagtggctcgatctgtggcgtcgacttttcgccgtacgcggcggtta

SEQ ID NO: 390 - protein, 17 aa, Artificial Sequence
  Description of Artificial Sequence Synthetic peptide
WRVARSVASTFRRTRRL

SEQ ID NO: 391 - DNA, 51 bp, Solanum tuberosum; identical to SEQ ID NO: 381

SEQ ID NO: 392 - DNA, 51 bp, Artificial Sequence
  Description of Artificial Sequence Synthetic oligonucleotide
taaccgccgcgtacggcgaaaagtcgacgccacagatcgagccactctcca

SEQ ID NO: 393 - DNA, 51 bp, Homo sapiens
  CDS (1)..(51)
atggattatttgctcatgattttctctctgctgtttgtggcttgccaagga

SEQ ID NO: 394 - protein, 17 aa, Homo sapiens
MDYLLMIFSLLFVACQG

SEQ ID NO: 395 - DNA, 51 bp, Artificial Sequence
  Description of Artificial Sequence Synthetic oligonucleotide
atggattatttgctcatgattttctgtccgctgtttgtggcttgccaagga

SEQ ID NO: 396 - protein, 17 aa, Artificial Sequence
  Description of Artificial Sequence Synthetic peptide
MDYLLMIFCPLFVACQG

SEQ ID NO: 397 - DNA, 51 bp, Homo sapiens
tccttggcaagccacaaacagcagagagaaaatcatgagcaaataatccat

SEQ ID NO: 398 - DNA, 51 bp, Artificial Sequence
  Description of Artificial Sequence Synthetic oligonucleotide
tccttggcaagccacaaacagcggacagaaaatcatgagcaaataatccat

SEQ ID NO: 399 - DNA, 63 bp, Hordeum vulgare
cccataatataagagcatgtttttacactacactagtgtaaaaaaacgctcttatattatggg

SEQ ID NO: 400 - DNA, 63 bp, Artificial Sequence
  Description of Artificial Sequence Synthetic oligonucleotide
cccataatataagagcatgtttttacactacacttgtgtaaaaaaacgctcttatattatgga

SEQ ID NO: 401 - DNA, 63 bp, Hordeum vulgare
cccataatataagagcgtttttttacactagtgtagtgtaaaaacatgctcttatattatggg

SEQ ID NO: 402 - DNA, 63 bp, Artificial Sequence
  Description of Artificial Sequence Synthetic oligonucleotide
tccataatataagagcgtttttttacacaagtgtagtgtaaaaacatgctcttatattatggg

SEQ ID NO: 403 - DNA, 63 bp, Rattus norvegicus
gtaacgaggtaacggccgtaatgcctggaacccgagactgacggtagcagggagcgtggcaag

SEQ ID NO: 404 - DNA, 63 bp, Artificial Sequence
  Description of Artificial Sequence Synthetic oligonucleotide
gtaacgcggtaacggccgtaatgcctggaacccgagactgacggtagcagggagcgtggcaag

SEQ ID NO: 405 - DNA, 63 bp, Rattus norvegicus
cttgccacgctccctgctaccgtcagtctcgggttccaggcattacggccgttacctcgttac

SEQ ID NO: 406 - DNA, 63 bp, Artificial Sequence
  Description of Artificial Sequence Synthetic oligonucleotide
cttgccacgctccctgctaccgtcagtctcgggttccaggcattacggccgttaccgcgttac

SEQ ID NO: 407 - DNA, 63 bp, Artificial Sequence
  Description of Artificial Sequence Synthetic oligonucleotide
atgcaaaaaactttcagaccattccaattaacgagaggctttacctcttccgtaaaaaacttc

SEQ ID NO: 408 - protein, 21 aa, Artificial Sequence
  Description of Artificial Sequence Synthetic peptide
MQKTFRPFQLTRGFTSSVKNF

SEQ ID NO: 409 - DNA, 63 bp, Artificial Sequence
  Description of Artificial Sequence Synthetic oligonucleotide
gaagttttttacggaagaggtaaagcctctcgttaattggaatggtctgaaagttttttgcat

SEQ ID NO: 410 - DNA, 63 bp, Artificial Sequence
  Description of Artificial Sequence Synthetic oligonucleotide
atggccgcacgcctcgcgctggtggcggcgctcctgtgctccggtgccacggccgccgcggcg

SEQ ID NO: 411 - protein, 21 aa, Artificial Sequence
  Description of Artificial Sequence Synthetic peptide
MAARLALVAALLCSGATAAAA

SEQ ID NO: 412 - DNA, 63 bp, Artificial Sequence
  Description of Artificial Sequence Synthetic oligonucleotide
cgccgcggcggccgtggcaccggagcacaggagcgccgccaccagcgcgaggcgtgcggccat

SEQ ID NO: 413 - DNA, 63 bp, Artificial Sequence
  Description of Artificial Sequence Synthetic oligonucleotide
atgaaaattagatgcttaatttgtctcgtaattgttcttacgatcattcatttgtctcaagct

SEQ ID NO: 414 - protein, 21 aa, Artificial Sequence
  Description of Artificial Sequence Synthetic peptide
MKIRCLICLVIVLTIIHLSQA

SEQ ID NO: 415 - DNA, 63 bp, Artificial Sequence
  Description of Artificial Sequence Synthetic oligonucleotide
agcttgagacaaatgaatgatcgtaagaacaattacgagacaaattaagcatctaattttcat

SEQ ID NO: 416 - DNA, 1023 bp, Arabidopsis thaliana
  CDS (1)..(1023)
atgttcagatctatgctcgtccgatcttctgcctccgcgaagcaggcggttatccgccgtagcttctcctccggctccgtccccgagcgtaaagtcgccatccttggtgccgccggtggaattggtcagcctcttgctctcctcatgaagcttaatcctcttgtctcttccctctccctctacgatatcgctaacactcctggagttgctgctgatgttggtcacatcaacaccagatctgaggttgttggatacatgggcgatgataacttggccaaagctcttgaaggagctgatctcgttatcattccagctggtgtaccaaggaagcctggtatgacccgtgacgatcttttcaacattaatgctggaattgtcaagaacctttgcactgccatcgccaagtactgcccacatgcgcttattaatatgatcagcaaccctgtgaactctactgttccaattgcagctgagatatttaagaaggctggtatgtacgatgaaaagaaattgtttggtgttaccactcttgacgtcgtcagggccaggactttctatgctggaaaggcaaatgtcccagttgcagaagttaatgttccggtgattggtggtcatgctggggttactattctccctctcttctctcaggcaactcctcaagccaacttgtcaagtgacatacttaccgcccttactaagcgtacccaagatggaggtacagaagtcgtggaggcaaaagcaggaaaaggttcagctacattgtccatggcctatgccggagcattgttcgctgatgcatgcttgaaaggactcaacggtgttccagatgtcatagaatgctcatacgtgcaatctacaatcaccgagcttcctttctttgcctcgaaggtgaggttggggaagaatggtgtggaggaggttcttgacttgggaccactctcagactttgagaaggaaggcttggaagcattgaagccagaactcaagtcctccatagaaaagggagtcaagtttgccaaccag

SEQ ID NO: 417 - protein, 341 aa, Arabidopsis thaliana
MFRSMLVRSSASAKQAVIRRSFSSGSVPERKVAILGAAGGIGQPLALLMKLNPLVSSLSLYDIANTPGVAADVGHINTRSEVVGYMGDDNLAKALEGADLVIIPAGVPRKPGMTRDDLFNINAGIVKNLCTAIAKYCPHALINMISNPVNSTVPIAAEIFKKAGMYDEKKLFGVTTLDVVRARTFYAGKANVPVAEVNVPVIGGHAGVTILPLFSQATPQANLSSDILTALTKRTQDGGTEVVEAKAGKGSATLSMAYAGALFADACLKGLNGVPDVIECSYVQSTITELPFFASKVRLGKNGVEEVLDLGPLSDFEKEGLEALKPELKSSIEKGVKFANQ

SEQ ID NO: 418 - DNA, 1023 bp, Artificial Sequence
  Description of Artificial Sequence Synthetic polynucleotide
atgttcagatctatgctcgtccgatcttctgcctccgcgaagcaggcggttatccgccgtagcttctcctccggctccgtccccgagcgtaaagtcgccatccttggtgccgccggtggaattggtcagcctcttgctctcctcatgaagcttaatcctcttgtctcttccctctccctctacgatatcgctaacactcctggagttgctgctgatgttggtcacatcaacaccagatctgaggttgttggatacatgggcgatgataacttggccaaagctcttgaaggagctgatctcgttatcattccagctggtgtaccaaggaagcctggtatgacccgtgacgatcttttcaacattaatgctggaattgtcaagaacctttgcactgccatcgccaagtactgcccacatgcgcttattaatatgatcagcaaccctgtgaactctactgttccaattgcagctgagatatttaagaaggctggtatgtacgatgaaaagaaattgtttggtgttaccactcttgacgtcgtcagggccaggactttctatgctggaaaggcaaatgtcccagttgcagaagttaatgttccggtgattggtggtcatgctggggttactattctccctctcttctctcaggcaactcctcaagccaacttgtcaagtgacatacttaccgcccttactaagcgtacccaagatggaggtacagaagtcgtggaggcaaaagcaggaaaaggttcagctacattgtccatggcctatgccggagcattgttcgctgatgcatgcttgaaaggactcaacggtgttccagatgtcatagaatgctcatacgtgcaatctacaatcaccgagtttcctttctttgcctcgaaggtgaggttggggaagaatggtgtggaggaggttcttgacttgggaccactctcagactttgagaaggaaggcttggaagcattgaagccagaactcaagtcctccatagaaaagggagtcaagtttgccaaccag

SEQ ID NO: 419 - protein, 341 aa, Artificial Sequence
  Description of Artificial Sequence Synthetic polypeptide
MFRSMLVRSSASAKQAVIRRSFSSGSVPERKVAILGAAGGIGQPLALLMKLNPLVSSLSLYDIANTPGVAADVGHINTRSEVVGYMGDDNLAKALEGADLVIIPAGVPRKPGMTRDDLFNINAGIVKNLCTAIAKYCPHALINMISNPVNSTVPIAAEIFKKAGMYDEKKLFGVTTLDVVRARTFYAGKANVPVAEVNVPVIGGHAGVTILPLFSQATPQANLSSDILTALTKRTQDGGTEVVEAKAGKGSATLSMAYAGALFADACLKGLNGVPDVIECSYVQSTITEFPFFASKVRLGKNGVEEVLDLGPLSDFEKEGLEALKPELKSSIEKGVKFANQ

SEQ ID NO: 420 - DNA, 1023 bp, Arabidopsis thaliana
ctggttggcaaacttgactcccttttctatggaggacttgagttctggcttcaatgcttccaagccttccttctcaaagtctgagagtggtcccaagtcaagaacctcctccacaccattcttccccaacctcaccttcgaggcaaagaaaggaagctcggtgattgtagattgcacgtatgagcattctatgacatctggaacaccgttgagtcctttcaagcatgcatcagcgaacaatgctccggcataggccatggacaatgtagctgaaccttttcctgcttttgcctccacgacttctgtacctccatcttgggtacgcttagtaagggcggtaagtatgtcacttgacaagttggcttgaggagttgcctgagagaagagagggagaatagtaaccccagcatgaccaccaatcaccggaacattaacttctgcaactgggacatttgcctttccagcatagaaagtcctggccctgacgacgtcaagagtggtaacaccaaacaatttcttttcatcgtacataccagccttcttaaatatctcagctgcaattggaacagtagagttcacagggttgctgatcatattaataagcgcatgtgggcagtacttggcgatggcagtgcaaaggttcttgacaattccagcattaatgttgaaaagatcgtcacgggtcataccaggcttccttggtacaccagctggaatgataacgagatcagctccttcaagagctttggccaagttatcatcgcccatgtatccaacaacctcagatctggtgttgatgtgaccaacatcagcagcaactccaggagtgttagcgatatcgtagagggagagggaagagacaagaggattaagcttcatgaggagagcaagaggctgaccaattccaccggcggcaccaaggatggcgactttacgctcggggacggagccggaggagaagctacggcggataaccgcctgcttcgcggaggcagaagatcggacgagcatagatctgaacat

SEQ ID NO: 421 - DNA, 1023 bp, Artificial Sequence
  Description of Artificial Sequence Synthetic polynucleotide
ctggttggcaaacttgactcccttttctatggaggacttgagttctggcttcaatgcttccaagccttccttctcaaagtctgagagtggtcccaagtcaagaacctcctccacaccattcttccccaacctcaccttcgaggcaaagaaaggaaactcggtgattgtagattgcacgtatgagcattctatgacatctggaacaccgttgagtcctttcaagcatgcatcagcgaacaatgctccggcataggccatggacaatgtagctgaaccttttcctgcttttgcctccacgacttctgtacctccatcttgggtacgcttagtaagggcggtaagtatgtcacttgacaagttggcttgaggagttgcctgagagaagagagggagaatagtaaccccagcatgaccaccaatcaccggaacattaacttctgcaactgggacatttgcctttccagcatagaaagtcctggccctgacgacgtcaagagtggtaacaccaaacaatttcttttcatcgtacataccagccttcttaaatatctcagctgcaattggaacagtagagttcacagggttgctgatcatattaataagcgcatgtgggcagtacttggcgatggcagtgcaaaggttcttgacaattccagcattaatgttgaaaagatcgtcacgggtcataccaggcttccttggtacaccagctggaatgataacgagatcagctccttcaagagctttggccaagttatcatcgcccatgtatccaacaacctcagatctggtgttgatgtgaccaacatcagcagcaactccaggagtgttagcgatatcgtagagggagagggaagagacaagaggattaagcttcatgaggagagcaagaggctgaccaattccaccggcggcaccaaggatggcgactttacgctcggggacggagccggaggagaagctacggcggataaccgcctgcttcgcggaggcagaagatcggacgagcatagatctgaacat

SEQ ID NO: 422 - DNA, 63 bp, Artificial Sequence
  Description of Artificial Sequence Synthetic oligonucleotide
atgaaactatttcttttactagttatctctgcttcaatgcttattgatggcttatttaatgct

SEQ ID NO: 423 - protein, 21 aa, Artificial Sequence
  Description of Artificial Sequence Synthetic peptide
MKLFLLLVISASMLIDGLFNA

SEQ ID NO: 424 - DNA, 63 bp, Artificial Sequence; identical to SEQ ID NO: 410
  Description of Artificial Sequence Synthetic oligonucleotide

SEQ ID NO: 425 - protein, 21 aa, Artificial Sequence
  Description of Artificial Sequence Synthetic peptide
MAARLALVAALLCSGATAAAA

SEQ ID NO: 426 - DNA, 63 bp, Rattus sp.
  CDS (1)..(63)
atgctgtccgctctcgcccgtcctgtcggtgccgctctccgccgcagcttcagcacttcagcc

SEQ ID NO: 427 - protein, 21 aa, Rattus sp.
MLSALARPVGAALRRSFSTSA

SEQ ID NO: 428 - DNA, 63 bp, Artificial Sequence
  Description of Artificial Sequence Synthetic oligonucleotide
atgctgtccgttctcgcccgtcctgtcggtgccgctctccgccgcagcttcagcacttcagcc

SEQ ID NO: 429 - protein, 21 aa, Artificial Sequence
  Description of Artificial Sequence Synthetic peptide
MLSVLARPVGAALRRSFSTSA

SEQ ID NO: 430 - DNA, 63 bp, Artificial Sequence
  Description of Artificial Sequence Synthetic oligonucleotide
atgctgtccgctctcgcccgtcctgtcggtgccgctcaccgccgcagcttcagcacttcagcc

SEQ ID NO: 431 - protein, 21 aa, Artificial Sequence
  Description of Artificial Sequence Synthetic peptide
MLSALARPVGAAHRRSFSTSA

SEQ ID NO: 432 - DNA, 63 bp, Artificial Sequence
  Description of Artificial Sequence Synthetic oligonucleotide
atgctgtccgctctcgcccgtcctgtcggtgccgctctccgccgaagcttcagcacttcagcc

SEQ ID NO: 433 - protein, 21 aa, Artificial Sequence
  Description of Artificial Sequence Synthetic peptide
MLSALARPVGAALRRSFSTSA

SEQ ID NO: 434 - protein, 21 aa, Artificial Sequence
  Description of Artificial Sequence Synthetic peptide
MLSALARPVGAALAKSFSTSA

SEQ ID NO: 435 - protein, 21 aa, Artificial Sequence
  Description of Artificial Sequence Synthetic peptide
MLSALAKPVGAALAKSFSTSA

SEQ ID NO: 436 - DNA, 63 bp, Artificial Sequence
  Description of Artificial Sequence Synthetic oligonucleotide
atgctgtccgctctcgccaaacctgtcggtgccgctctcgctcgaagcttcagcacttcagcc

SEQ ID NO: 437 - DNA, 63 bp, Artificial Sequence
  Description of Artificial Sequence Synthetic oligonucleotide
atgctgtccgctctcgccaaacctgtcggtgccgctctcgcccgaagcttcagcacttcagcc

SEQ ID NO: 438 - DNA, 63 bp, Artificial Sequence
  Description of Artificial Sequence Synthetic oligonucleotide
atgctgtccgctctcgccaaacctgtcggtgccgctctcgcacgaagcttcagcacttcagcc

SEQ ID NO: 439 - DNA, 63 bp, Artificial Sequence
  Description of Artificial Sequence Synthetic oligonucleotide
atgctgtccgctctcgccaaacctgtcggtgccgctctcgcgcgaagcttcagcacttcagcc

SEQ ID NO: 440 - DNA, 63 bp, Unknown; identical to SEQ ID NO: 439
  Description of Unknown Malate dehydrogenase oligonucleotide

SEQ ID NO: 441 - DNA, 63 bp, Artificial Sequence
  Description of Artificial Sequence Synthetic oligonucleotide
atgctgtccgctctcgccaaacctgtcggtgccgcgctcgcgcgaagcttcagcacttcagcc

SEQ ID NO: 442 - DNA, 63 bp, Artificial Sequence
  Description of Artificial Sequence Synthetic oligonucleotide
atgctgtccgctctcgccaagcctgtcggtgccgctctcgctcgaagcttcagcacttcagcc

SEQ ID NO: 443 - DNA, 63 bp, Artificial Sequence
  Description of Artificial Sequence Synthetic oligonucleotide
atgctgtccgctctcgccaagcctgtcggtgccgctctcgcccgaagcttcagcacttcagcc

SEQ ID NO: 444 - DNA, 63 bp, Artificial Sequence
  Description of Artificial Sequence Synthetic oligonucleotide
atgctgtccgctctcgccaagcctgtcggtgccgctctcgcacgaagcttcagcacttcagcc

SEQ ID NO: 445 - DNA, 63 bp, Artificial Sequence
  Description of Artificial Sequence Synthetic oligonucleotide
atgctgtccgctctcgccaagcctgtcggtgccgctctcgcgcgaagcttcagcacttcagcc

SEQ ID NO: 446 - DNA, 63 bp, Artificial Sequence
  Description of Artificial Sequence Synthetic oligonucleotide
atgctgtccgctctcgcccgtcctgtcggtgccgctctcgctaaaagcttcagcacttcagcc

SEQ ID NO: 447 - DNA, 63 bp, Artificial Sequence
  Description of Artificial Sequence Synthetic oligonucleotide
atgctgtccgctctcgcccgtcctgtcggtgccgctctcgccaaaagcttcagcacttcagcc

SEQ ID NO: 448 - DNA, 63 bp, Artificial Sequence
  Description of Artificial Sequence Synthetic oligonucleotide
atgctgtccgctctcgcccgtcctgtcggtgccgctctcgcaaaaagcttcagcacttcagcc

SEQ ID NO: 449 - DNA, 63 bp, Artificial Sequence
  Description of Artificial Sequence Synthetic oligonucleotide
atgctgtccgctctcgcccgtcctgtcggtgccgctctcgcgaaaagcttcagcacttcagcc

SEQ ID NO: 450 - DNA, 63 bp, Artificial Sequence
  Description of Artificial Sequence Synthetic oligonucleotide
atgctgtccgctctcgcccgtcctgtcggtgccgctctcgctaagagcttcagcacttcagcc

SEQ ID NO: 451 - DNA, 63 bp, Artificial Sequence
  Description of Artificial Sequence Synthetic oligonucleotide
atgctgtccgctctcgcccgtcctgtcggtgccgctctcgccaagagcttcagcacttcagcc

SEQ ID NO: 452 - DNA, 63 bp, Artificial Sequence
  Description of Artificial Sequence Synthetic oligonucleotide
atgctgtccgctctcgcccgtcctgtcggtgccgctctcgcaaagagcttcagcacttcagcc

SEQ ID NO: 453 - DNA, 63 bp, Artificial Sequence
  Description of Artificial Sequence Synthetic oligonucleotide
atgctgtccgctctcgcccgtcctgtcggtgccgctctcgcgaagagcttcagcacttcagcc

SEQ ID NO: 454 - DNA, 63 bp, Artificial Sequence
  Description of Artificial Sequence Synthetic oligonucleotide
atgctgtccgctctcgccaaacctgtcggtgccgctctcgctaaaagcttcagcacttcagcc

SEQ ID NO: 455 - DNA, 63 bp, Artificial Sequence
  Description of Artificial Sequence Synthetic oligonucleotide
atgctgtccgctctcgccaaacctgtcggtgccgctctcgccaaaagcttcagcacttcagcc

SEQ ID NO: 456 - DNA, 63 bp, Artificial Sequence
  Description of Artificial Sequence Synthetic oligonucleotide
atgctgtccgctctcgccaaacctgtcggtgccgctctcgcaaaaagcttcagcacttcagcc

SEQ ID NO: 457 - DNA, 63 bp, Artificial Sequence
  Description of Artificial Sequence Synthetic oligonucleotide
atgctgtccgctctcgccaaacctgtcggtgccgctctcgcgaaaagcttcagcacttcagcc

SEQ ID NO: 458 - DNA, 63 bp, Artificial Sequence
  Description of Artificial Sequence Synthetic oligonucleotide
atgctgtccgctctcgccaagcctgtcggtgccgctctcgctaaaagcttcagcacttcagcc

SEQ ID NO: 459 - DNA, 63 bp, Artificial Sequence
  Description of Artificial Sequence Synthetic oligonucleotide
atgctgtccgctctcgccaagcctgtcggtgccgctctcgccaaaagcttcagcacttcagcc

SEQ ID NO: 460 - DNA, 63 bp, Artificial Sequence
  Description of Artificial Sequence Synthetic oligonucleotide
atgctgtccgctctcgccaagcctgtcggtgccgctctcgcaaaaagcttcagcacttcagcc

SEQ ID NO: 461 - DNA, 63 bp, Artificial Sequence
  Description of Artificial Sequence Synthetic oligonucleotide
atgctgtccgctctcgccaagcctgtcggtgccgctctcgcgaaaagcttcagcacttcagcc

SEQ ID NO: 462 - DNA, 63 bp, Artificial Sequence
  Description of Artificial Sequence Synthetic oligonucleotide
atgctgtccgctctcgccaaacctgtcggtgccgctctcgctaagagcttcagcacttcagcc

SEQ ID NO: 463 - DNA, 63 bp, Artificial Sequence
  Description of Artificial Sequence Synthetic oligonucleotide
atgctgtccgctctcgccaaacctgtcggtgccgctctcgccaagagcttcagcacttcagcc

SEQ ID NO: 464 - DNA, 63 bp, Artificial Sequence
  Description of Artificial Sequence Synthetic oligonucleotide
atgctgtccgctctcgccaaacctgtcggtgccgctctcgcaaagagcttcagcacttcagcc

SEQ ID NO: 465 - DNA, 63 bp, Artificial Sequence
  Description of Artificial Sequence Synthetic oligonucleotide
atgctgtccgctctcgccaaacctgtcggtgccgctctcgcgaagagcttcagcacttcagcc

SEQ ID NO: 466 - DNA, 63 bp, Artificial Sequence
  Description of Artificial Sequence Synthetic oligonucleotide
atgctgtccgctctcgccaagcctgtcggtgccgctctcgctaagagcttcagcacttcagcc

SEQ ID NO: 467 - DNA, 63 bp, Artificial Sequence
  Description of Artificial Sequence Synthetic oligonucleotide
atgctgtccgctctcgccaagcctgtcggtgccgctctcgccaagagcttcagcacttcagcc

SEQ ID NO: 468 - DNA, 63 bp, Artificial Sequence
  Description of Artificial Sequence Synthetic oligonucleotide
atgctgtccgctctcgccaagcctgtcggtgccgctctcgcaaagagcttcagcacttcagcc

SEQ ID NO: 469 - DNA, 63 bp, Artificial Sequence
  Description of Artificial Sequence Synthetic oligonucleotide
atgctgtccgctctcgccaagcctgtcggtgccgctctcgcgaagagcttcagcacttcagcc

SEQ ID NO: 470 - DNA, 62 bp, Artificial Sequence
  Description of Artificial Sequence Synthetic oligonucleotide
ttcagatccgcgcttgtccgatcctccgcctggcgaagcagtcgcttctccgccgcagcttc

(19) United States
(12) Patent Application Publication
(10) Pub. No.: US 2011/0172975 A1
(43) Pub. Date: Jul. 14, 2011

(54) GENERATION AND REPRODUCTION OF DNA SEQUENCES AND ANALYSIS OF POLYMORPHISMS AND MUTATIONS BY USING ERROR-CORRECTING CODES

(75) Inventors: Marcio de Castro SILVA FILHO, Piracicaba (BR); Reginaldo PALAZZO, JR., Campinas (BR); Andrea Santos LEITE DA ROCHA, Campinas (BR); Luzinete Cristina BONANI DE FARIA, Campinas (BR); Joao Henrique KLEINSCHMIDT, Santo Andre (BR)

(73) Assignees: UNIVERSITY OF SAO PAULO, Sao Paulo (BR); STATE UNIVERSITY OF CAMPINAS, Sao Paulo (BR); FUNDACAO DE AMPARO A PESQUISA DO ESTADO DE SAO PAULO, Sao Paulo (BR)

(21) Appl. No.: 12/859,697

(22) Filed: Aug. 19, 2010

Related U.S. Application Data
(60) Provisional application No. 61/272,129, filed on Aug. 19, 2009.

(51) Int. Cl. G06F 17/10; C07H 21/04

(52) U.S. Cl. 703/2; 536/23.1

(57) ABSTRACT

The present invention relates to a method that uses error-coding codes for validating polymorphisms and mutations/alterations in a DNA sequence which encodes a polypeptide sequence. The present invention also relates to a digital communication system for carrying out the method, employing a model for the biological coding system which resembles the most efficient digital communication. The method and digital communication system may be useful for the predictive analysis of diseases originated by mutations or polymorphisms in genes.

### CROSS REFERENCE TO RELATED APPLICATIONS

This application claims priority under 35 U.S.C. §119(e) to U.S. Provisional Patent Application No. 61/272,129 filed on Aug. 19, 2009, the entire disclosure of which is incorporated by reference herein in its entirety.

### SEQUENCE LISTING

The instant application contains a Sequence Listing which has been submitted via EFS-Web and is hereby incorporated by reference in its entirety. Said ASCII copy, created on Jul. 29, 2010, is named Q119440.txt and is 232,826 bytes in size.

### FIELD OF THE INVENTION

The present invention refers to a systematic procedure that uses error-correcting codes for the generation and reproduction of DNA sequences. Substitutions of either nucleotide(s) or amino acid(s) in these sequences provide the means to realize the analysis of either polymorphism(s) or mutation(s).

More specifically, this method is useful in the investigation of new functionalities associated with DNA sequences regarding, inter alia, commercial and scientific purposes.

### BACKGROUND OF THE INVENTION

Certain patents and printed publications have been referred to in the present disclosure, the teachings of which are hereby each incorporated in their respective entireties by reference.

Error-correcting codes are used whenever one wants either to transmit or to store information. A well known example is the biological system which stores and transmits information by use of the genetic code. FIG. 1 illustrates the existing similarities between the communication system central dogma and the molecular biology central dogma where the following associations are depicted:

1) In a communication system the information is processed in the transmitter whereas in a biological system the DNA in the nucleus is responsible for that;

2) The transcription process has the purpose of selecting the information to be transmitted. During this process errors may occur (for instance: mutations or interferences) leading to a possible alteration in the information content. From the communication system point of view, we may visualize the interference process in the transcription and translation as random errors being introduced by the channel;

3) The receiver is the place where the transmitted information is directed. In a biological system, the information to be transmitted is the protein and the receiver may be one of the organelles (mitochondrion, endoplasmic reticulum and chloroplast).

From the similarities between the flow of information in the biological system and in the communication system, several models were proposed. Yockey, [15], proposes a model of a digital communication system which represents the one associated with a genetic expression. Forsdyke, [16-17], considered the possibility that the introns could be the parity-check digits associated with the exons. On the other hand, Rzeszowska-Wolny, [30], proposes that an appropriated arrangement of the DNA in nucleosomes may be relevant to the operationality of these systems. Liebovitch, [18], proposes a procedure that makes it possible to determine if a certain type of error-correcting code is or is not present in a DNA sequence. Rosen, [19], presented a method for the detection of linear block codes that explains the possibility of insertions and deletions in the DNA sequences. Battail, [20], argues on the existence of nested codes in the DNA, since the length of the human genome is far greater than that necessary to specify the characteristics of each person. May et. al., [21], propose the use of block and convolutional codes in the initialization process of the translation in procarionte organisms. Mac Donnaill, [29], proposed a parity-check code related to the composition of nucleotides. Sánchez et. al., [31], proposed the construction of a vector space associated with the genetic code having as a mathematical structure the Galois field with 64 elements identifying each amino acid with a binary sequence, providing a geometric characterization associated with the genetic code. The approach of the two latter papers is solely related with the genetic code.

A question always present in the majority of the research being done on genomic coding is the following: Is there any form of error-correcting code underlying the DNA structure? However, the previous works were not able to furnish the fundamentals on the existence of error-correcting codes in the DNA sequences.

To the best of our knowledge there is no known mathematical method able to foresee mutations in DNA sequences, either through biological evolution, in vitro evolution or by genetic manipulation.

### BRIEF DESCRIPTION OF THE INVENTION

The present invention addresses in a positive manner, an answer to this question having as premises the fact that if the genome consists of regions which include exons, introns, promoters, repetitive DNA, and so on, and that each one of these regions may be reproduced by a specific code, then the genome consists of nested codes, that is, instead of looking at all the genome we have to focus on its parts. One possible interpretation of Shannon's Channel Coding Theorem, regarding the flow of information from the source to the sink, is that the mutual information of the discrete channel, (FIG. 2), be as close as possible to the entropy of the source. To achieve this goal, an error-correcting code is used. Therefore, the transmitter in the digital communication system model consists of two cascade blocks, one block associated with an encoder and the other one associated with a modulator (FIG. 2).

The biological coding system of the present invention is characterized in one aspect as follows: The codeword at the encoder output is related to the mature mRNA, whereas the output of the modulator is related to the protein. Although the matching, by the tRNA, of each codon in the mature mRNA strand with its corresponding anticodon from the genetic code is well known in the biological context, it needs a mathematical characterization. However, in a digital communication system context this very same process exists and it is called matched mapping. This mathematical property, in addition to implying that the underlying algebraic structure of the encoder and the signal constellation are the same up to an isomorphism, guarantees the least overall system complexity.

The class of codes satisfying this property is known as geometrically uniform codes, and an important subclass is the G-linear codes, where G denotes an algebraic group. Therefore, the encoder consists of a mapper and an encoder of an error-correcting code. The modulator consists of the genetic code, the tRNA and the rRNA, (FIG. 3). The genetic code may be viewed as a signal constellation, where each codon is considered as a signal in the signal constellation, the tRNA realizes the matched mapping, whereas the rRNA behaves as a digital signal processor. We call the attention to the fact that to the best of our knowledge, the characterization used in the present proposal for modelling a biological coding system was not considered previously in the open literature. Therefore, we do not know about the existence of any related technology to the present invention.

The expression “error correcting code” should be understood as a code with the ability to detect the presence of errors caused by noise or other impairments or mutations during transmission from the transmitter/nucleus to the receiver/organelle. It has the additional ability to reconstruct the original data, error-free. However, there are classes of codes with the purpose of detecting errors only which are less complex than the error-correcting codes.

Historically, the error-correcting codes have been classified as tree codes where the two main classes are the block codes and the trellis codes, in general either over Galois field or ring extensions. Each one of these classes may be further classified as linear and nonlinear. The class of linear trellis codes is well known in the literature as the class of convolutional codes. The distinguishing feature for this particular classification is the presence or absence of memory in the encoder [4], [5], [32] and [33].

An encoder of a block code accepts information in successive k-bit blocks; for each block, it adds n−k redundant bits that are algebraically related to the k message bits, thereby producing an overall encoded block of n bits, where n>k.

In a convolutional code, the encoding operation may be viewed as the discrete time convolution of the input sequence with the impulse response of the encoder. The duration of the impulse response equals the memory of the encoder. Accordingly, the encoder for a convolutional code operates on the incoming message sequence, using a “sliding window” equal in duration to its own memory. This, in turn, means that in a convolutional code, unlike a block code, the channel encoder accepts message bits as a continuous sequence and thereby generates a continuous sequence of encoded bits at a higher rate.

Suitable examples of error correcting codes according to the present invention include, without limitation, Hamming Codes, BCH codes, Alternant codes, Goppa codes, Golay code, Group codes, Reed-Muller code, Hagelbarger code, Lexicographic code, Low-density parity-check code, Turbo code, Berger code, Erasure codes, such as Tornado codes, LT codes, Online codes, Raptor codes, Reed-Solomon codes. Additional examples of suitable error correcting codes include the teachings of U.S. Pat. No. 4,908,827, US 2005/0193312, U.S. Pat. No. 7,162,678, which is incorporated by its entirety herein by references.

In a preferred aspect, the present invention uses BCH codes. In general, let S be a set of geometrically uniform signal set (GU) (lattices, Slepian codes, G-linear codes, etc) consisting of a set of points in an n-dimensional Euclidean space having a transitive group of symmetries, that is, given any two points s₁ and s₂ in S, there exists na isometry that takes s₁ into s₂, leaving S invariant [27] and [35]. A generator group U(S) of S is a subgroup of the symmetry group of S, denoted by Γ(S), which is minimally sufficient to generate S from an initial point s₀ in S. A geometrically uniform partition S/S′ is a partition of a GU signal set with generator group U(S) which is induced by a normal subgroup U′ of U(S). The elements of the partition are the subsets of S corresponding to the cosets of U′ in U(S). Let G be an abstract group isomorphic to U(S)/U′. An isometrically labelling m: G→S/S′ is a labelling of points of S by elements of G induced by the isomorphism between G and U(S)/U′.

Let G be a group, I an index set, C a code (subgroup of the labelling space G^{I}), a geometrically unform partition S/S′ is a labelling m: G^{I}→(S/S′)^{I} (extension of the isometric labelling m: G→S/S′). Hence, a generalized coset code, denoted by C(S/S′; C), is a disjoint union of the set of sequences of subsets m(c)={m(cₖ), k in I}, c in C, that is, m(c) is the sequence of subsets selected by the labelling sequence c in C via the labelling mapping m, [27] and [35].

With the necessity of reduction time and costs with laboratorial tests, the present invention proposes a mathematical approach capable of generating and reproducing DNA sequences, leading to a methodology to realize mutational analysis in these sequences (proteins, targeting sequences, repetitive DNA, introns, protein motifs, hormones, proteins of the bacteria and viruses, proteins of the plasmid, ncRNA, etc), implying in a considerable reduction in extensive laboratorial tests. This method may be applied in drugs design, and research aiming at creating new functionalities to specific DNA sequences by use of mutations as far as the commercial and scientific needs are concerned.

Furthermore, the invention is useful for generating mutations with protein functional gain, with greater stability, greater substrate affinity, greater specific activity, etc.

The present invention aims at the characterization of a mathematical method for the determination and validation of polymorphisms and mutations/alterations in DNA sequences which encode polypeptide sequences. This invention also provides ways to analyze which, among the mutations, will be synonymous, critical and radical to the system in which it interfere, with applications in genetic engineering.

According to the present invention, a systematic procedure provides the necessary elements for the validation of the mutations in DNA sequences by use of the following nonlimiting steps:

1. Determine the alphabet and the code mathematical structure;

2. Determine the Galois ring extension;

3. Selection of a primitive polynomial related to the extension;

4. Determine the field extension;

5. Determine the ring extension (Only for the ring case);

6. Determine the group of units;

7. Determine the generator polynomial g(x), the generator matrix G(x) and its transpose G^{T}(x);

8. Determine the generator polynomial of the dual code h(x), the generator matrix H(x) and its transpose H^{T}(x);

9. Label the DNA sequences using the code alphabet;

10. Check if the DNA sequence is a codeword of G(x);

11. Label all the codewords by use of the alphabet of the genetic code;

12. Compare the code words generated by the code with the original DNA sequence;

13. Define the labelling of the DNA sequence and show where the differences are located.

In the present invention, we are using the expression nucleotide errors to mean the differences being pointed out by the error-correcting code in those referred positions.

The present invention also shows in terms of tables the DNA sequences and their corresponding code words with the respectives mappings and labellings.

The present invention also allows generating new sequences with functionalities similar to those of the DNA sequences.

One object of the present invention is to generate DNA sequences by use of error-correcting codes over ring and field, providing in this way the identification and classification of the DNA sequences (cyclic linear sequences, noncyclic linear sequences, cyclic nonlinear sequences, and noncyclic nonlinear sequences) according to its mathematical structures. This systematic procedure allows the evaluation of mutations, however, by preserving the mathematical structure of the error-correcting code. This procedure allows the realization of screenings of mutants with the objective to improve the properties of the proteic sequences. This process allows the realization and selection of mutations to be biologically tested.

An additional object of the present invention is the reproduction of DNA sequences (cyclic linear sequences) by use of simple linear feedback shift-register.

An additional object of the present invention is the generation of DNA sequences (noncyclic linear sequences) by use of the generator matrix of the corresponding cyclic linear error-correcting codes with the inclusion of new columns or even the deletion of some previous columns.

An additional object of the present invention is the reproduction of the DNA sequences (cyclic nonlinear sequences) by means of the composition between Boolean functions and linear error-correcting codes.

Still another object of the present invention is the reproduction of DNA sequences (noncyclic nonlinear sequences) by the composition between Boolean functions and nonlinear error-correcting codes.

An additional object of the present invention refers to the use of the mapping between the genetic code alphabet and the error-correcting code, from the permutations between the nucleotide set (A,C,G,T) and the code alphabet (0, 1, 2, 3) for ring and (0,1,a,b) for field. This mapping infer about the secondary structure inherent to the DNA sequences. Hence, it is possible to correlate the tridimensional structure of the proteins with the algebraic structures derived from Boolean functions. This procedure infers in a possible utilization of mathematical structures of the error-correcting code in the identification of the ligand and receptors of proteins and peptides.

An additional object of the present invention refers to the validation of the mutation(s) in a DNA sequence which point the position out and the amino acid which will or will not be replaced in order to guarantee the information content of this sequence.

An additional object of the present invention is to provide a low cost computational procedure for the manipulation of amino acid changes in preselected positions in the DNA sequences, according to the interest of the application. The method in consideration allows either a scientist or a lab technician to analyze the consequences of the mutations considered.

An additional object of the present invention is to infer if organelle protein import will or will not occur by the manipulation of the amino acids in the targeting sequences.

An additional object of the present invention is indicating the code words (DNA sequences) to be utilized in the filogenetic study in order to verify the homology and ancestrality of the analyzed sequences.

An additional object of the present invention is to allow the generation of the mutations with gains on proteins functionality, with greater stability, greater affinity per substrate, and greater specific activities, etc.

Objects and advantages of the invention set forth herein and will also be readily appreciated here from, or may be learned by practice with the invention. These objects and advantages are realized and obtained by means of instrumentalities and combinations pointed out in the specification and claims.

### BRIEF DESCRIPTION OF THE FIGURES

The patent or application file contains at least one drawing executed in color. Copies of this patent or patent application publication with color drawings will be provided by the Office upon request and payment of the necessary fee.

FIG. 1. Molecular biology and communication system central dogmas.

FIG. 2. Communication system model.

FIG. 3. Biological coding system.

FIG. 4. Model of a communication system for the transport of proteins to organelles. (1) Source—In a communication system the source is where the messages is generated. In a biological system, however, the DNA and mRNA are the ones responsible for generating and transmitting information, respectively. (2) Transmitter (Encoder)—The transcription process occurs in the cytosol and its objective is to guarantee the continuity of the genetic information. In this process, errors may occur, and they are called mutations. (3) Channel—it is the means by which the information is transmitted in a communication system, where errors may occur due to interference when considering the message being transmitted. (4) Receiver—it may be interpreted as one of the organelles, for it represents the local where the information is being sent. In this specific case, the information is the targeting sequence.

FIG. 5. Mapper Z₄—Binary representation associated with each one of the labels 0-00; 1-10; 2-11; 3-01. However, the association of the complementary nucleotides A-T and C-G with the labels is what differentiate them. In the case of the label A, any of the nucleotides to reach its complementary has to walk two edges, whereas the remaining ones just one edge. All the permutations associated with label A characterize the code as Z₄-linear; all permutations associated with label B characterize the code as Z₂×Z₂-linear; whereas all the permutations associated with label C characterize the code as Klein-linear.

FIG. 6. Labelling D

FIG. 7. Algebraic representation of a targeting sequence: N. tabacum—Endoplasmic reticulum—Pathogen—and wound-inducible antifungal protein CBP20*—Loci: S72452—The coding region of the genomic DNA of a protein consists of a code word of the G-linear code. This code word is obtained from a BCH code with generator polynomial g₁(x) resulting from the labeling A and of a primitive polynomial p₁(x) with degree r which is used in the Galois ring extension GR(4, r). The complementary strand is generated by a code word obtained from a BCH code with the reciprocal of the previous generator polynomial, denoted by g₁*(x), resulting from the same label and also with the reciprocal of the previous primitive polynomial, denoted by p₁*(x). Note that the transfer RNA (tRNA) realizes the matched mapping between each one of the codons in this sequence with the corresponding amino-acids. Figure discloses SEQ ID NOS 15-18, respectively, in order of appearance.

FIG. 8. Computer Program Flow-Chart

FIG. 9 depicts Table 1 that shows the nucleotide sequence of the coding and non-coding strands of B. napus—Mitochondrial—Malate dehydrogenase*—GI number 899225. Figure discloses SEQ ID NOS 7, 19, 10, 11, 20, and 21, respectively, in order of appearance.

FIG. 10 depicts Table 2 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 22, 23, 16, and 24, respectively, in order of appearance.

FIG. 11A depicts Table 3 that shows the nucleotide sequence of coding strand of A. thaliana—Mitochondrial genome—GI number 26556996. Figure discloses SEQ ID NOS 26, 25, 27, and 28, respectively, in order of appearance.

FIG. 11B depicts Table 3 that shows the nucleotide sequence of the non-coding strand of A. thaliana—Mitochondrial genome—GI number 26556996. Figure discloses SEQ ID NOS 29 and 30, respectively, in order of appearance.

FIG. 12 depicts Table 4 that shows the nucleotide sequence of the coding and non-coding strands of A. thaliana—Mitochondrial—Pathogenesis related protein 4*—GI number 186509758. Figure discloses SEQ ID NOS 32, 31, and 33-36, respectively, in order of appearance.

FIG. 13 depicts Table 5 that shows the nucleotide sequence of the coding and non-coding strands of M. martensii—Endoplasmic reticulum—anti-epilepsy peptide precursor—GI number 16740522. Figure discloses SEQ ID NOS 38, 37, and 39-42, respectively, in order of appearance.

FIG. 14 depicts Table 6 that shows the nucleotide sequence of the coding and non-coding strands of S. cerevisiae—OXA 1—protein motifs—GI number 832917. Figure discloses SEQ ID NOS 44, 43, and 45-48, respectively, in order of appearance.

FIG. 15 depicts Table 7 that shows the nucleotide sequence of the coding and non-coding strands of I. batatas—Mitochondrial—F1-ATPase delta subunit—GI number 217937. Figure discloses SEQ ID NOS 50, 49, and 51-54, respectively, in order of appearance.

FIG. 16 depicts Table 8 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, and 57-60, respectively, in order of appearance.

FIG. 17 depicts Table 9 that shows the nucleotide sequence of the coding and non-coding strands of P. dominulus—Endoplasmic reticulum—Allergen Pol d 5—GI number 51093376. Figure discloses SEQ ID NOS 62, 61, and 63-66, respectively, in order of appearance.

FIG. 18 depicts Table 10 that shows the nucleotide sequence of the coding and non-coding strands of P. dominulus—Endoplasmic reticulum—Allergen Pol d 5—GI number 51093376. Figure discloses SEQ ID NOS 62, 61, 67, 68, 65, and 69, respectively, in order of appearance.

FIG. 19 depicts Table 11 that shows the nucleotide sequence of the coding and non-coding strands of A. thaliana—Mitochondrial—Malate dehydrogenase 1—GI number 30695458. Figure discloses SEQ ID NOS 71, 70, and 72-75, respectively, in order of appearance.

FIG. 20 depicts Table 12 that shows the nucleotide sequence of the coding and non-coding strands of S. cerevisiae—Mitochondrial—54S ribosomal protein—GI number 45269853. Figure discloses SEQ ID NOS 77, 76, and 78-81, respectively, in order of appearance.

FIG. 21 depicts Table 13 that shows the nucleotide sequence of the coding and non-coding strands of H. sapiens—Mitochondrial—ATPase delta-subunit—GI number 12587. Figure discloses SEQ ID NOS 83, 82, and 84-87, respectively, in order of appearance.

FIG. 22 depicts Table 14 that shows the nucleotide sequence of the coding and non-coding strands of I. batatas—Mitochondrial—F1-ATPase delta subunit—GI number 217937—[1]. Figure discloses SEQ ID NOS 50, 49, 88, 89, 53, and 90, respectively, in order of appearance.

FIG. 23 depicts Table 15 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, and 93-96, respectively, in order of appearance.

FIG. 24 depicts Table 16 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 97, 94, 95, and 98, respectively, in order of appearance.

FIG. 25 depicts Table 17 that shows the nucleotide sequence of the coding and non-coding strands of B. taurus—Mitochondria—Aminomethyltransferase—GI number 31343489—[13]. Figure discloses SEQ ID NOS 100, 99, and 101-104, respectively, in order of appearance.

FIG. 26 depicts Table 18 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, and 107-110, respectively, in order of appearance.

FIG. 27 depicts Table 19 that shows the nucleotide sequence of the coding and non-coding strands of C. sinensis—Chloroplast—Chlorophyllase-1—GI number 7328566—[16]. Figure discloses SEQ ID NOS 112, 111, and 113-116, respectively, in order of appearance.

FIG. 28 depicts Table 20 that shows the nucleotide sequence of the coding and non-coding strands of A. thaliana—Mitochondrial—Malate dehydrogenase 1—GI number 30695458—[4]. Figure discloses SEQ ID NOS 71, 70, 117, 118, 74, and 119, respectively, in order of appearance.

FIG. 29 depicts Table 21 that shows the nucleotide sequence of the coding and non-coding strands of A. thaliana—Mitochondrial—Malate dehydrogenase 1—GI number 30695458—[4]. Figure discloses SEQ ID NOS 71, 70, 120, 118, 74, and 121, respectively, in order of appearance.

FIG. 30 depicts Table 22 that shows the nucleotide sequence of the coding and non-coding strands of A. thaliana—Mitochondrial—Malate dehydrogenase 1—GI number 30695458—[4]. Figure discloses SEQ ID NOS 71, 70, 122, 118, 74, and 123, respectively, in order of appearance.

FIG. 31 depicts Table 23 that shows the nucleotide sequence of the coding and non-coding strands of A. thaliana—Mitochondria—Malate dedhydrogenase 2—GI number 15010581—[17]. Figure discloses SEQ ID NOS 125, 124, and 126-129, respectively, in order of appearance.

FIG. 32 depicts Table 24 that shows the nucleotide sequence of the coding and non-coding strands of A. thaliana—Mitochondria—Malate dedhydrogenase 2—GI number 15010581—[17]. Figure discloses SEQ ID NOS 125, 124, 130, 127, 128, and 131, respectively, in order of appearance.

FIG. 33 depicts Table 25 that shows the nucleotide sequence of the coding and non-coding strands of A. thaliana—Mitochondria—Malate dedhydrogenase 2—GI number 15010581—[17]. Figure discloses SEQ ID NOS 125, 124, 132, 127, 128, and 133, respectively, in order of appearance.

FIG. 34 depicts Table 26 that shows the nucleotide sequence of the coding and non-coding strands of R. norvegicus—Mitochondria—ATP synthase subunit delta—GI number 457928—[18]. Figure discloses SEQ ID NOS 135, 134, and 136-139, respectively, in order of appearance.

FIG. 35 depicts Table 27 that shows the nucleotide sequence of the coding and non-coding strands of S. cerevisiae—Mitochondria—ATP sunthase sununit delta—GI number 433619—[19]. Figure discloses SEQ ID NOS 141, 140, and 142-145, respectively, in order of appearance.

FIG. 36 depicts Table 28 that shows the nucleotide sequence of the coding and non-coding strands of Phaseolus vulgaris—Endoplasmatic reticulum—Arcelin 5—GI number—[20]. Figure discloses SEQ ID NOS 147, 146, and 148-151, respectively, in order of appearance.

FIG. 37 depicts Table 29 that shows the nucleotide sequence of the coding and non-coding strands of Phaseolus vulgaris—Endoplasmatic reticulum—Arcelin 5—GI number—[20]. Figure discloses SEQ ID NOS 147, 146, 152, 149, 150, and 153, respectively, in order of appearance.

FIG. 38 depicts Table 30 that shows the nucleotide sequence of the coding and non-coding strands of Phaseolus vulgaris—Endoplasmatic reticulum—Arcelin 5—GI number—[20]. Figure discloses SEQ ID NOS 147, 146, 154, 149, 150, and 155, respectively, in order of appearance.

FIG. 39 depicts Table 31 that shows the nucleotide sequence of the coding and non-coding strands of B. napus—Mitochondrial—Malate dehydrogenase*—GI number 899225. Figure discloses SEQ ID NOS 7, 19, 156, 11, 20, and 157, respectively, in order of appearance.

FIG. 40 depicts Table 32 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 158, 94, 95, and 159, respectively, in order of appearance.

FIG. 41 depicts Table 33 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 160, 161, 59, and 162, respectively, in order of appearance.

FIG. 42 depicts Table 34 that shows the nucleotide sequence of the coding and non-coding strands of S. oleracea—Chloroplast—37 kDa inner envelope membrane protein—GI number 21227—[12]. Figure discloses SEQ ID NOS 164, 163, and 165-168, respectively, in order of appearance.

FIG. 43 depicts Table 35 that shows the nucleotide sequence of the coding and non-coding strands of S. cerevisiae—Mitochondrial—54S ribosomal protein—GI number 45269853—[5]. Figure discloses SEQ ID NOS 77, 76, 169, 170, 80, and 171, respectively, in order of appearance.

FIG. 44 depicts Table 36 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, 172, 108, 109, and 173, respectively, in order of appearance.

FIG. 45 depicts Table 37 that shows the nucleotide sequence of the coding and non-coding strands of A. thaliana—Mitochondrial—Malate dehydrogenase 1—GI number 30695458—[4]. Figure discloses SEQ ID NOS 71, 70, 174, 118, 74, and 175, respectively, in order of appearance.

FIG. 46 depicts Table 38 that shows the nucleotide sequence of the coding and non-coding strands of A. thaliana—Mitochondrial—Malate dehydrogenase 1—GI number 30695458—[4]. Figure discloses SEQ ID NOS 71, 70, 176, 118, 74, and 177, respectively, in order of appearance.

FIG. 47 depicts Table 39 that shows the nucleotide sequence of the coding and non-coding strands of R. norvegicus—Mitochondria—ATP synthase subunit delta—GI number 457928—[18]. Figure discloses SEQ ID NOS 135, 134, 178, 137, 138, and 179, respectively, in order of appearance.

FIG. 48 depicts Table 40 that shows the nucleotide sequence of the coding and non-coding strands of S. cerevisiae—Mitochondria—ATP sunthase sununit delta—GI number 433619—[19]. Figure discloses SEQ ID NOS 141, 140, 180, 143, 144, and 181, respectively, in order of appearance.

FIG. 49 depicts Table 41 that shows the nucleotide sequence of the coding and non-coding strands of H. sapiens—Mitochondrial—ATPase delta-subunit—GI number 12587—[6]. Figure discloses SEQ ID NOS 83, 82, 182, 183, 86, and 184, respectively, in order of appearance.

FIG. 50 depicts Table 42 that shows the nucleotide sequence of the coding and non-coding strands of Phaseolus vulgaris—Endoplasmatic reticulum—Arcelin 5—GI number—[20]. Figure discloses SEQ ID NOS 147, 146, 185, 149, 150, and 186, respectively, in order of appearance.

FIG. 51 depicts Table 43 that shows the nucleotide sequence of the coding and non-coding strands of P. dominulus—Endoplasmic reticulum—Allergen Pol d 5—GI number 51093376—[3]. Figure discloses SEQ ID NOS 62, 61, 187, 188, 150, and 186, respectively, in order of appearance.

FIG. 52 depicts Table 44 that shows the nucleotide sequence of the coding and non-coding strands of B. napus—Mitochondrial—Malate dehydrogenase*—GI number 899225. Figure discloses SEQ ID NOS 7, 19, 189, 11, 20, and 190, respectively, in order of appearance.

FIG. 53 depicts Table 45 that shows the nucleotide sequence of the coding and non-coding strands of A. thaliana—Mitochondrial—Pathogenesis related protein 4*—GI number 186509758. Figure discloses SEQ ID NOS 32, 31, 191, 192, 35, and 193, respectively, in order of appearance.

FIG. 54 depicts Table 46 that shows the nucleotide sequence of the coding and non-coding strands of A. thaliana—Mitochondrial—Pathogenesis related protein 4*—GI number 186509758. Figure discloses SEQ ID NOS 32, 31, 194, 192, 35, and 195, respectively, in order of appearance.

FIG. 55 depicts Table 47 that shows the nucleotide sequence of the coding and non-coding strands of A. thaliana—Mitochondrial—Pathogenesis related protein 4*—GI number 186509758. Figure discloses SEQ ID NOS 32, 31, 196, 192, 35, and 197, respectively, in order of appearance.

FIG. 56 depicts Table 48 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 198, 23, 16, and 199, respectively, in order of appearance.

FIG. 57 depicts Table 49 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 200, 94, 95, and 201, respectively, in order of appearance.

FIG. 58 depicts Table 50 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 202, 94, 95, and 203, respectively, in order of appearance.

FIG. 59 depicts Table 51 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 204, 94, 95, and 205, respectively, in order of appearance.

FIG. 60 depicts Table 52 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 206, 94, 95, and 207, respectively, in order of appearance.

FIG. 61 depicts Table 53 that shows the nucleotide sequence of the coding and non-coding strands of S. oleracea—Chloroplast—37 kDa inner envelope membrane protein—GI number 21227—[12]. Figure discloses SEQ ID NOS 164, 163, 208, 166, 167, and 209, respectively, in order of appearance.

FIG. 62 depicts Table 54 that shows the nucleotide sequence of the coding and non-coding strands of S. cerevisiae—Mitochondrial—54S ribosomal protein—GI number 45269853—[5]. Figure discloses SEQ ID NOS 77, 76, 210, 170, 80, and 211, respectively, in order of appearance.

FIG. 63 depicts Table 55 that shows the nucleotide sequence of the coding and non-coding strands of S. cerevisiae—Mitochondrial—54S ribosomal protein—GI number 45269853—[5]. Figure discloses SEQ ID NOS 77, 76, 212, 170, 80, and 213, respectively, in order of appearance.

FIG. 64 depicts Table 56 that shows the nucleotide sequence of the coding and non-coding strands of B. taurus—Mitochondria—ATP synthase delta chain—GI number 109—[14]. Figure discloses SEQ ID NOS 215, 214, and 216-219, respectively, in order of appearance.

FIG. 65 depicts Table 57 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, 220, 108, 221, 109, 222, and 223, respectively, in order of appearance.

FIG. 66 depicts Table 58 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, 224, 108, 109, and 225, respectively, in order of appearance.

FIG. 67 depicts Table 59 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, 226, 108, 109, and 227, respectively, in order of appearance.

FIG. 68 depicts Table 60 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, 228, 108, 109, and 229, respectively, in order of appearance.

FIG. 69 depicts Table 61 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, 230, 108, 109, and 231, respectively, in order of appearance.

FIG. 70 depicts Table 62 that shows the nucleotide sequence of the coding and non-coding strands of C. sinensis—Chloroplast—Chlorophyllase-1—GI number 7328566—[16]. Figure discloses SEQ ID NOS 112, 111, 232, 114, 115, and 233, respectively, in order of appearance.

FIG. 71 depicts Table 63 that shows the nucleotide sequence of the coding and non-coding strands of R. norvegicus—Mitochondria—ATP synthase subunit delta—GI number 457928—[18]. Figure discloses SEQ ID NOS 135, 134, 234, 137, 138, and 235, respectively, in order of appearance.

FIG. 72 depicts Table 64 that shows the nucleotide sequence of the coding and non-coding strands of R. norvegicus—Mitochondria—ATP synthase subunit delta—GI number 457928—[18]. Figure discloses SEQ ID NOS 135, 134, 236, 137, 138, 237, respectively, in order of appearance.

FIG. 73 depicts Table 65 that shows the nucleotide sequence of the coding and non-coding strands of R. norvegicus—Mitochondria—ATP synthase subunit delta—GI number 457928—[18]. Figure discloses SEQ ID NOS 135, 134, 238, 137, 138, and 239, respectively, in order of appearance.

FIG. 74 depicts Table 66 that shows the nucleotide sequence of the coding and non-coding strands of R. norvegicus—Mitochondria—ATP synthase subunit delta—GI number 457928—[18]. Figure discloses SEQ ID NOS 135, 134, 240, 137, 138, and 241, respectively, in order of appearance.

FIG. 75 depicts Table 67 that shows the nucleotide sequence of the coding and non-coding strands of S. cerevisiae—Mitochondria—ATP sunthase sununit delta—GI number 433619—[19]. Figure discloses SEQ ID NOS 141, 140, 242, 143, 144, and 243, respectively, in order of appearance.

FIG. 76 depicts Table 68 that shows the nucleotide sequence of the coding and non-coding strands of H. sapiens—Mitochondrial—ATPase delta-subunit—GI number 12587—[6]. Figure discloses SEQ ID NOS 83, 82, 244, 183, 86, and 245, respectively, in order of appearance.

FIG. 77 depicts Table 69 that shows the nucleotide sequence of the coding and non-coding strands of Phaseolus vulgaris—Endoplasmatic reticulum—Arcelin 5—GI number—[20]. Figure discloses SEQ ID NOS 147, 146, 246, 149, 150, and 247, respectively, in order of appearance.

FIG. 78 depicts Table 70 that shows the nucleotide sequence of the coding and non-coding strands of P. dominulus—Endoplasmic reticulum—Allergen Pol d 5—GI number 51093376—[3]. Figure discloses SEQ ID NOS 62, 61, 248, 188, 65, and 249, respectively, in order of appearance.

FIG. 79 depicts Table 71 that shows the nucleotide sequence of the coding and non-coding strands of B. napus—Mitochondrial—Malate dehydrogenase*—GI number 899225. Figure discloses SEQ ID NOS 7, 19, 250, 11, 20, and 251, respectively, in order of appearance.

FIG. 80 depicts Table 72 that shows the nucleotide sequence of the coding and non-coding strands of B. napus—Mitochondrial—Malate dehydrogenase*—GI number 899225. Figure discloses SEQ ID NOS 7, 19, 252, 11, 20, and 253, respectively, in order of appearance.

FIG. 81 depicts Table 73 that shows the nucleotide sequence of the coding and non-coding strands of B. napus—Mitochondrial—Malate dehydrogenase*—GI number 899225. Figure discloses SEQ ID NOS 7, 19, 254, 11, 20, and 255, respectively, in order of appearance.

FIG. 82 depicts Table 74 that shows the nucleotide sequence of the coding and non-coding strands of A. thaliana—Mitochondrial—Pathogenesis related protein 4*—GI number 186509758. Figure discloses SEQ ID NOS 32, 31, 256, 192, 35, and 257, respectively, in order of appearance.

FIG. 83 depicts Table 75 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 258, 94, 95, and 259, respectively, in order of appearance.

FIG. 84 depicts Table 76 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 260, 161, 59, and 261, respectively, in order of appearance.

FIG. 85 depicts Table 77 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 262, 161, 59, and 263, respectively, in order of appearance.

FIG. 86 depicts Table 78 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 264, 161, 59, and 265, respectively, in order of appearance.

FIG. 87 depicts Table 79 that shows the nucleotide sequence of the coding and non-coding strands of I. batatas—Mitochondrial—F1-ATPase delta subunit—GI number 217937—[1]. Figure discloses SEQ ID NOS 50, 49, 266, 89, 53, and 267, respectively, in order of appearance.

FIG. 88 depicts Table 80 that shows the nucleotide sequence of the coding and non-coding strands of I. batatas—Mitochondrial—F1-ATPase delta subunit—GI number 217937—[1]. Figure discloses SEQ ID NOS 50, 49, 268, 89, 53, and 269, respectively, in order of appearance.

FIG. 89 depicts Table 81 that shows the nucleotide sequence of the coding and non-coding strands of I. batatas—Mitochondrial—F1-ATPase delta subunit—GI number 217937—[1]. Figure discloses SEQ ID NOS 50, 49, 270, 89, 53, and 271, respectively, in order of appearance.

FIG. 90 depicts Table 83 that shows the nucleotide sequence of the coding and non-coding strands of I. batatas—Mitochondrial—F1-ATPase delta subunit—GI number 217937—[1]. Figure discloses SEQ ID NOS 50, 49, 272, 89, 53, and 273, respectively, in order of appearance.

FIG. 91 depicts Table 83 that shows the nucleotide sequence of the coding and non-coding strands of I. batatas—Mitochondrial—F1-ATPase delta subunit—GI number 217937—[1]. Figure discloses SEQ ID NOS 50, 49, 274, 89, 53, and 275, respectively, in order of appearance.

FIG. 92 depicts Table 84 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 276, 23, 16, and 277, respectively, in order of appearance.

FIG. 93 depicts Table 85 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 278, 23, 16, and 279, respectively, in order of appearance.

FIG. 94 depicts Table 86 that shows the nucleotide sequence of the coding and non-coding strands of H. vulgare—Endoplasmatic reticulum—Pathogenesis-related protein 4—GI number 1808650—[11]. Figure discloses SEQ ID NOS 92, 91, 280, 94, 95, and 281, respectively, in order of appearance.

FIG. 95 depicts Table 87 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 282, 161, 59, and 283, respectively, in order of appearance.

FIG. 96 depicts Table 88 that shows the nucleotide sequence of the coding and non-coding strands of S. oleracea—Chloroplast—37 kDa inner envelope membrane protein—GI number 21227—[12]. Figure discloses SEQ ID NOS 164, 163, 284, 166, 167, and 285, respectively, in order of appearance.

FIG. 97 depicts Table 89 that shows the nucleotide sequence of the coding and non-coding strands of G. max—Mitochondria—Methylcrotonoyl-CoA carboxylase subunit alpha—GI number 497233—[15]. Figure discloses SEQ ID NOS 106, 105, 286, 108, 109, and 287, respectively, in order of appearance.

FIG. 98 depicts Table 90 that shows the nucleotide sequence of the coding and non-coding strands of C. sinensis—Chloroplast—Chlorophyllase-1—GI number 7328566—[16]. Figure discloses SEQ ID NOS 112, 111, 288, 114, 115, and 289, respectively, in order of appearance.

FIG. 99 depicts Table 91 that shows the nucleotide sequence of the coding and non-coding strands of C. sinensis—Chloroplast—Chlorophyllase-1—GI number 7328566—[16]. Figure discloses SEQ ID NOS 112, 111, 290, 114, 115, and 291, respectively, in order of appearance.

FIG. 100 depicts Table 92 that shows the nucleotide sequence of the coding and non-coding strands of C. sinensis—Chloroplast—Chlorophyllase-1—GI number 7328566—[16]. Figure discloses SEQ ID NOS 112, 111, 292, 114, 115, and 293, respectively, in order of appearance.

FIG. 101 depicts Table 93 that shows the nucleotide sequence of the coding and non-coding strands of C. sinensis—Chloroplast—Chlorophyllase-1—GI number 7328566—[16]. Figure discloses SEQ ID NOS 112, 111, 294, 114, 115, and 295, respectively, in order of appearance.

FIG. 102 depicts Table 94 that shows the nucleotide sequence of the coding and non-coding strands of R. norvegicus—Mitochondria—ATP synthase subunit delta—GI number 457928—[18]. Figure discloses SEQ ID NOS 135, 134, 296, 137, 138, and 297, respectively, in order of appearance.

FIG. 103 depicts Table 95 that shows the nucleotide sequence of the coding and non-coding strands of H. sapiens—Mitochondrial—ATPase delta-subunit—GI number 12587—[6]. Figure discloses SEQ ID NOS 83, 82, 298, 183, 86, and 299, respectively, in order of appearance.

FIG. 104 depicts Table 96 that shows the nucleotide sequence of the coding and non-coding strands of H. sapiens—Mitochondrial—ATPase delta-subunit—GI number 12587—[6]. Figure discloses SEQ ID NOS 83, 82, 300, 183, 86, and 301, respectively, in order of appearance.

FIG. 105 depicts Table 97 that shows the nucleotide sequence of the coding and non-coding strands of M. martensii—Endoplasmic reticulum—anti-epilepsy peptide precursor—GI number 16740522—[2]. Figure discloses SEQ ID NOS 38, 37, 302, 303, 41, and 304, respectively, in order of appearance.

FIG. 106 depicts Table 98 that shows the nucleotide sequence of the coding and non-coding strands of Phaseolus vulgaris—Endoplasmatic reticulum—Arcelin 5—GI number—[20]. Figure discloses SEQ ID NOS 147, 146, 305, 149, 150, and 306, respectively, in order of appearance.

FIG. 107 depicts Table 99 that shows the nucleotide sequence of the coding and non-coding strands of P. dominulus—Endoplasmic reticulum—Allergen Pol d 5—GI number 51093376—[3]. Figure discloses SEQ ID NOS 62, 61, 307, 188, 65, and 308, respectively, in order of appearance.

FIG. 108 depicts Table 100 that shows the nucleotide sequence of the coding and non-coding strands of B. napus—Mitochondrial—Malate dehydrogenase*—GI number 899225. Figure discloses SEQ ID NOS 7, 19, 309, 11, 20, and 310, respectively, in order of appearance.

FIG. 109 depicts Table 101 that shows the nucleotide sequence of the coding and non-coding strands of I. batatas—Mitochondrial—F1-ATPase delta subunit—GI number 217937—[1]. Figure discloses SEQ ID NOS 50, 49, 311, 89, 53, and 312, respectively, in order of appearance.

FIG. 110 depicts Table 102 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 313, 23, 53, and 314, respectively, in order of appearance.

FIG. 111 depicts Table 103 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 315, 23, 16, and 316, respectively, in order of appearance.

FIG. 112 depicts Table 104 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 317, 161, 59, and 318, respectively, in order of appearance.

FIG. 113 depicts Table 105 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 319, 161, 59, and 320, respectively, in order of appearance.

FIG. 114 depicts Table 106 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 321, 161, 59, and 322, respectively, in order of appearance.

FIG. 115 depicts Table 107 that shows the nucleotide sequence of the coding and non-coding strands of S. oleracea—Chloroplast—37 kDa inner envelope membrane protein—GI number 21227—[12]. Figure discloses SEQ ID NOS 164, 163, 323, 166, 167, and 324, respectively, in order of appearance.

FIG. 116 depicts Table 108 that shows the nucleotide sequence of the coding and non-coding strands of B. taurus—Mitochondria—Aminomethyltransferase—GI number 31343489—[13]. Figure discloses SEQ ID NOS 100, 99, 325, 102, 103, and 326, respectively, in order of appearance.

FIG. 117 depicts Table 109 that shows the nucleotide sequence of the coding and non-coding strands of B. taurus—Mitochondria—Aminomethyltransferase—GI number 31343489—[13]. Figure discloses SEQ ID NOS 100, 99, 327, 102, 103, and 328, respectively, in order of appearance.

FIG. 118 depicts Table 110 that shows the nucleotide sequence of the coding and non-coding strands of C. sinensis—Chloroplast—Chlorophyllase-1—GI number 7328566—[16]. Figure discloses SEQ ID NOS 112, 111, 329, 114, 115, and 330, respectively, in order of appearance.

FIG. 119 depicts Table 111 that shows the nucleotide sequence of the coding and non-coding strands of S. cerevisiae—Mitochondria—ATP sunthase sununit delta—GI number 433619—[19]. Figure discloses SEQ ID NOS 141, 140, 331, 143, 144, and 332, respectively, in order of appearance.

FIG. 120 depicts Table 112 that shows the nucleotide sequence of the coding and non-coding strands of S. cerevisiae—Mitochondria—ATP sunthase sununit delta—GI number 433619—[19]. Figure discloses SEQ ID NOS 141, 140, 333, 143, 144, and 334, respectively, in order of appearance.

FIG. 121 depicts Table 113 that shows the nucleotide sequence of the coding and non-coding strands of H. sapiens—Mitochondrial—ATPase delta-subunit—GI number 12587—[6]. Figure discloses SEQ ID NOS 83, 82, 335, 183, 86, and 336, respectively, in order of appearance.

FIG. 122 depicts Table 114 that shows the nucleotide sequence of the coding and non-coding strands of M. martensii—Endoplasmic reticulum—anti-epilepsy peptide precursor—GI number 16740522—[2]. Figure discloses SEQ ID NOS 38, 37, 337, 303, 41, and 338, respectively, in order of appearance.

FIG. 123 depicts Table 115 that shows the nucleotide sequence of the coding and non-coding strands of Phaseolus vulgaris—Endoplasmatic reticulum—Arcelin 5—GI number—[20]. Figure discloses SEQ ID NOS 147, 146, 339, 149, 150, and 340, respectively, in order of appearance.

FIG. 124 depicts Table 116 that shows the nucleotide sequence of the coding and non-coding strands of P. dominulus—Endoplasmic reticulum—Allergen Pol d 5—GI number 51093376—[3]. Figure discloses SEQ ID NOS 62, 61, 341, 188, 65, and 342, respectively, in order of appearance.

FIG. 125 depicts Table 117 that shows the nucleotide sequence of the coding and non-coding strands of P. dominulus—Endoplasmic reticulum—Allergen Pol d 5—GI number 51093376—[3]. Figure discloses SEQ ID NOS 62, 61, 343, 188, 65, and 344, respectively, in order of appearance.

FIG. 126 depicts Table 118 that shows the nucleotide sequence of the coding and non-coding strands of P. dominulus—Endoplasmic reticulum—Allergen Pol d 5—GI number 51093376—[3]. Figure discloses SEQ ID NOS 62, 61, 345, 188, 65, and 346, respectively, in order of appearance.

FIG. 127 depicts Table 119 that shows the nucleotide sequence of the coding and non-coding strands of Petunia×hybrida hydroxyproline-rich systemin precursor—GI number 146762153. Figure discloses SEQ ID NOS 348, 347, and 349-352, respectively, in order of appearance.

FIG. 128 depicts Table 120 that shows the nucleotide sequence of the coding and non-coding strands of A. thaliana—Mitochondrial—Malate dehydrogenase 1—GI number 30695458—[4]. Figure discloses SEQ ID NOS 71, 70, 353, 354, 74, and 355, respectively, in order of appearance.

FIG. 129 depicts Table 121 that shows the nucleotide sequence of the coding and non-coding strands of S. cerevisiae—Mitochondrial—54S ribosomal protein—GI number 45269853—[5]. Figure discloses SEQ ID NOS 77, 76, 356, 357, 80, and 358, respectively, in order of appearance.

FIG. 130 depicts Table 122 that shows the nucleotide sequence of the coding and non-coding strands of C. sinensis—Chloroplast—Chlorophyllase-1—GI number 7328566—[16]. Figure discloses SEQ ID NOS 112, 111, 359, 360, 115, and 361, respectively, in order of appearance.

FIG. 131 depicts Table 123 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 362, 363, 16, and 364, respectively, in order of appearance.

FIG. 132 depicts Table 124 that shows the nucleotide sequence of the coding and non-coding strands of N. tabacum—Endoplasmic reticulum—Pathogen and wound-inducible antifungal protein CBP20*—GI number 632733. Figure discloses SEQ ID NOS 18, 15, 365, 366, 16, and 367, respectively, in order of appearance.

FIG. 133 depicts Table 125 that shows the nucleotide sequence of the coding and non-coding strands of I. batatas—Mitochondrial—F1-ATPase delta subunit—GI number 217937—[1]. Figure discloses SEQ ID NOS 50, 49, 368, 369, 53, and 370, respectively, in order of appearance.

FIG. 134 depicts Table 126 that shows the nucleotide sequence of the coding and non-coding strands of A. thaliana—ATHSP23.6-MITO (MITOCHONDRION-LOCALIZED SMALL HEAT SHOCK PROTEIN 23.6)—GI number 30686795. Figure discloses SEQ ID NOS 372, 371, and 373-376, respectively, in order of appearance.

FIG. 135 depicts Table 127 that shows the nucleotide sequence of the coding and non-coding strands of S. tuberosum—Mitochondria—Precursor of the 59 kDa subunit of the mitochondrial NAD+-dependent malic enzyme—GI number 438130—[21]. Figure discloses SEQ ID NOS 378, 377, and 379-382, respectively, in order of appearance.

FIG. 136 depicts Table 128 that shows the nucleotide sequence of the coding and non-coding strands of S. tuberosum—Mitochondria—Serine hydroxymethyltransferase—GI number 438246—[33]. Figure discloses SEQ ID NOS 384, 383, and 385-388, respectively, in order of appearance.

FIG. 137 depicts Table 129 that shows the nucleotide sequence of the coding and non-coding strands of S. tuberosum—Mitochondria—Precursor of the 59 kDa subunit of the mitochondrial NAD+-dependent malic enzyme—GI number 438130—[21]. Figure discloses SEQ ID NOS 378, 377, and 389-392, respectively, in order of appearance.

FIG. 138 depicts Table 130 that shows the nucleotide sequence of the coding and non-coding strands of H. sapiens—Endoplasmatic reticulum—preproendothelin 1; preproET-1—GI number 298590—[22]. Figure discloses SEQ ID NOS 394, 393, and 395-398, respectively, in order of appearance.

FIG. 139 depicts Table 131 that shows the nucleotide sequence of the coding and non-coding strands of Hordeum vulgare—Mla locus—GI number 20513849. Figure discloses SEQ ID NOS 399-402, respectively, in order of appearance.

FIG. 140 depicts Table 133 that shows the nucleotide sequence of the coding and non-coding strands of R. norvegicus—NADH ubiquinone oxidoreductase subunit (IP13) gene—GI number 600528—[7]. Figure discloses SEQ ID NOS 403-406, respectively, in order of appearance.

FIG. 141 depicts Table 133 that shows the nucleotide sequence of the coding and non-coding strands of S. cerevisiae—Mitochondrial—54S ribosomal protein—GI number 45269853—[5]. Figure discloses SEQ ID NOS 77, 76, 407, 408, 80, and 409, respectively, in order of appearance.

FIG. 142 depicts Table 134 that shows the nucleotide sequence of the coding and non-coding strands of T. sativum—Endoplasmic reticulum—wPR4g gene for putative vacuolar defense protein—GI number 78096542. Figure discloses SEQ ID NOS 56, 55, 410, 411, 59, and 412, respectively, in order of appearance.

FIG. 143 depicts Table 135 that shows the nucleotide sequence of the coding and non-coding strands of P. dominulus—Endoplasmic reticulum—Allergen Pol d 5—GI number 51093376—[3]. Figure discloses SEQ ID NOS 62, 61, 413, 414, 65, and 415, respectively, in order of appearance.

FIG. 144A depicts Table 136a that shows the nucleotide sequence of the coding and non-coding strands of A. thaliana—Mitochondrial—Malate dehydrogenase 1—GI number 30695458—[10]. Figure discloses residues 1-168 of SEQ ID NO: 417, nucleotides 1-504 of SEQ ID NO: 416, nucleotides 1-504 of SEQ ID NO: 418, and residues 1-168 of SEQ ID NO: and 419, respectively, in order of appearance.

FIG. 144B depicts Table 136B that shows the nucleotide sequence of the coding and non-coding strands of A. thaliana—Mitochondrial—Malate dehydrogenase 1—GI number 30695458—[10]. Figure discloses residues 169-341 of SEQ ID NO: 417, nucleotides 505-1,023 of SEQ ID NO: 416, nucleotides 505-1,023 of SEQ ID NO: 418, and residues 169-341 of SEQ ID NO: 419, respectively, in order of appearance.

FIG. 144C depicts Table 136C that shows the nucleotide sequence of the coding and non-coding strands of A. thaliana—Mitochondrial—Malate dehydrogenase 1—GI number 30695458—[10]. Figure discloses nucleotides 1-504 of SEQ ID NO: 420 and nucleotides 1-504 of SEQ ID NO: 421, respectively, in order of appearance.

FIG. 144D depicts Table 136D that shows the nucleotide sequence of the coding and non-coding strands of A. thaliana—Mitochondrial—Malate dehydrogenase 1—GI number 30695458—[10]. Figure discloses nucleotides 505-1,023 of SEQ ID NO: 420 and nucleotides 505-1,023 of SEQ ID NO: 421, respectively, in order of appearance.

FIG. 145 depicts Program label_inv.m function for a 1 nucleotide error. Figure discloses SEQ ID NOS 470, 7, 19, 10, and 11, respectively, in order of appearance.

FIG. 146 depicts Program label_inv.m function for 2 nucleotide errors. Figure discloses SEQ ID NOS 146, 38, 37, 422, and 423, respectively, in order of appearance.

FIG. 147 depicts Program label_invc.m function for 1 nucleotide difference. Figure discloses SEQ ID NOS 14, 56, 55, 424, and 425 respectively, in order of appearance.

FIG. 148 shows Rat mRNA for mitochondrial malate dehydrogenase—Locus X04240. Figure discloses SEQ ID NOS 427, 426, 426, and 426, respectively, in order of appearance.

FIG. 149 shows simulations with changes in the mdh1-21 generated by the (63,57,3) bch code over Z₄-galois ring gr(4,6) based on the paper—case 1—A labeling. Figure discloses SEQ ID NOS 427, 426, 428, and 429, respectively, in order of appearance.

FIG. 150 shows simulations with changes in the mdh1-21 generated by the (63,57,3) bch code over Z₄-galois ring gr(4,6) based on the paper—Case 2—Labelling B. Figure discloses SEQ ID NOS 427, 426, 430, and 431, respectively, in order of appearance.

FIG. 151 shows simulations with changes in the mdh1-21 generated by the (63,57,3) bch code over Z₄-galois ring gr(4,6) based on the paper—Case 3—Labelling C—MDH1-21*. Figure discloses SEQ ID NOS 427, 426, 432, 433, 433, and 432, respectively, in order of appearance.

FIG. 152 shows a cases that was analyzed the eighth possible combinations between the nucleotides of: K, A and R, according to the changes realized in the paper [6]. Figure discloses SEQ ID NO: 1.

FIG. 153 shows the analysis of the eighth possible combinations between the nucleotides of: R, A and K, according to the changes realized in the paper [6]. Figure discloses SEQ ID NO: 434.

FIG. 154 shows the analysis of the sixteen possible combinations between the nucleotides of: K, A and K. Figure discloses SEQ ID NO: 435.

FIGS. 155-162 each show an analysis of MDH1-21 sequence MLSALAKPVGAALARSFSTSA (SEQ ID NO: 1) for one of the eight possible combinations between the nucleotides of: K, A and R at positions 7, 14 and 15 respectively (where 7° aa (R) is replaced by Lysine (K) encoded by AAA or AAG, and 14° aa (R) is replaced by Alanine (A) encoded by GCT or GCC or GCA or GCG and 15° aa is (R)). FIG. 155 discloses SEQ ID NOS 427, 426, 436, and 1, respectively, in order of appearance. FIG. 156 discloses SEQ ID NOS 427, 426, 437, and 1, respectively, in order of appearance. FIG. 157 discloses SEQ ID NOS 427, 426, 438, and 1, respectively, in order of appearance. FIG. 158 discloses SEQ ID NOS 427, 426, 439, 1, 1, 440, 441, and 1, respectively, in order of appearance. FIG. 159 discloses SEQ ID NOS 427, 426, 442, and 1, respectively, in order of appearance. FIG. 160 discloses SEQ ID NOS 427, 426, 443, and 1, respectively, in order of appearance. FIG. 161 discloses SEQ ID NOS 427, 426, 444, and 1, respectively, in order of appearance. FIG. 162 discloses SEQ ID NOS 427, 426, 445, and 1, respectively, in order of appearance.

FIGS. 163-170 each show an analysis of MDH1-21 sequence MLSALAKPVGAALARSFSTSA (SEQ ID NO: 1) for one of the eight possible combinations between the nucleotides of: R, A and K at positions 7, 14 and 15 respectively (where 7° aa is (R), 14° aa (R) is replaced by Alanine (A) encoded by GCT or GCC or GCA or GCG., 15° aa (R) is replaced by Lysine (K) encoded by AAA or AAG). FIG. 163 discloses SEQ ID NOS 427, 426, 446, and 434, respectively, in order of appearance. FIG. 164 discloses SEQ ID NOS 427, 426, 447, and 434, respectively, in order of appearance. FIG. 165 discloses SEQ ID NOS 427, 426, 448, and 434, respectively, in order of appearance. FIG. 166 discloses SEQ ID NOS 427, 426, 449, and 434, respectively, in order of appearance. FIG. 167 discloses SEQ ID NOS 427, 426, 450, and 434, respectively, in order of appearance. FIG. 168 discloses SEQ ID NOS 427, 426, 451, and 434, respectively, in order of appearance. FIG. 169 discloses SEQ ID NOS 427, 426, 452, and 434, respectively, in order of appearance. FIG. 170 discloses SEQ ID NOS 427, 426, 453, and 434, respectively, in order of appearance.

FIGS. 171-186 each show an analysis of MDH1-21 sequence MLSALAKPVGAALARSFSTSA (SEQ ID NO: 1) for one of the sixteen possible combinations between the nucleotides of: K, A and K at positions 7, 14 and 15 respectively (where 7° aa (R) is replaced by Lysine (K) encoded by AAA or AAG, 14° aa (R) is replaced by Alanine (A) encoded by GCT or GCC or GCA or GCG and 15° aa (R) is replaced by Lysine (K) encoded by AAA or AAG). FIG. 171 discloses SEQ ID NOS 427, 426, 454, and 435, respectively, in order of appearance. FIG. 172 discloses SEQ ID NOS 427, 426, 455, and 435, respectively, in order of appearance. FIG. 173 discloses SEQ ID NOS 427, 426, 456, and 435, respectively, in order of appearance. FIG. 174 discloses SEQ ID NOS 427, 426, 457, and 435, respectively, in order of appearance. FIG. 175 discloses SEQ ID NOS 427, 426, 458, and 435, respectively, in order of appearance. FIG. 176 discloses SEQ ID NOS 427, 426, 459, and 435, respectively, in order of appearance. FIG. 177 discloses SEQ ID NOS 427, 426, 460, and 435, respectively, in order of appearance. FIG. 178 discloses SEQ ID NOS 427, 426, 461, and 435, respectively, in order of appearance. FIG. 179 discloses SEQ ID NOS 427, 426, 462, and 435, respectively, in order of appearance. FIG. 180 discloses SEQ ID NOS 427, 426, 463, and 435, respectively, in order of appearance. FIG. 181 discloses SEQ ID NOS 427, 426, 464, and 435, respectively, in order of appearance. FIG. 182 discloses SEQ ID NOS 427, 426, 465, and 435, respectively, in order of appearance. FIG. 183 discloses SEQ ID NOS 427, 426, 466, and 435, respectively, in order of appearance. FIG. 184 discloses SEQ ID NOS 427, 426, 467, and 435, respectively, in order of appearance. FIG. 185 discloses SEQ ID NOS 427, 426, 468, and 435, respectively, in order of appearance. FIG. 186 discloses SEQ ID NOS 427, 426, 469, and 435, respectively, in order of appearance.

FIG. 187: Phenogram inferred using the Neighbor-Joining method with the evolutionary distances computed using the Jukes-Cantor model. The percentage of replicate trees in which the associated taxa clustered together in the bootstrap test (1000 replicates).

FIG. 188: Phylogenetic tree inferred by Bayesian analysis from the data set. Values close to the branches indicate Bayesian posterior probability.

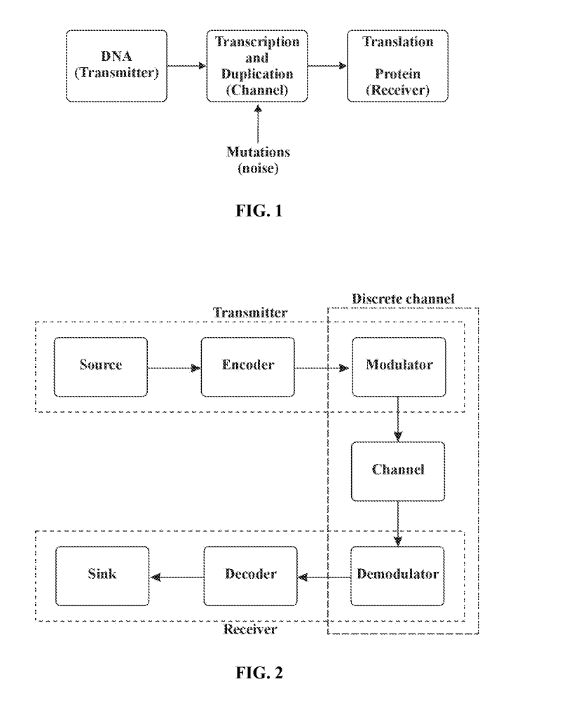

### DETAILED DESCRIPTION OF THE INVENTION

While the invention has been described in detail and with reference to specific aspects thereof, it will be apparent to one of ordinary skill in the art that various changes and modifications can be made thereto without departing from the spirit and scope thereof.

The primitive and non primitive BCH codes used in the generation of the DNA sequences, described in the present invention, are constructed over the algebraic structures of field and ring and its Galois extensions. The theoretical background for the construction of these codes, as well as the definitions and algebraic properties of the expressions such as “primitive BCH code”, “non primitive BCH code”, “field”, “ring” and “Galois extensions” employed in the present invention, may be found in [4], [5], [28], [34] and [35].

In a digital communication system information is carried out from the transmitter to the receiver by a string of bits through a transmission channel. In eukaryotic cells, genetic information in the nucleus moves to the cytosol through mRNA intermediates, which are further translated into proteins. It is conceivable that a “mathematical code” used for error-correction in data transmission through a noisy channel might be applied to DNA sequences (FIG. 4).

The overwhelming amount of DNA sequences available in genomic databases requires the development of mathematical models to describe and characterize biological systems. The establishment of systematic procedures to identify coding and non-coding regions in the DNA structure is one of the major goals in Information Theory [14], [22]-[25]. The primary goal in Coding Theory is to establish the proper mathematical structure and model for the identification of sequences in the coding regions as codewords of error-correcting codes. Although several studies have been made in order to associate DNA sequences with codewords of error-correcting codes [16]-[21], it seems that no success has been achieved so far. Here we propose a model for the biological coding system which resembles the most efficient digital communication system. This remarkable finding shows the existence of error-correcting codes associated with DNA sequences. It is then possible to develop a systematic approach to be employed in mutational and polymorphism analysis with applications in genetic engineering.

### Biological Coding System Model

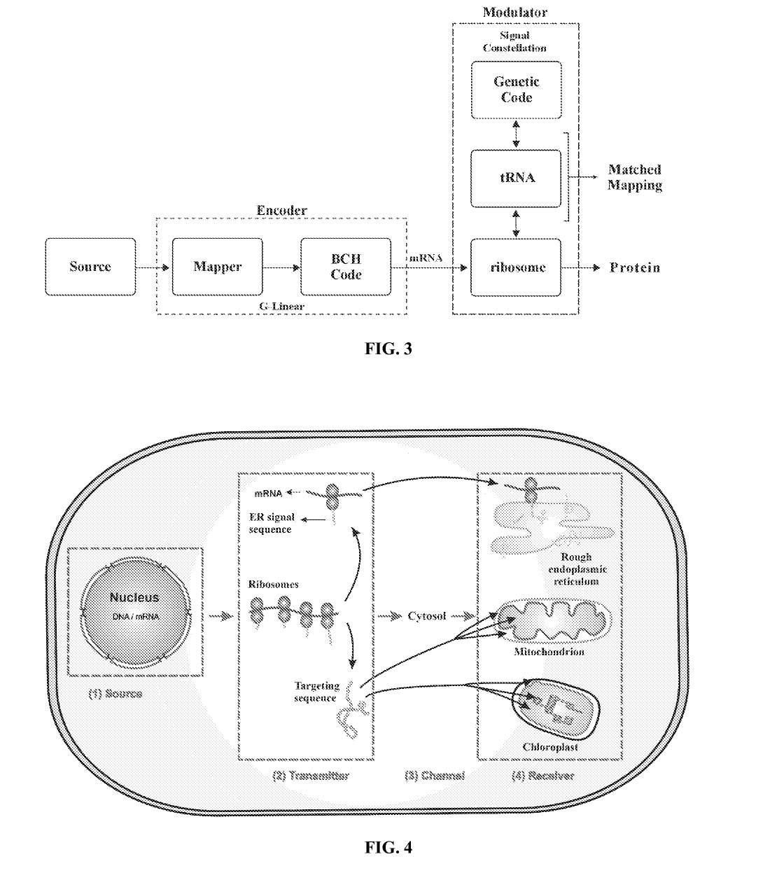

One possible interpretation of Shannon's Channel Coding Theorem [1], regarding the flow of information from the source to the sink, is that the mutual information of the discrete channel, (FIG. 2), be as close as possible to the entropy of the source. To achieve this goal, an error correcting code is used. Therefore, the transmitter in the digital communication system model consists of two cascade blocks, one block associated with an encoder and the other one associated with a modulator, signal constellation, (FIG. 2).

The codeword at the encoder output is related to the mature mRNA, whereas the output of the modulator is related to the protein. Although the matching, by the tRNA, of each codon in the mature mRNA strand with its corresponding anticodon is well known in the biological context, it needs a mathematical characterization. However, in a digital communication system context this very same process exists and it is called matched mapping [26]. This mathematical property, in addition to implying that the underlying algebraic structure of the encoder and the signal constellation are the same up to an isomorphism, guarantees the least overall system complexity. The class of codes satisfying this property is known as geometrically uniform codes [27], and an important subclass is the G-linear codes, [8], [12] and [13] where G denotes an algebraic group.

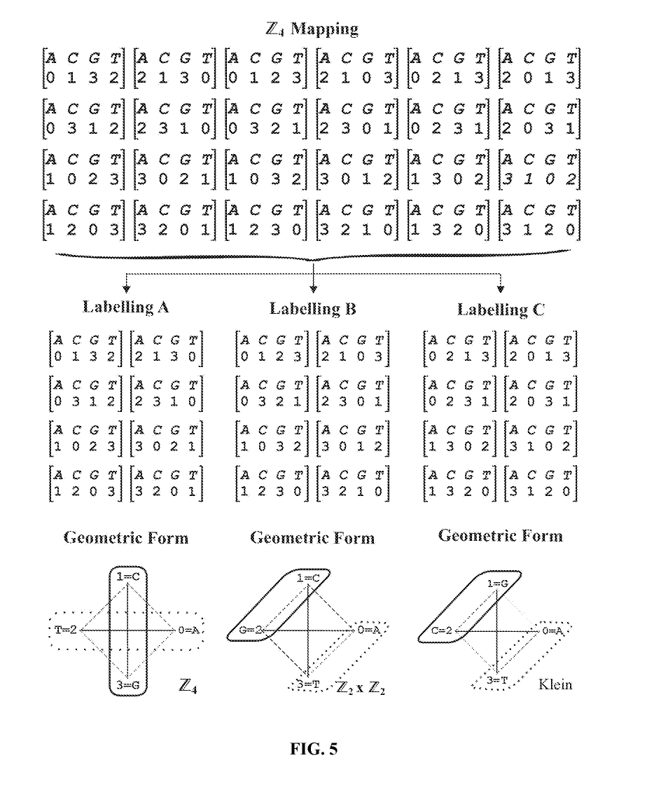

Therefore, the encoder consists of a mapper and an encoder of a linear block code. The modulator consists of the genetic code, the tRNA and the ribosome (FIG. 3). The genetic code may be viewed as a signal constellation, where each codon is considered as a signal in the signal constellation, the tRNA realizes the matched mapping, whereas the ribosome behaves as a digital signal processor.

Code alphabet and mapper

The 4-ary alphabet at the source output is related to the set of nucleotides, denoted by N={A, C, G, T/U}, corresponding to the bases adenine (A), cytosine (C), guanine (G) and thymine (T) or uracil (U). On the other hand, the 4-ary alphabet of the linear block code is denoted by Z₄={0, 1, 2, 3} for the integer residue ring and by GF(4)={0,1,α,α²} for the Galois field satisfying the operations of addition and multiplication according to the corresponding mathematical structure. As the mappings between N→Z₄ and N→GF(4) are unknown, we consider every possible permutation between the elements of each one of these sets. In the case of the mapping N→Z₄, we have noticed that there are three sets where each set contains eight permutations. Each one of these sets defines a labelling denoted by A, B, and C which are associated with geometrical arrangements (FIG. 5). These labelling classify the DNA sequences as nonlinear (labelling A) and linear (labelling B and C). In the case of the mapping N→GF(4) we observe that the twenty four permutations define a unique labelling (FIG. 6). These mappings are employed in order to determine which is the best association of each one of the symbols in the set N with the corresponding symbol in the set Z₄ and GF(4), and vice-versa.

### Codes and Mathematical Structures

According to the aforementioned model, the following questions still require answers: 1) Among the several codes employed in the transmission of information, is there one capable of reproducing the DNA sequences and the corresponding complementary strands? 2) If so, what is the proper mathematical structure to construct such a code?

An answer to the first question starts with the well known fact in coding theory that the Nordstrom-Robinson's [2] and Preparata's [3] nonlinear codes have greater error correction capability than the corresponding linear codes [4], albeit their loss of some structural properties. Consequently, the complexity of the decoding process is greater than that of the linear codes. However, when G is isomorphic to Z₄ some of the Z₄-linear codes [13] are exactly the Nordstrom-Robinson and Preparata nonlinear codes. Thus, the Z₄-linear codes in addition to inheriting the advantages of the encoding and decoding processes of the linear codes, due to the use of the linear block codes, they may maintain the error correction capability of the aforementioned nonlinear codes by the inclusion of a mapper. If G is isomorphic either to Z₂×Z₂ or to the “Klein group” then the corresponding Z₂×Z₂-linear and Klein-linear codes are linear. Consequently, the DNA sequences reproduced by the previous codes are classified accordingly. Hence, the encoder of a G-linear code consists of a mapper and a linear block code [8], [12] and [13].

An answer to the second question is related to the fact that in general the complexity associated with the construction method of an error-correcting code depends on the algebraic structure and, when required, some additional properties. Thus, the lesser the complexity of the encoding and decoding processes the more efficient the code will be in the transmission of information. An important class of error-correcting codes [4] and [5] satisfying the previous premises is the class of cyclic codes, where the BCH code is one of its constituents. The BCH code may be generated in Galois ring extensions [7], [9]-[11] and [34] and Galois field extensions [4] and [5]. In particular, we consider the integer residue ring, Z₄, and the Galois field GF(4). A primitive BCH code over GF(q), where q is a power of a prime, is characterized by its codeword length, n, being n=q−1. This value of n accounts for the number of nonzero or invertible elements which are used either in the Galois field GF(4^{r/2}) or in the Galois ring GR(4,r). These elements are part of a mathematical structure called group of units, denoted by GF*(4^{r/2}) and GR*(4,r), all of them being the roots of unity, xⁿ−1=0. Contrary to what happens in GF(4^{r/2}) where every nonzero element has its inverse, in GR(4,r) some of the nonzero elements are zero divisors and the remaining ones are not. Therefore, the necessary condition for the unique factorization of xⁿ−1 over GR(4,r) is that the length of the DNA sequence be an odd number. Hence, identifying the cyclic property associated with such sequences is required.

The primitive element is such that all the nonzero elements of a field are a power of it. Thus, if a polynomial has as one of its roots a primitive element this polynomial is called a primitive polynomial [4] and [5]. This is an important and also a simplifier fact, for through the primitive element we may establish which elements will be selected in the encoding process. In this direction, the Galois field GF(2^{r}) is obtained as an extension of the Galois field GF(2) by an ideal (a set consisting of all the polynomials which are multiple of a specific polynomial) generated by any of the primitive polynomials of degree r. Once a primitive polynomial is fixed, it has to be used in the Galois ring extension. It is this ring which contains the group of units of interest, that is, the GR*(4,r) and that will be used in the generation of the DNA sequences. The previous considerations related to the primitive polynomial are applicable to the reciprocal of the primitive polynomial.

### Procedure for the DNA Sequence Generation

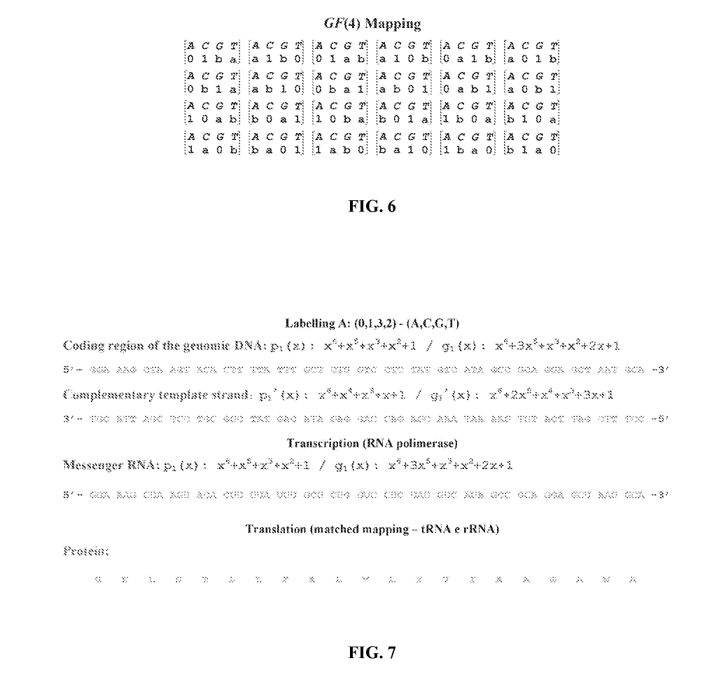

The coding region of the genomic DNA of a protein consists of a codeword of a G-linear code. This codeword is obtained by use of the BCH code over Z₄ generated by the polynomial g(x) with the corresponding labelling A, B or C and the primitive polynomial of degree r used in the Galois ring extension GR(4, r). The complementary strand is generated by a codeword obtained from the BCH code over Z₄ generated by the reciprocal of the generator polynomial g*(x) having the same previous labelling and the reciprocal of the primitive polynomial. Note that the transfer RNA (tRNA) realizes the matched mapping between each one of the codons in this sequence with the corresponding anticodons (FIG. 7).

A primitive BCH code with parameters (m, k, d) over GR(4,r) is such that n=2^{r}−1. A detailed construction of BCH codes over Galois fields and ring may be found in [4], [5], [7], [9], [10], [11] and [34].

The parameters of the BCH code are denoted by: n=the codeword length (the length of the DNA sequences); k=the dimension of the code (length of the information sequence responsible for the generation of the DNA sequence) and d=the minimum distance of the code (the least number of positions in which any two code words differ). The BCH code with parameters (m, k, d) has its minimum distance given by d=2t+1, where t denotes the number of errors. The results show that the BCH codes with parameters (m, k, 3) are able to reproduce DNA sequences with t=1 nucleotide error. As a consequence of d=3 the degree of the generator polynomial g(x), n−k, is equal to the degree of the Galois ring extension, r, that is, n−k=r. Hence, g(x)=g₀+g₁x+g₂x²+ . . . +gᵣx^{r}, where gᵢεGR*(4,r), the invertible elements of GR(4,r). It is from the generator polynomial g(x) that the generator matrix, G, of the BCH code is determined, as well as the parity-check matrix H, this one obtained from the polynomial h(x)=(xⁿ−1)/g(x). We call the attention to the fact that for each primitive polynomial used in the generation of the ring GR(4,r) corresponds to a different generator polynomial g(x). Thus, we have to consider it when looking for a new code.

Since the error correction capability of a code is related to the number of codeword, in the case in consideration 4^{k}, where k=n−r, then for a given value of n, the lesser the value of r is the greater will be the number of codewords and therefore, the greater will be the computational complexity in generating all the 4^{k} codewords.

In order to overcome this problem, which is classified as an NP-complete problem, instead of generating all the code words to compare with the given DNA sequence, we consider the DNA sequence, under the action of each one of the twenty four permutations, as a codeword. Hence, to determine if each one of the twenty four possibilities is in fact a codeword we use the relation v.H^{T}=0, where v is a possible codeword and H^{T} is the transpose of the parity-check matrix. To analyze the DNA sequence differing one nucleotide of the code word, we consider the three other possibilities of nucleotides in each position in the sequence for each permutation and again we use the relation v.H^{T}=0 to check if v is a codeword.

BCH codes over GF(4^{r/2}) with parameters (m, k, 3) were also constructed with the objective to determine the best mathematical structure, ring or field, which is capable of reproducing the majority of the DNA sequences.

The examples shown next illustrate one of the several forms of realizing the invention. However, these are not restrictive forms of seeing the present invention but illustrative ones.

### Example of the Construction of a Code Capable of Generating and Reproducing DNA Sequences

### BCH Codes Over Rings

Here we present a non-limiting algorithm which shows in detail the construction steps of a BCH code over ring with parameters (n,k,d)=(63,57,3) capable of reproducing DNA sequences of length n=2^{r}−1. We call the attention to the fact that to the cases where the sequence length is given by n=2^{r}+2, then the DNA sequences that have methionine in their first position, may be disregarded, since the generator matrix will have a column with the same elements.

The parameters of the code are denoted as follows: n=codeword length (length of the DNA sequences); k=the dimension of the code (length of the information sequence responsible for the generation of the DNA sequence) and d=the minimum distance of the code (the least number of positions in that any two codewords differ).

The main difference between the construction of the cyclic codes over rings and the construction of cyclic codes over fields is the fact that the roots of the generator polynomial of the cyclic codes over rings are in the extension of the ring Z_{q}, instead of being in the extension of the field GF(p^{r}).

If the characteristic of the field is p and the codeword length is n are such that the gcd(p,n)=1, then xⁿ−1 does not have multiple roots.

Construction of an (n, k, d)=(63, 57, 3) Primitive BCH Code Over GR(4,r)

Step 1—Determining the Alphabet and the Code Mathematical Structure

The 4-ary alphabet at the source output is related to the set of nucleotides, denoted by N={A, C, G, T/U}, corresponding to the bases adenine (A), cytosine (C), guanine (G) and thymine (T) or uracil (U). On the other hand, the 4-ary alphabet of the linear block code is denoted by Z₄={0, 1, 2, 3} for the integer residue ring and by GF(4)={0, 1, α, α²} for the Galois field satisfying the operations of addition and multiplication according to the corresponding mathematical structure.

Step 2—Determining the Galois Ring Extension

The necessary condition for the unique factorization of xⁿ−1 over GR*(4, r), the group of units, is that the DNA sequence length be an odd number of the form n=2^{r}−1. In the cases where the DNA sequences have length of the form n=2^{r}+2 the methionine, without loss of generality, may be discarded.

In this non-limiting example, we consider the targeting sequence: ATP synthase subunit delta', mitochondrial—Locus Q40089, whose length is n=63 nucleotides. Hence, the degree of the primitive polynomial to be used in the Galois field extension of GF(2) is r=6, for n=2^{r}−1=2⁶−1=63. Therefore, this value of r=6 will be used in the field extension in Step 4.

Step 3—Primitive Polynomials Related to the Galois Extension

In this non-limiting step, every primitive polynomial of degree r=6 is listed. The following are the primitive polynomials known in the open literature.

x⁶+x⁵+x³+x²+1

x⁶+x+1

x⁶+x⁵+x²+x+1

x⁶+x⁴+x³+x+1

x⁶+x⁵+x⁴+x+1

x⁶+x⁵+1

Step 4—GF(2) Galois Extension

The Galois field GF(2^{r}) is obtained from the extension of GF(2) by an ideal generated by any one of the primitive polynomials of degree r=6. In this step, we realize the extension of GF(2) in the following way:

Consider the Galois field GF(2^{r})=GF(2⁶)=GF(64)=F₆₄ given by

$$\frac{{F}_{2} [ x ]}{〈 p ( x ) 〉} ≅ \frac{{f}_{2} [ x ]}{〈 {x}^{6} + {x}^{5} + {x}^{3} + {x}^{2} + 1 〉} = \{ {a}_{0} + {a}_{1} x + {a}_{2} {x}^{2} + … + {a}_{5} {x}^{5} \text{:} {a}^{′} s ∈ {F}_{2} \} ,$$

where p(x) is a primitive polynomial from Step 3.

Let α be a primitive element in GF(64), equivalently, α is a root of x⁶+x⁵+x³+x²+1, that is, α⁶+α⁵+α³+α²+1=0 implying that α⁶=−α⁵−α³−α²−1. Now, since the coefficients of the polynomials that form the set of elements of F₆₄ belong to F₂, and from the modulo 2 reduction of these coefficients we arrive at α⁶=+α⁵+α³+α²+1. The elements of F₆₄ are listed in Table A.

TABLE A
Elements of GF(64) and its binary representation

| Elements of F₆₄ | (1 α α² α³ α⁴ α⁵) | Elements of F₆₄ | (1 α α² α³ α⁴ α⁵) |
|---|---|---|---|
| 0 | (000000) | α¹⁰ | (001100) |
| 1 | (100000) | ∂ | ∂ |
| α | (010000) | α⁵⁵ | (001001) |
| α² | (001000) | α⁵⁶ | (101001) |
| α³ | (000100) | α⁵⁷ | (111001) |
| α⁴ | (000010) | α⁵⁸ | (110001) |
| α⁵ | (000001) | α⁵⁹ | (110101) |
| α⁶ | (101101) | α⁶⁰ | (110111) |
| α⁷ | (111011) | α⁶¹ | (110110) |
| α⁸ | (110000) | α⁶² | (011011) |
| α⁹ | (011000) | α⁶³ | (100000) |

Step 5—Galois Ring Extension of Z₄

Consider the ring GR(4,6) as being the quotient of Z₄[x](set of all polynomials with coefficients over Z₄) by the ideal generated by the same primitive polynomial p(x) used in the Galois field extension in Step 4, that is,

$$\begin{matrix}\frac{{Z}_{4} [ x ]}{〈 p ( x ) 〉} ≅ \frac{{Z}_{4} [ x ]}{〈 {x}^{6} + {x}^{5} + {x}^{3} + {x}^{2} + 1 〉} \\ = \{ {b}_{0} + {b}_{1} x + {b}_{2} {x}^{2} + … + {b}_{5} {x}^{5} \text{:} {b}_{i}^{′} s ∈ {Z}_{4} \} .\end{matrix}$$

Next, we determine the elements in GR*(4,6). We know that the operations in GR*(4,6) are modulo (x⁶+x⁵+x³+x²+1). As α is a root of the primitive polynomial used in the field extension as well as in the ring extension, then α⁶=−α⁵−α³−α²−1. Since the coefficients of the polynomials in GR(4,6) are over Z₄, it follows that α⁶=3α⁵+3α³+3α²+3. Considering f=(010000)=α, all the invertible and nonzero elements in GR(4,6) are determined as the power of f, as shown in Table B.

TABLE B
Elements of GR*(4,6) and its 4-ary representations

| GR*(4,6) | (1 α α² α³ α⁴ α⁵) | GR*(4,6) | (1 α α² α³ α⁴ α⁵) |
|---|---|---|---|
| 1 | (100000) | f⁹ = x⁹ = α⁹ | (233002) |
| f = x = α | (010000) | ∂ | ∂ |
| f² = x² = α² | (001000) | f¹²⁰ = x¹²⁰ = α¹²⁰ | (331023) |
| f³ = x³ = α³ | (000100) | f¹²¹ = x¹²¹ = α¹²¹ | (130203) |
| f⁴ = x⁴ = α⁴ | (000010) | f¹²² = x¹²² = α¹²² | (110121) |
| f⁵ = x⁵ = α⁵ | (000001) | f¹²³ = x¹²³ = α¹²³ | (310311) |
| f⁶ = x⁶ = α⁶ | (303303) | f¹²⁴ = x¹²⁴ = α¹²⁴ | (330330) |
| f⁷ = x⁷ = α⁷ | (131031) | f¹²⁵ = x¹²⁵ = α¹²⁵ | (033033) |
| f⁸ = x⁸ = α⁸ | (312002) | f¹²⁶ = x¹²⁶ = α¹²⁶ | (100000) |

Step 6—Determining the Group of Units

From Step 5 we have that f generates a cyclic group of order n.d in GR*(4,6), where d≧1εZ and f_{d} generates a cyclic subgroup whose order is 63 in GR*(4,6). Hence, we have n.d=63.d=126 implying that d=2. Consequently, f²=(001000)=α² generates a cyclic subgroup of order 63 in GR*(4,6). Thus, β=α² is the primitive element that generates the cyclic subgroup Gₙ=G₆₃ as shown in Table C. This primitive element is used in the construction of a BCH code of length n=63 over Z₄.

TABLE C
Elements of G₆₃

| G₆₃ | (1 α α² α³ α⁴ α⁵) |
|---|---|
| β = x² = α² | (001000) |
| β² = x⁴ = α⁴ | (000010) |
| β³ = x⁶ = α⁶ | (303303) |
| β⁴ = x⁸ = α⁸ | (310202) |
| ∂ | ∂ |
| β⁶¹ = x¹²² = α¹²² | (110121) |
| β⁶² = x¹²⁴ = α¹²⁴ | (330330) |
| β⁶³ = x¹²⁶ = α¹²⁶ | (100000) |

Step 7—Determining the Generator Polynomial of Matrix G(x)

We may construct a BCH code of length n over Z₄, by considering the code minimum distance is at most equal to the code's length, that is, d≦n. The algorithm will analyze all possible values that d can take on and which are related to the error correction capability established by the inequality d≦2t+1, where t denotes the number of errors. The case in consideration, we have that n=63 and so the number of possible errors to be analyzed is 1≦t≦31.

Considering the code minimum distance is d=3, then any two consecutive powers of β may be used in the process of obtaining the generator polynomial of the BCH code. Without any loss of generality, choose β and β² as the two such consecutive powers. Thus, the generator polynomial g(x) is given by g(x)=1 cm(M₁(x), M₂(x)), where Mᵢ(x) is the minimal polynomial associated with the element βⁱ, i=1, 2 over GR*(4,6) (where β is a primitive element in G.) that has as its roots all the elements in the sequence,

[βⁱ, (βⁱ)^{p}, (βⁱ)^{p}^{²}, . . . , (βⁱ)^{p}^{^{r-1}}]

Hence, M₁(x)=M₂(x)=(x−β)(x−β²)(x−β⁴)(x−β⁸)(x−β¹⁶)(x−β³²). Therefore, g(x)=x⁶+3x⁵+x³+x²+2x+1 generates the desired code and it is related with the generator matrix G(x) of the BCH code over Z₄ with parameters (n,k,d)=(63,57,3).

Step 8—Determining the Generator Polynomial of Matrix H(x)

The generator polynomial of the parity-check matrix H(x) is, for example, obtained as follows:

$$\begin{matrix} h ( x ) = \frac{{x}^{n} - 1}{g ( x )} \\ = \frac{{x}^{63} - 1}{{x}^{6} + 3 {x}^{5} + {x}^{3} + {x}^{2} + 2 x + 1}\end{matrix}$$

$$h ( x ) = {x}^{57} + {x}^{56} + {x}^{55} + 2 {x}^{53} + 2 {x}^{52} + 2 {x}^{51} + {x}^{50} + 3 {x}^{47} + {x}^{43} + 3 {x}^{42} + 3 {x}^{40} + 3 {x}^{39} + 2 {x}^{38} + 3 {x}^{36} + {x}^{34} + 3 {x}^{33} + 2 {x}^{32} + 3 {x}^{31} + {x}^{29} + {x}^{28} + 3 {x}^{27} + 2 {x}^{26} + {x}^{25} + 3 {x}^{24} + 3 {x}^{23} + {x}^{22} + 2 {x}^{21} + {x}^{19} + {x}^{18} + 2 {x}^{17} + 3 {x}^{14} + 2 {x}^{13} + {x}^{12} + 3 {x}^{10} + 2 {x}^{9} + 2 {x}^{8} + 3 {x}^{7} + {x}^{6} + 3 {x}^{5} + 3 {x}^{4} + {x}^{3} + 2 x + 3 ,$$

where the coefficients of the polynomial h(x) belong to Z₄.

Step 9—Determining Matrix G(x) and its Transpose G^{T}(x):

Once the generator polynomial is determined in Step 7, the generator matrix is constructed as follows: Consider: g(x)=g₀+g₁x+g₂x²+ . . . +x^{n-k}, then the code generator matrix is given by:

$$G = ( \begin{matrix}{g}_{0} & {g}_{1} & {g}_{2} & {g}_{2} & 1 & 0 & 0 & … & 0 \\ 0 & {g}_{0} & {g}_{1} & {g}_{1} & {g}_{n - k - 1} & 1 & 0 & … & 0 \\ 0 & 0 & {g}_{0} & {g}_{0} & {g}_{n - k - 2} & {g}_{n - k - 1} & 1 & … & 0 \\ ⋮ & ⋮ & ⋮ & ⋱ & ⋮ & ⋮ & ⋮ & ⋱ & ⋮ \\ 0 & 0 & 0 & … & {g}_{0} & {g}_{1} & {g}_{2} & … & 1\end{matrix} )$$

By shifting the coefficients of the polynomial g(x) from the left to the right, we obtain matrix G(x) with dimension 57×63:

$$G ( x ) = \\ 121103100000000000000000000000000000000000000000000000000000000$$

$$012110310000000000000000000000000000000000000000000000000000000$$

$$001211031000000000000000000000000000000000000000000000000000000$$

$$000121103100000000000000000000000000000000000000000000000000000$$

$$000012110310000000000000000000000000000000000000000000000000000$$

$$000001211031000000000000000000000000000000000000000000000000000$$

$$000000121103100000000000000000000000000000000000000000000000000$$

$$000000012110310000000000000000000000000000000000000000000000000$$

$$000000001211031000000000000000000000000000000000000000000000000$$

$$000000000121103100000000000000000000000000000000000000000000000$$

$$000000000012110310000000000000000000000000000000000000000000000$$

$$000000000001211031000000000000000000000000000000000000000000000$$

$$000000000000121103100000000000000000000000000000000000000000000$$

$$000000000000012110310000000000000000000000000000000000000000000$$

$$000000000000001211031000000000000000000000000000000000000000000$$

$$000000000000000121103100000000000000000000000000000000000000000$$

$$000000000000000012110310000000000000000000000000000000000000000$$

$$000000000000000001211031000000000000000000000000000000000000000$$

$$000000000000000000121103100000000000000000000000000000000000000$$

$$000000000000000000012110310000000000000000000000000000000000000$$

$$000000000000000000001211031000000000000000000000000000000000000$$

$$000000000000000000000121103100000000000000000000000000000000000$$

$$000000000000000000000012110310000000000000000000000000000000000$$

$$000000000000000000000001211031000000000000000000000000000000000$$

$$000000000000000000000000121103100000000000000000000000000000000$$

$$000000000000000000000000012110310000000000000000000000000000000$$

$$000000000000000000000000001211031000000000000000000000000000000$$

$$000000000000000000000000000121103100000000000000000000000000000$$

$$000000000000000000000000000012110310000000000000000000000000000$$

$$000000000000000000000000000001211031000000000000000000000000000$$

$$000000000000000000000000000000121103100000000000000000000000000$$

$$000000000000000000000000000000012110310000000000000000000000000$$

$$000000000000000000000000000000001211031000000000000000000000000$$

$$000000000000000000000000000000000121103100000000000000000000000$$

$$000000000000000000000000000000000012110310000000000000000000000$$

$$000000000000000000000000000000000001211031000000000000000000000$$

$$000000000000000000000000000000000000121103100000000000000000000$$

$$000000000000000000000000000000000000012110310000000000000000000$$

$$000000000000000000000000000000000000001211031000000000000000000$$

$$000000000000000000000000000000000000000121103100000000000000000$$

$$000000000000000000000000000000000000000012110310000000000000000$$

$$000000000000000000000000000000000000000001211031000000000000000$$

$$000000000000000000000000000000000000000000121103100000000000000$$

$$000000000000000000000000000000000000000000012110310000000000000$$

$$000000000000000000000000000000000000000000001211031000000000000$$

$$000000000000000000000000000000000000000000000121103100000000000$$

$$000000000000000000000000000000000000000000000012110310000000000$$

$$000000000000000000000000000000000000000000000001211031000000000$$

$$000000000000000000000000000000000000000000000000121103100000000$$

$$000000000000000000000000000000000000000000000000012110310000000$$

$$000000000000000000000000000000000000000000000000001211031000000$$

$$000000000000000000000000000000000000000000000000000121103100000$$

$$000000000000000000000000000000000000000000000000000012110310000$$

$$000000000000000000000000000000000000000000000000000001211031000$$

$$000000000000000000000000000000000000000000000000000000121103100$$

$$000000000000000000000000000000000000000000000000000000012110310$$

$$000000000000000000000000000000000000000000000000000000001211031$$

Matrix G^{T}(x) with dimension 63×57 is determined by changing the elements of each row as the elements of the column.

Step 10—Determining Matrix H(x) and its Transpose H^{T}(x)

Once the polynomial h(x) is obtained in Step 8, matrix H(x) is determined by realizing the displacement of the coefficients of the generator polynomial h(x) from the right to the left. Matrix H(x) with dimension 6×63 is given by:

$$H ( x ) = \\ 000001110222100300013033203013230113213312011200321032231331123$$

$$000011102221003000130332030132301132133120112003210322313311230$$

$$000111022210030001303320301323011321331201120032103223133112300$$

$$001110222100300013033203013230113213312011200321032231331123000$$

$$011102221003000130332030132301132133120112003210322313311230000$$

$$111022210030001303320301323011321331201120032103223133112300000$$

Matrix H^{T}(x) with dimension 63×6 is determined by changing the elements of each row as the elements of the column.

Step 11—Labelling the DNA Sequence by Use of the Code Alphabet

In this non-limiting example, we analyze if the BCH code over ring is capable of reproducing the targeting sequence of the organism: Ipomoea potatoes, locus: [Q40089], protein: ATP synthase subunit delta', organelle: mitochondrion, subcompartment mitochondrial: internal membrane, length: 63 nucleotides. As the mapping N→Z₄ is unknown, we consider all the permutations between these two sets. Therefore, this step determines all the 24 permutations between the genetic code alphabet N={A,C,G,T} and the BCH code alphabet Z₄={0,1,2,3} of the targeting sequence to be analyzed. The rows of matrix P correspond to the 24 permutations of the targeting sequence, SD.

(SEQ ID NO: 2)
SD = ATGTTCAGGCACTCTTCTCGACTCCTAGCTCGCGCCACCACAA
TGGGGTGGCGTCGCCCCTTC

032331022101313313120131130213121211011010032222322123121111331 023221033101212212130121120312131311011010023333233132131111221 031332011202323323210232230123212122022020031111311213212222332 013112033202121121230212210321232322022020013333133231232222112 012113022303131131320313310231323233033030012222122321323333113 021223011303232232310323320132313133033030021111211312313333223 132330122010303303021030031203020200100101132222322023020000330 123220133010202202031020021302030300100101123333233032030000220 130332100212323323201232231023202022122121130000300203202222332 103002133212020020231202201320232322122121103333033230232222002 120223100313232232301323321032303033133131120000200302303333223 102003122313030030321303301230323233133131102222022320323333003 231330211020303303012030032103010100200202231111311013010000330 213110233020101101032010012301030300200202213333133031030000110 230331200121313313102131132013101011211212230000300103101111331 203001233121010010132101102310131311211212203333033130131111001 210113200323131131302313312031303033233232210000100301303333113 201003211323030030312303302130313133233232201111011310313333003 321220311030202202013020023102010100300303321111211012010000220 312110322030101101023010013201020200300303312222122021020000110 320221300131212212103121123012101011311313320000200102101111221 302001322131010010123101103210121211311313302222022120121111001 310112300232121121203212213021202022322323310000100201202222112 301002311232020020213202203120212122322323301111011210212222002

Step 12—Verifying if the DNA Sequence is a Codeword of G(x);

In this non-limiting step, we consider that the DNA sequence under the action of each one of the 24 permutations from Step 11 is a codeword. Hence, in order to determine if each one of these 24 possibilities is in fact a codeword we use the relationship v.H^{T}=0, where v is a possible codeword and H^{T} is the transpose of the parity-check matrix found in Step 10. Yet in this step we analyze the DNA sequences with one nucleotide error, by considering the 3 other possibilities of nucleotides in each position in the sequence for each permutation. Finally, we analyze all possible combinations involving two nucleotide errors in each permutation.

Step 13—Go to Step 7 and Determine Another Generator Polynomial;

In this non-limiting step, we determine another value of the minimum distance d=5 and use the same procedure to calculate the generator polynomial corresponding to this distance.

Step 14—Repeat Step 8 Through Step 12 for the Generator Polynomial Obtained in Step 13, Until all the Possibilities of the Generator Polynomial are Realized;

In this non-limiting step, the algorithm determines all the codewords found with no nucleotide differences, differing in one nucleotide, and differing in two nucleotides, by use of all the generator polynomials corresponding to the minimum distance 3≦d≦63, and store the results.

Step 15—go to Step 3 and Choose Another Primitive Polynomial;

Step 16—Repeat all the Steps from Step 4 Up to Step 14 Until all the Primitive Polynomials have been Used in Step 3;

Step 17—Label all the Codewords Using the Alphabet of the Genetic Code;

In this non-limiting step, all the stored codewords are labeled using the code alphabet, Z₄={0, 1, 2, 3}, and they will be converted in nucleotides using the labelling of the genetic code N={A,C,G,T}.

Step 18—Compare all Code Words Stored in Step 17 with the Original DNA Sequence;

Step 19—Define the Labelling of the DNA Sequence and Show where the Differences have Occurred, End.

### Computer Program Description

The present invention is, in one aspect, a method of analyzing polymorphisms and mutations in DNA sequences. One aspect of the invention resides in a digital communication system comprising an apparatus for analyzing polymorphisms and mutations in DNA sequences. As used herein, the apparatus is to be understood as comprising a “computer system,” wherein the computer system includes at least a memory and a processor. Generally, the memory will store, at one time or another, instructions, including at least portions of an executable program code, which can be thereafter read by the processor, thereby enabling the computer system to carry out operations, including at least analyzing polymorphisms and mutations in DNA sequences. Generally, the processor will read and carry out one or more of the instructions included in the executable program code. The memory and the processor may be physically located in the same place, or may be physically located in separate places.

Another aspect of the present invention is a computer readable medium having embodied thereon a computer program that includes at least the executable program code that enables the computer system to carry out operations, including at least analyzing polymorphisms and mutations in DNA sequences. The computer program that includes at least the executable program code may be supplied on any one of a variety of media. An artisan skilled in the field of computers will appreciate that term “media” may be interchangeable with the phrases “computer-readable media” or “recording medium.” The media on which the computer program that includes at least the executable program code may reside, may include a diskette, a tape, a compact disc (CD), a digital versatile disks (DVD), an integrated circuit, a read-only memory (ROM), a cartridge such as a memory stick, or any other similar medium useable by computers. Further, the media on which the computer program that includes at least the executable program may reside, may include a remote transmission through a communications circuit so that the computer program may be distributed over network coupled or connected computer systems. Thus, the terms “media,” “computer-readable media,” or “recording medium” are intended to include all of the foregoing and any other medium by which software may be provided to a computer.

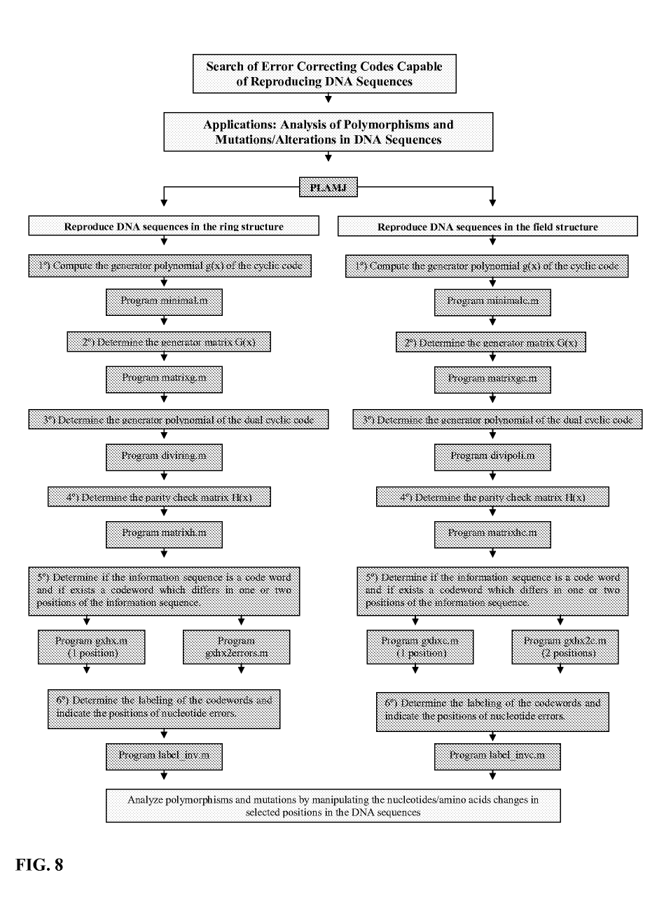

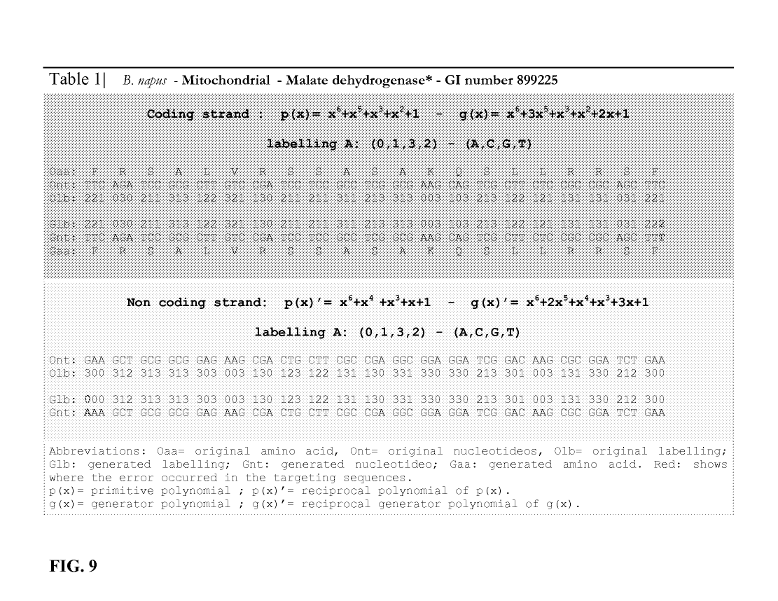

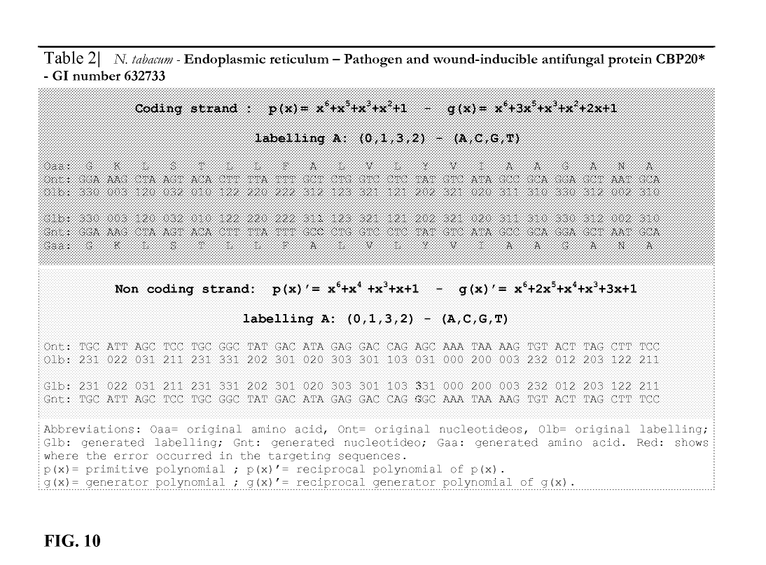

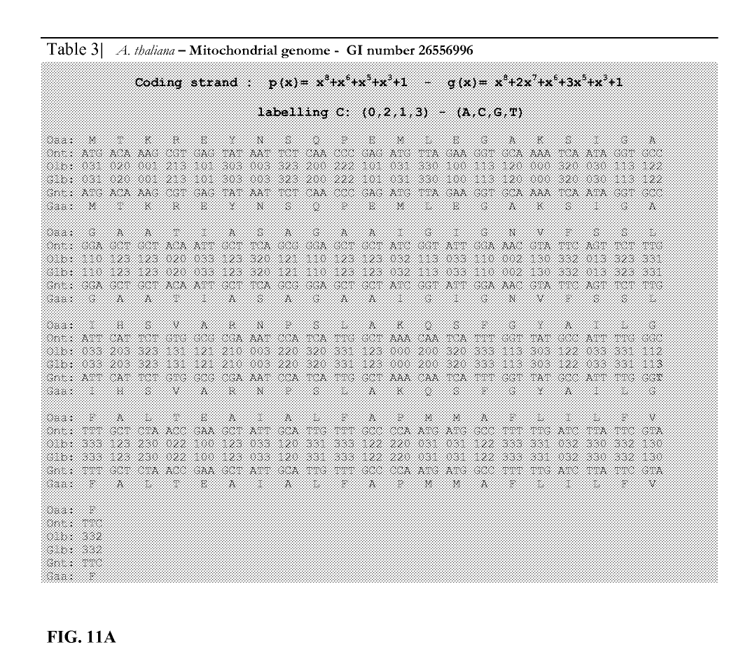

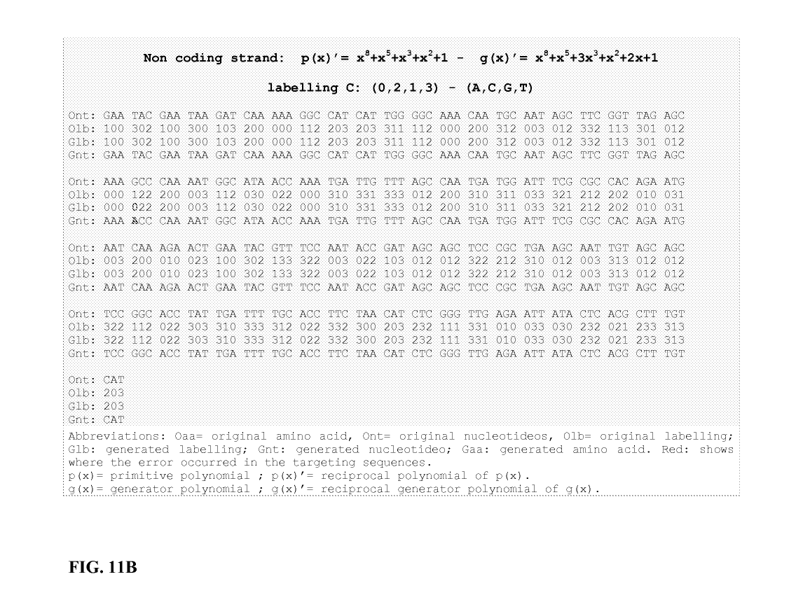

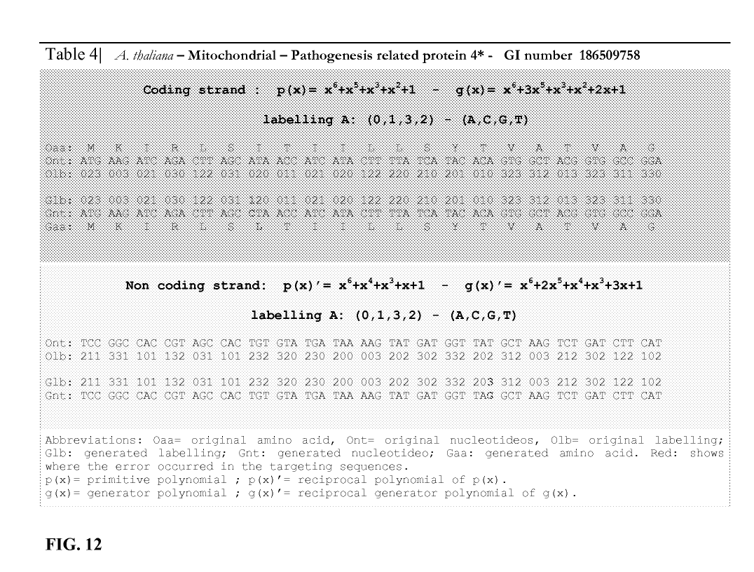

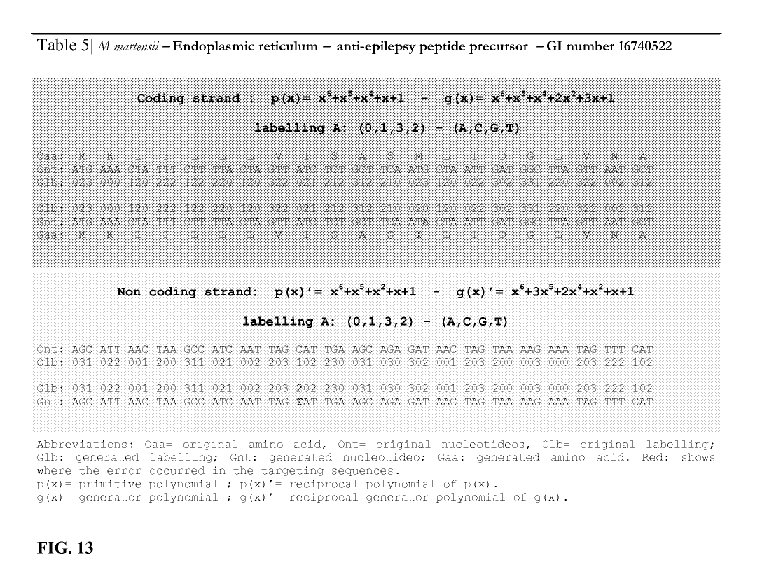

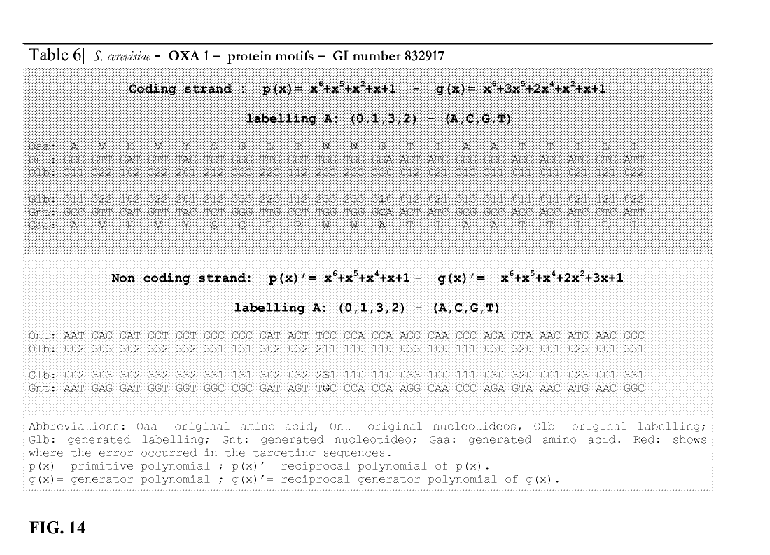

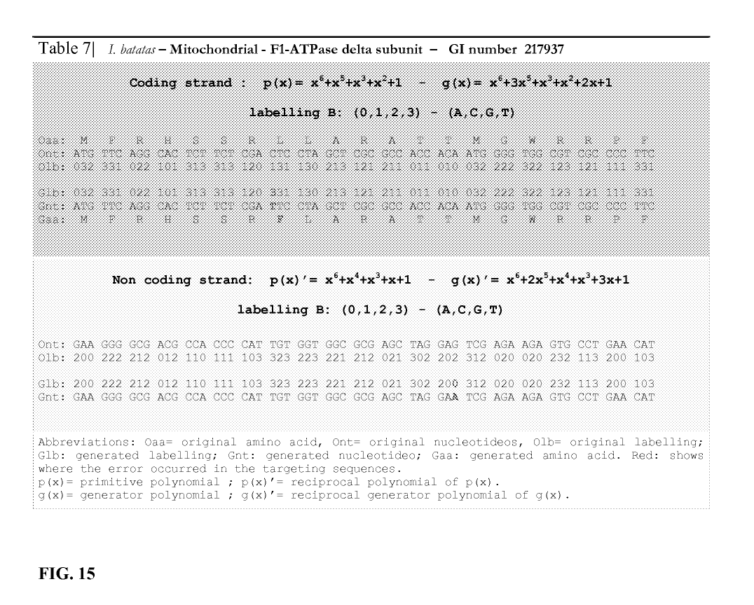

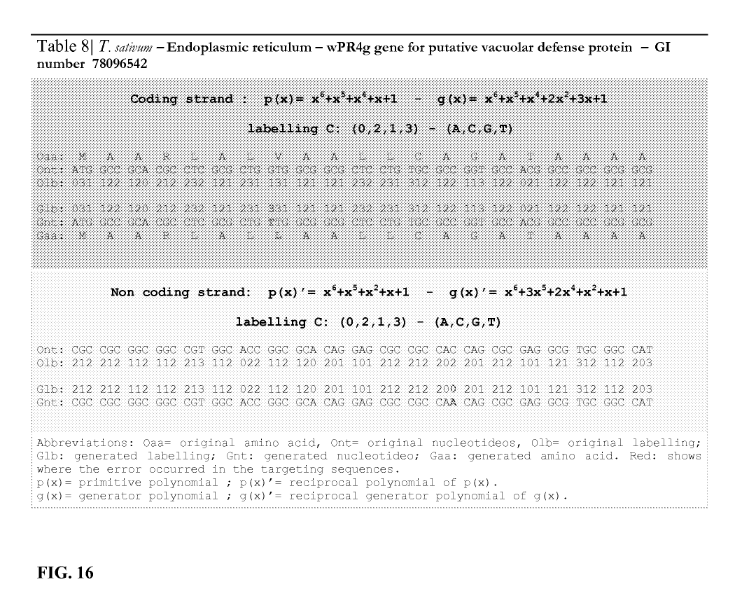

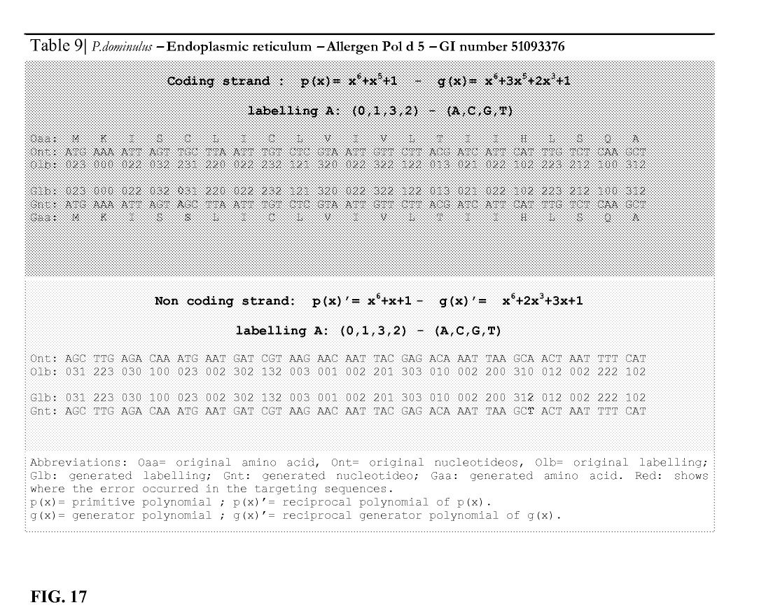

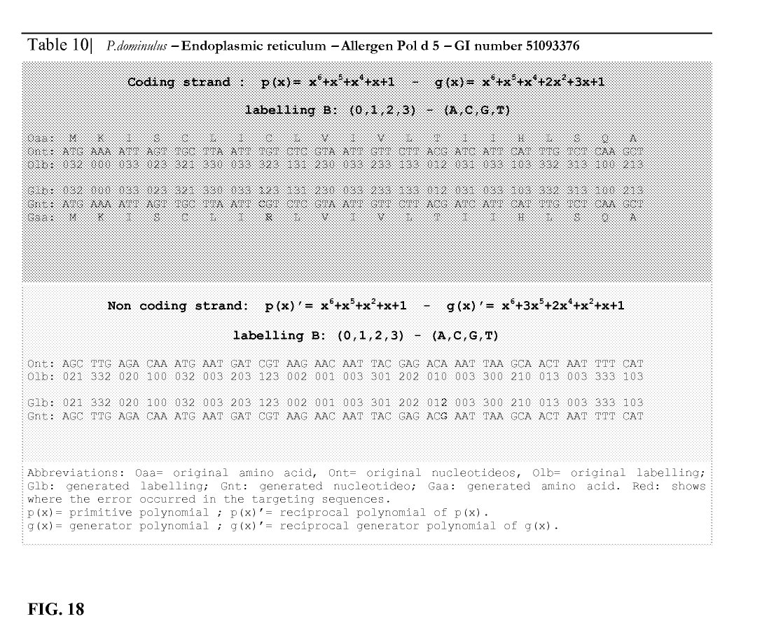

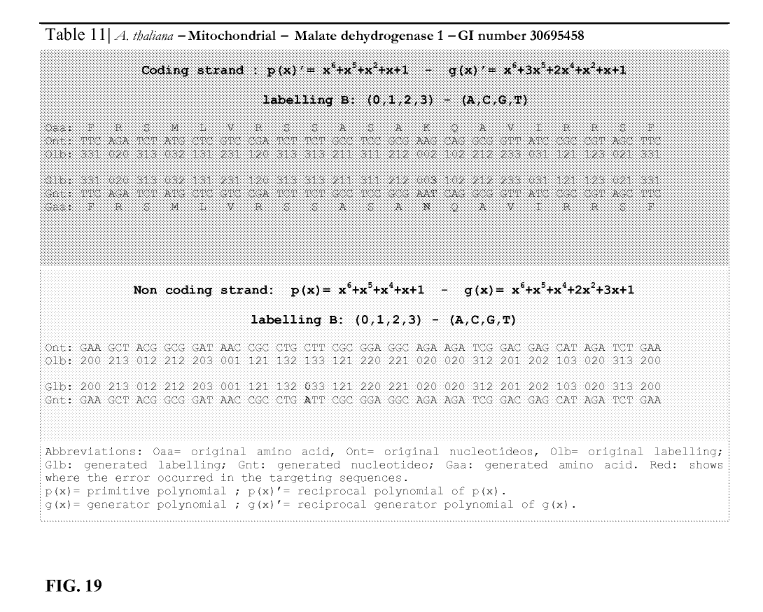

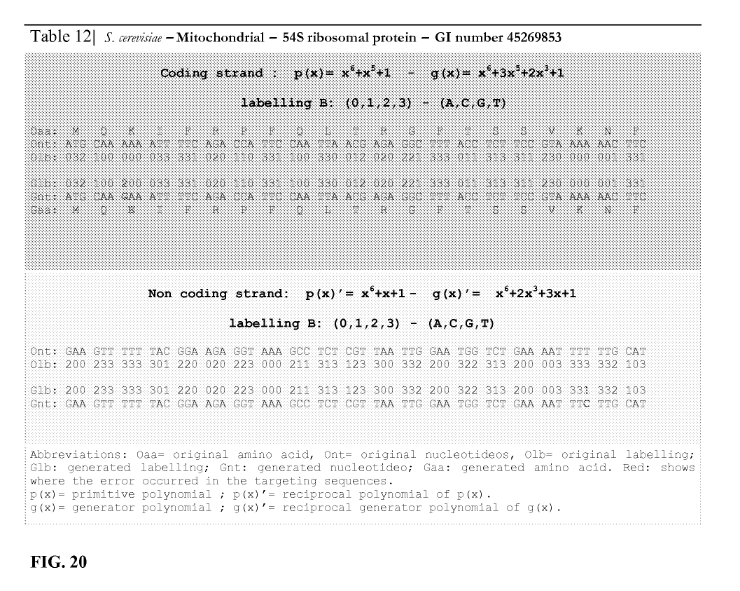

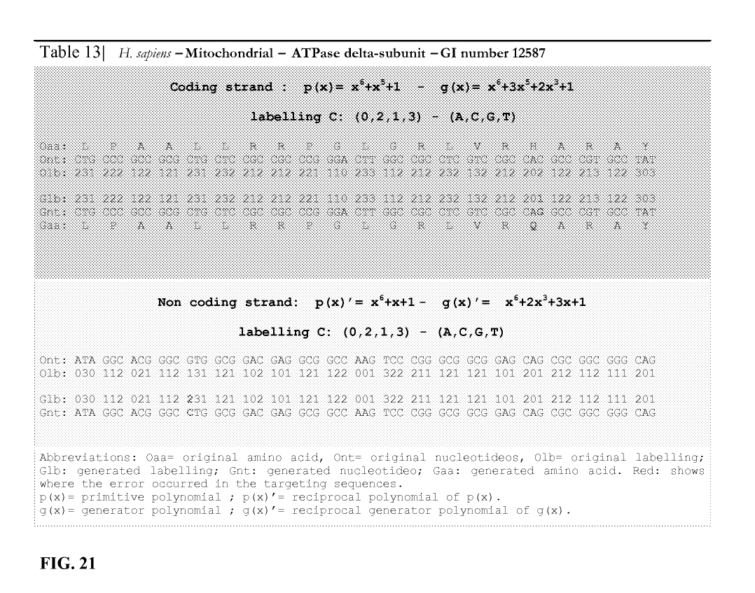

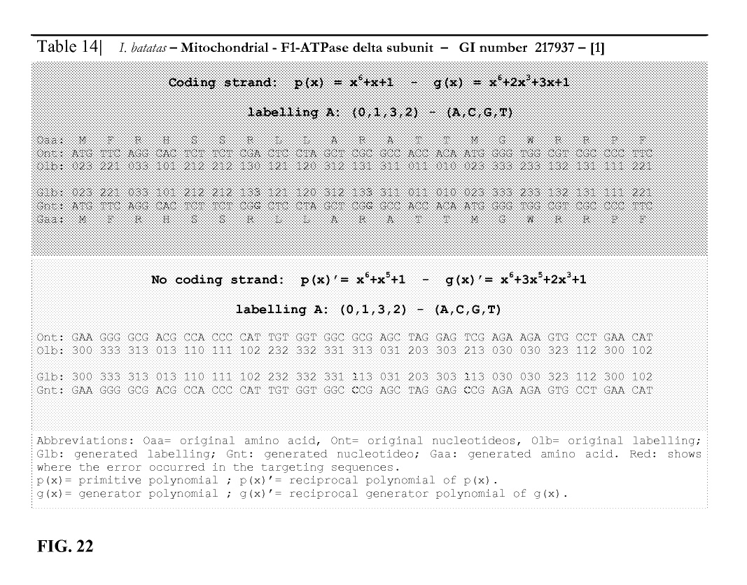

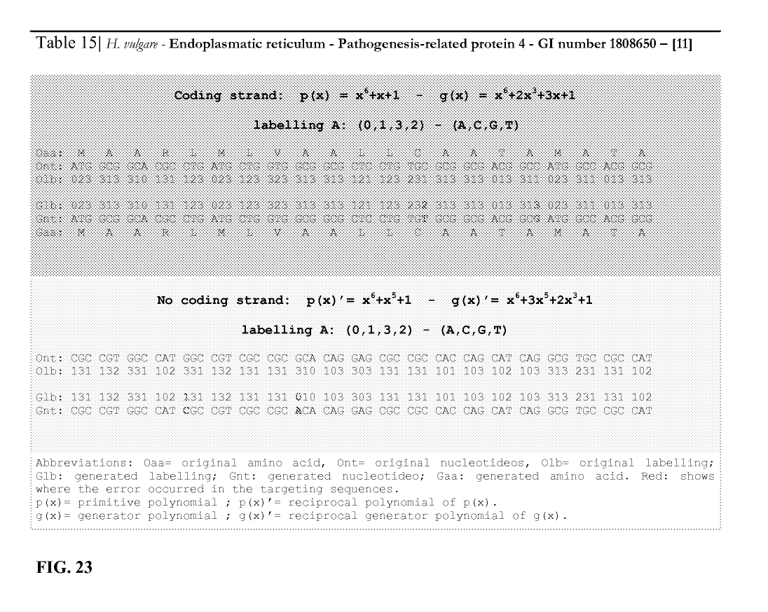

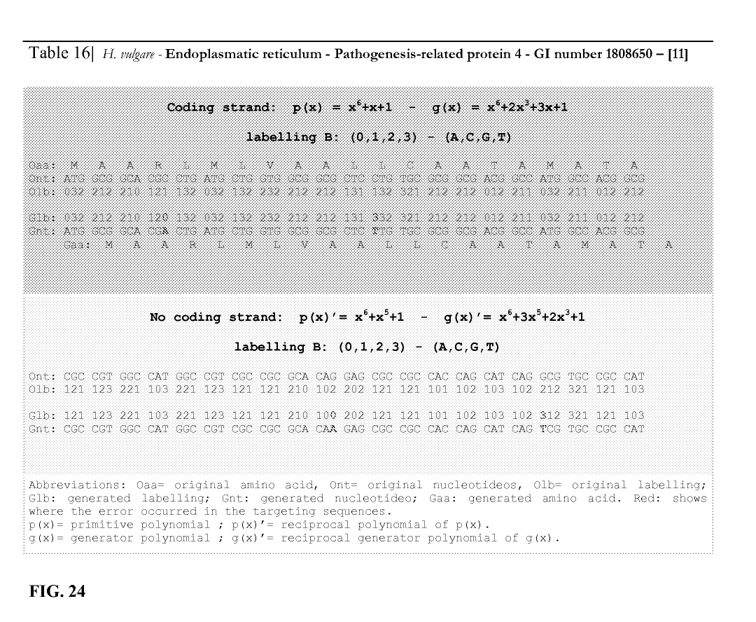

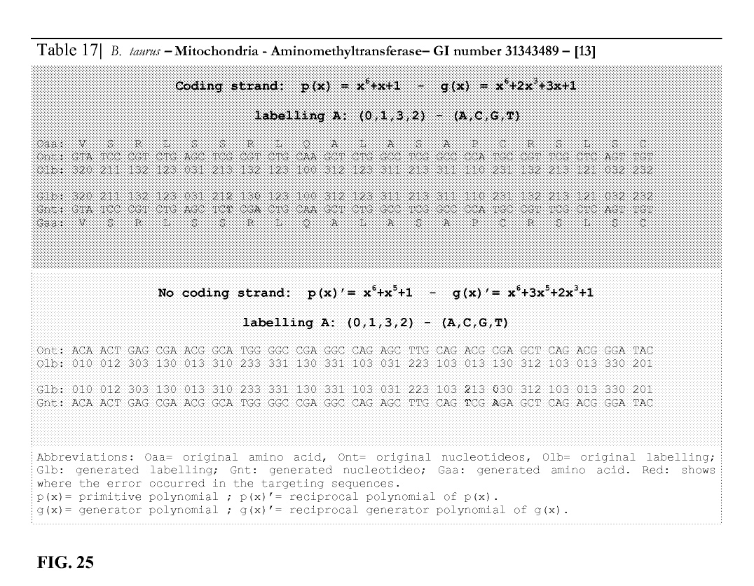

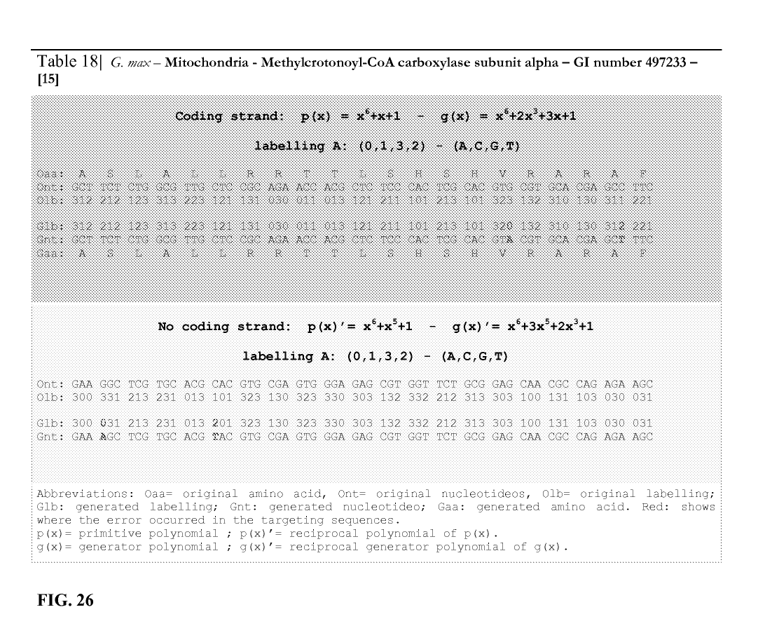

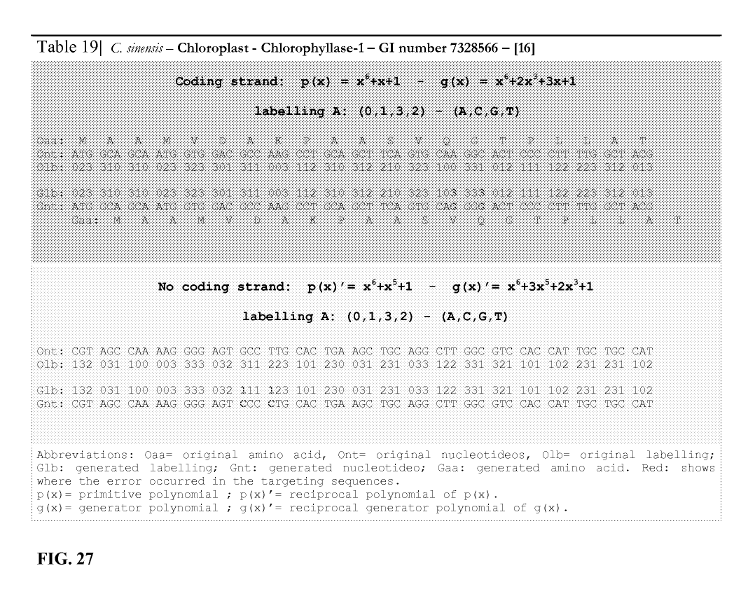

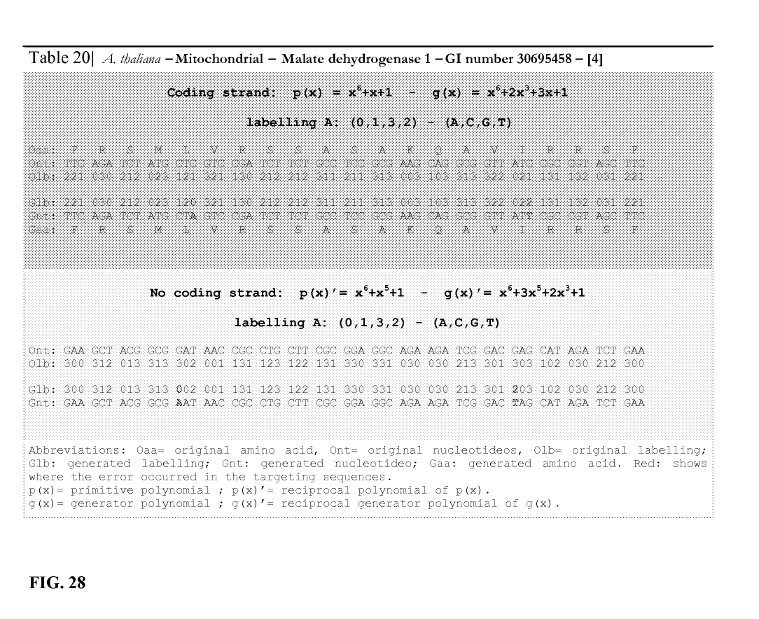

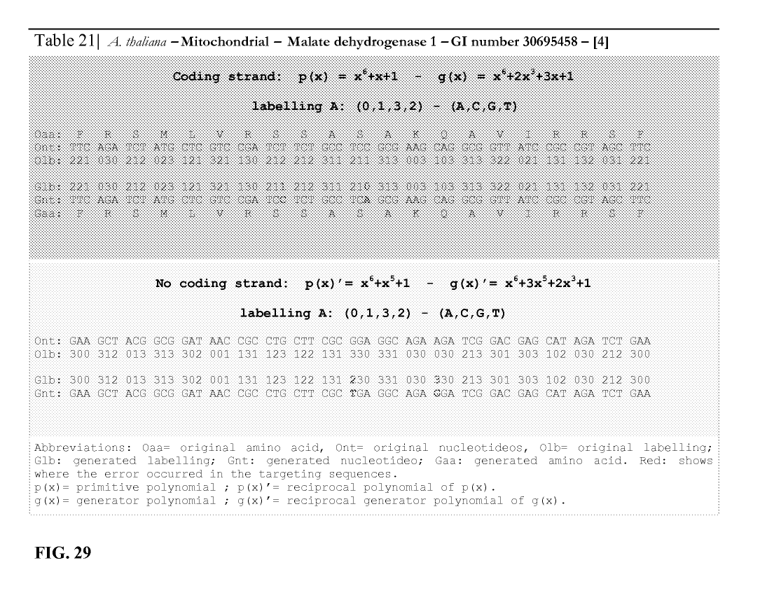

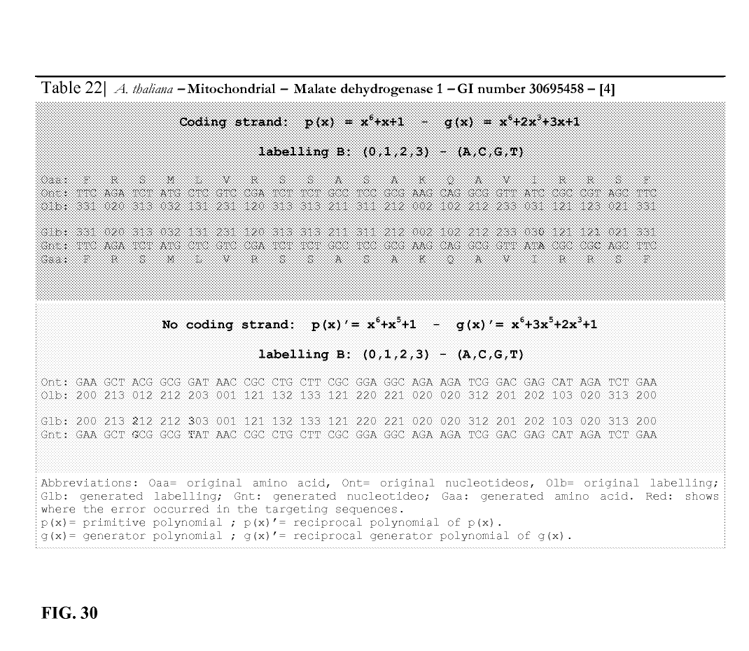

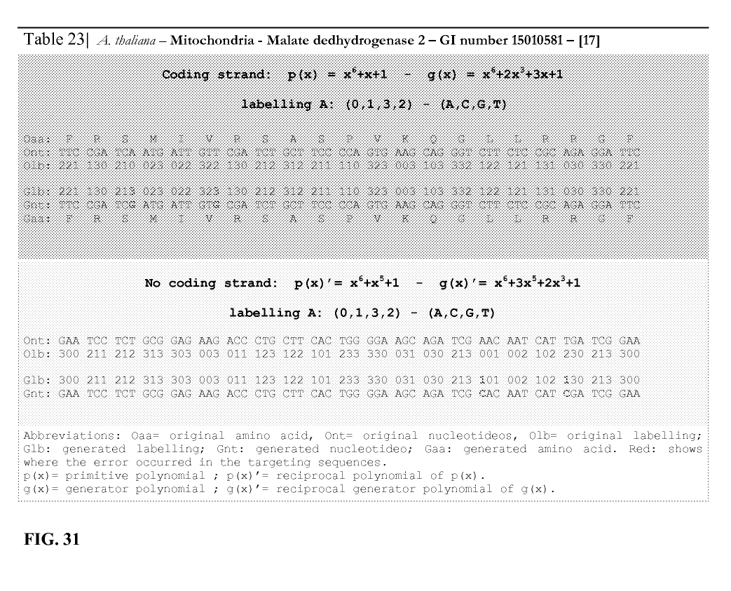

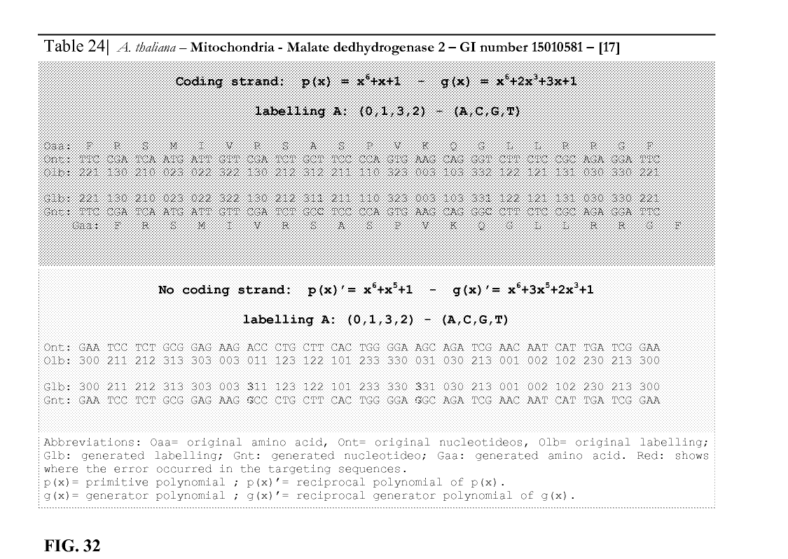

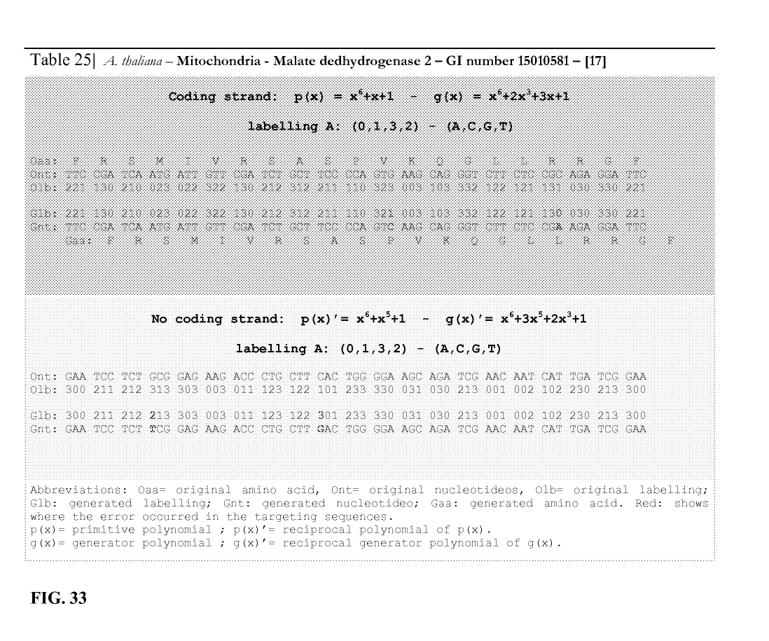

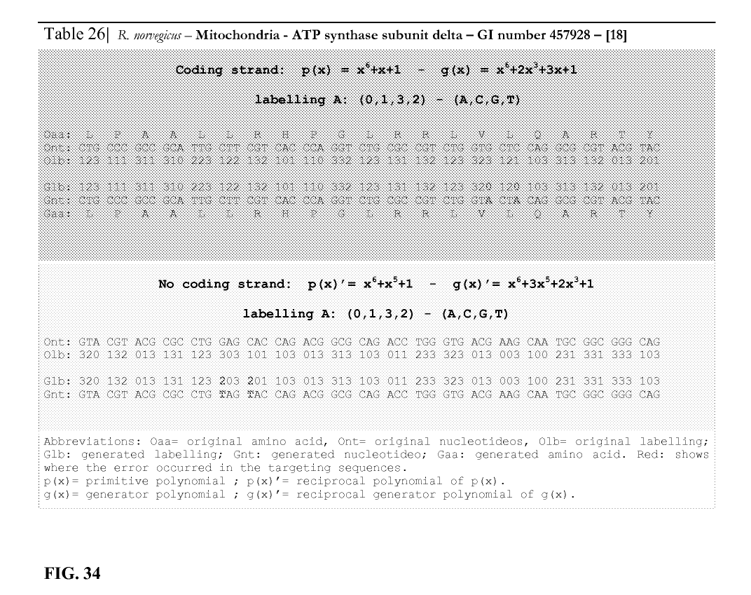

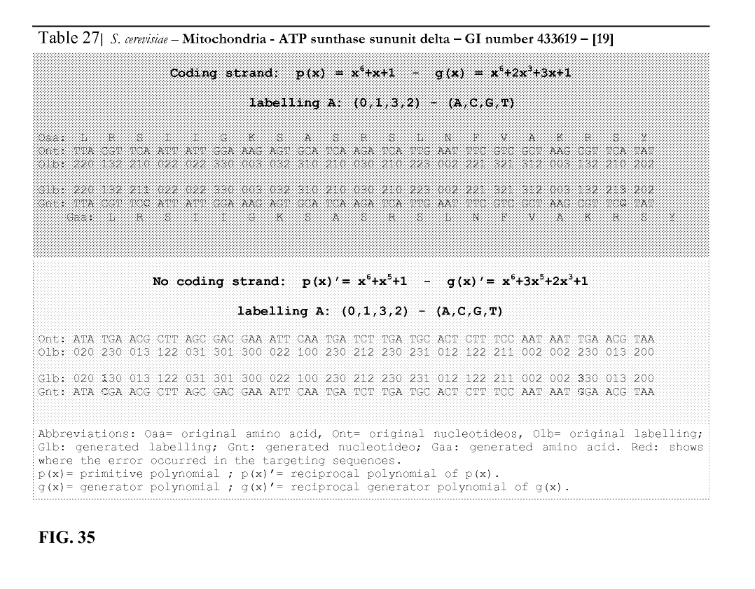

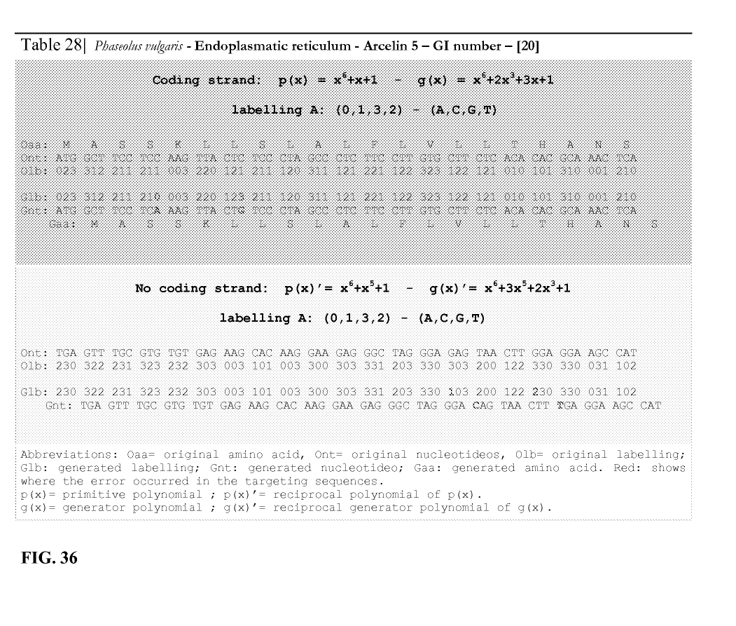

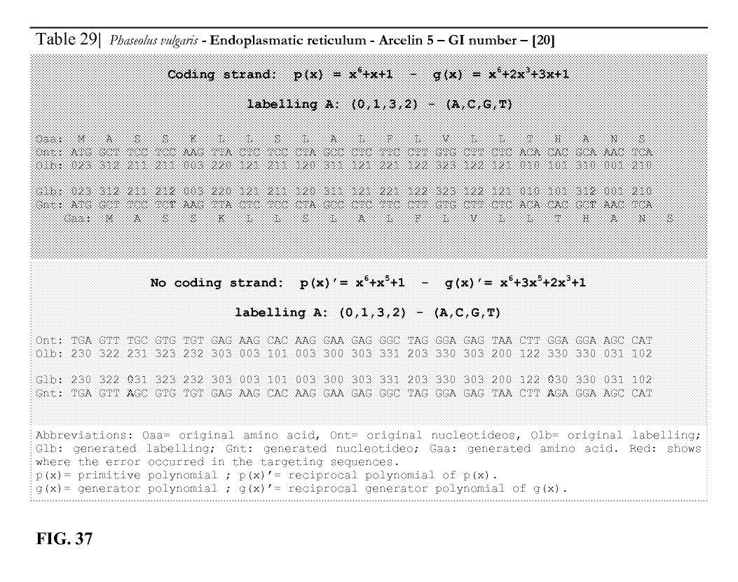

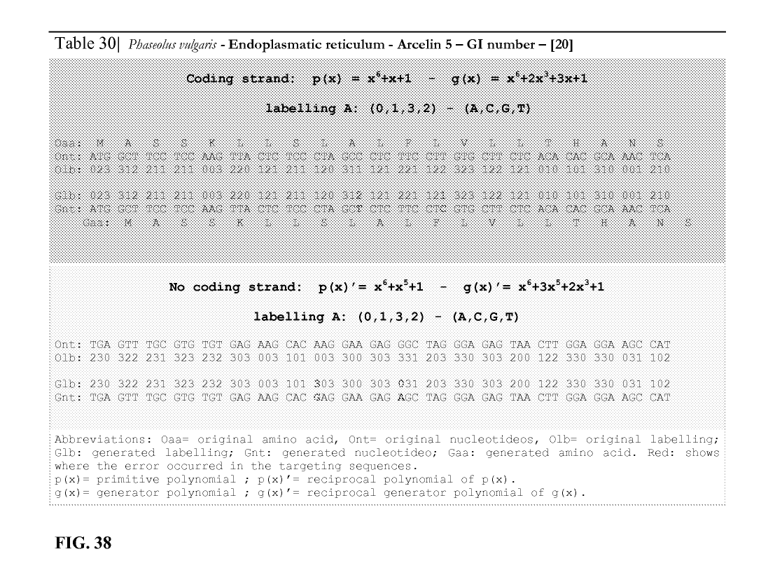

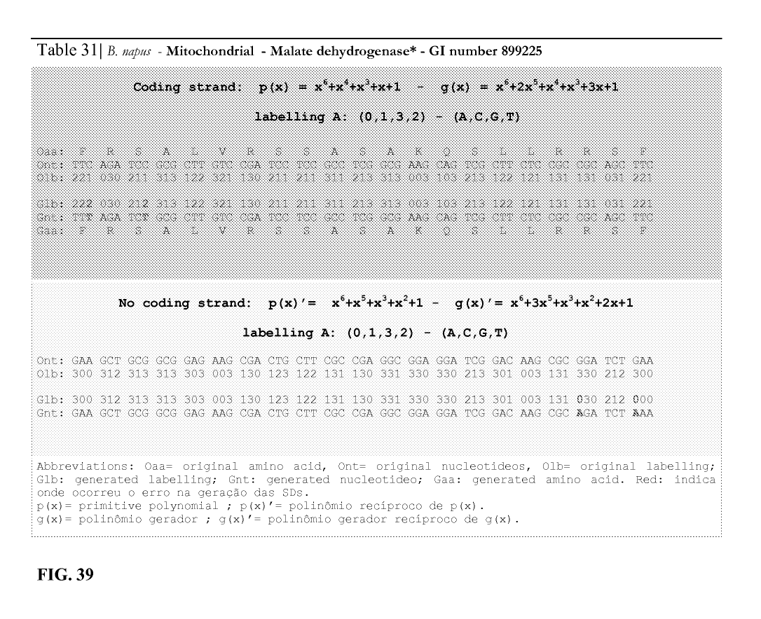

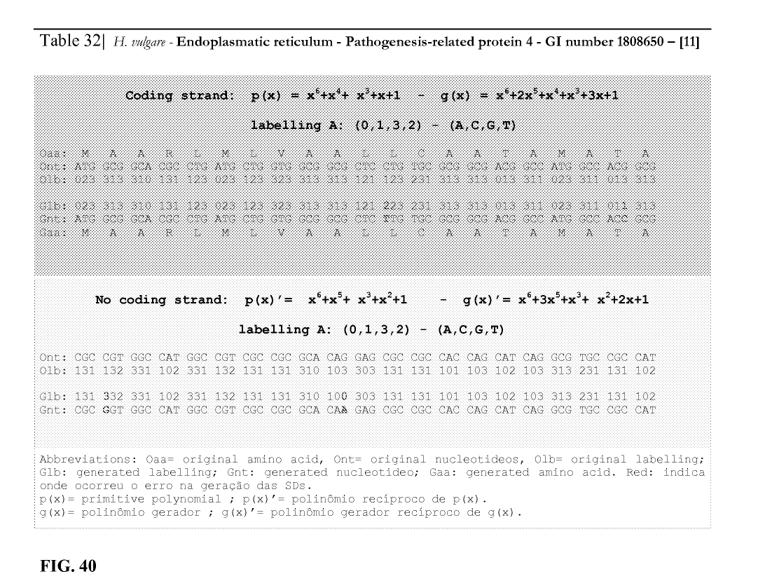

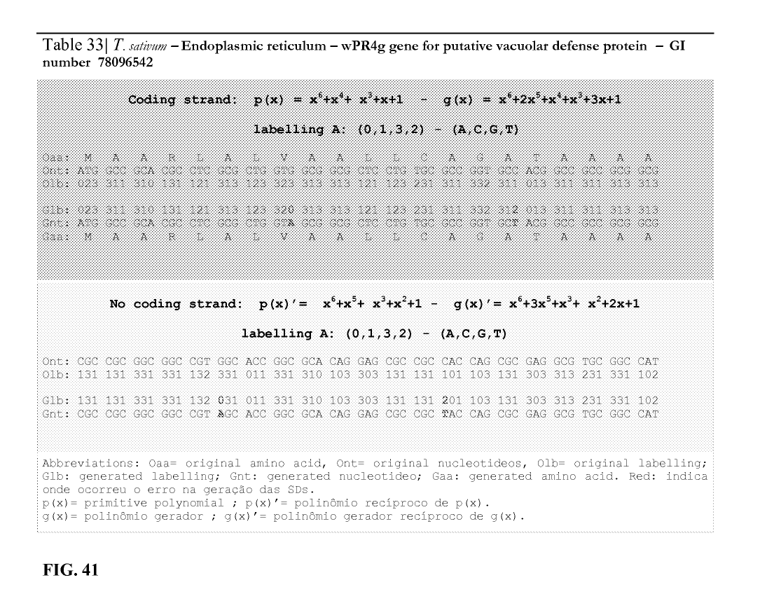

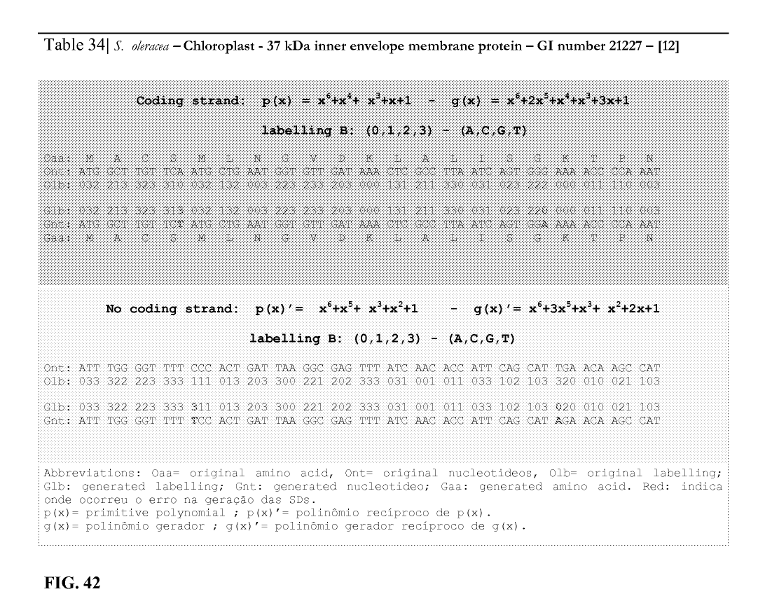

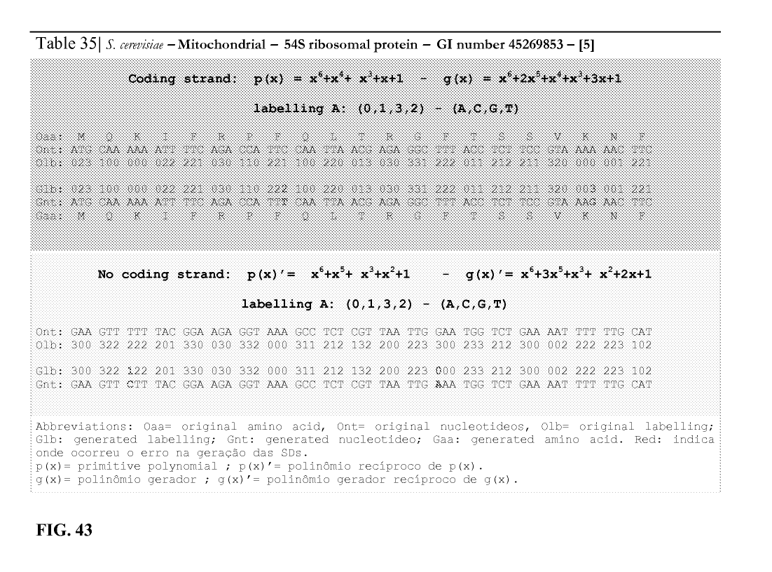

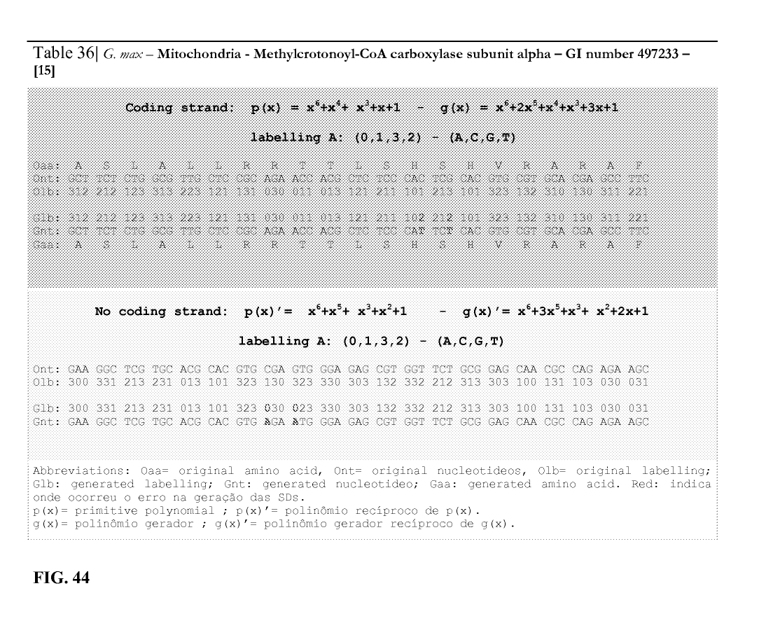

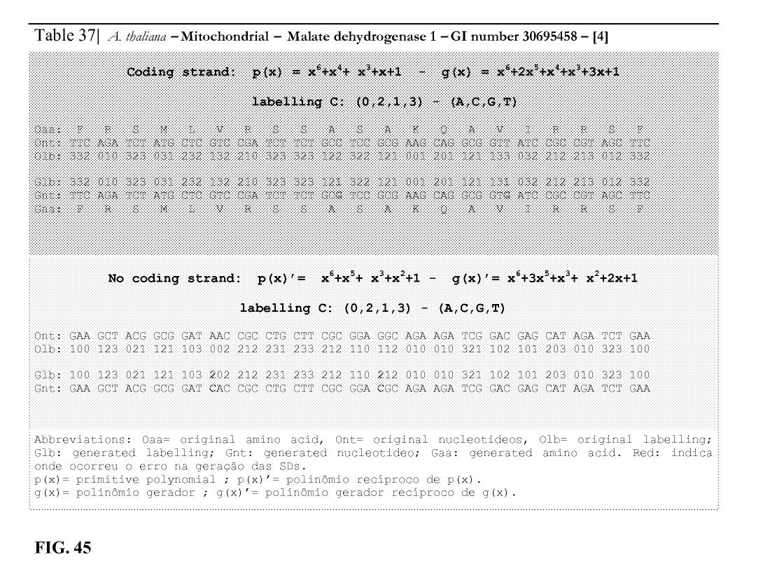

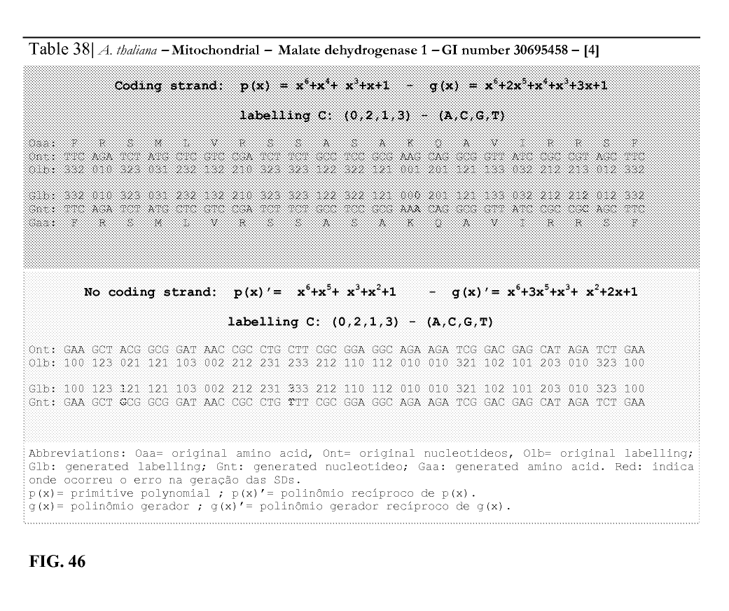

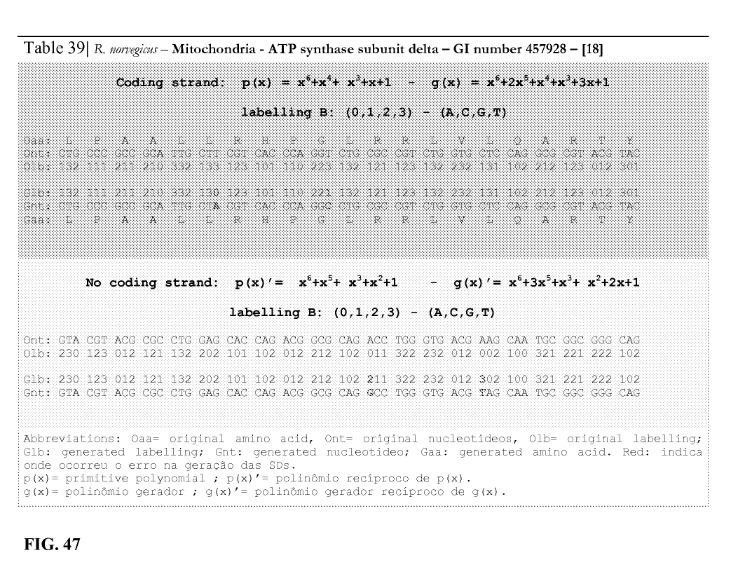

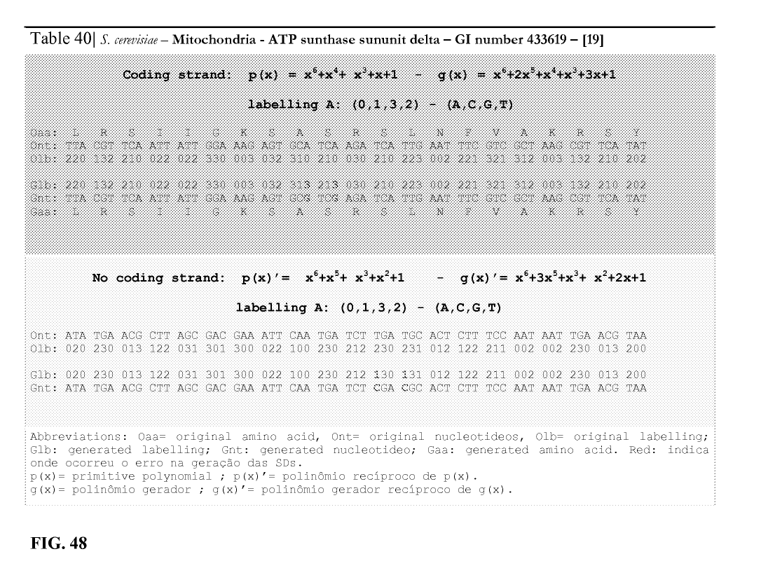

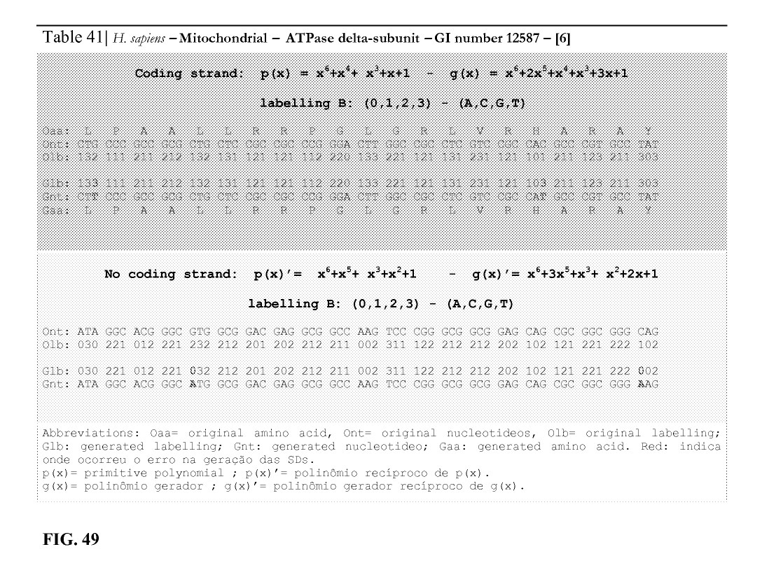

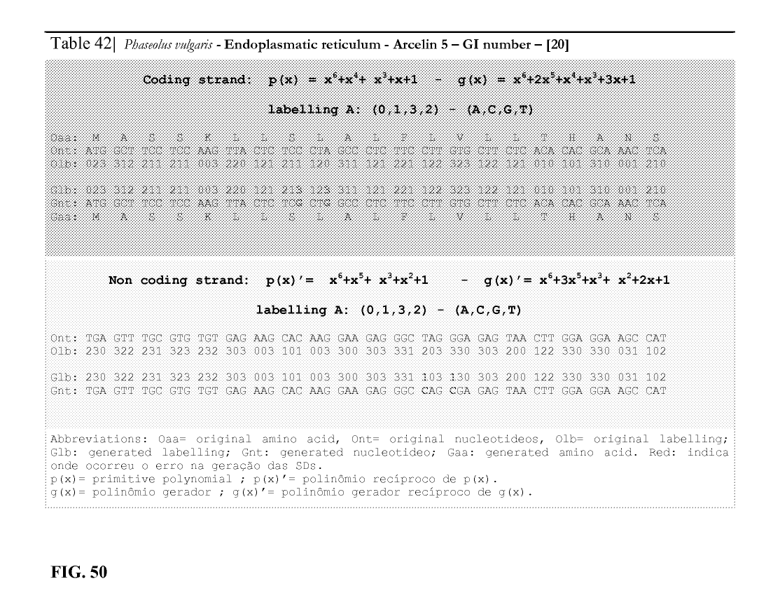

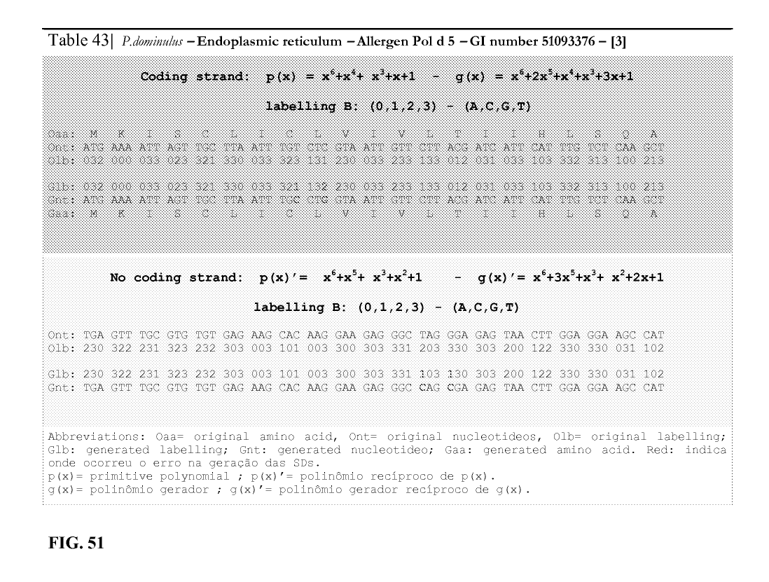

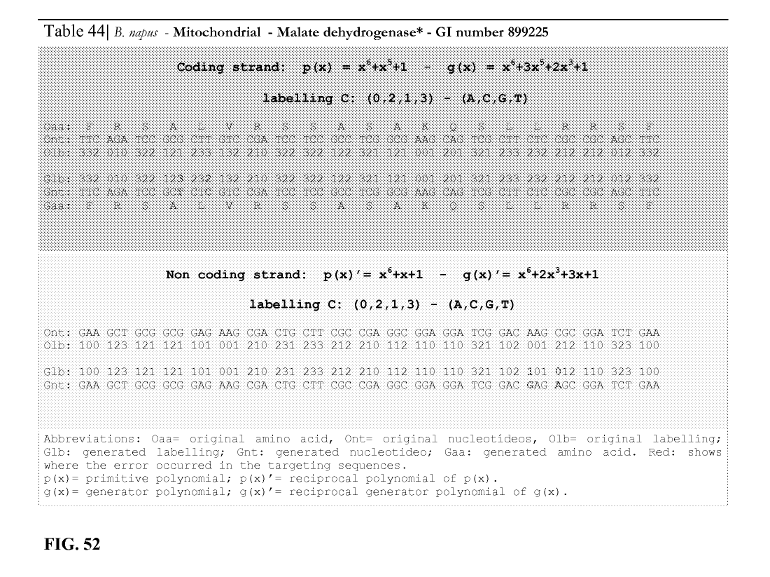

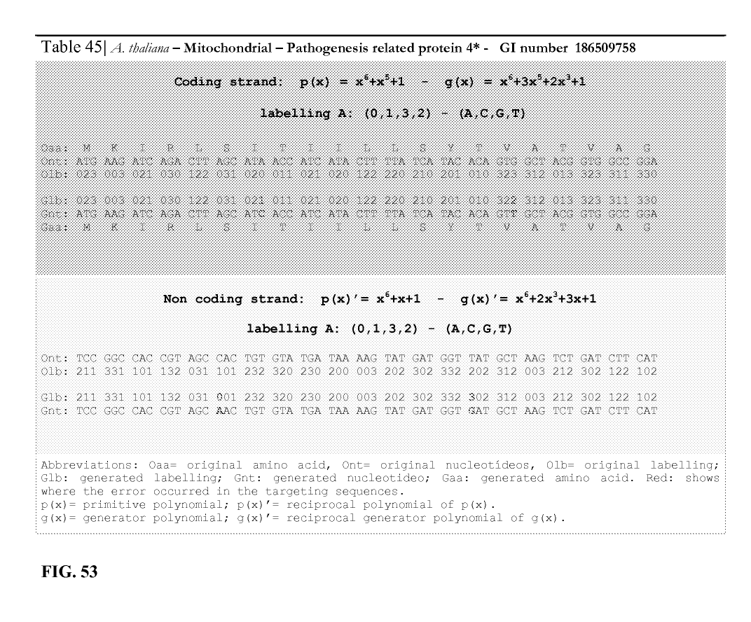

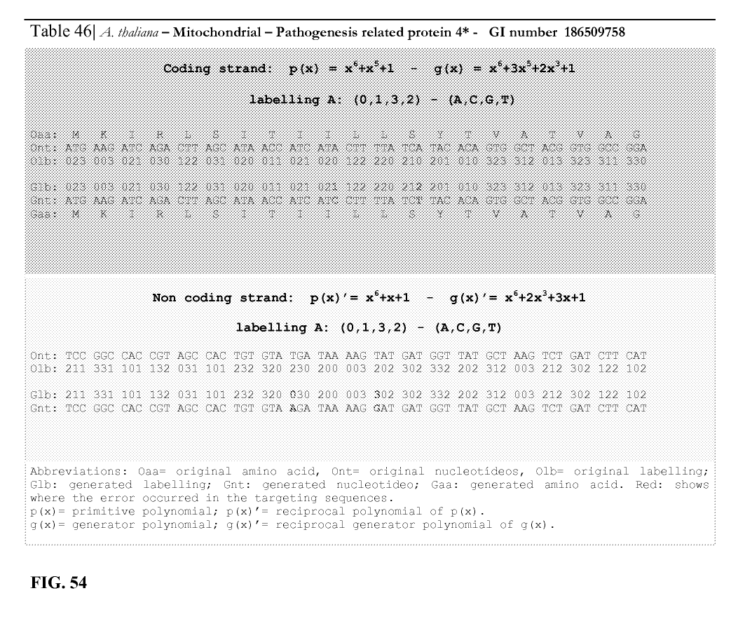

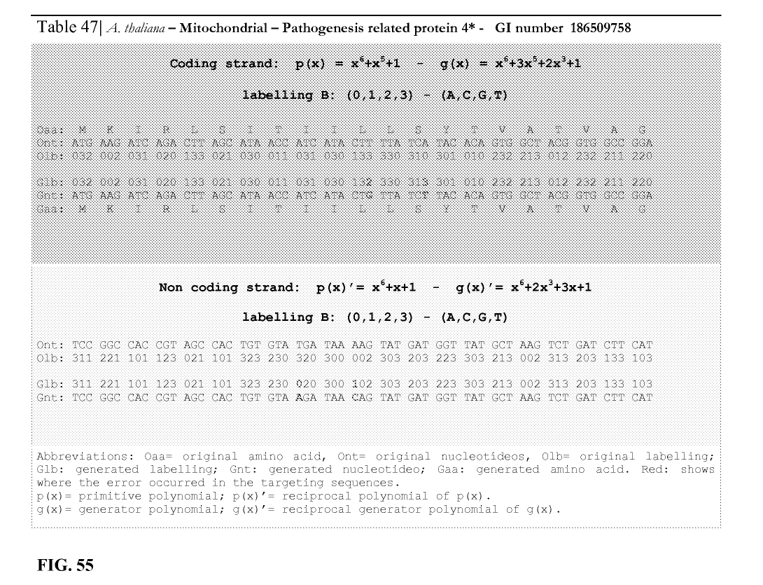

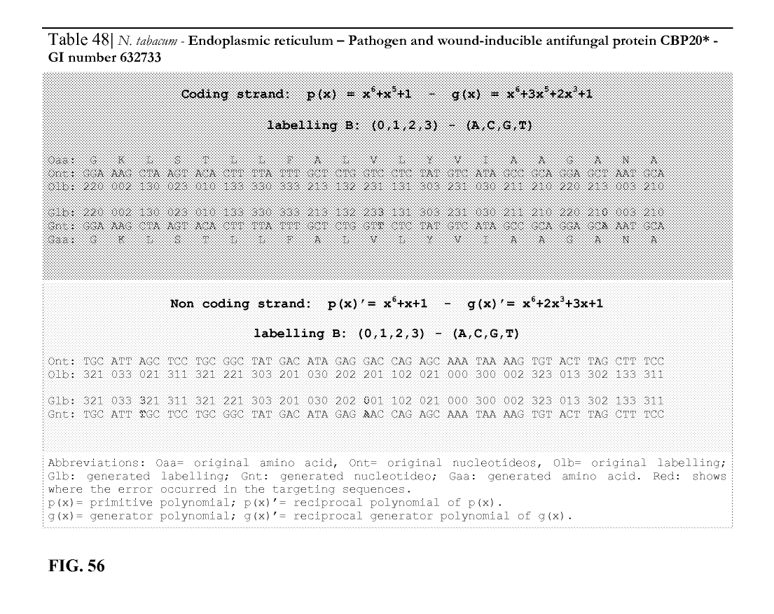

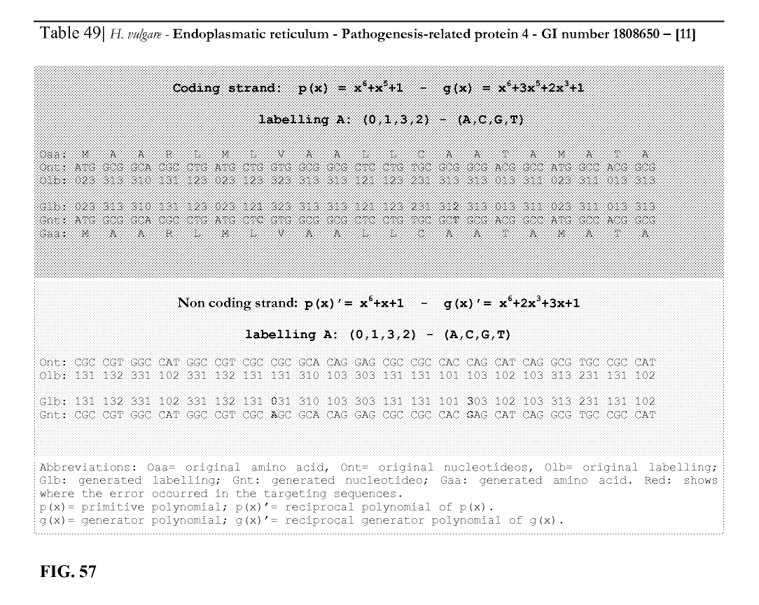

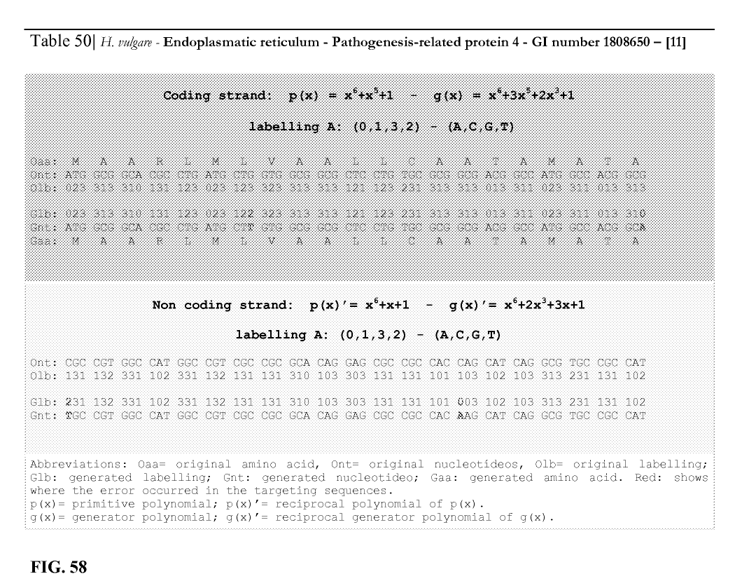

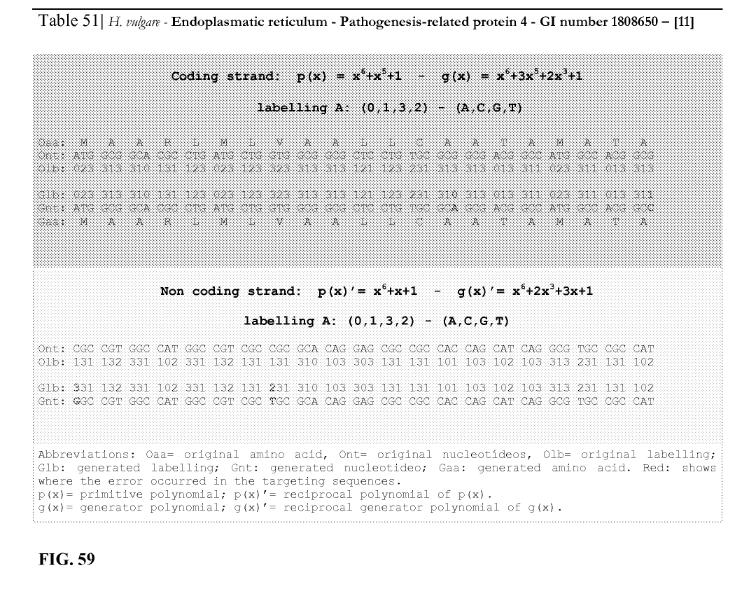

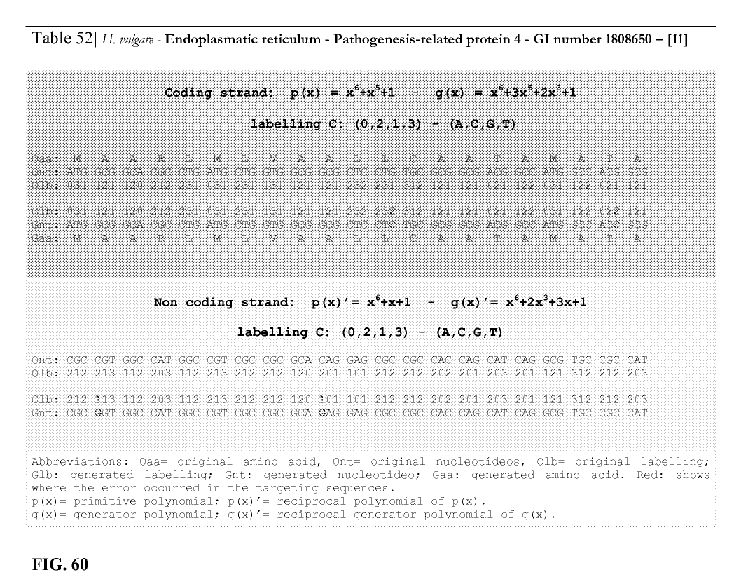

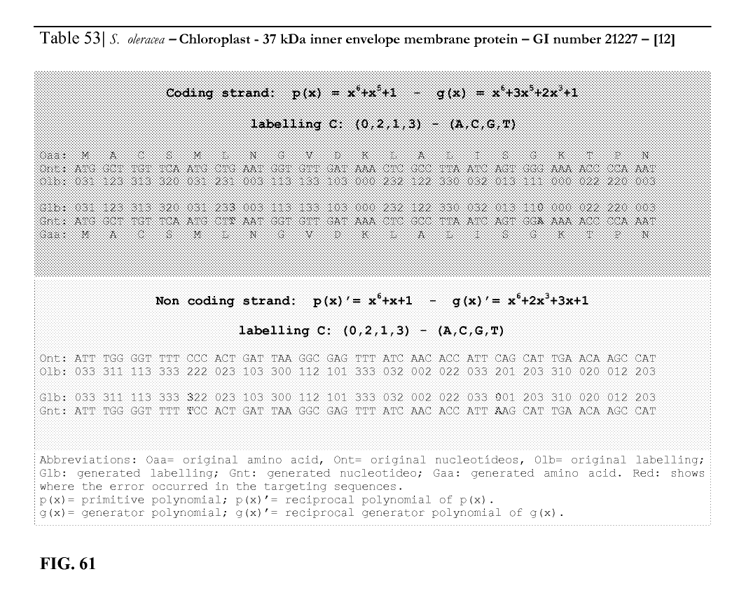

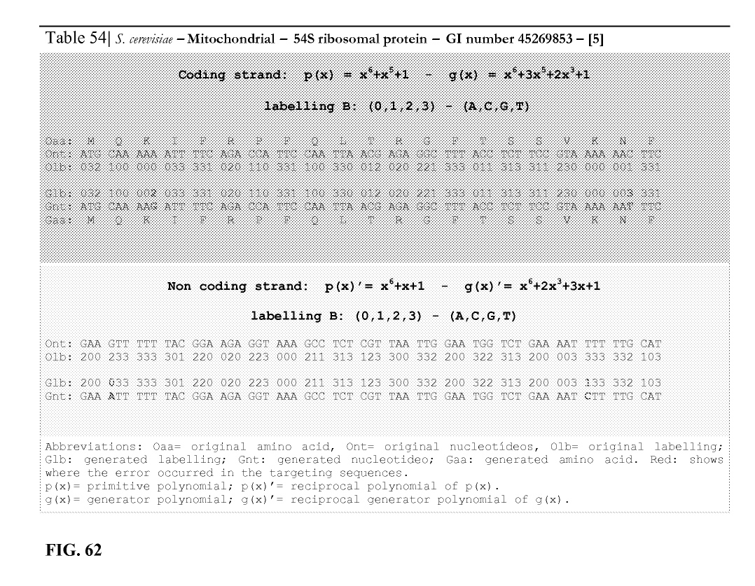

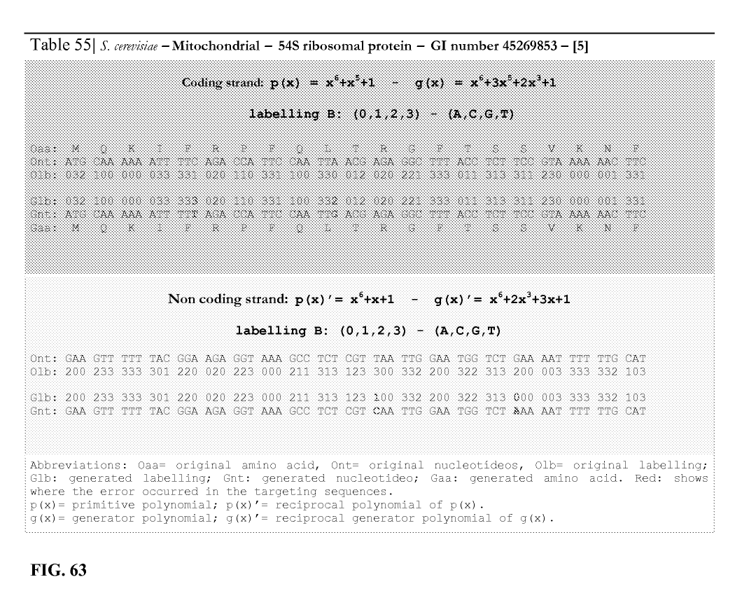

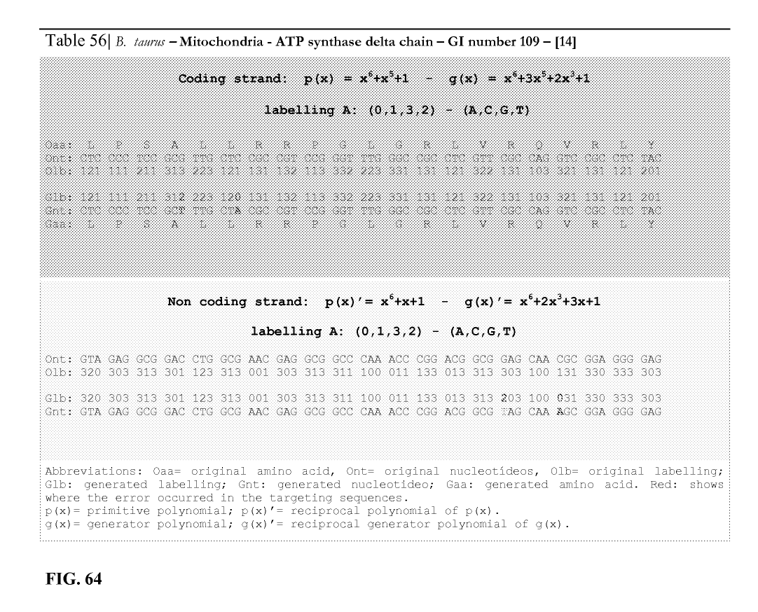

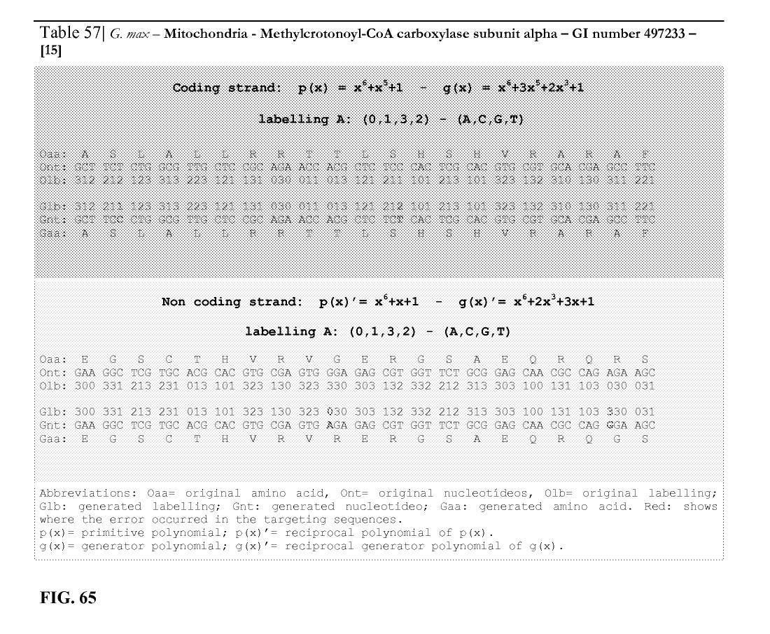

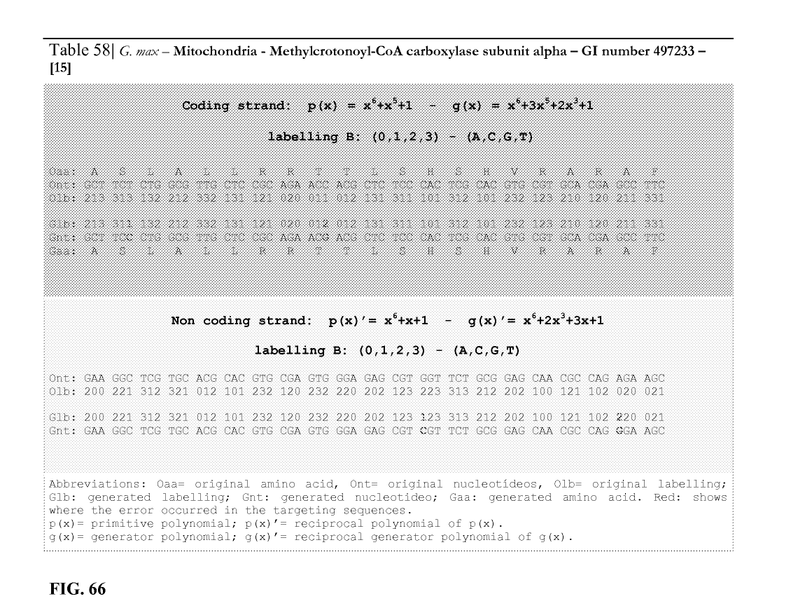

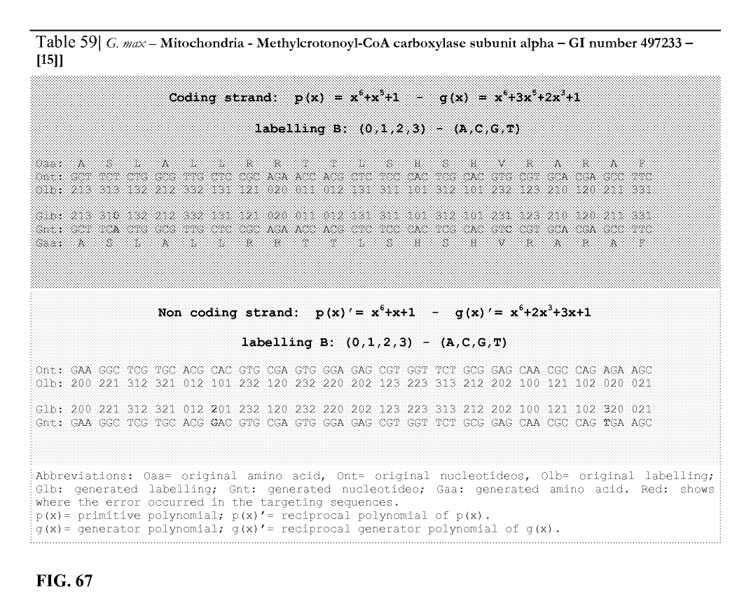

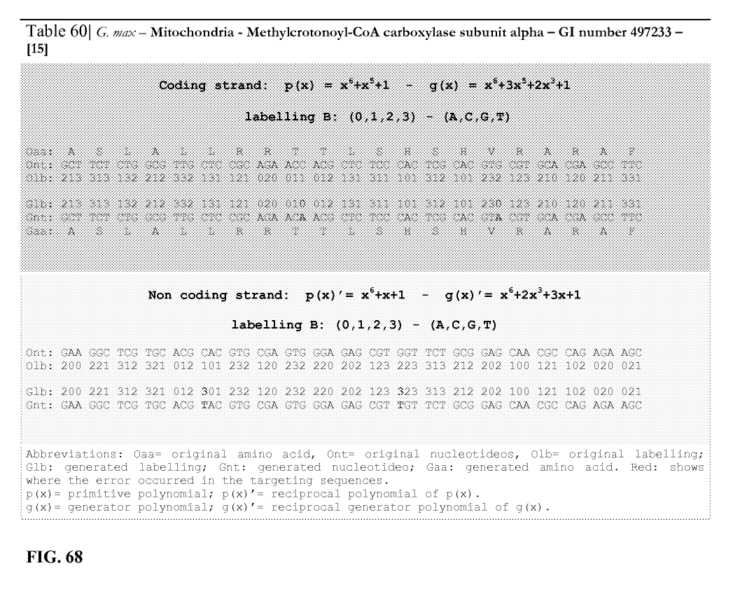

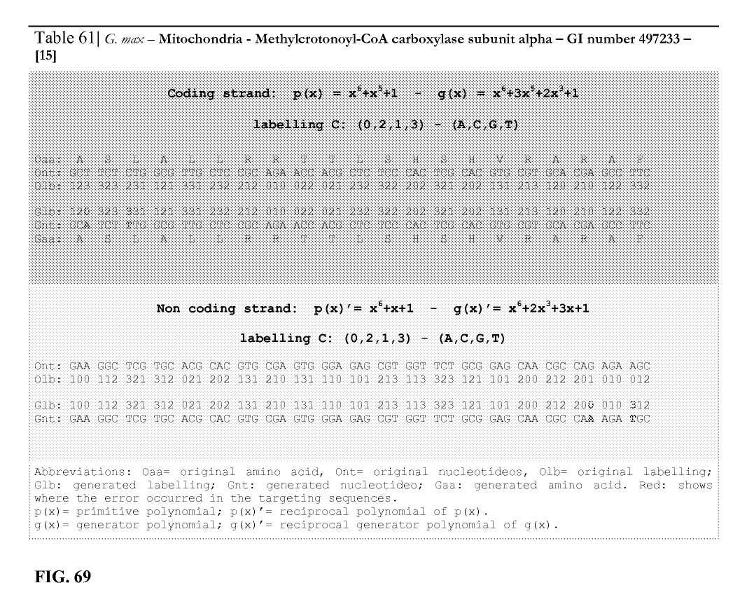

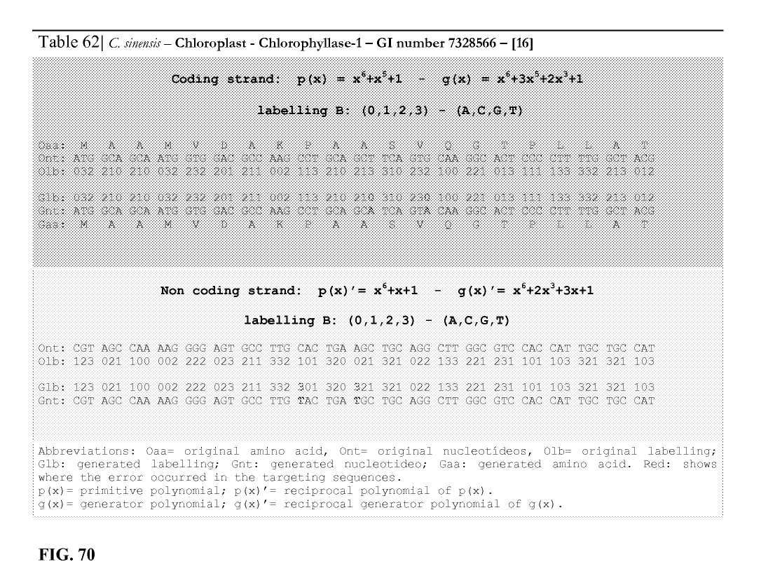

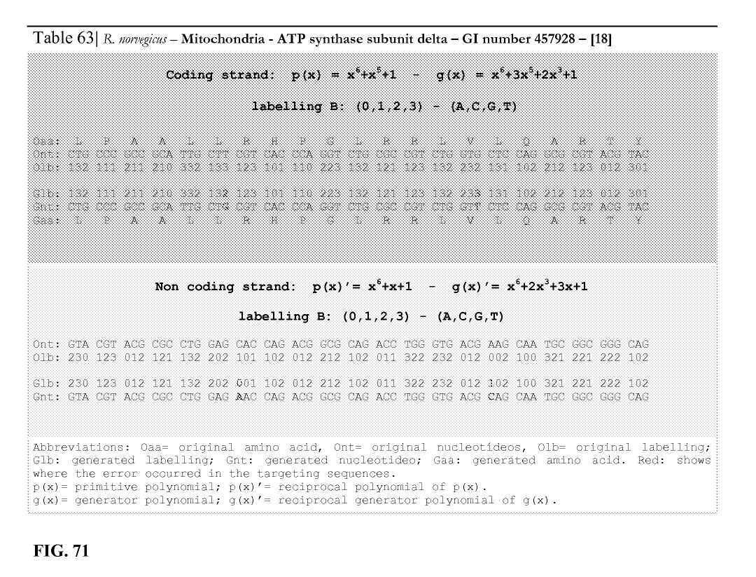

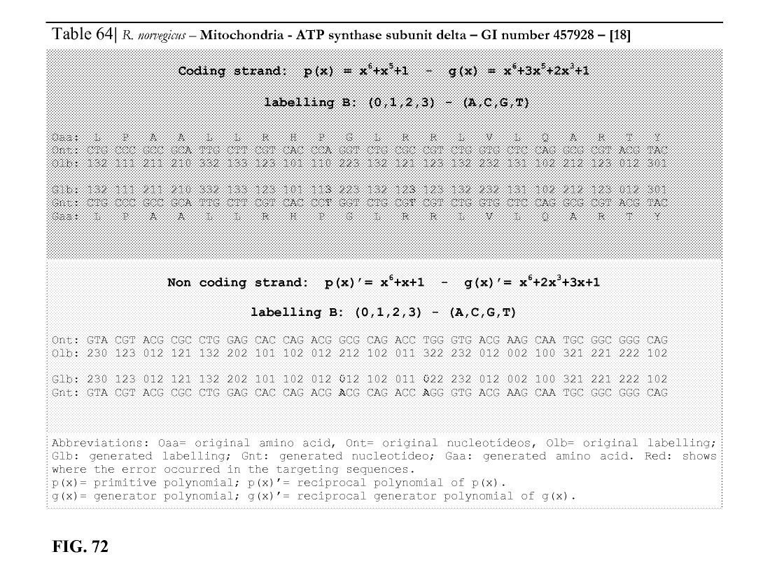

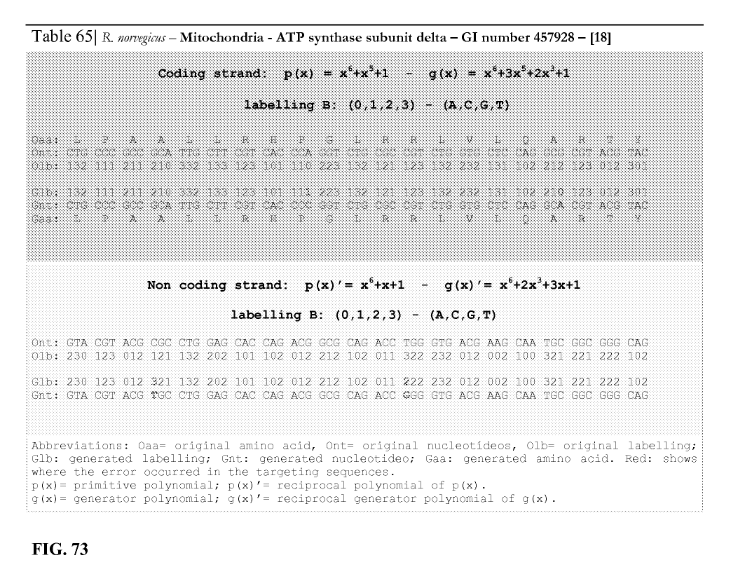

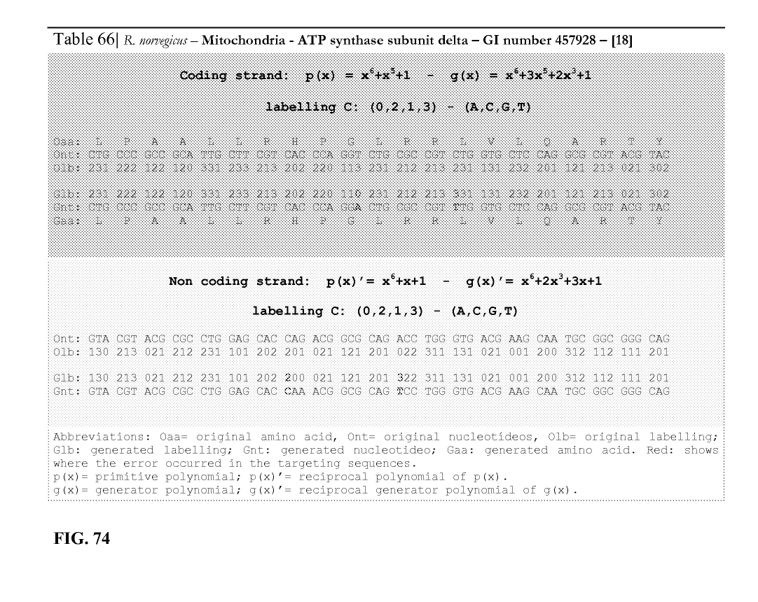

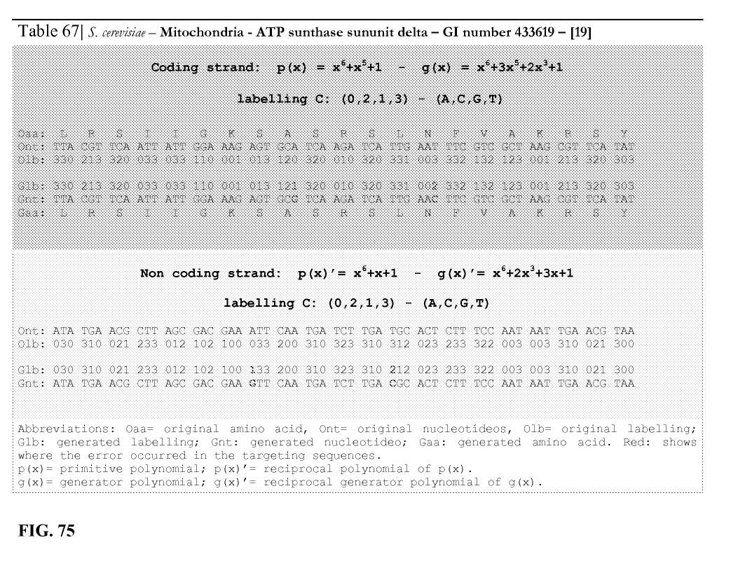

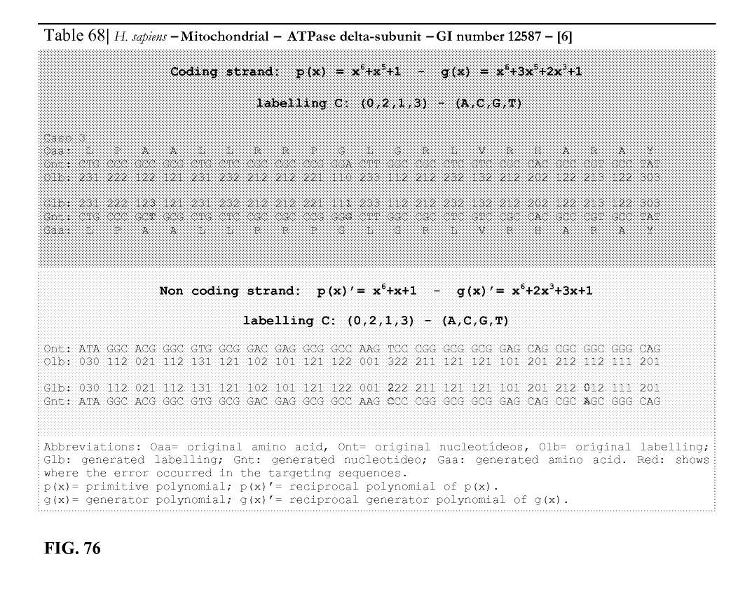

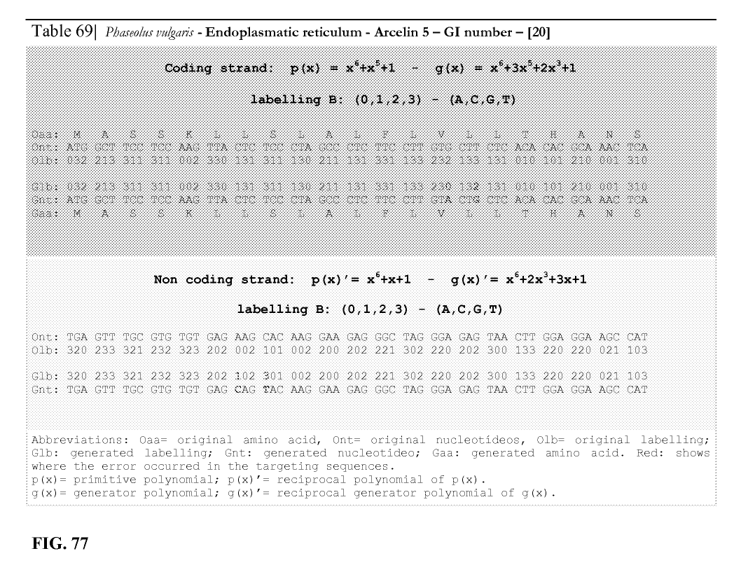

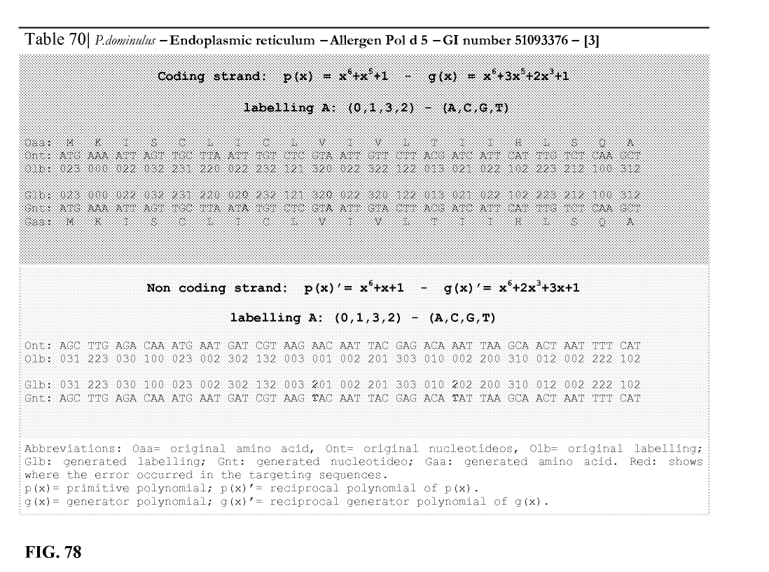

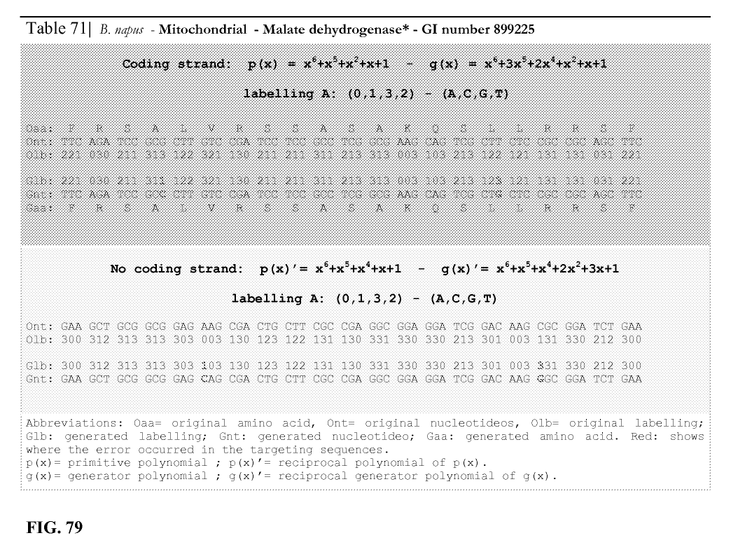

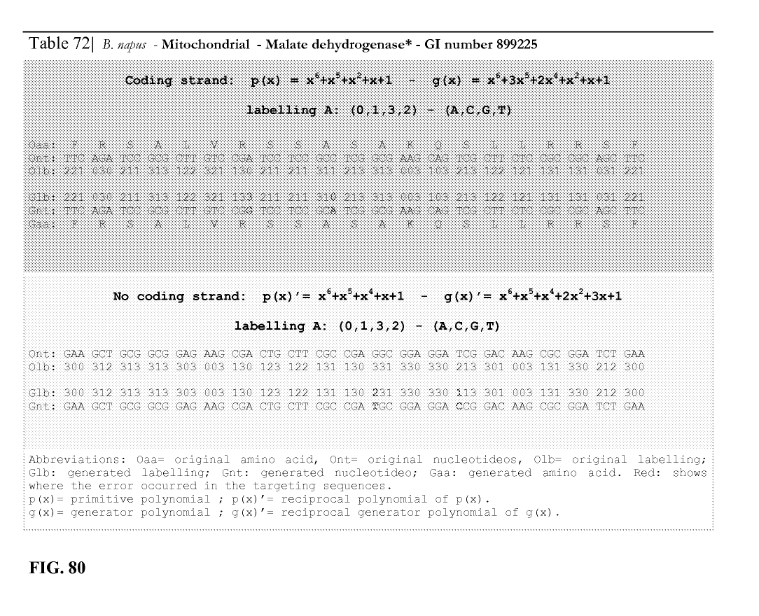

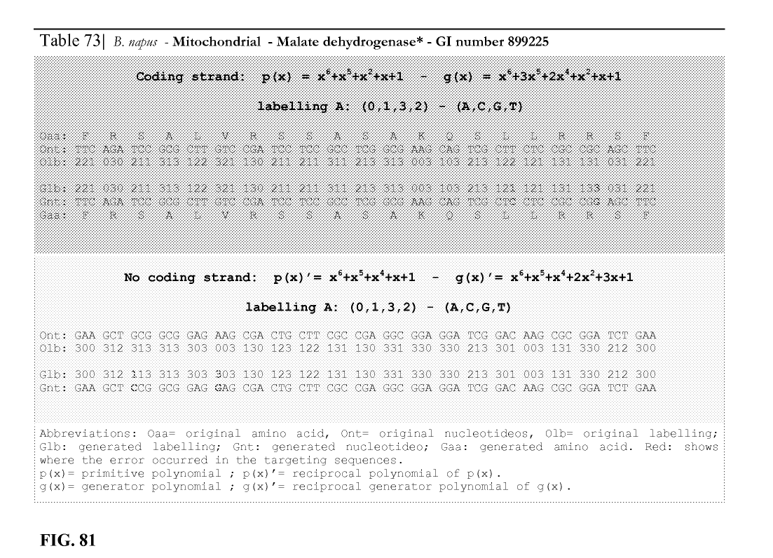

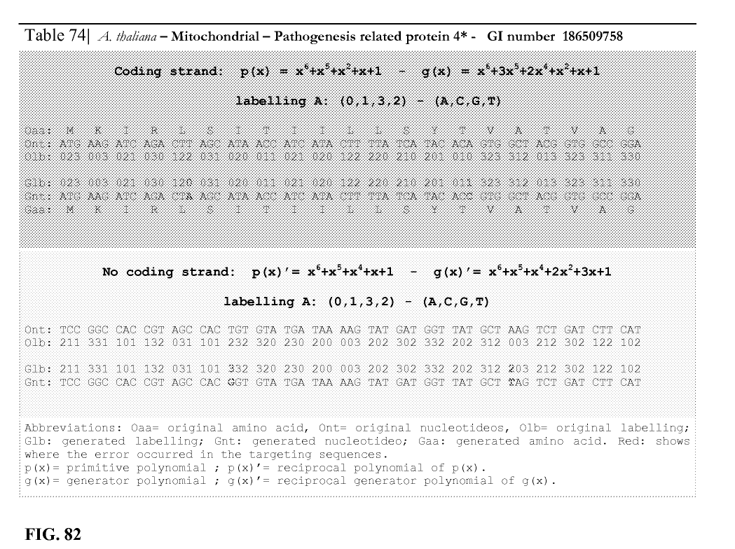

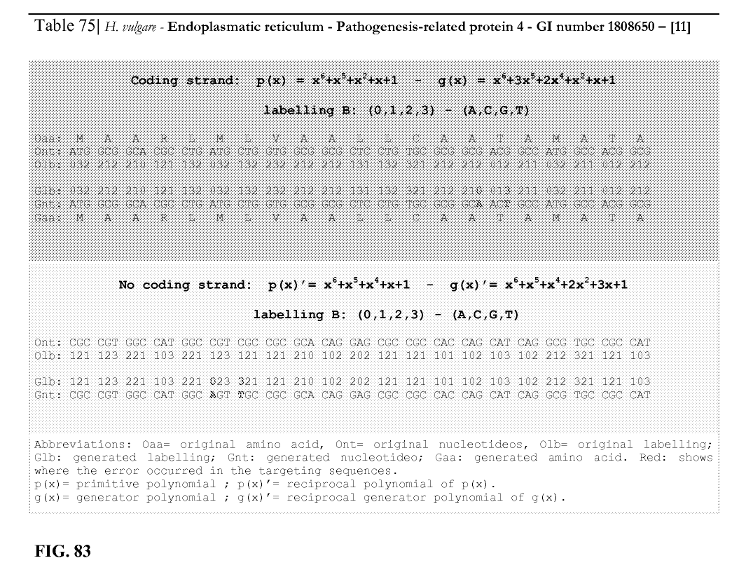

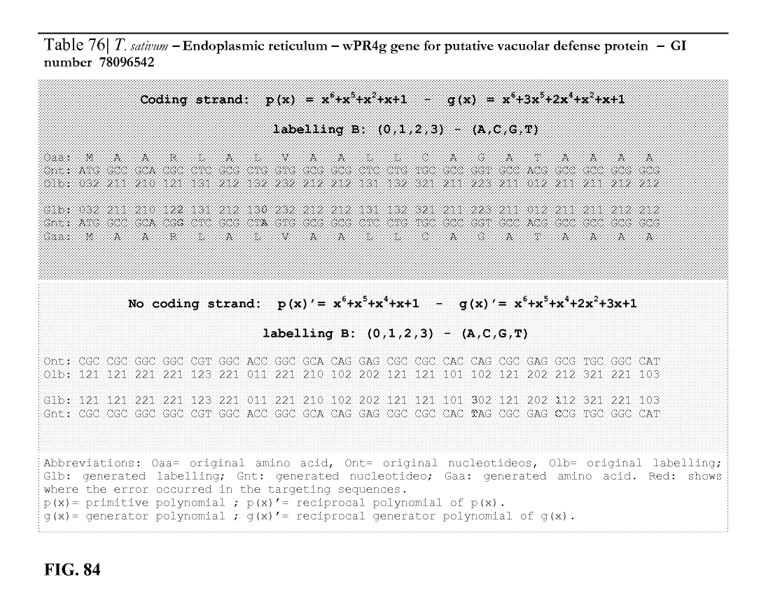

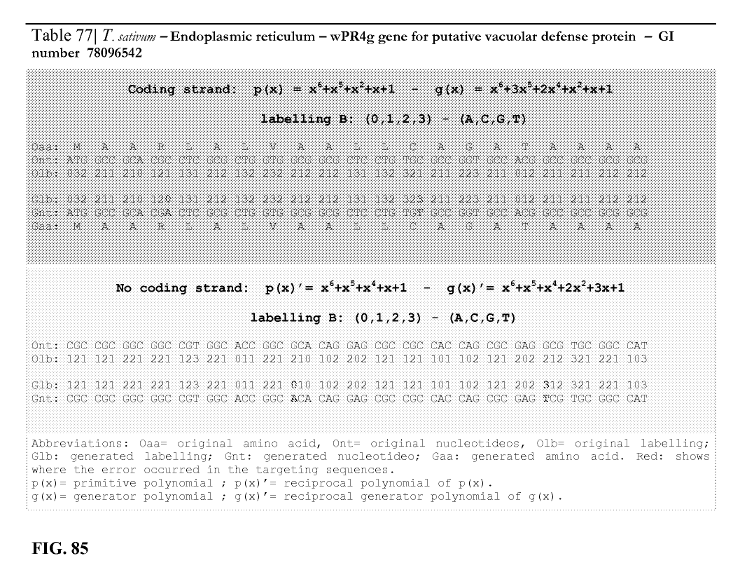

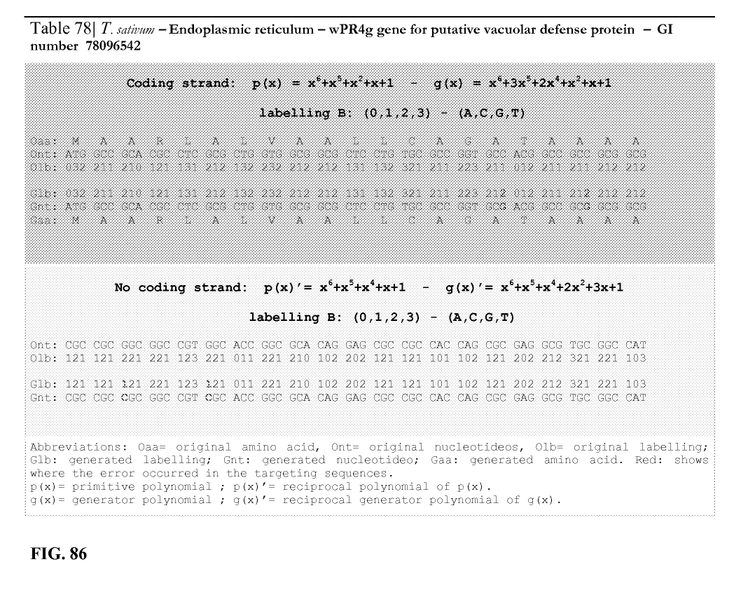

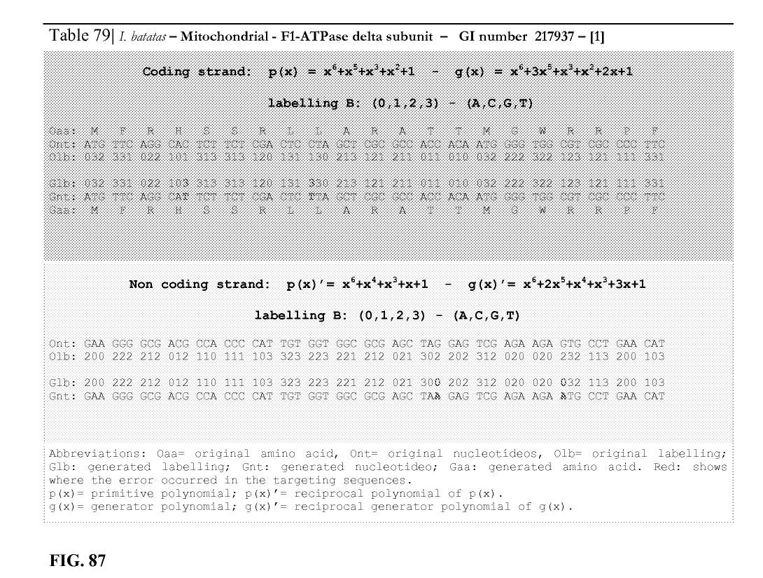

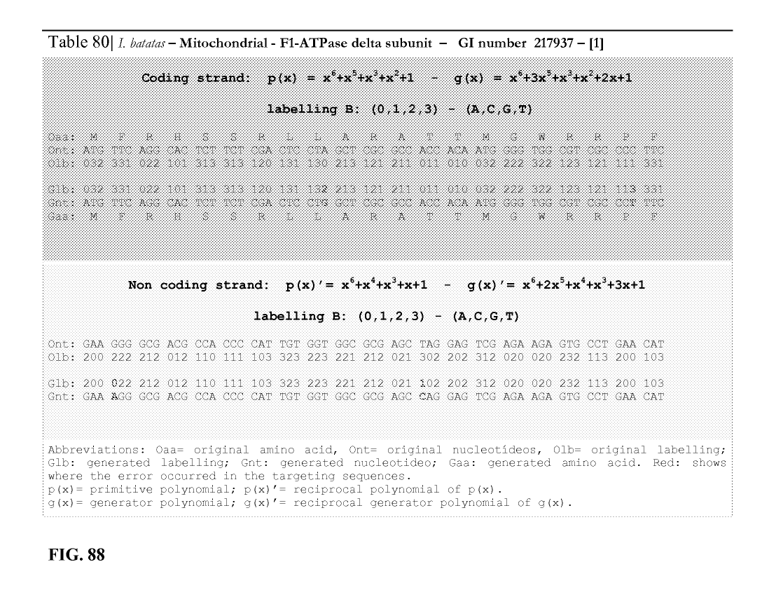

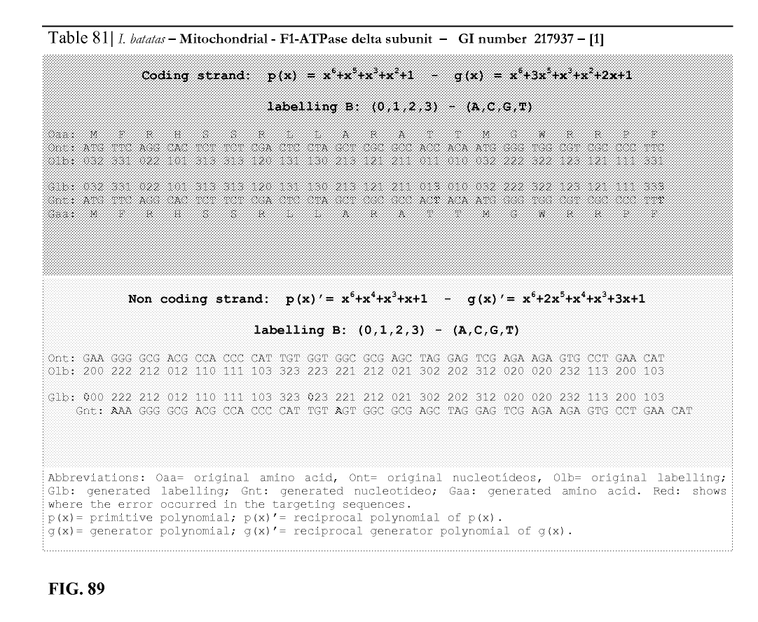

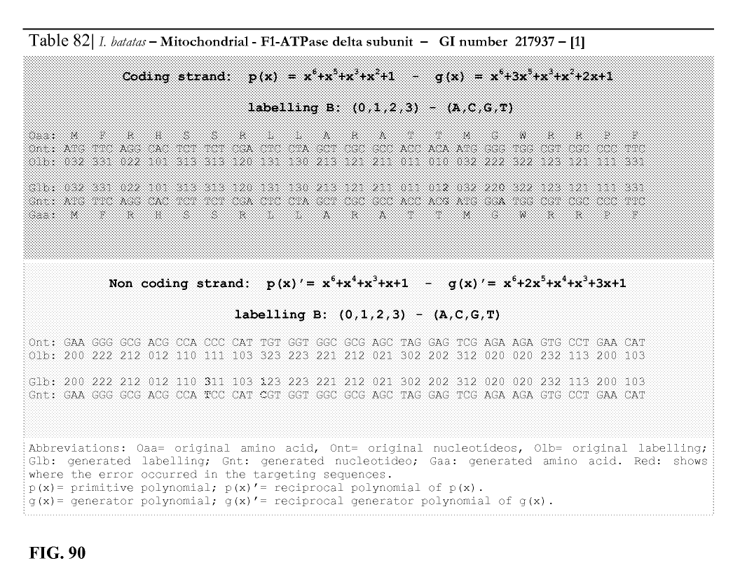

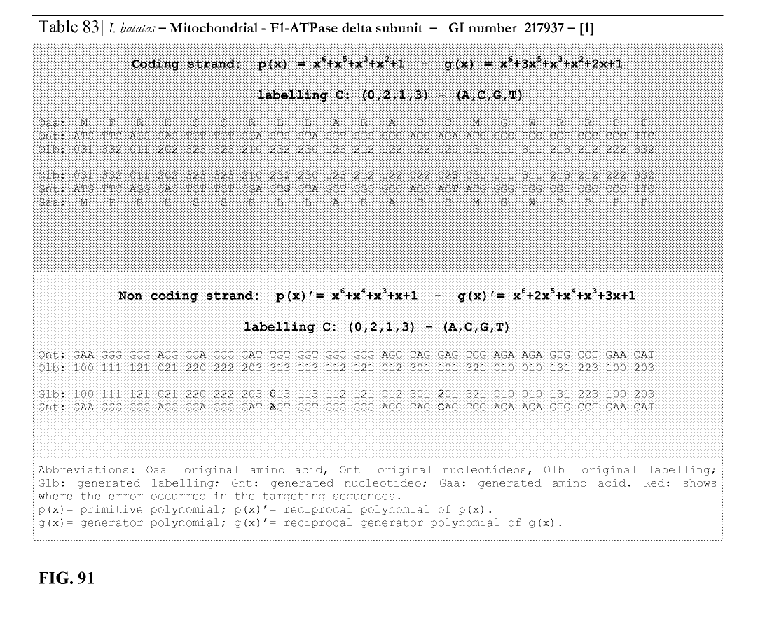

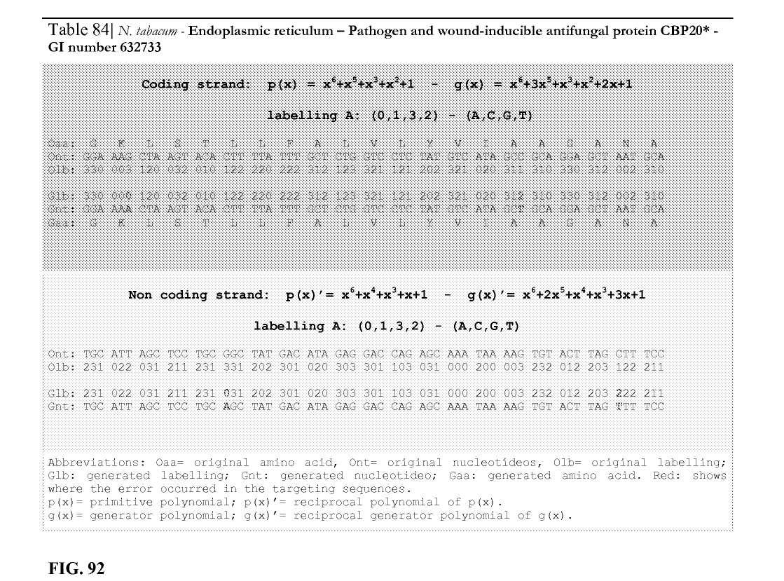

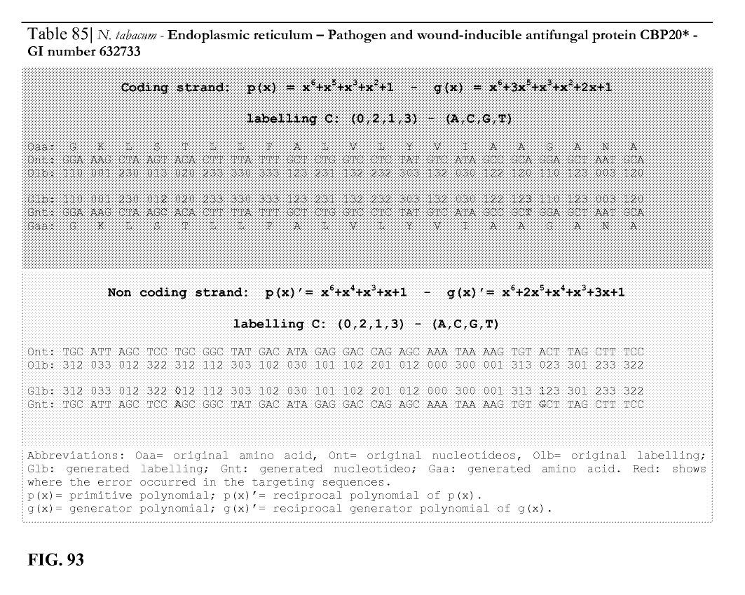

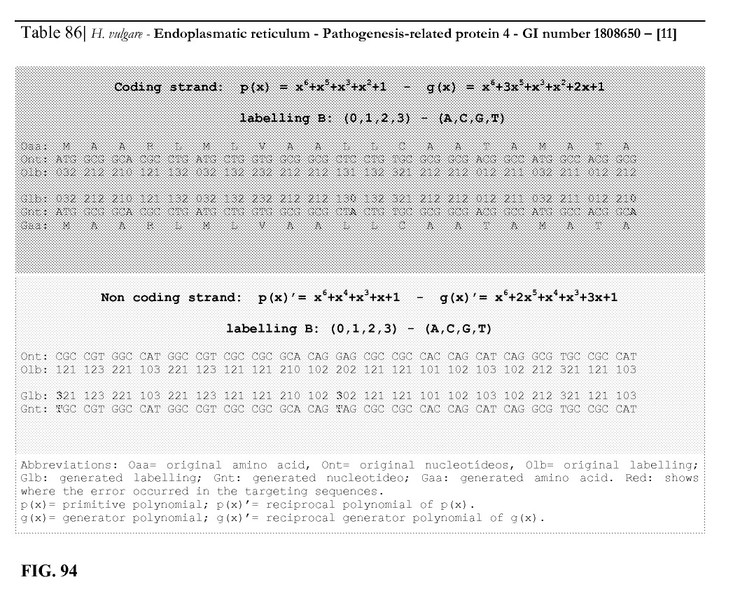

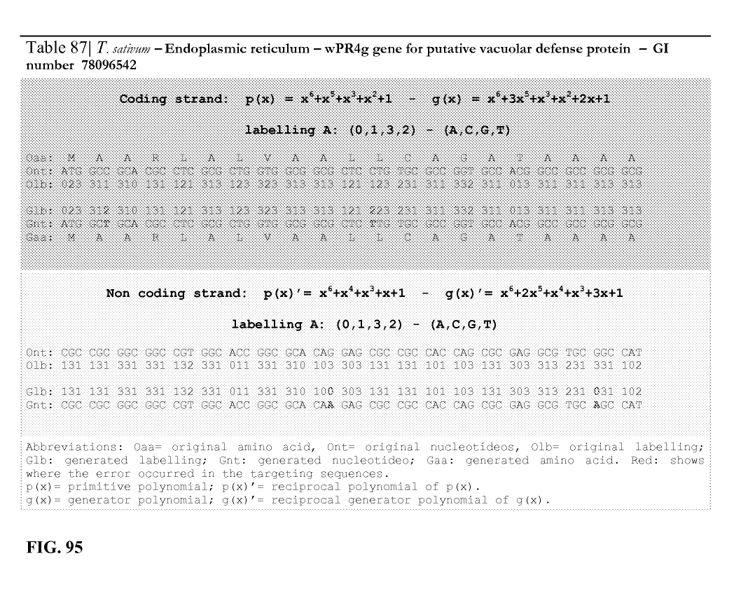

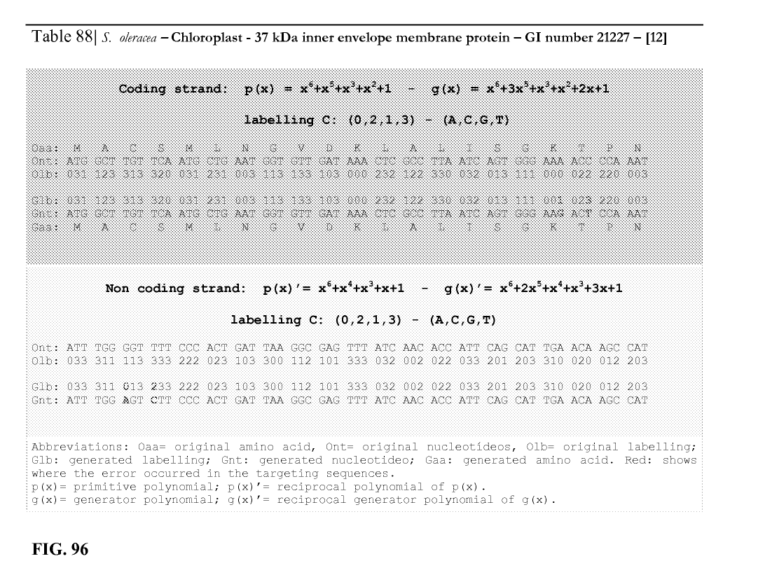

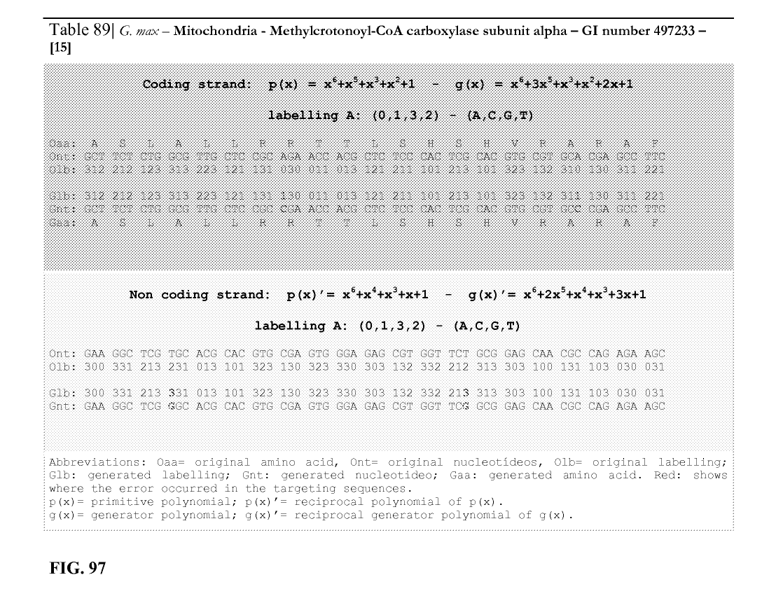

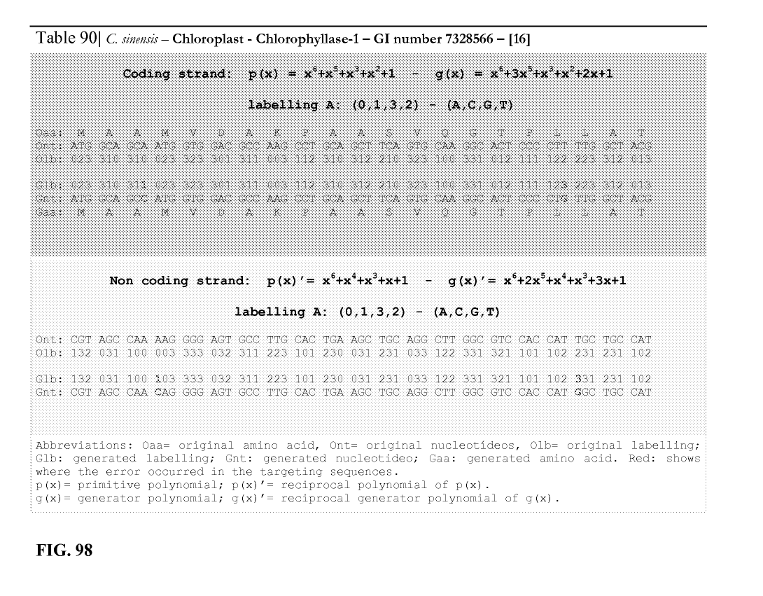

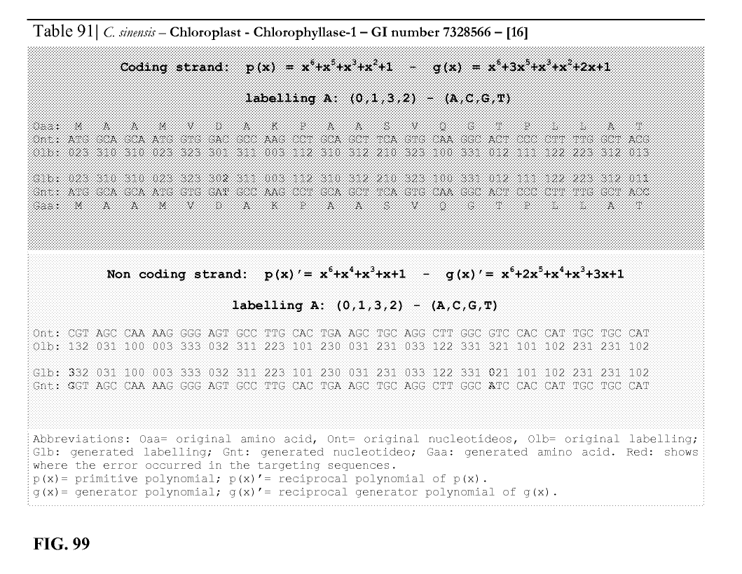

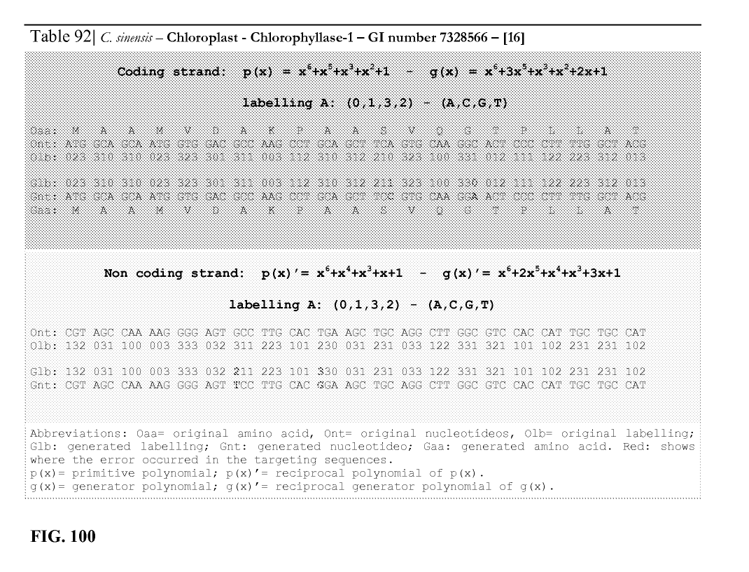

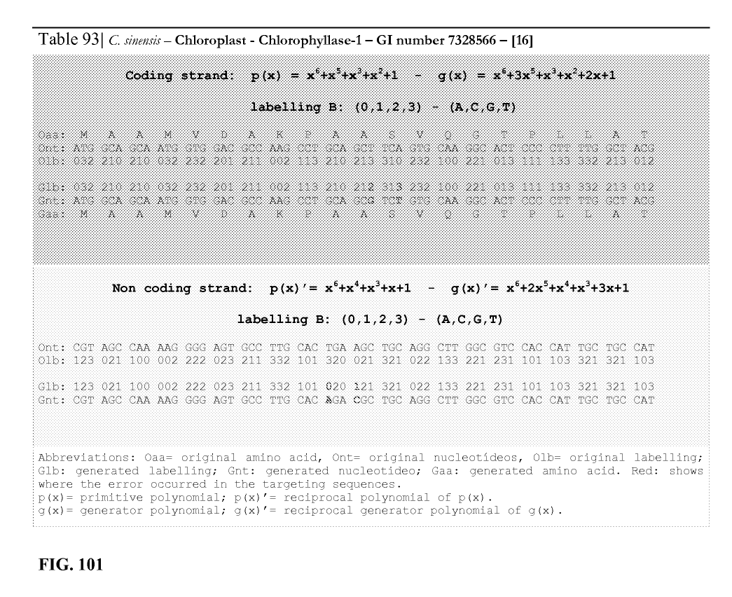

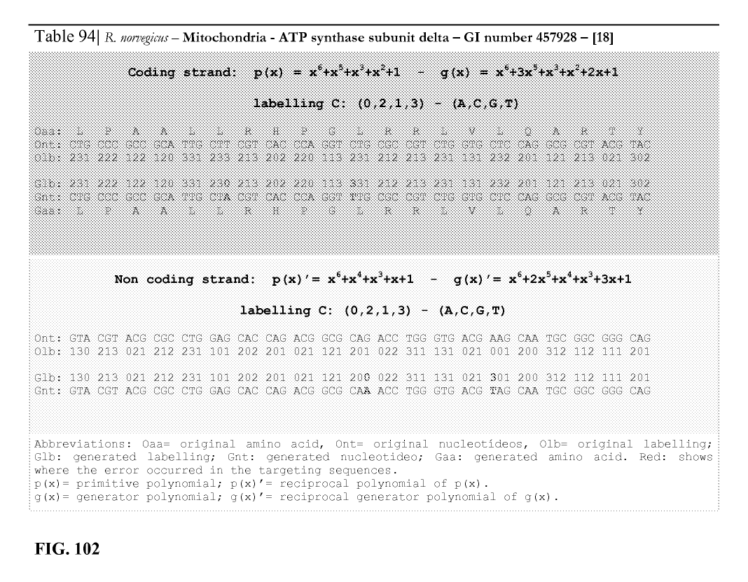

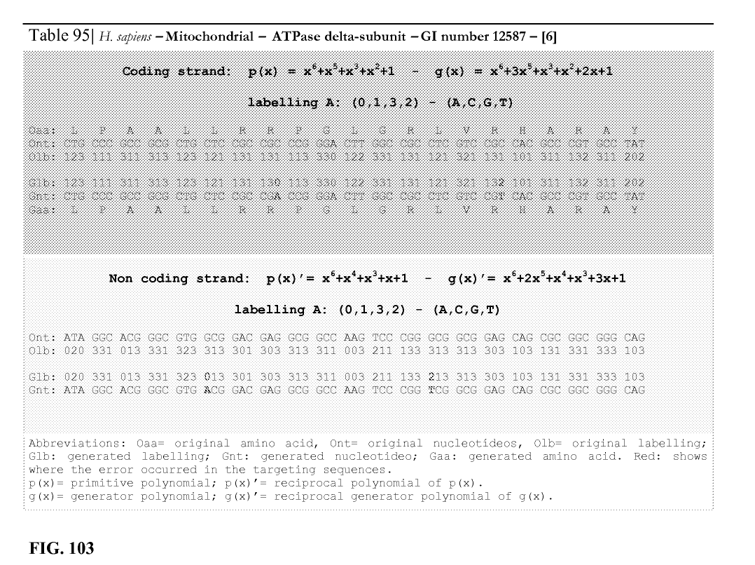

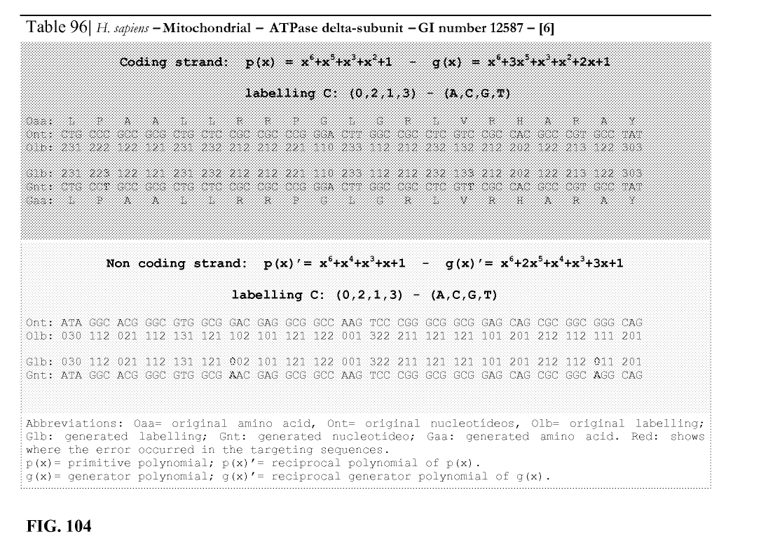

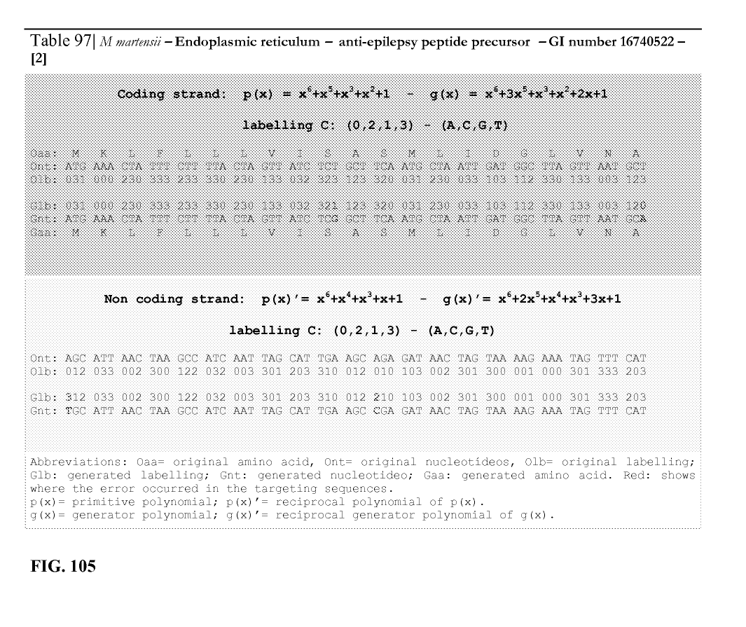

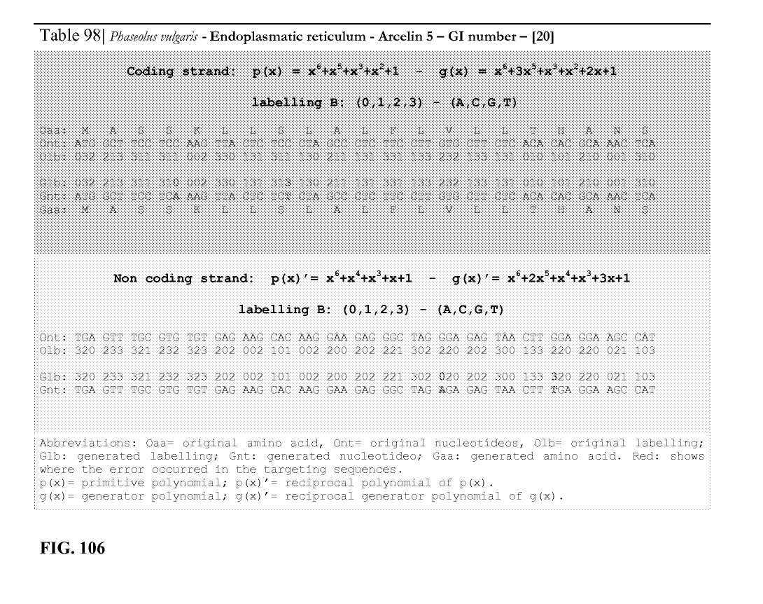

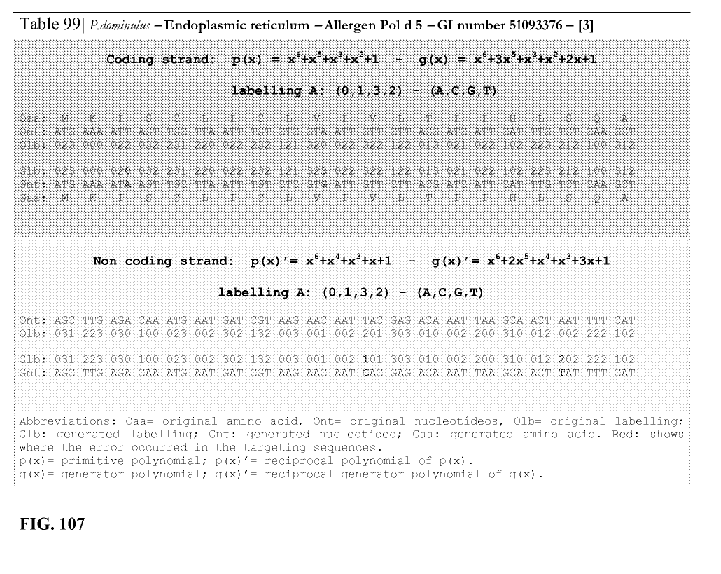

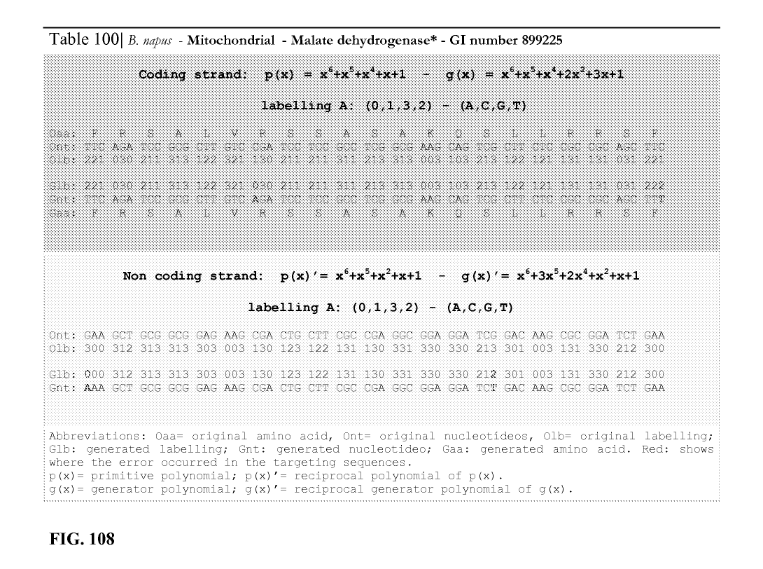

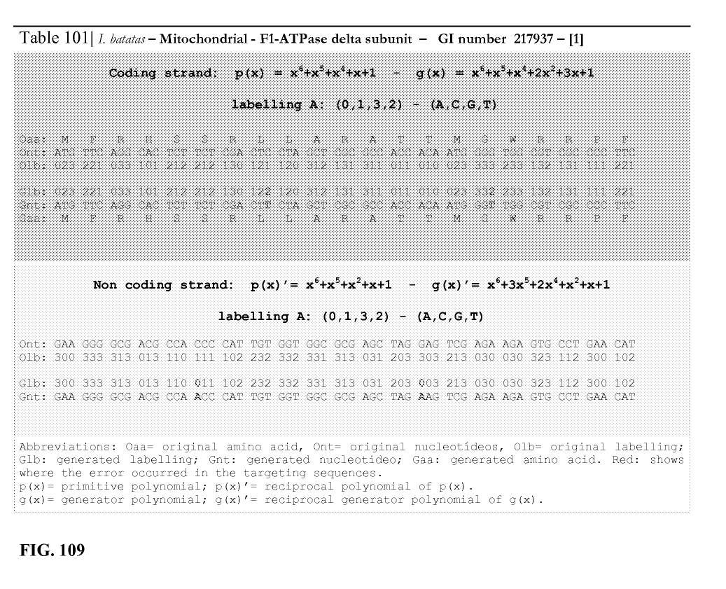

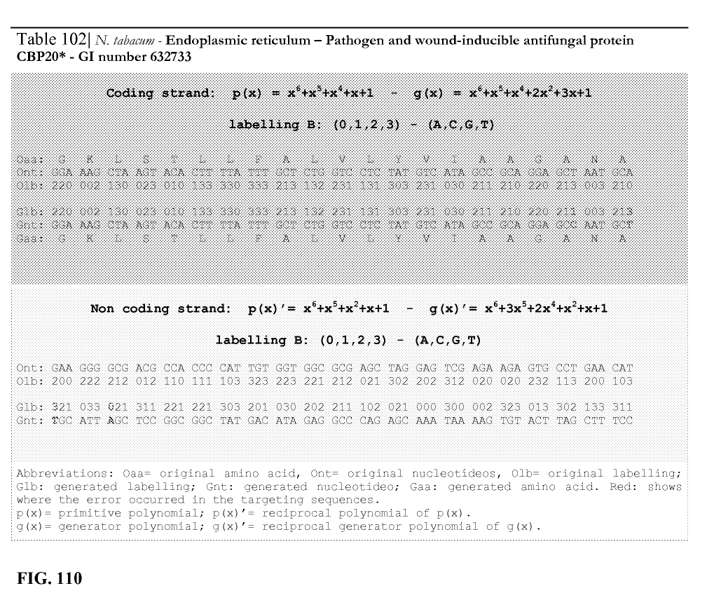

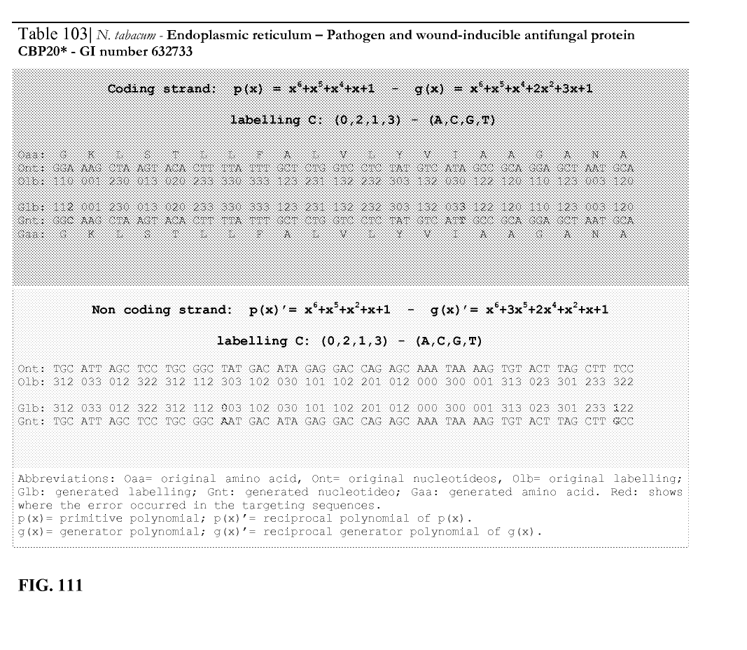

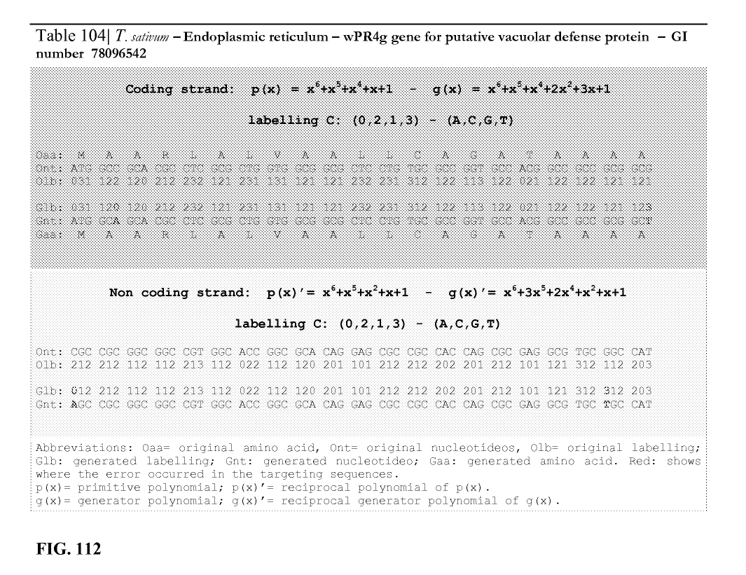

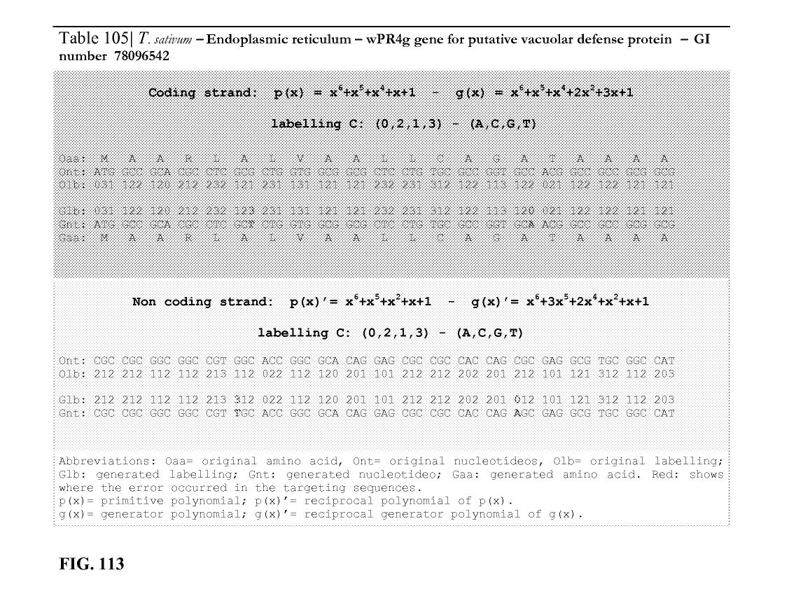

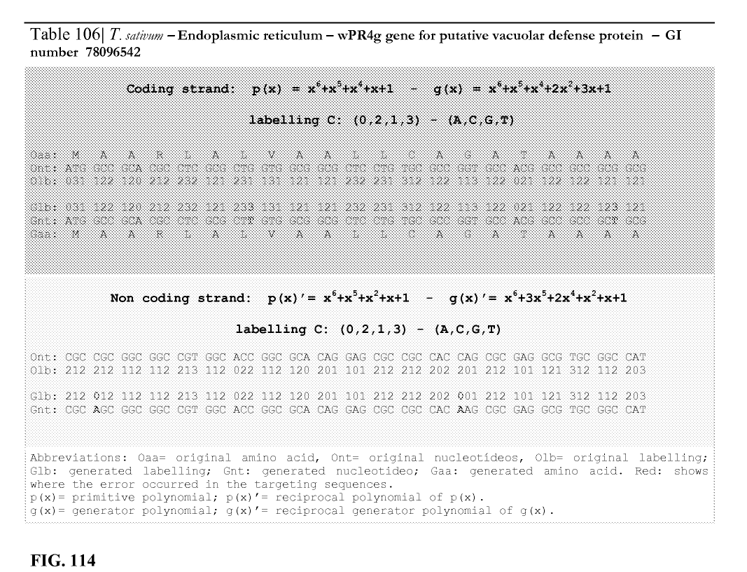

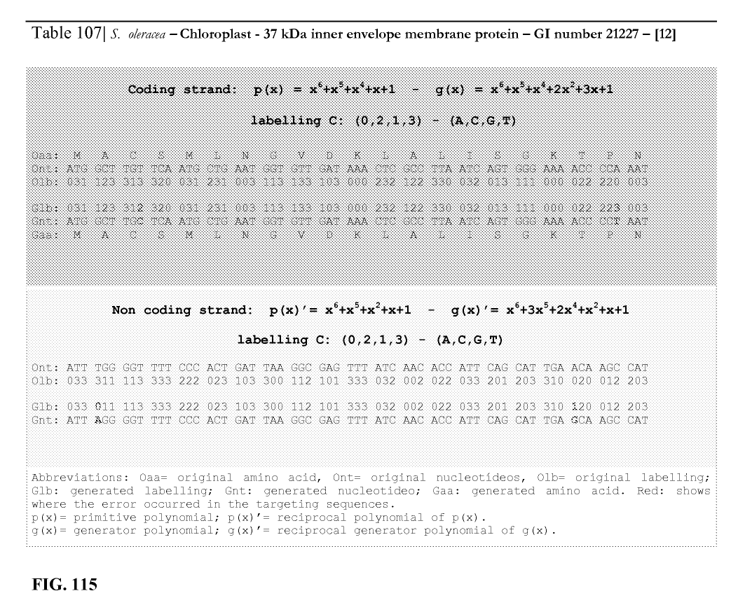

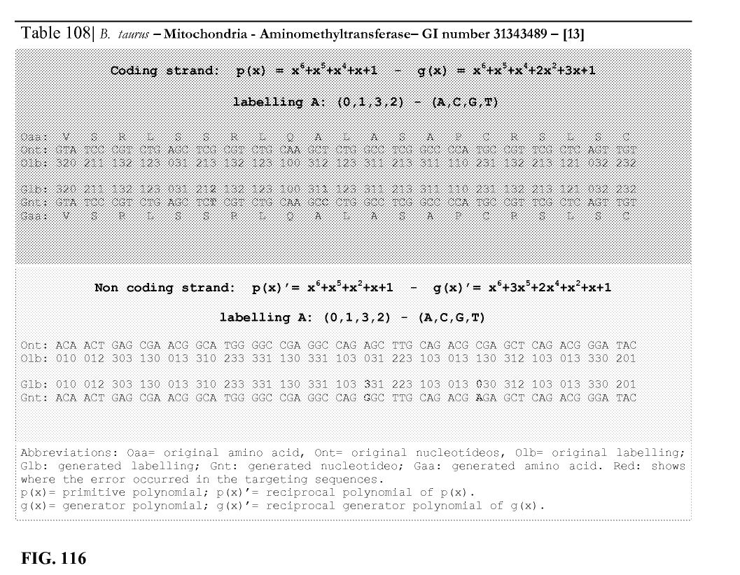

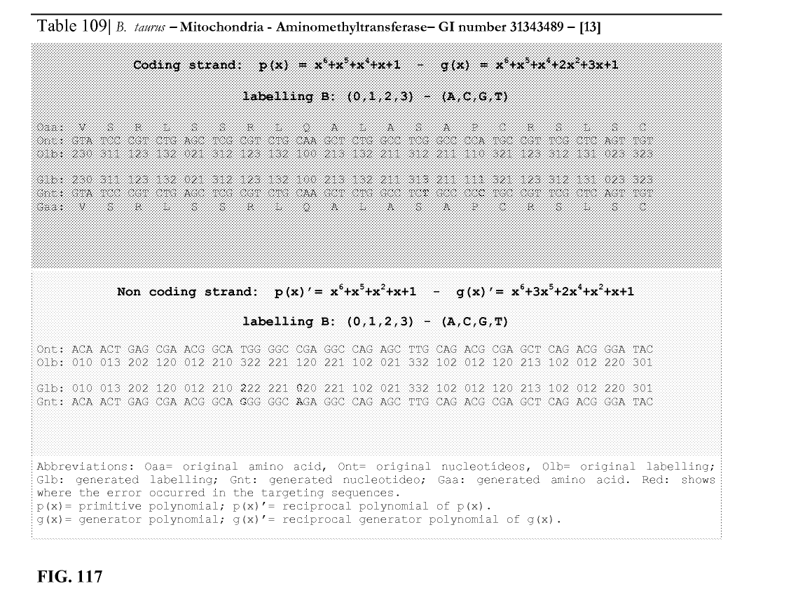

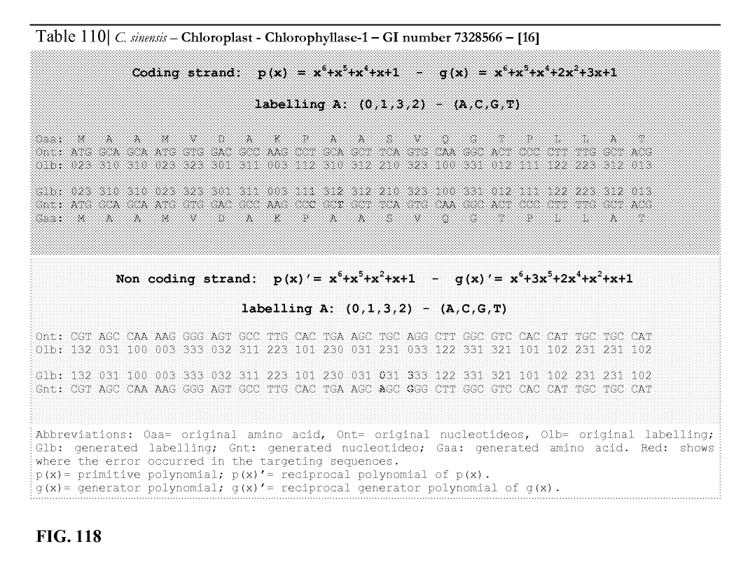

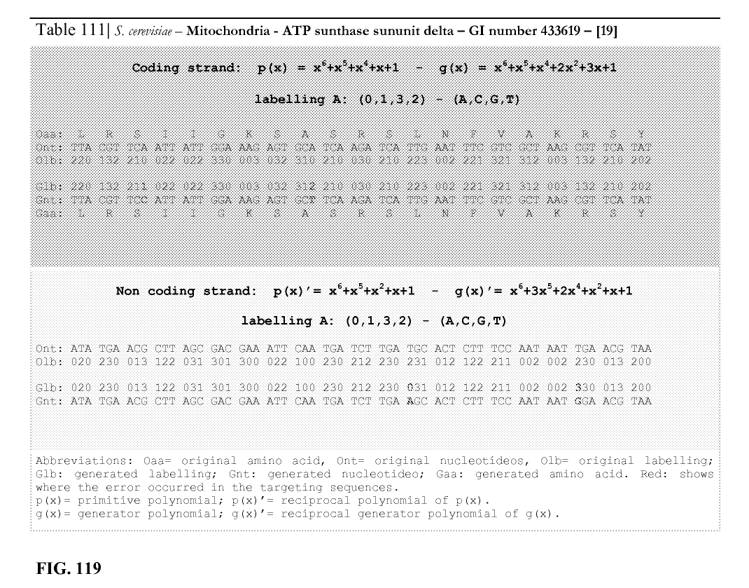

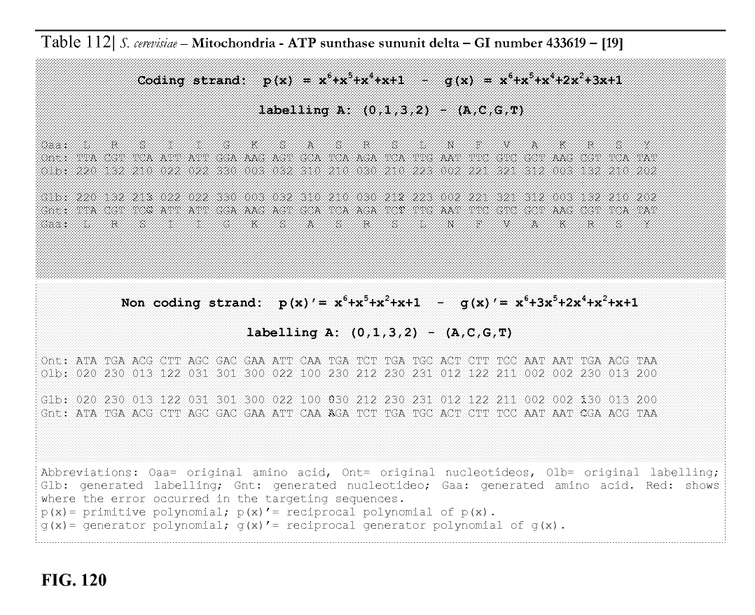

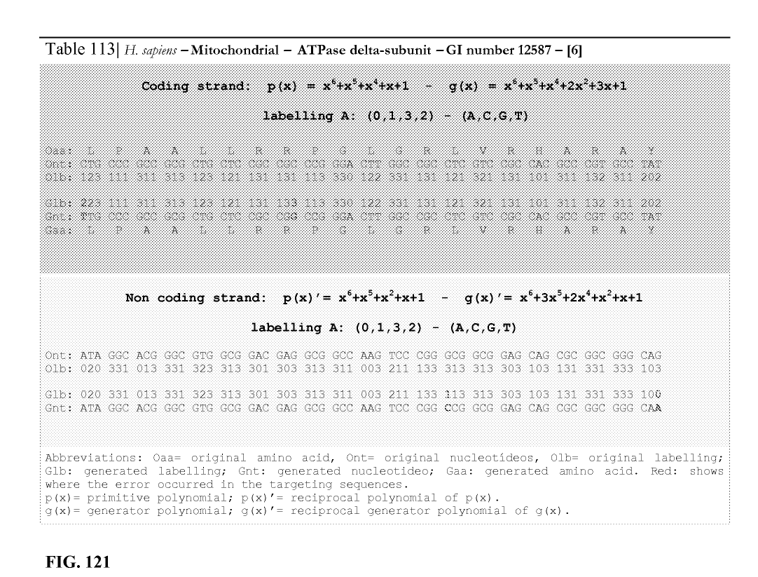

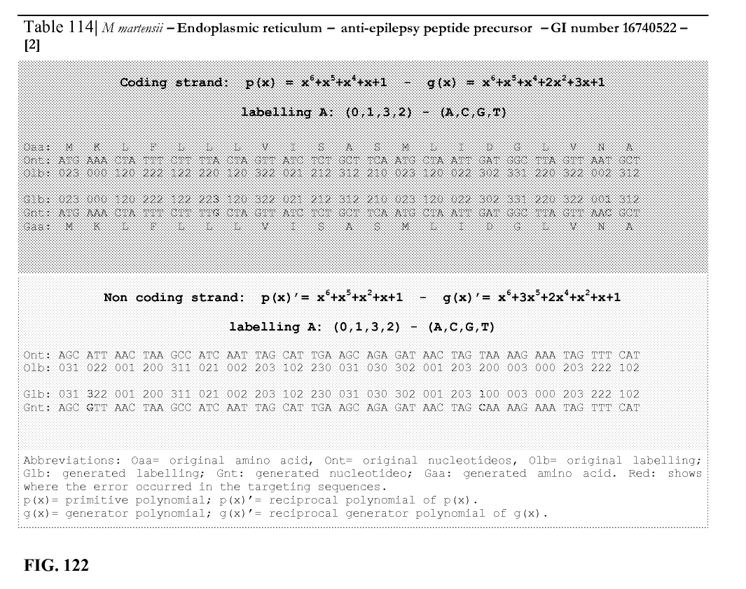

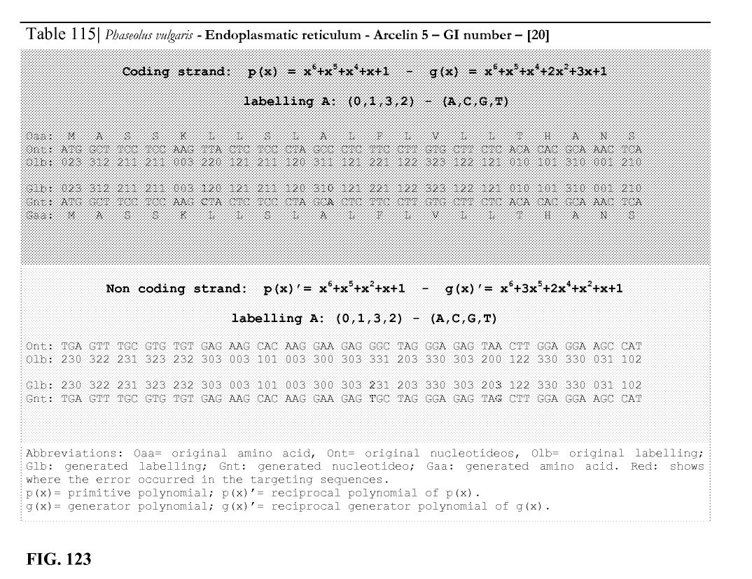

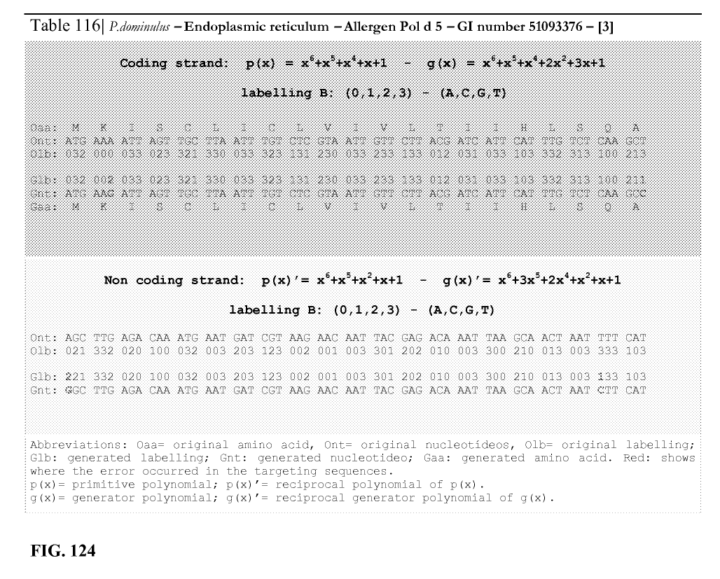

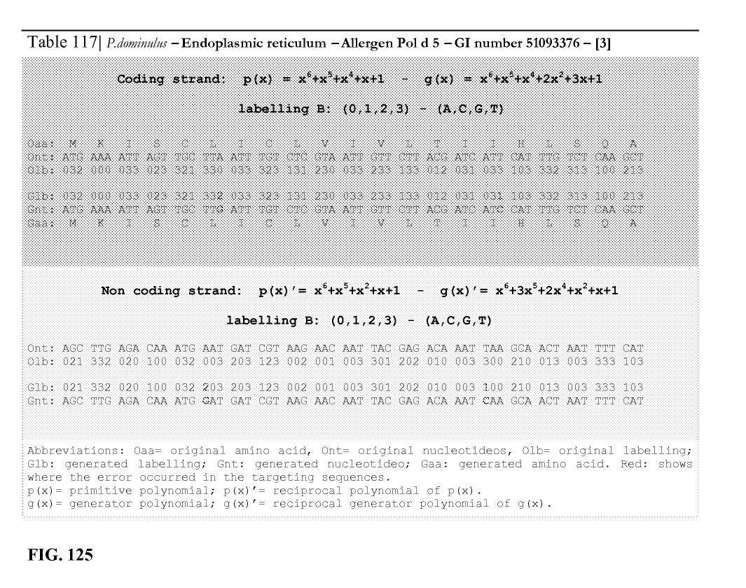

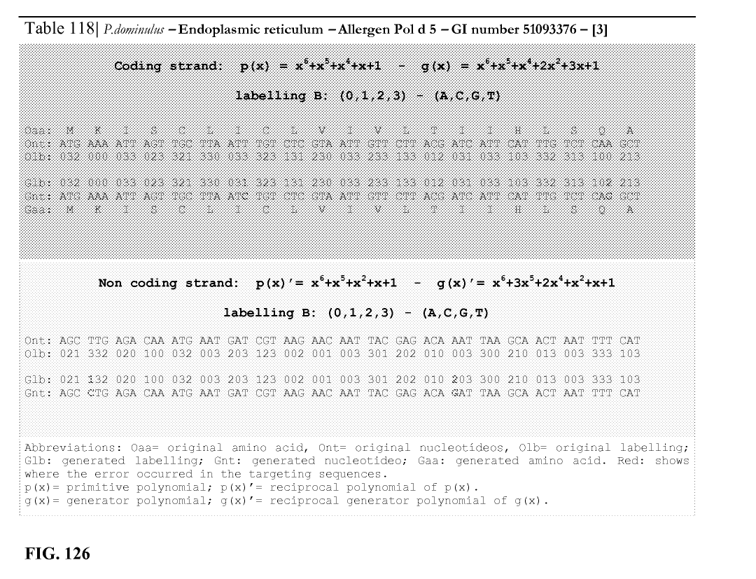

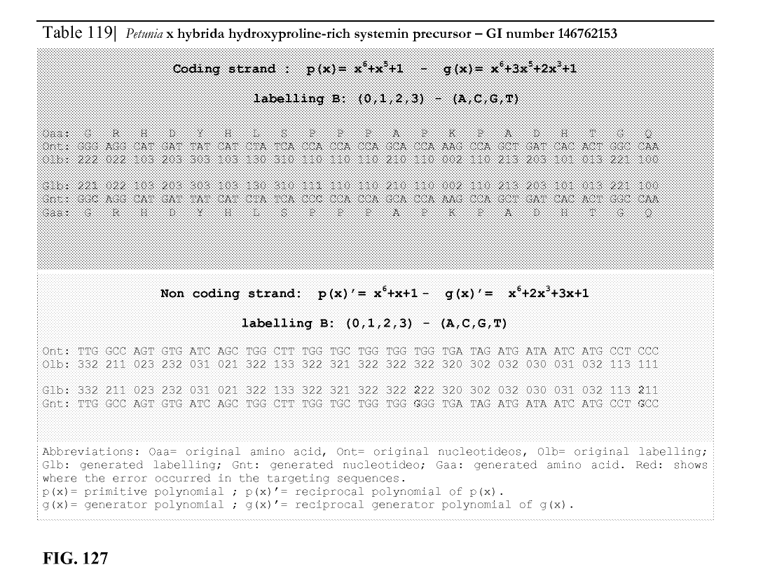

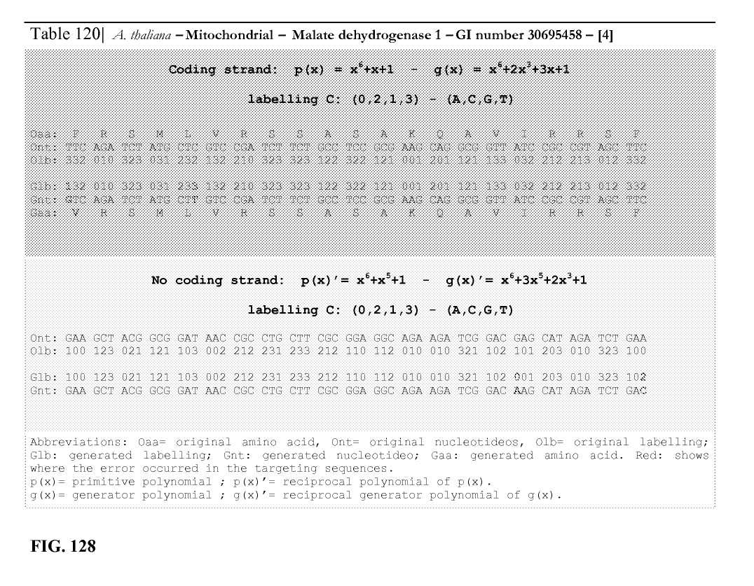

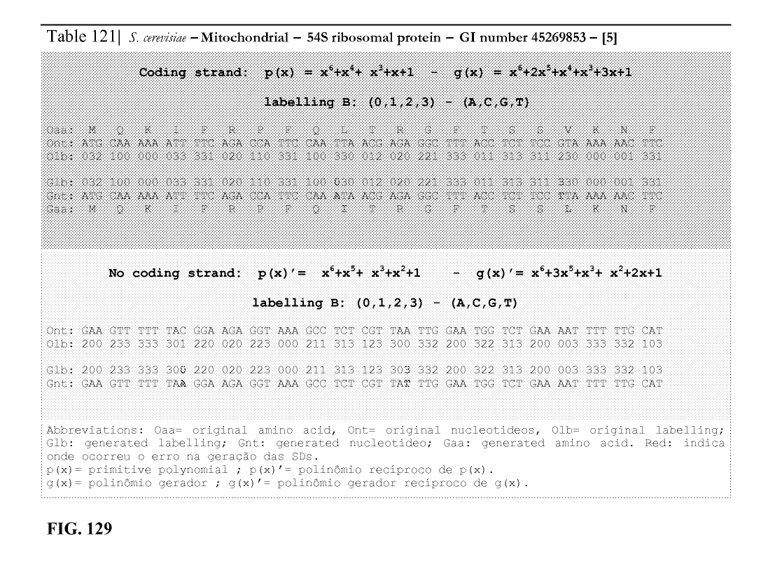

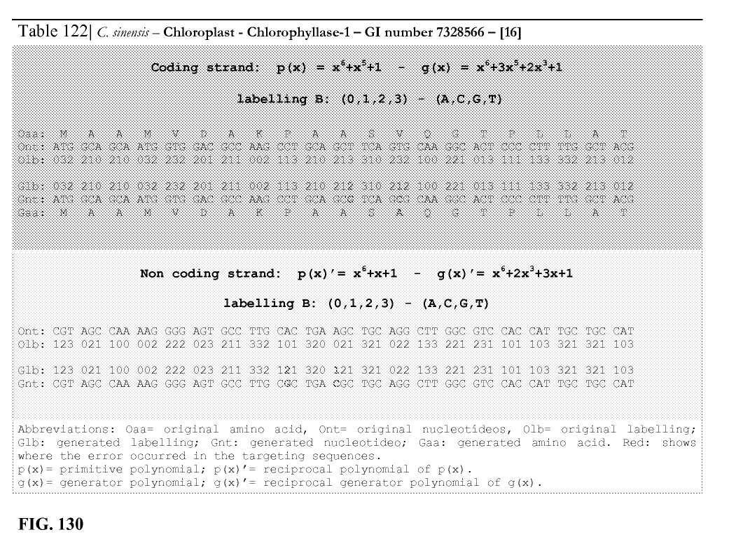

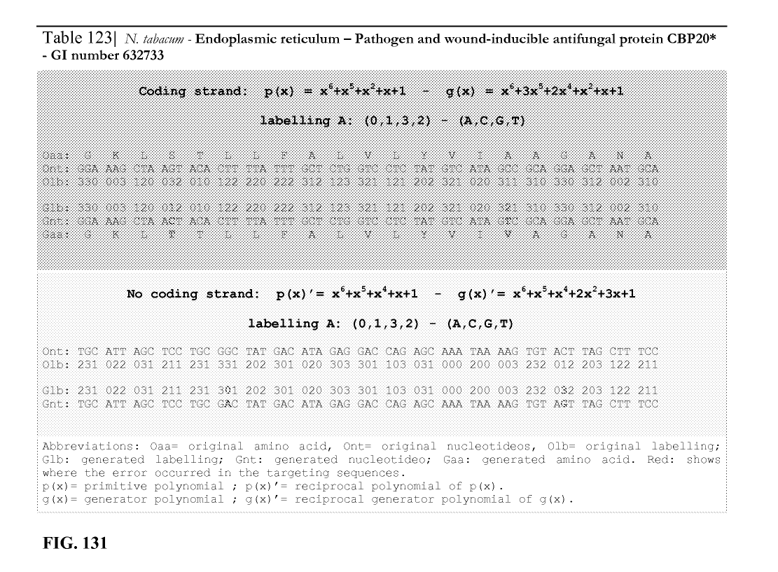

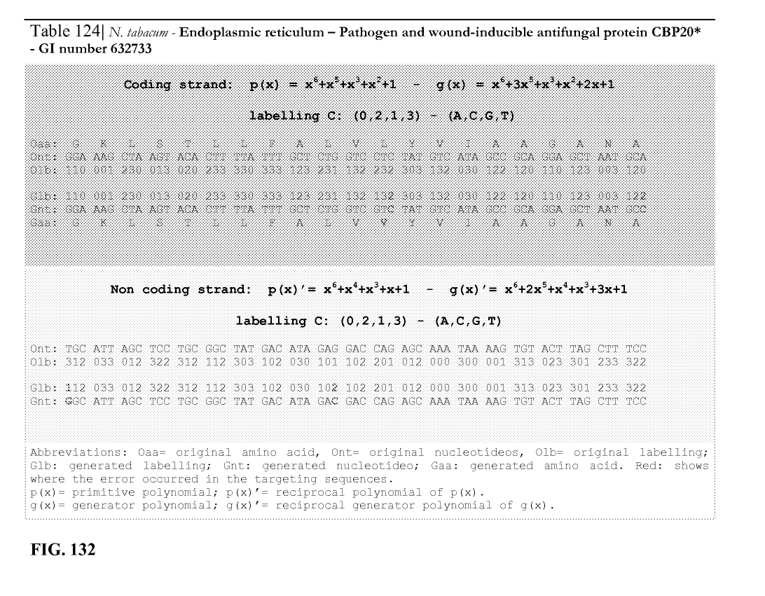

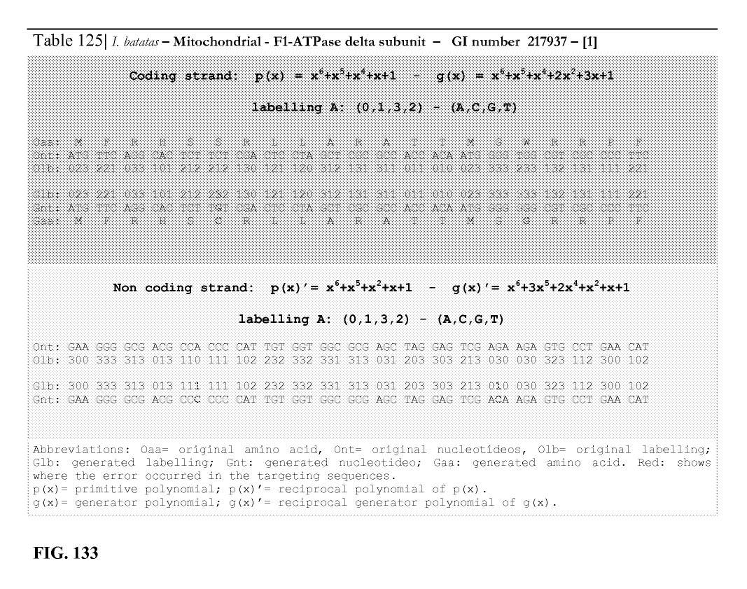

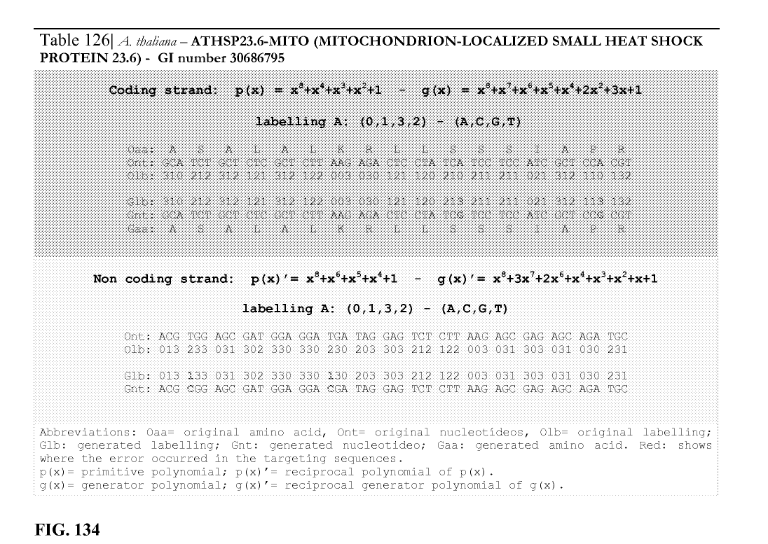

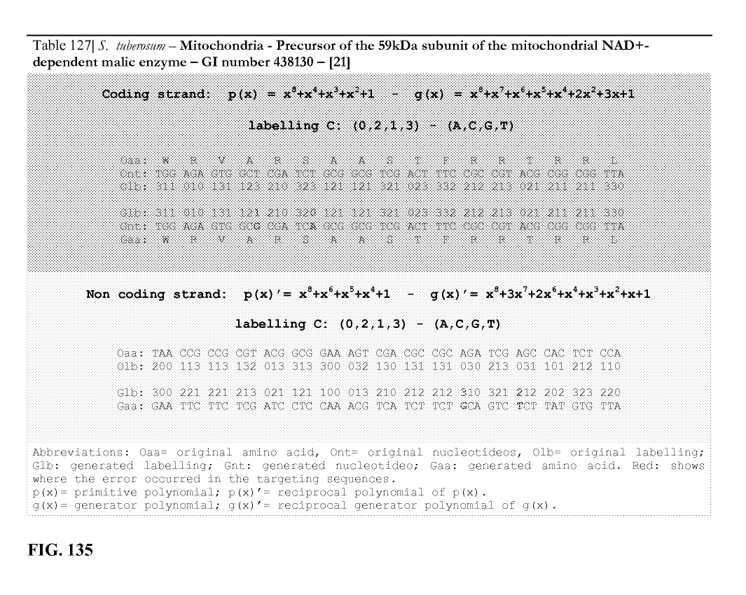

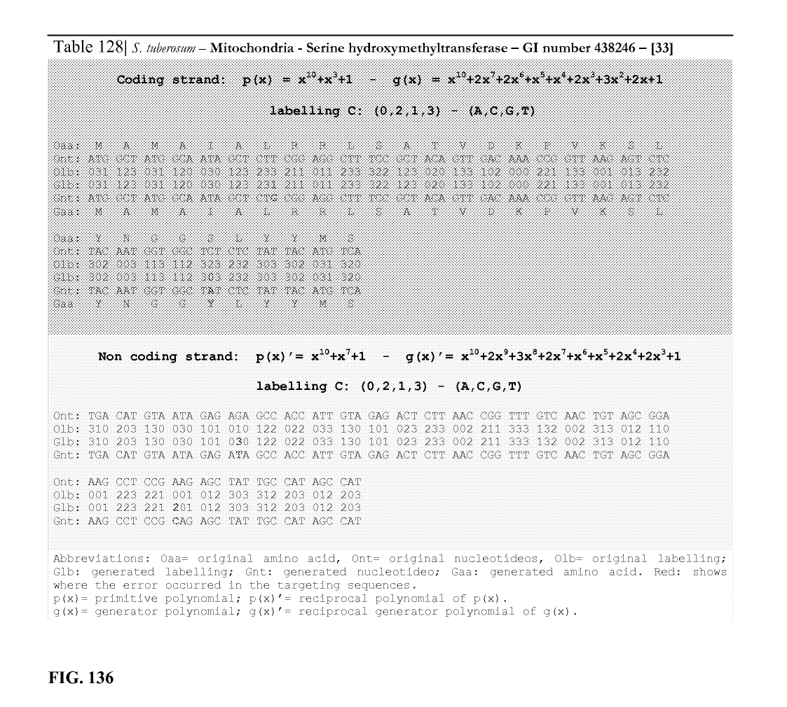

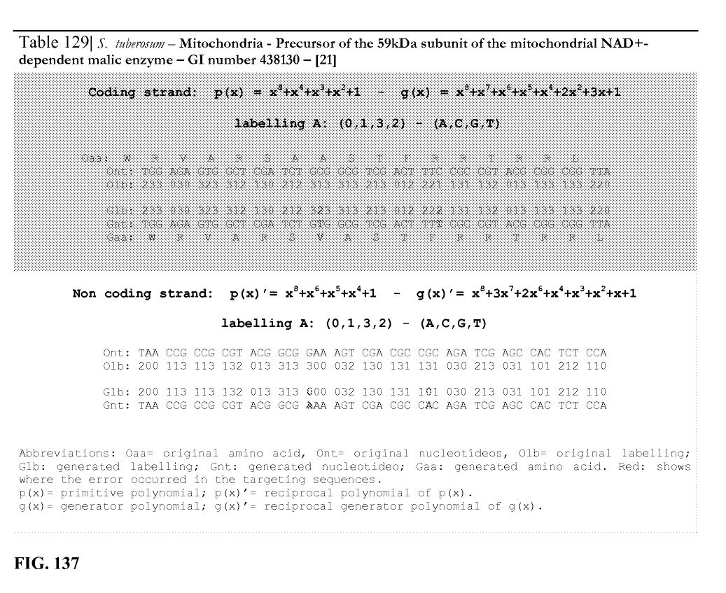

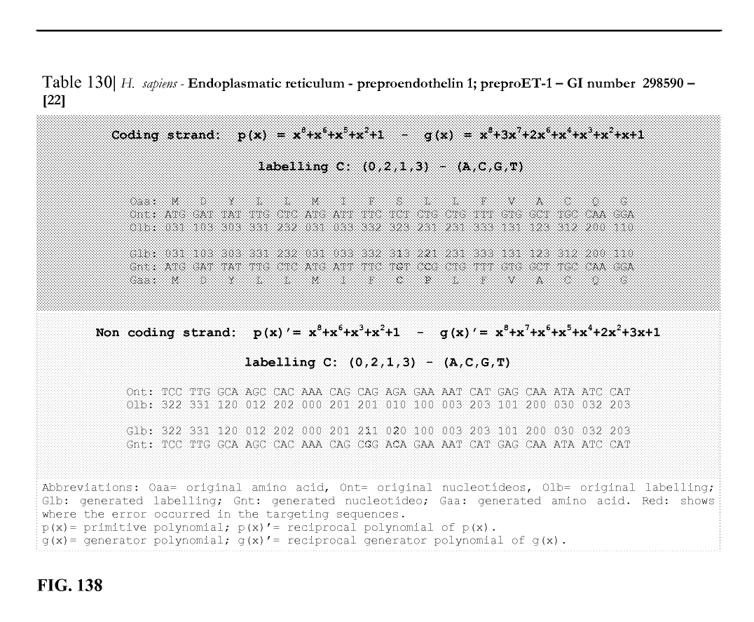

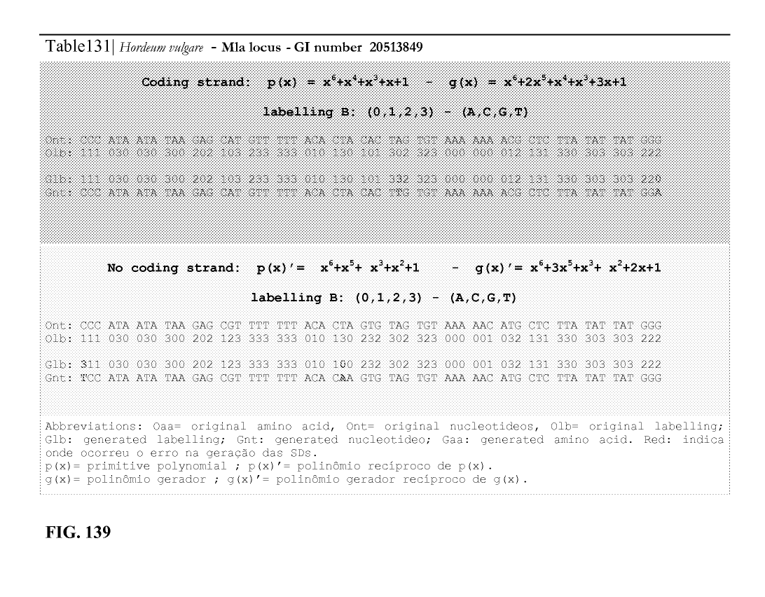

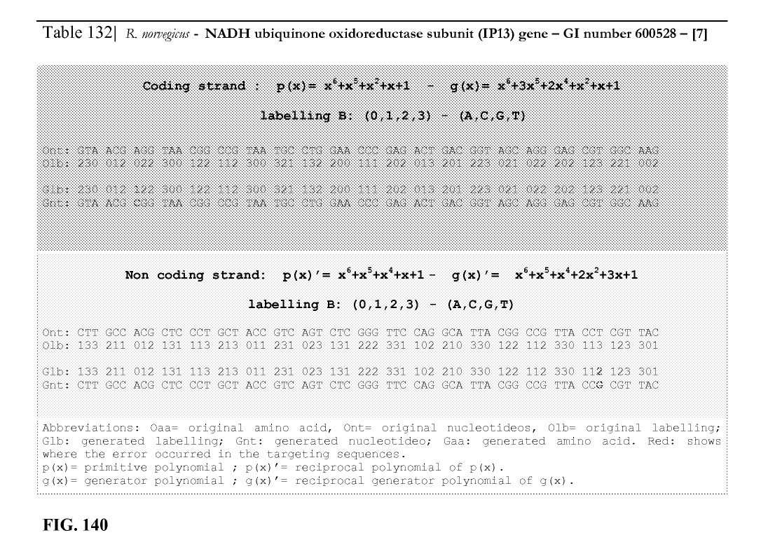

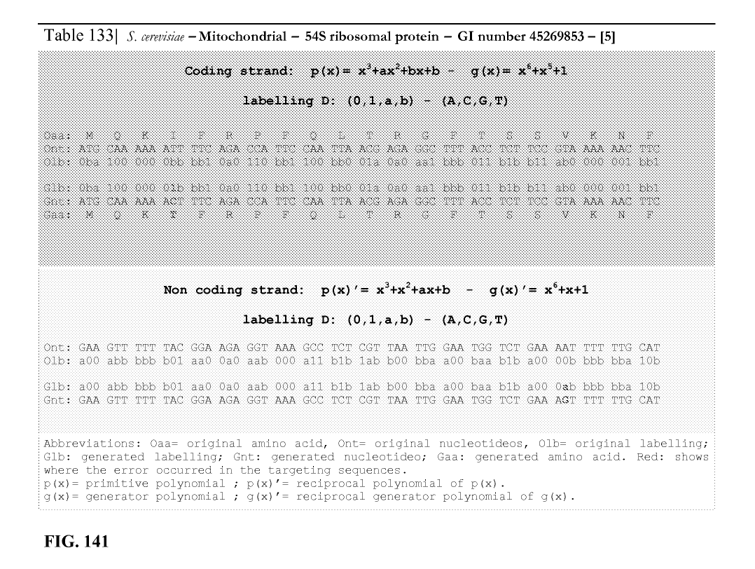

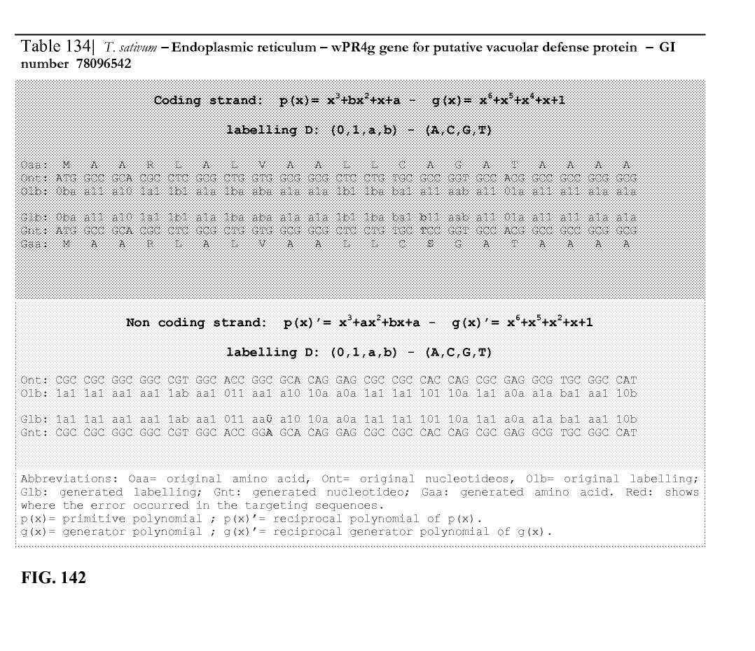

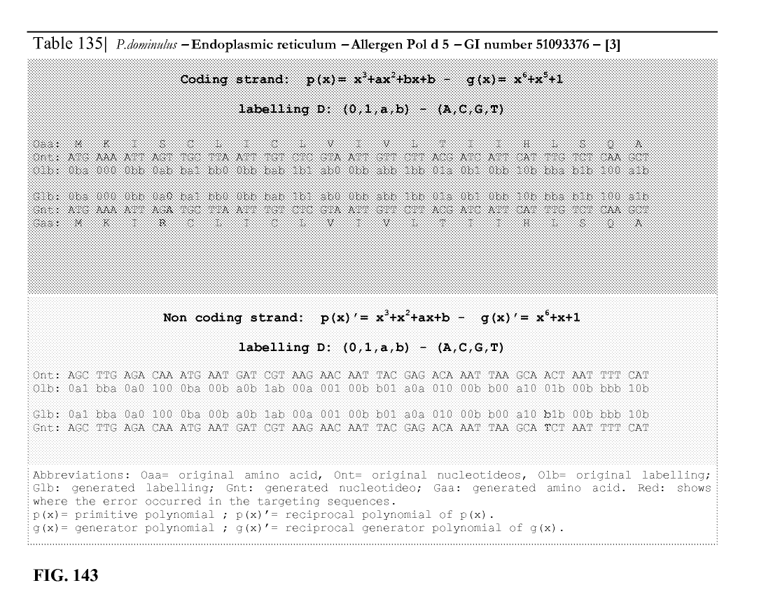

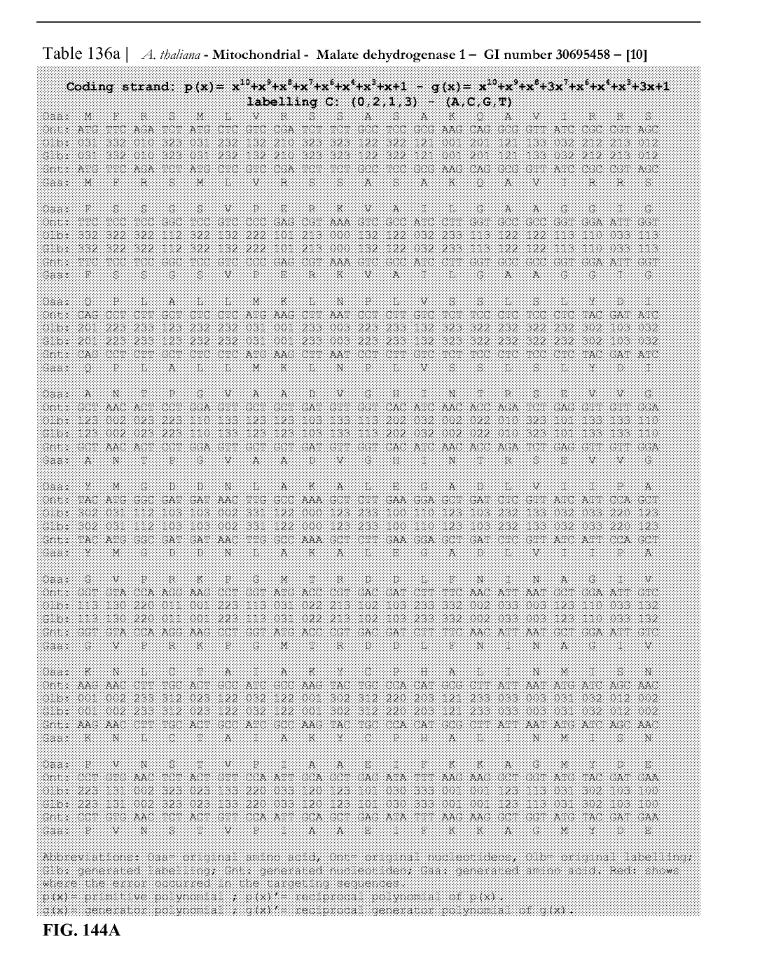

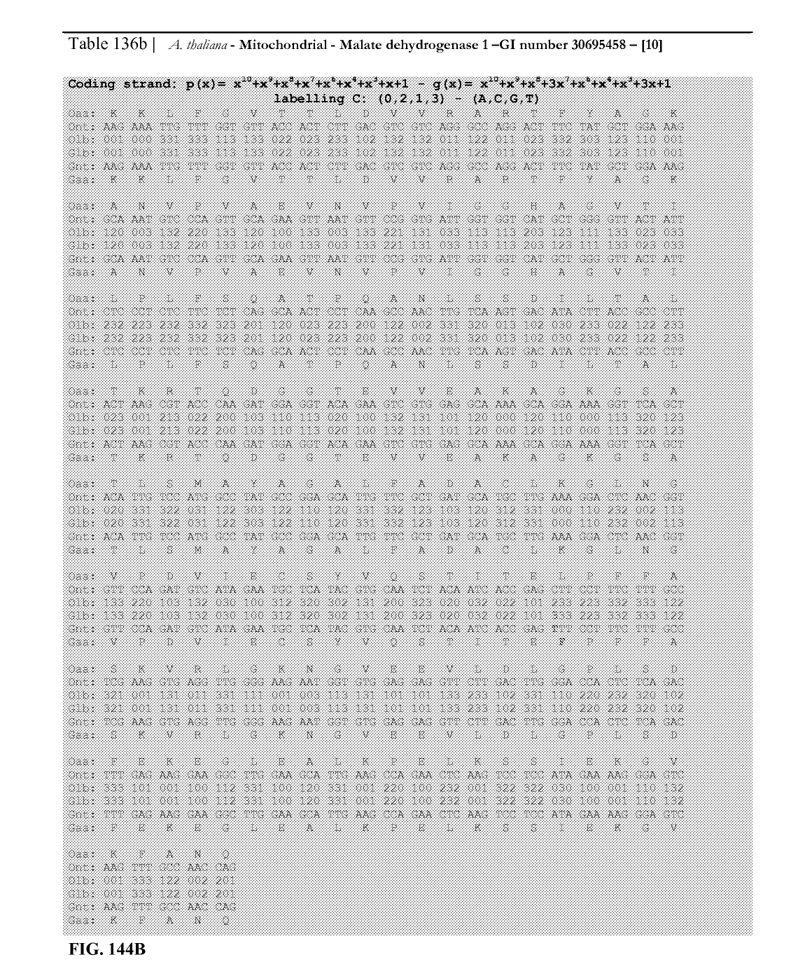

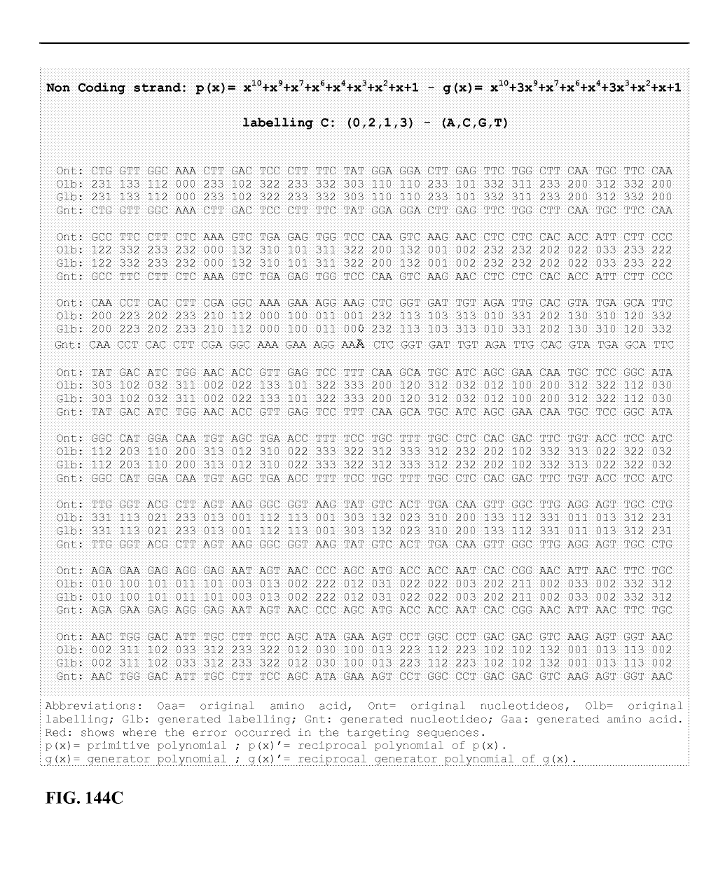

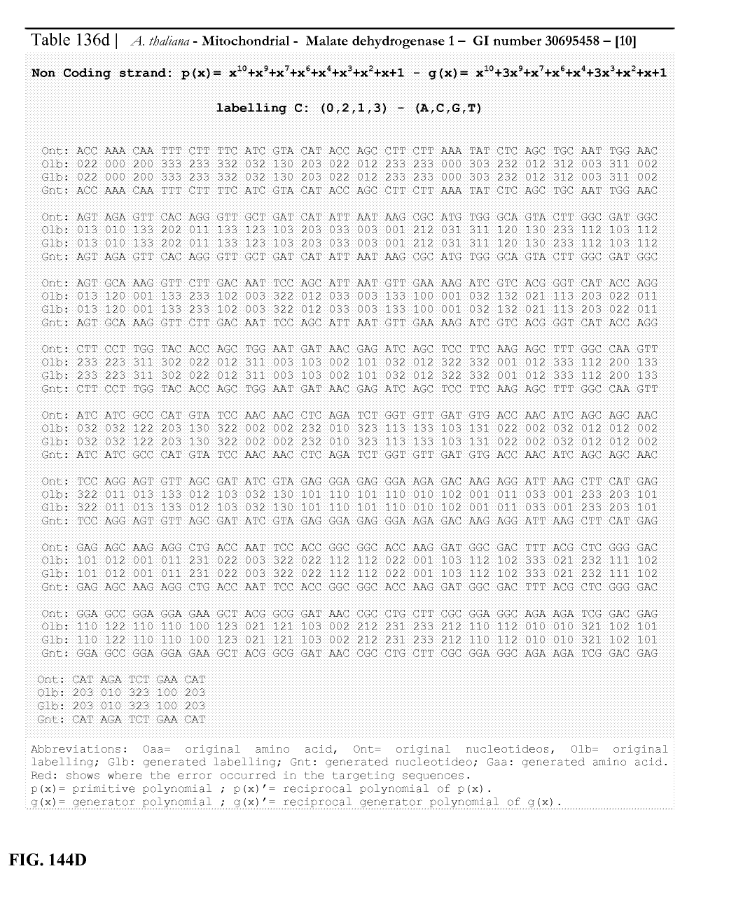

The way data flows through various programs is named, for example, PLAMJ and it is shown in FIG. 8. The yellow rectangles show the goal of the present invention. The gray rectangles are related to the mathematical operations which the program must perform. The pink rectangles surround the names of programs of the present invention that have been executed.

One exemplary system for implementing the present invention includes a computing device. One having ordinary skill appreciates in light of the present specification that various computing devices suitable for carrying out the present invention are available. A computing device includes at least one processing unit and one system memory. Depending on the configuration and type of computing device, a system memory may be volatile (e.g., RAM), non-volatile (e.g., ROM, flash memory, etc.) or combinations thereof. System memory includes an operating system, one or more applications, and may include program data. In one aspect, the application may include, among others, a method for analyzing polymorphisms and mutations in DNA sequences. In another aspect, the application may be a method for analyzing polymorphisms and mutations in DNA sequences program when a computing device is configured as a server. A computing device may have additional features or functionality, e.g., additional data storage devices (removable and/or non-removable) such as, magnetic disks, optical disks, or tape; and removable storage and non-removable storage. The computer storage media includes volatile and nonvolatile, removable and non-removable media implemented in any method or technology for storage of information, such as computer readable instructions, data structures, program modules, genetic information, DNA sequences, nucleic acids, amino acids, or any other data. The system memory, removable storage and non-removable storage are examples of computer storage media. The computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing the device. Any such computer storage media may be part of the device. The computing device may have input device(s) such as keyboard, mouse, pen, voice input device, touch input device, etc. Output device(s) such as a display, speakers, printer, etc. may also be included. The computing device may also contain communication connections that allow the device to communicate with other computing devices, such as over a network. A communication connection is one example of communication media. Communication media may typically be embodied by computer readable instructions, data structures, program modules, or other data in a modulated data signal, such as a carrier wave or other transport mechanism, and includes information delivery media. The term “modulated data signal” includes a signal that has one or more of its characteristics set or changed in such a manner as to encode information in the signal. For example, a communication media includes wired media such as a wired network or direct-wired connection, and wireless media such as acoustic, RF, infrared and other wireless media. The term computer readable media includes both storage media and communication media.

A mobile computing device may be used in one exemplary aspect of the present invention. One exemplary system for implementing the invention includes a mobile computing device. The mobile computing device includes a processor, memory, display, and keypad. The memory generally includes both volatile memory (e.g., RAM) and non-volatile memory (e.g., ROM, Flash Memory, or the like). The mobile computing device includes an operating system, which is resident in the memory and executes on the processor. The keypad includes a push button numeric dialing pad, a multi-key keyboard. The display includes a liquid crystal display, or any other type of display commonly used in mobile computing devices. The display may be touch-sensitive, and acts as an input device. One or more application programs are loaded into memory and run on the operating system. The method for analyzing polymorphisms and mutations in DNA sequences, among other applications resides on a mobile computing device and is programmed to interact with a program located on a server. The mobile computing device also includes a non-volatile storage within the memory. Non-volatile storage is used to store persistent information which should not be lost if the mobile computing device is powered down. The mobile computing device includes a power supply, which is implemented as one or more batteries. The power supply might further include an external power source, such as an AC adapter or a powered docking cradle that supplements or recharges the batteries. The mobile computing device includes two types of optional external notification mechanisms: a LED and an audio interface. These devices may be directly coupled to the power supply so that when activated, they remain on for a duration dictated by the notification mechanism even though the processor and other components might shut down to conserve battery power. The audio interface is used to provide audible signals to and receive audible signals from the user. For example, the audio interface may be coupled to a speaker for providing audible output and to a microphone for receiving audible input, such as to facilitate a telephone conversation. The mobile computing device also includes one or more communications connections, such as a wireless interface layer, that performs the function of transmitting and receiving communications. The communications connection facilitates wireless connectivity between the mobile computing device and the outside world. In one aspect, transmissions to and from the communications connection are conducted under control of the operating system.

### Programs for the Generation of DNA Sequences by Use of BCH Codes Over Ring

1. Program minimal.m
gx=minimal(n, d, p, r, pr, step)
Function: Compute the generator polynomial g(x) of the cyclic code.

Input Parameters:
n=code word length;
d=code distance (d=2t+1, where t is the error correction capability of the code)
p=prime number;
r=Galois extension;
pr=primitive polynomial of degree r, irreducible over GF(p) and consequently over Z_{q}. Remark: the representation of the coefficients of the polynomial pr is from the greatest to the least exponent;
step=integer number greater than or equal to 1 which divides the ring order, generating a cyclic subgroup Gₙ of order n.

Output Parameters:
gx=generator polynomial of the cyclic code.

Program Description:

The program minimal computes the generator polynomial of the matrix G(x). The first step is to determine the βⁱ's roots of the minimal polynomial Mᵢ(x) over the group of units of the ring, where β is a primitive element in G. and the roots are in the sequence; the powers of β are reduced modulo n. For this step it is used the routine root.m with input parameters (n, d, p and r). The next step is to compute the cyclic subgroup G. with order equal to n. This step makes use of the routine tab.m with input parameters r, pr and step. Finally, the generator polynomial g(x) is obtained through the 1 cm (least common multiple) of the minimal polynomials, that is, g(x)=1 cm (M₁(x), M₂(x), . . . , M₂ₜ(x)), where t is the error correction capability of the code. For this it is used the routine gx.m with the minimal polynomials as input parameters. The coefficients of the polynomials in the computations are reduced modulo q, where q=p^{k}, for k≧2.
Ex: gx=minimal(63, 3, 2, 6, [3 0 3 3 0 3], 2)
gx=[1 3 0 1 1 2 1] (coefficients of the polynomial g(x) from the greatest to the least exponent)

2. Program matrixg.m
mat=matrixg(m, gx)
Function: Determine the generator matrix G(x).

Input Parameters:
n=code word length;
gx=generator polynomial computed by the program minimal.m.

Output Parameters:
mat=generator matrix G(x).

Program Description:

The generator matrix G(x) is obtained by shifting the coefficients of the generator polynomial g(x) from the left to the right, one column in each row. Matrix G(x) has k rows and n columns, where k=n−g, where g is the degree of the polynomial g(x).

$$Ex \text{:} mat = matrixg ( 63 , gx )$$

$$mat = \\ 121103100000000000000000000000000000000000000000000000000000000$$

$$012110310000000000000000000000000000000000000000000000000000000$$

$$001211031000000000000000000000000000000000000000000000000000000$$

$$000121103100000000000000000000000000000000000000000000000000000$$

$$000012110310000000000000000000000000000000000000000000000000000$$

$$000001211031000000000000000000000000000000000000000000000000000$$

$$000000121103100000000000000000000000000000000000000000000000000$$

$$000000012110310000000000000000000000000000000000000000000000000$$

$$000000001211031000000000000000000000000000000000000000000000000$$

$$000000000121103100000000000000000000000000000000000000000000000$$

$$000000000012110310000000000000000000000000000000000000000000000$$

$$000000000001211031000000000000000000000000000000000000000000000$$

$$000000000000121103100000000000000000000000000000000000000000000$$

$$000000000000012110310000000000000000000000000000000000000000000$$

$$000000000000001211031000000000000000000000000000000000000000000$$

$$000000000000000121103100000000000000000000000000000000000000000 \\ \begin{matrix}⋮ & ⋮ & ⋮\end{matrix} \\ 000000000000000000000000000000000000000000000000121103100000000 \\ 000000000000000000000000000000000000000000000000012110310000000 \\ 000000000000000000000000000000000000000000000000001211031000000 \\ 000000000000000000000000000000000000000000000000000121103100000 \\ 000000000000000000000000000000000000000000000000000012110310000 \\ 000000000000000000000000000000000000000000000000000001211031000 \\ 000000000000000000000000000000000000000000000000000000121103100 \\ 000000000000000000000000000000000000000000000000000000012110310 \\ 000000000000000000000000000000000000000000000000000000001211031$$

3. Program diviring.m
[hx, r]=diviring(pl, gx)
Function: Determine the generator polynomial of the dual cyclic code.

Input Parameters:
pl=polynomial xⁿ−1, where n=code word length;
gx=generator polynomial computed by the program minimal.m.

Output Parameters:
hx=generator polynomial of the parity-check matrix H(x);
r=remainder of the division.

Program Description:

The generator polynomial h(x) of the parity-check matrix H(x) is determined by the division of the polynomial pl=xⁿ−1 by the generator polynomial g(x).
Ex: [hx, r]=diviring(x⁶³−1,gx) hx=[1 1 1 0 2 2 2 1 0 0 3 0 0 0 1 3 0 3 3 2 0 3 0 1 3 2 3 0 1 1 3 2 1 3 3 1 2 0 1 2 0 0 3 2 1 0 3 2 2 3 1 3 3 1 1 2 3] (polynomial coefficients from the greatest to the least exponent)
r=0

4. Program matrixh.m
math=matrixh(hx, n)
Function: Determine the parity-check matrix H(x).

Input Parameters:
hx=generator polynomial of matrix H(x) determined by the program diviring.m;
n=code word length.

Output Parameters:
math=parity-check matrix H(x).

Program Description:

The parity-check matrix H(x) is obtained by shifting the coefficients of the polynomial h(x) from the right to the left, one column in each row.

$$Ex \text{:} math = matrixh ( hx , 63 )$$

$$math = \\ 000001110222100300013033203013230113213312011200321032231331123$$

$$000011102221003000130332030132301132133120112003210322313311230$$

$$000111022210030001303320301323011321331201120032103223133112300$$

$$001110222100300013033203013230113213312011200321032231331123000$$

$$011102221003000130332030132301132133120112003210322313311230000$$

$$111022210030001303320301323011321331201120032103223133112300000$$

5. Program gxhx.m—for 1 Nucleotide Error
[vetg, veth]=gxhx(prot, n, gx, hx)

Function: Determine if the desired information sequence is a code word and if there is a code word which differs in only one position from the desired information sequence.

Input Parameters:
prot=desired information sequence;
n=code word length;
gx=generator polynomial calculated by the program minimal.m;
hx=generator polynomial of matrix H(x) calculated by the program diviring.m.

Output Parameters:
vetg=code words of the matrix H(x) without errors or that differ in only one position from the desired information sequence;
veth=code words of the matrix G(x) without errors or that differ in only one position from the desired information sequence.

Program Description:

The program gxhx.m uses the routine label.m to generate the 24 possible permutations between the genetic alphabet (A, C, G, T) and the code alphabet (0, 1, 2, 3). Thus, the 24 possible cases of the labeling/mapping are generated by the information sequence (prot) without nucleotide errors. The next step is to generate all the possible code words with 1 error for the 24 cases. These code words differ in only one position from the information sequence. Finally, all these possible code words without errors or with 1 nucleotide error are multiplied by G(x) and H(x) matrices. If the multiplication of the possible code word by the matrix H(x) is 0 (zero), then this is a code word (without error or with 1 nucleotide error) of the generator matrix G(x). In the same way, if the multiplication of the possible code word by the matrix G(x) is 0 (zero), then this possible code word is a code word (without error or with 1 nucleotide error) of the matrix H(x).
Ex: [vetg, veth]=gxhx

(′TTCAGATCCGCGCTTGTCCGATCCTCCGCCTCGGCGAAGCAGTC
GCTTCTCCGCCGCAGCTTC′ (SEQ ID NO: 3), 63, gx,hx)

$$vetg = \\ Columns 1 through 16$$

$$\begin{matrix}0 & 0 & 0 & 0 & 0 & 0 & 0 & 0 & 0 & 0 & 0 & 0 & 0 & 0 & 0 & 0 \\ ⋮ & & & & & & & & ⋮ & & & & & & & ⋮\end{matrix}$$

$$Columns 49 through 63$$

$$0 \qquad 0 \qquad 0 \qquad 0 \qquad 0 \qquad 0 \qquad 0 \qquad 0 \qquad 0 \qquad 0 \qquad 0 \qquad 0 \qquad 0 \qquad 0 \qquad 0$$

$$veth = \\ Columns 1 through 16$$

$$\begin{matrix}2 & 2 & 1 & 0 & 3 & 0 & 2 & 1 & 1 & 3 & 1 & 3 & 1 & 2 & 2 & 3 \\ 2 & 2 & 3 & 0 & 1 & 0 & 2 & 3 & 3 & 1 & 3 & 1 & 3 & 2 & 2 & 1 \\ 3 & 3 & 0 & 1 & 2 & 1 & 3 & 0 & 0 & 2 & 0 & 2 & 0 & 3 & 3 & 2 \\ 3 & 3 & 2 & 1 & 0 & 1 & 3 & 2 & 2 & 0 & 2 & 0 & 2 & 3 & 3 & 0 \\ 0 & 0 & 1 & 2 & 3 & 2 & 0 & 1 & 1 & 3 & 1 & 3 & 1 & 0 & 0 & 3 \\ 0 & 0 & 3 & 2 & 1 & 2 & 0 & 3 & 3 & 1 & 3 & 1 & 3 & 0 & 0 & 1 \\ 1 & 1 & 0 & 3 & 2 & 3 & 1 & 0 & 0 & 2 & 0 & 2 & 0 & 1 & 1 & 2 \\ 1 & 1 & 2 & 3 & 0 & 3 & 1 & 2 & 2 & 0 & 2 & 0 & 2 & 1 & 1 & 0\end{matrix}$$

$$Columns 17 through 32$$

$$\begin{matrix}2 & 1 & 1 & 3 & 0 & 2 & 1 & 1 & 2 & 1 & 1 & 3 & 1 & 1 & 2 & 1 \\ 2 & 3 & 3 & 1 & 0 & 2 & 3 & 3 & 2 & 3 & 3 & 1 & 3 & 3 & 2 & 3 \\ 3 & 0 & 0 & 2 & 1 & 3 & 0 & 0 & 3 & 0 & 0 & 2 & 0 & 0 & 3 & 0 \\ 3 & 2 & 2 & 0 & 1 & 3 & 2 & 2 & 3 & 2 & 2 & 0 & 2 & 2 & 3 & 2 \\ 0 & 1 & 1 & 3 & 2 & 0 & 1 & 1 & 0 & 1 & 1 & 3 & 1 & 1 & 0 & 1 \\ 0 & 3 & 3 & 1 & 2 & 0 & 3 & 3 & 0 & 3 & 3 & 1 & 3 & 3 & 0 & 3 \\ 1 & 0 & 0 & 2 & 3 & 1 & 0 & 0 & 1 & 0 & 0 & 2 & 0 & 0 & 1 & 0 \\ 1 & 2 & 2 & 0 & 3 & 1 & 2 & 2 & 1 & 2 & 2 & 0 & 2 & 2 & 1 & 2\end{matrix}$$

$$Columns 33 through 48$$

$$\begin{matrix}3 & 3 & 1 & 3 & 0 & 0 & 3 & 1 & 0 & 3 & 2 & 1 & 3 & 1 & 2 & 2 \\ 1 & 1 & 3 & 1 & 0 & 0 & 1 & 3 & 0 & 1 & 2 & 3 & 1 & 3 & 2 & 2 \\ 2 & 2 & 0 & 2 & 1 & 1 & 2 & 0 & 1 & 2 & 3 & 0 & 2 & 0 & 3 & 3 \\ 0 & 0 & 2 & 0 & 1 & 1 & 0 & 2 & 1 & 0 & 3 & 2 & 0 & 2 & 3 & 3 \\ 3 & 3 & 1 & 3 & 2 & 2 & 3 & 1 & 2 & 3 & 0 & 1 & 3 & 1 & 0 & 0 \\ 1 & 1 & 3 & 1 & 2 & 2 & 1 & 3 & 2 & 1 & 0 & 3 & 1 & 3 & 0 & 0 \\ 2 & 2 & 0 & 2 & 3 & 3 & 2 & 0 & 3 & 2 & 1 & 0 & 2 & 0 & 1 & 1 \\ 0 & 0 & 2 & 0 & 3 & 3 & 0 & 2 & 3 & 0 & 1 & 2 & 0 & 2 & 1 & 1\end{matrix}$$

$$Columns 49 through 63$$

$$\begin{matrix}1 & 2 & 1 & 1 & 3 & 1 & 1 & 3 & 1 & 0 & 3 & 1 & 2 & 2 & 2 \\ 3 & 2 & 3 & 3 & 1 & 3 & 3 & 1 & 3 & 0 & 1 & 3 & 2 & 2 & 2 \\ 0 & 3 & 0 & 0 & 2 & 0 & 0 & 2 & 0 & 1 & 2 & 0 & 3 & 3 & 3 \\ 2 & 3 & 2 & 2 & 0 & 2 & 2 & 0 & 2 & 1 & 0 & 2 & 3 & 3 & 3 \\ 1 & 0 & 1 & 1 & 3 & 1 & 1 & 3 & 1 & 2 & 3 & 1 & 0 & 0 & 0 \\ 3 & 0 & 3 & 3 & 1 & 3 & 3 & 1 & 3 & 2 & 1 & 3 & 0 & 0 & 0 \\ 0 & 1 & 0 & 0 & 2 & 0 & 0 & 2 & 0 & 3 & 2 & 0 & 1 & 1 & 1 \\ 2 & 1 & 2 & 2 & 0 & 2 & 2 & 0 & 2 & 3 & 0 & 2 & 1 & 1 & 1\end{matrix}$$

These results show that in H(x) there are no code words without nucleotide differences or that differ in only one position from the desired information sequence (vetg=0). It can also be observed that there are 8 code words of the matrix G(x). In this case, they differ in only one position from the information sequence (parameter veth).

5.1 Program gxhx2errors.m—for 2 Nucleotide Errors
[vetg, veth]=gxhx2errors(prot, n, gx, hx, case)

Function: Determine if exists a code word that differs in two positions from the desired information sequence.

Input Parameters:
prot=desired information sequence;
n=code word length;
gx=generator polynomial calculated by the program minimal.m;
hx=generator polynomial of matrix H(x) calculated by the program diviring.m
case=labelling case

Output Parameters:
vetg=code words of matrix H(x) that differ in two positions from the desired information sequence;
veth=code words of matrix G(x) that differ in two positions from the desired information sequence.

Program Description:

The program gxhx2errors.m uses the routine label.m for the labelling between the genetic alphabet (A, C, G, T) and the code alphabet (0, 1, 2, 3) to the specified case. Thus, it is generated one possible case of labelling for the information sequence (prot) without nucleotide errors. The next step is to generate all the possible code words differing in 2 positions. These code words differ in two positions from the information sequence. Finally, all these possible code words differing in 2 nucleotides are multiplied by G(x) and H(x) matrices. If the multiplication of the possible code word by the matrix H(x) is 0 (zero), then this is a code word (differing in 2 nucleotides) of the generator matrix G(x). In the same way, if the multiplication of the possible code word by the matrix G(x) is 0 (zero), then this possible code word is a code word (differing in 2 nucleotides) of matrix H(x).
Ex.: [vetg, veth]=gxhx2errors

gxhx2errors
(′ATGAAACTATTTCTTTTACTAGTTATCTCTGCTTCAATGCTAAT
TGATGGCTTAGTTAATGCT′ (SEQ ID NO: 4),63,gx,hx,2)

$$veth = \\ Columns 1 through 21$$

$$\begin{matrix}0 & 2 & 3 & 0 & 0 & 0 & 1 & 2 & 0 & 2 & 2 & 2 & 1 & 0 & 2 & 2 & 2 & 0 \\ 1 & 2 & 0 & & & & & & & & & & & & & & & \\ 0 & 2 & 3 & 0 & 0 & 0 & 1 & 2 & 0 & 2 & 2 & 2 & 1 & 2 & 2 & 2 & 2 & 0 \\ 1 & 2 & 0 & & & & & & & & & & & & & & & \\ 0 & 2 & 3 & 0 & 0 & 0 & 1 & 2 & 0 & 2 & 2 & 2 & 1 & 2 & 2 & 2 & 2 & 0 \\ 1 & 2 & 0 & & & & & & & & & & & & & & & \end{matrix}$$

$$Columns 22 through 42$$

$$\begin{matrix}3 & 2 & 2 & 0 & 2 & 1 & 2 & 1 & 2 & 3 & 1 & 2 & 2 & 1 & 0 & 0 & 2 & 3 \\ 1 & 2 & 0 & & & & & & & & & & & & & & & \\ 3 & 2 & 2 & 0 & 2 & 1 & 2 & 2 & 2 & 3 & 1 & 2 & 2 & 1 & 3 & 0 & 2 & 3 \\ 1 & 2 & 0 & & & & & & & & & & & & & & & \\ 3 & 2 & 2 & 0 & 2 & 1 & 2 & 1 & 2 & 3 & 1 & 2 & 2 & 1 & 0 & 0 & 2 & 3 \\ 1 & 2 & 2 & & & & & & & & & & & & & & & \end{matrix}$$

$$Columns 43 through 63$$

$$\begin{matrix}0 & 2 & 2 & 3 & 0 & 2 & 3 & 3 & 1 & 2 & 2 & 0 & 0 & 2 & 2 & 0 & 0 & 2 \\ 3 & 1 & 2 & & & & & & & & & & & & & & & \\ 0 & 2 & 2 & 3 & 0 & 2 & 3 & 3 & 1 & 2 & 2 & 0 & 3 & 2 & 2 & 0 & 0 & 2 \\ 3 & 1 & 2 & & & & & & & & & & & & & & & \\ 0 & 2 & 2 & 3 & 0 & 2 & 3 & 3 & 1 & 2 & 2 & 0 & 2 & 2 & 2 & 0 & 0 & 2 \\ 3 & 1 & 2 & & & & & & & & & & & & & & & \end{matrix}$$

6. Program label_inv.m—for 1 and 2 Nucleotide Errors
result=label_inv (codeword, n, case, prot)

Function: Determine in which permutations the code words were found and show if there are nucleotide differences. In the case of differing in one position, the program shows in which position the Ont (nucleotides of the desired sequence) and Gnt sequences (nucleotides of the sequence generated by the code) differ from each other. Consequently, the Oaa and Gaa sequences present the differences in amino acids.

Input Parameters:
codeword=code word differing in one position, obtained with the program gxhx.m, or differing in 2 positions obtained with the program gxhx2errors;
n=code word length;
case=labelling case (1 to 24);
prot=desired information sequence.

Output Parameters:
result=amino acids, nucleotides and labelling of the desired sequence and the generated sequence

Program Description:

The first step is to label the code word in the genetic alphabet (A, T, C, G) for the specified case. This nucleotide sequence is converted to the correspondent sequence in amino acids using the routine pro2ami.m. The desired information sequence is also converted to its correspondent sequence in amino acids by the routine pro2ami.m. The program label.m is used for the conversion of the desired information sequence in the code alphabet. All this information is stored in result.

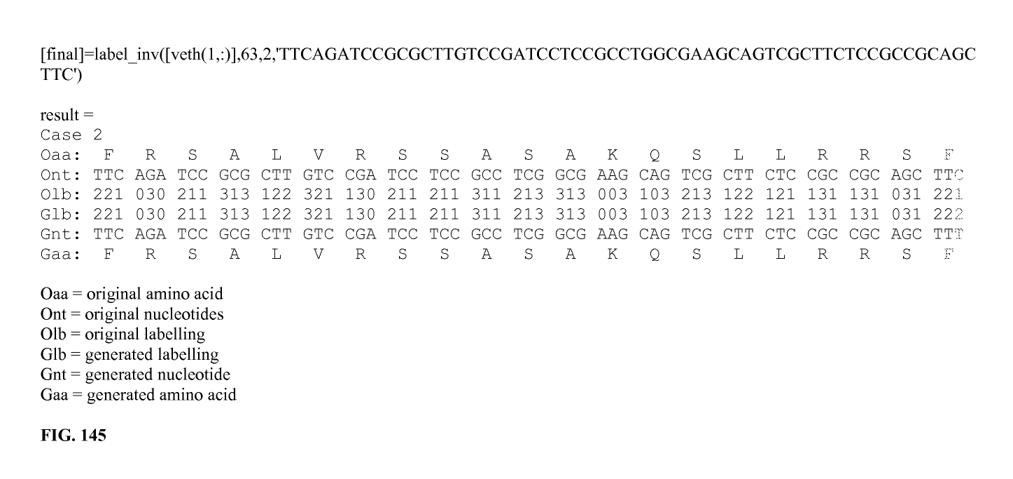

Ex 1:1 Nucleotide Difference: see FIG. 145

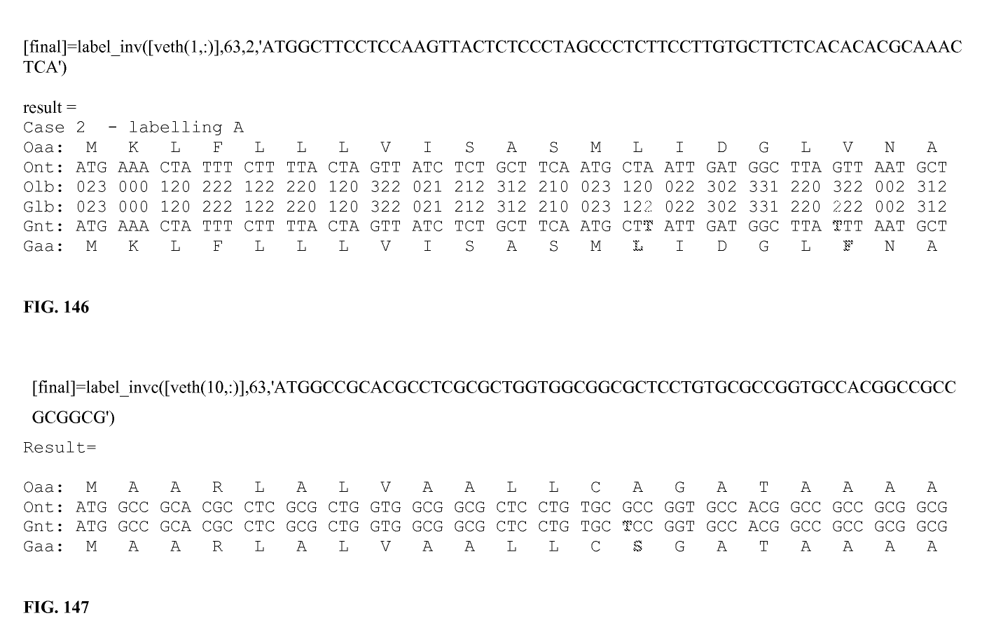

Ex 2: 2 Nucleotide Differences: see FIG. 146

7. Program system.m
[errors, result]=system(mat,prot)

Function: Compute the vector u that multiplies the matrix G(x) in order to determine the sequence generated by the code and to show in which labelling no differences were found.

Input Parameters:
mat=generator matrix G(x) determined by the program matrixg.m
prot=generated information sequence.

Output Parameters:
errors=differences found for each labelling case
result=vector u, amino acids, nucleotides and labelling of the desired sequence and the generated sequence

Program Description:

The program system.m uses the routine label.m to generate the 24 possible permutations between the genetic alphabet and the code alphabet. Thus, the 24 possible cases of the labelling are generated by the information sequence (prot). Using matrix G(x) and the labelling, a system of modular equations to determine the vector u is formed. For each one of the 24 labelling cases it is formed and solved a system of modular equations is established with the aim to finding the corresponding vector u. This vector is multiplied by the matrix G(x) to generate a code word which is compared with the information sequence (prot). The program system.m uses the routines pro2ami.m and label_inv2.m for conversions from nucleotides to amino acids and from the code alphabet to the genetic alphabet, respectively.
[errors,result]=system(mat,

′TTCAGATCCGCGCTTGTCCGATCCTCCGCCTCGGCG
AAGCAGTCGCTTCTCCGCCGCAGCTTT′ (SEQ ID NO: 5))

result=

### Case 1—(0,1,2,3)=(A,C,G,T)—4 Errors/2 aa

u={310 011 222 330 321 332 202 102 010 023 031 303 231 313 231 013 330 013 232}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 331 020 311 212 133 231 120 311 311 211 312 212 002 102 312 133 131 121 121 021 333

### Glb: 331 020 311 212 133 231 120 311 311 211 312 212 002 102 312 133 131 121 121 033 312

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

### Case 2—(0,1,3,2)=(A,C,G,T)—0 Errors/0 aa

u={223 221 031 203 012 020 022 233 113 012 121 310 100 230 021 203 300 021 202}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 221 030 211 313 122 321 130 211 211 311 213 313 003 103 213 122 121 131 131 031 222

### Glb: 221 030 211 313 122 321 130 211 211 311 213 313 003 103 213 122 121 131 131 031 222

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa: F R S A L V R S S A S A K Q S L L R R S F

### Case 3—(0,2,1,3)=(A,C,G,T)—3 Errors/1 aa

u={311 232 013 333 313 312 002 133 101 011 332 013 111 323 012 010 230 210 012}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 332 010 322 121 233 132 210 322 322 122 321 121 001 201 321 233 232 212 212 012 333

### Glb: 332 010 322 121 233 132 210 322 322 122 321 121 001 201 321 233 232 212 212 202 332

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 4—(0,2,3,1)=(A,C,G,T)—3 Errors/1 aa

u={133 212 031 111 131 132 002 311 303 033 112 031 333 121 032 030 210 230 032}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 112 030 122 323 211 312 230 122 122 322 123 323 003 203 123 211 212 232 232 032 111

### Glb: 112 030 122 323 211 312 230 122 122 322 123 323 003 203 123 211 212 232 232 202 112

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 5—(0,3,2,1)=(A,C,G,T)—4 Errors/2 aa

u={130 033 222 110 123 112 202 302 030 021 013 101 213 131 213 031 110 031 212}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 113 020 133 232 311 213 320 133 133 233 132 232 002 302 132 311 313 323 323 023 111

### Glb: 113 020 133 232 311 213 320 133 133 233 132 232 002 302 132 311 313 323 323 011 132

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

### Case 6—(0,3,1,2)=(A,C,G,T)—0 Errors/0 aa

u={221 223 013 201 032 020 022 211 331 032 323 130 300 210 023 201 100 023 202}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 223 010 233 131 322 123 310 233 233 133 231 131 001 301 231 322 323 313 313 013 222

### Glb: 223 010 233 131 322 123 310 233 233 133 231 131 001 301 231 322 323 313 313 013 222

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa: F R S A L V R S S A S A K Q S L L R R S F

### Case 7—(1,0,2,3)=(A,C,G,T)—0 Errors/0 aa

u={313 302 200 101 300 200 300 201 033 313 003 230 013 221 230 313 321 332 123}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 330 121 300 202 033 230 021 300 300 200 302 202 112 012 302 033 030 020 020 120 333

### Glb: 330 121 300 202 033 230 021 300 300 200 302 202 112 012 302 033 030 020 020 120 333

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa: F R S A L V R S S A S A K Q S L L R R S F

### Case 8—(1,0,3,2)=(A,C,G,T)—4 Errors/2 aa

u={222 112 013 010 031 332 120 332 132 302 133 201 322 102 020 103 331 300 133}

(SEQ ID NO; 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 220 131 200 303 022 320 031 200 200 300 203 303 113 013 203 022 020 030 030 130 222

### Glb: 220 131 200 303 022 320 031 200 200 300 203 303 113 013 203 022 020 030 030 122 203

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

### Case 9—(1,2,0,3)=(A,C,G,T)—0 Errors/0 aa

u={311 300 222 103 320 200 300 223 211 333 201 010 213 201 232 311 121 330 123}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 332 101 322 020 233 032 201 322 322 022 320 020 110 210 320 233 232 202 202 102 333

### Glb: 332 101 322 020 233 032 201 322 322 022 320 020 110 210 320 233 232 202 202 102 333

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa: F R S A L V R S S A S A K Q S L L R R S F

### Case 10—(1,2,3,0)=(A,C,G,T)—4 Errors/2 aa

u={002 130 013 230 233 112 120 132 112 300 111 003 300 320 002 121 111 322 113}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 002 131 022 323 200 302 231 022 022 322 023 323 113 213 023 200 202 232 232 132 000

### Glb: 002 131 022 323 200 302 231 022 022 322 023 323 113 213 023 200 202 232 232 100 023

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

### Case 11—(1,3,0,2)=(A,C,G,T)—3 Errors/1 aa

u={221 331 222 011 003 312 320 301 001 310 232 131 002 132 203 102 031 103 313}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 223 101 233 030 322 023 301 233 233 033 230 030 110 310 230 322 323 303 303 103 222

### Glb: 223 101 233 030 322 023 301 233 233 033 230 030 110 310 230 322 323 303 303 313 223

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 12—(1,3,2,0)=(A,C,G,T)—3 Errors/1 aa

u={003 311 200 233 221 132 320 123 203 332 012 113 220 330 223 122 011 123 333}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 003 121 033 232 300 203 321 033 033 233 032 232 112 312 032 300 303 323 323 123 000

### Glb: 003 121 033 232 300 203 321 033 033 233 032 232 112 312 032 300 303 323 323 313 003

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 13—(2,0,1,3)=(A,C,G,T)—3 Errors/1 aa

u={313 010 013 311 311 132 202 331 103 231 312 231 111 103 010 210 212 012 230}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 330 212 300 101 033 130 012 300 300 100 301 101 221 021 301 033 030 010 010 210 333

### Glb: 330 212 300 101 033 130 012 300 300 100 301 101 221 021 301 033 030 010 010 020 330

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 14—(2,0,3,1)=(A,C,G,T)—3 Errors/1 aa

u={131 030 031 133 133 312 202 113 301 213 132 213 333 301 030 230 232 032 210}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 110 232 100 303 011 310 032 100 100 300 103 303 223 023 103 011 010 030 030 230 111

### Glb: 110 232 100 303 011 310 032 100 100 300 103 303 223 023 103 011 010 030 030 020 110

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 15—(2,1,0,3)=(A,C,G,T)—4 Errors/2 aa

u={310 231 200 310 303 112 002 322 230 223 213 301 031 113 231 211 112 213 010}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 331 202 311 010 133 031 102 311 311 011 310 010 220 120 310 133 131 101 101 201 333

### Glb: 331 202 311 010 133 031 102 311 311 011 310 010 220 120 310 133 131 101 101 233 310

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

### Case 16—(2,1,3,0)=(A,C,G,T)—0 Errors/0 aa

u={001 021 031 001 212 020 222 231 131 230 123 330 122 232 001 021 102 201 000}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 001 232 011 313 100 301 132 011 011 311 013 313 223 123 013 100 101 131 131 231 000

### Glb: 001 232 011 313 100 301 132 011 011 311 013 313 223 123 013 100 101 131 131 231 000

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa: F R S A L V R S S A S A K Q S L L R R S F

### Case 17—(2,3,0,1)=(A,C,G,T)—4 Errors/2 aa

u={130 213 200 130 101 332 002 122 210 221 231 103 013 331 213 233 332 231 030}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 113 202 133 030 311 013 302 133 133 033 130 030 220 320 130 311 313 303 303 203 111

### Glb: 113 202 133 030 311 013 302 133 133 033 130 030 220 320 130 311 313 303 303 211 130

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

### Case 18—(2,3,1,0)=(A,C,G,T)—0 Errors/0 aa

u={003 023 013 003 232 020 222 213 313 210 321 110 322 212 003 023 302 203 000}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 003 212 033 131 300 103 312 033 033 133 031 131 221 321 031 300 303 313 313 213 000

### Glb: 003 212 033 131 300 103 312 033 033 133 031 131 221 321 031 300 303 313 313 213 000

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa:F R S A L V R S S A S A K Q S L L R R S F

### Case 19—(3,0,1,2)=(A,C,G,T)—4 Errors/2 aa

u={222 332 031 030 013 112 320 112 312 102 311 203 122 302 020 301 113 100 311}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 220 313 200 101 022 120 013 200 200 100 201 101 331 031 201 022 020 010 010 310 222

### Glb: 220 313 200 101 022 120 013 200 200 100 201 101 331 031 201 022 020 010 010 322 201

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

### Case 20—(3,0,2,1)=(A,C,G,T)—0 Errors/0 aa

u={131 102 200 303 100 200 100 203 011 131 001 210 031 223 210 131 123 112 321}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 110 323 100 202 011 210 023 100 100 200 102 202 332 032 102 011 010 020 020 320 111

### Glb: 110 323 100 202 011 210 023 100 100 200 102 202 332 032 102 011 010 020 020 320 111

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa: F R S A L V R S S A S A K Q S L L R R S F

### Case 21—(3,1,0,2)=(A,C,G,T)—3 Errors/1 aa

u={223 113 222 033 001 132 120 103 003 130 212 313 002 312 201 302 013 301 131}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 221 303 211 010 122 021 103 211 211 011 210 010 330 130 210 122 121 101 101 301 222

### Glb: 221 303 211 010 122 021 103 211 211 011 210 010 330 130 210 122 121 101 101 131 221

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 22—(3,1,2,0)=(A,C,G,T)—3 Errors/1 aa

u={001 133 200 211 223 312 120 321 201 112 032 331 220 110 221 322 033 321 111}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 001 323 011 212 100 201 123 011 011 211 012 212 332 132 012 100 101 121 121 321 000

### Glb: 001 323 011 212 100 201 123 011 011 211 012 212 332 132 012 100 101 121 121 131 001

(SEQ ID NO: 12)
Gnt: TTC AGA TCC GCG CTT GTC CGA TCC TCC GCC TCG
GCG AAG CAG TCG CTT CTC CGC CGC CAC TTC
(SEQ ID NO: 13)
Gaa: F R S A L V R S S A S A K Q S L L R R H F

### Case 23—(3,2,0,1)=(A,C,G,T)—0 Errors/0 aa

u={133 100 222 301 120 200 100 221 233 111 203 030 231 203 212 133 323 110 321}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 112 303 122 020 211 012 203 122 122 022 120 020 330 230 120 211 212 202 202 302 111

### Glb: 112 303 122 020 211 012 203 122 122 022 120 020 330 230 120 211 212 202 202 302 111

(SEQ ID NO: 10)
Gnt: TTC AGA TCC GCG CTT GTC CGA TCC TCC GCC TCG
GCG AAG CAG TCG CTT CTC CGC CGC AGC TTT
(SEQ ID NO: 11)
Gaa:F R S A L V R S S A S A K Q S L L R R S F

### Case 24—(3,2,1,0)=(A,C,G,T)—4 Errors/2 aa

u={002 310 031 210 211 332 320 312 332 100 333 001 100 120 002 323 333 122 331}

(SEQ ID NO: 7)
Oaa: F R S A L V R S S A S A K Q S L L R R S F
(SEQ ID NO: 6)
Ont: TTC AGA TCC GCG CTT GTC CGA TCC TCC GCC TCG
GCG AAG CAG TCG CTT CTC CGC CGC AGC TTT

### Olb: 002 313 022 121 200 102 213 022 022 122 021 121 331 231 021 200 202 212 212 312 000

### Glb: 002 313 022 121 200 102 213 022 022 122 021 121 331 231 021 200 202 212 212 300 021

(SEQ ID NO: 8)
Gnt: TTC AGA TCC GCG CTT GTC CGA TCC TCC GCC TCG
GCG AAG CAG TCG CTT CTC CGC CGC ATT TCG
(SEQ ID NO: 9)
Gaa: F R S A L V R S S A S A K Q S L L R R I S

errors=
4 59 60 62 63/2 aa/S F→I S
0 0 0 0 0/0aa/S F→S F
3 58 59 63 0/1 aa/S F→H F
3 58 59 63 0/1 aa/S F→H F
4 59 60 62 63/2 aa/S F→I S
0 0 0 0 0/0aa/S F→S F
0 0 0 0 0/0aa/S F→S F
4 59 60 62 63/2 aa/S F→I S
0 0 0 0 0/0aa/S F→S F
4 59 60 62 63/2 aa/S F→I S
3 58 59 63 0/1 aa/S F→H F
3 58 59 63 0/1 aa/S F→H F
3 58 59 63 0/1 aa/S F→H F
3 58 59 63 0/1 aa/S F→H F
4 59 60 62 63/2 aa/S F→I S
0 0 0 0 0/0aa/S F→S F
4 59 60 62 63/2 aa/S F→I S
0 0 0 0 0/0aa/S F→S F
4 59 60 62 63/2 aa/S F→I S
0 0 0 0 0/0aa/S F→S F
3 58 59 63 0/1 aa/S F→H F
3 58 59 63 0/1 aa/S F→H F
0 0 0 0 0/0aa/S F→S F
4 59 60 62 63/2 aa/S F→I S

Programs for the Generation of DNA Sequences by Use of BCH Codes Over Field

1. Program minimalc.m
gx=minimalc(m, d, p, r, pr, step)
Function: Compute the generator polynomial g(x) of matrix G(x).

Input Parameters:
n=code word length;
d=code distance (d=2t+1, where t is the error correction capability of the code)
p=prime number;
r=Galois extension;
pr=primitive polynomial of degree r, irreducible over GF(p). Remark: The representation of the coefficients of the polynomial pr is from the greatest to the least exponent;
step=integer number greater than or equal to 1 which divides the field order, generating a cyclic subgroup Gₙ of order n.

Output Parameters:
gx=generator polynomial of the G(x) matrix.

Program Description:

The program minimalc computes the generator polynomial of the matrix G(x). The first step is to determine the βⁱ's roots of the minimal polynomial Mᵢ(x) over the group of units of the field, where β is a primitive element in Gₙ and the roots are in the sequence; the powers of β are reduced modulo p^{r}−1. For this step it is used the routine rootc.m with input parameters (m, d, p and r). The next step is to compute the cyclic subgroup G. with order equal to n. This step makes use of the routine tabc.m with input parameters r, pr and step. Finally, the generator polynomial g(x) is obtained through the lcm (least common multiple) of the minimal polynomials, that is, g(x)=lcm (M₁(x), M₂(x), . . . , M₂ₜ(x)), where t is the error correction capability of the code. For this it is used the routine gxc.m with the minimal polynomials as input parameters.
Ex: gx=[poc,gx,equal]=minimalc(63,3,4,3,3,b*x̂2+x+a,1)

2. Program matrixgc.m
mat=matrixgc(m, gx)
Function: Determine the generator matrix G(x).

Input Parameters:
n=code word length;
gx=generator polynomial computed by the program minimalc.m.

Output Parameters:
mat=generator matrix G(x).

Program Description:

The generator matrix G(x) is obtained by shifting the coefficients of the generator polynomial g(x) from the left to the right, one column in each row. Matrix G(x) has k rows and n columns, where k=n−g, where g is the degree of the polynomial g(x).
Ex: [mat]=matrizgc(63,gx)

3. Program divipoli.m
[hx, r]=divipoli(pl, gx)
Function: Determine the generator polynomial of the parity-check matrix H(x).

Input Parameters:
pl=polynomial xⁿ−1, where n=code word length;
gx=generator polynomial computed by the program minimalc.m.

Output Parameters:
hx=generator polynomial of the parity-check matrix H(x);
r=remainder of the division.

Program Description:

The generator polynomial h(x) of the parity-check matrix H(x) is determined by the division of the polynomial pl=xⁿ−1 by the generator polynomial g(x).
Ex: [q,r]=divipoli(x̂63-1,gx)

4. Program matrixhc.m
math=matrixhc(hx, n)
Function: Determine the parity-check matrix H(x).

Input Parameters:
hx=generator polynomial of matrix H(x) determined by the program divipoli.m;
n=code word length.

Output Parameters:
math=parity-check matrix H(x).

Program Description:

The parity-check matrix H(x) is obtained by shifting the coefficients of the polynomial h(x) from the right to the left, one column in each row.
Ex: [math]=matrizhxc(q,63)

5. Program gxhxc.m—for 1 Nucleotide Difference
[vetg, veth]=gxhxc(prot, n, gx, hx)
Function: Determine if the desired information sequence is a code word and if there is a code word which differs in only one position from the desired information sequence.

Input Parameters:
prot=desired information sequence;
n=code word length;
gx=generator polynomial calculated by the program minimalc.m;
hx=generator polynomial of matrix H(x) calculated by the program divipoli.m.

Output Parameters:
vetg=code words of the matrix H(x) without errors or that differ in only one position from the desired information sequence;
veth=code words of the matrix G(x) without errors or that differ in only one position from the desired information sequence.

Program Description:

The program gxhxc.m uses the routine labelc.m to generate the 24 possible permutations between the genetic alphabet (A, C, G, T) and the code alphabet (0, 1, a=α, b=α²). Thus, the 24 possible cases of the labelling are generated for the information sequence (prot) without nucleotide errors. The next step is to generate all the possible code words differing in one position for the 24 cases. These code words differ in only one position from the information sequence. Finally, all these possible code words without errors or with 1 nucleotide error are multiplied by G(x) and H(x) matrices. If the multiplication of the possible code word by the matrix H(x) is 0 (zero), then this is a code word (without error or differing in one nucleotide) of the generator matrix G(x). In the same way, if the multiplication of the possible code word by the matrix G(x) is 0 (zero), then this possible code word is a code word (without error or differing in one nucleotide) of matrix H(x).
Ex: [vetg,veth]=

(SEQ ID NO: 14)
gxhxc(′ATGGCCGCACGCCTCGCGCTGGTGGCGGCGCTCCTGTGCGCC
GGTGCCACGGCCGCCGCGGCG′,
63, gx, q)

vetg=[0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0, 0]
veth=[0, b, a, 0, 0, 0, 0, b, b, 0, a, 0, b, a, 1, b, b, 0, 0, b, b, b, a, b, 1, b, 1, a, b, 0, 0, b, b, a, b, b, 1, b, b, 0, 1, a, 0, b, 1, 0, b, b, 1, 0, b, b, b, a, b, 1, b, 1, 0, 0, a, 1, b]
[0, a, b, 0, 0, 0, 0, a, a, 0, b, 0, a, b, 1, a, a, 0, 0, a, a, a, b, a, 1, a, 1, b, a, 0, 0, a, a, b, a, a, 1, a, a, 0, 1, b, 0, a, 1, 0, a, a, 1, 0, a, a, a, b, a, 1, a, 1, 0, 0, b, 1, a]
[0, b, 1, 0, 0, 0, 0, b, b, 0, 1, 0, b, 1, a, b, b, 0, 0, b, b, b, 1, b, a, b, a, 1, b, 0, 0, b, b, 1, b, b, a, b, b, 0, a, 1, 0, b, a, 0, b, b, a, 0, b, b, b, 1, b, a, b, a, 0, 0, 1, a, b]
[0, 1, b, 0, 0, 0, 0, 1, 1, 0, b, 0, 1, b, a, 1, 1, 0, 0, 1, 1, 1, b, 1, a, 1, a, b, 1, 0, 0, 1, 1, b, 1, 1, a, 1, 1, 0, a, b, 0, 1, a, 0, 1, 1, a, 0, 1, 1, 1, b, 1, a, 1, a, 0, 0, b, a, 1]
[0, 1, a, 0, 0, 0, 0, 1, 1, 0, a, 0, 1, a, b, 1, 1, 0, 0, 1, 1, 1, a, 1, b, 1, b, a, 1, 0, 0, 1, 1, a, 1, 1, b, 1, 1, 0, b, a, 0, 1, b, 0, 1, 1, b, 0, 1, 1, 1, a, 1, b, 1, b, 0, 0, a, b, 1]
[0, a, 1, 0, 0, 0, 0, a, a, 0, 1, 0, a, 1, b, a, a, 0, 0, a, a, a, 1, a, b, a, b, 1, a, 0, 0, a, a, 1, a, a, b, a, a, 0, b, 1, 0, a, b, 0, a, a, b, 0, a, a, a, 1, a, b, a, b, 0, 0, 1, b, a]
[1, b, a, 1, 1, 1, 1, b, b, 1, a, 1, b, a, 0, b, b, 1, 1, b, b, b, a, b, 0, b, 0, a, b, 1, 1, b, b, a, b, b, 0, b, b, 1, 0, a, 1, b, 0, 1, b, b, 0, 1, b, b, b, a, b, 0, b, 0, 1, 1, a, 0, b]
[1, a, b, 1, 1, 1, 1, a, a, 1, b, 1, a, b, 0, a, a, 1, 1, a, a, a, b, a, 0, a, 0, b, a, 1, 1, a, a, b, a, a, 0, a, a, 1, 0, b, 1, a, 0, 1, a, a, 0, 1, a, a, a, b, a, 0, a, 0, 1, 1, b, 0, a]
[1, b, 0, 1, 1, 1, 1, b, b, 1, 0, 1, b, 0, a, b, b, 1, 1, b, b, b, 0, b, a, b, a, 0, b, 1, 1, b, b, 0, b, b, a, b, b, 1, a, 0, 1, b, a, 1, b, b, a, 1, b, b, b, 0, b, a, b, a, 1, 1, 0, a, b]
[1, 0, b, 1, 1, 1, 1, 0, 0, 1, b, 1, 0, b, a, 0, 0, 1, 1, 0, 0, 0, b, 0, a, 0, a, b, 0, 1, 1, 0, 0, b, 0, 0, a, 0, 0, 1, a, b, 1, 0, a, 1, 0, 0, a, 1, 0, 0, 0, b, 0, a, 0, a, 1, 1, b, a, 0]
[1, a, 0, 1, 1, 1, 1, a, a, 1, 0, 1, a, 0, b, a, a, 1, 1, a, a, a, 0, a, b, a, b, 0, a, 1, 1, a, a, 0, a, a, b, a, a, 1, b, 0, 1, a, b, 1, a, a, b, 1, a, a, a, 0, a, b, a, b, 1, 1, 0, b, a]
[1, 0, a, 1, 1, 1, 1, 0, 0, 1, a, 1, 0, a, b, 0, 0, 1, 1, 0, 0, 0, a, 0, b, 0, b, a, 0, 1, 1, 0, 0, a, 0, 0, b, 0, 0, 1, b, a, 1, 0, b, 1, 0, 0, b, 1, 0, 0, 0, a, 0, b, 0, b, 1, 1, a, b, 0]
[a, b, 1, a, a, a, a, b, b, a, 1, a, b, 1, 0, b, b, a, a, b, b, b, 1, b, 0, b, 0, 1, b, a, a, b, b, 1, b, b, 0, b, b, a, 0, 1, a, b, 0, a, b, b, 0, a, b, b, b, 1, b, 0, b, 0, a, a, 1, 0, b]
[a, 1, b, a, a, a, a, 1, 1, a, b, a, 1, b, 0, 1, 1, a, a, 1, 1, 1, b, 1, 0, 1, 0, b, 1, a, a, 1, 1, b, 1, 1, 0, 1, 1, a, 0, b, a, 1, 0, a, 1, 1, 0, a, 1, 1, 1, b, 1, 0, 1, 0, a, a, b, 0, 1]
[a, b, 0, a, a, a, a, b, b, a, 0, a, b, 0, 1, b, b, a, a, b, b, b, 0, b, 1, b, 1, 0, b, a, a, b, b, 0, b, b, 1, b, b, a, 1, 0, a, b, 1, a, b, b, 1, a, b, b, b, 0, b, 1, b, 1, a, a, 0, 1, b]
[a, 0, b, a, a, a, a, 0, 0, a, b, a, 0, b, 1, 0, 0, a, a, 0, 0, 0, b, 0, 1, 0, 1, b, 0, a, a, 0, 0, b, 0, 0, 1, 0, 0, a, 1, b, a, 0, 1, a, 0, 0, 1, a, 0, 0, 0, b, 0, 1, 0, 1, a, a, b, 1, 0]
[a, 1, 0, a, a, a, a, 1, 1, a, 0, a, 1, 0, b, 1, 1, a, a, 1, 1, 1, 0, 1, b, 1, b, 0, 1, a, a, 1, 1, 0, 1, 1, b, 1, 1, a, b, 0, a, 1, b, a, 1, 1, b, a, 1, 1, 1, 0, 1, b, 1, b, a, a, 0, b, 1]
[a, 0, 1, a, a, a, a, 0, 0, a, 1, a, 0, 1, b, 0, 0, a, a, 0, 0, 0, 1, 0, b, 0, b, 1, 0, a, a, 0, 0, 1, 0, 0, b, 0, 0, a, b, 1, a, 0, b, a, 0, 0, b, a, 0, 0, 0, 1, 0, b, 0, b, a, a, 1, b, 0]
[b, a, 1, b, b, b, b, a, a, b, 1, b, a, 1, 0, a, a, b, b, a, a, a, 1, a, 0, a, 0, 1, a, b, b, a, a, 1, a, a, 0, a, a, b, 0, 1, b, a, 0, b, a, a, 0, b, a, a, a, 1, a, 0, a, 0, b, b, 1, 0, a]
[b, 1, a, b, b, b, b, 1, 1, b, a, b, 1, a, 0, 1, 1, b, b, 1, 1, 1, a, 1, 0, 1, 0, a, 1, b, b, 1, 1, a, 1, 1, 0, 1, 1, b, 0, a, b, 1, 0, b, 1, 1, 0, b, 1, 1, 1, a, 1, 0, 1, 0, b, b, a, 0, 1]
[b, a, 0, b, b, b, b, a, a, b, 0, b, a, 0, 1, a, a, b, b, a, a, a, 0, a, 1, a, 1, 0, a, b, b, a, a, 0, a, a, 1, a, a, b, 1, 0, b, a, 1, b, a, a, 1, b, a, a, a, 0, a, 1, a, 1, b, b, 0, 1, a]
[b, 0, a, b, b, b, b, 0, 0, b, a, b, 0, a, 1, 0, 0, b, b, 0, 0, 0, a, 0, 1, 0, 1, a, 0, b, b, 0, 0, a, 0, 0, 1, 0, 0, b, 1, a, b, 0, 1, b, 0, 0, 1, b, 0, 0, 0, a, 0, 1, 0, 1, b, b, a, 1, 0]
[b, 1, 0, b, b, b, b, 1, 1, b, 0, b, 1, 0, a, 1, 1, b, b, 1, 1, 1, 0, 1, a, 1, a, 0, 1, b, b, 1, 1, 0, 1, 1, a, 1, 1, b, a, 0, b, 1, a, b, 1, 1, a, b, 1, 1, 1, 0, 1, a, 1, a, b, b, 0, a, 1]
[b, 0, 1, b, b, b, b, 0, 0, b, 1, b, 0, 1, a, 0, 0, b, b, 0, 0, 0, 1, 0, a, 0, a, 1, 0, b, b, 0, 0, 1, 0, 0, a, 0, 0, b, a, 1, b, 0, a, b, 0, 0, a, b, 0, 0, 0, 1, 0, a, 0, a, b, b, 1, a, 0]

5.1 Program gxhx2c.m—for 2 Nucleotide Errors
[vetg, veth]=gxhx2c(prot, n, gx, hx, case)
Function: Determine if exists a code word that differs in two positions from the desired information sequence.

Input Parameters:
prot=desired information sequence;
n=code word length;
gx=generator polynomial calculated by the program minimalc.m;
hx=generator polynomial of matrix H(x) calculated by the program divipoli.m
case=labelling case

Output Parameters:
vetg=code words of matrix H(x) that differ in two positions from the desired information sequence;
veth=code words of matrix G(x) that differ in two positions from the desired information sequence.

Program Description:

The program gxhx2c.m uses the routine labelc.m for the labelling between the genetic alphabet (A, C, G, T) and the code alphabet (0, 1, a, b) to the specified case. Thus, it is generated one possible case of labelling for the information sequence (prot) without nucleotide errors. The next step is to generate all the possible code words differing in 2 positions. These code words differ in two positions from the information sequence. Finally, all these possible code words differing in 2 nucleotides are multiplied by G(x) and H(x) matrices. If the multiplication of the possible code word by the matrix H(x) is 0 (zero), then this is a code word (differing in 2 nucleotides) of the generator matrix G(x). In the same way, if the multiplication of the possible code word by the matrix G(x) is 0 (zero), then this possible code word is a code word (differing in 2 nucleotides) of matrix H(x).

6. Program label_invc.m—for 1 and 2 Nucleotide Differences
result=label_invc (codeword, n, prot)
Function: Determine in which permutations the code words were found and show if there are nucleotide differences. In the case of differing either in 1 or 2 positions, the program shows in which positions the PSN (nucleotides of the desired sequence) and PSA sequences (nucleotides of the sequence generated by the code) differ from each other. Consequently, the AA1 and AA2 sequences present the differences in amino acids.

Input Parameters:
codeword=code word differing in one position, obtained in the program gxhxc.m, or differing in 2 positions obtained in the program gxhx2c;
n=code word length;
prot=desired information sequence.

Output Parameters:
result=amino acids, nucleotides and labelling of the desired sequence and the generated sequence

Program Description:

The first step is to label the code word in the genetic alphabet (A, T, C, G) for the 24 labelling cases. These nucleotide sequences are converted to the corresponding sequence in amino acids using the routine pro2ami.m. The desired information sequence is also converted to its corresponding amino acids sequence by the routine pro2ami.m. All this information is stored in result.

Ex 1:1 Nucleotide Difference: see FIG. 147

7. Program convert.m
coef=convert(poli)
Function: simplify the coefficients of the field equations using the addition and multiplication operations modulo GF(4), as shown in Tables D and E.

TABLE D
Addition in GF(4)

| | | | |
|---|---|---|---|
| 0 + 0 = 0 | 1 + 0 = 1 | a + 0 = a | b + 0 = b |
| 0 + 1 = 1 | 1 + 1 = 2 | a + 1 = b | b + 1 = a |
| 0 + a = a | 1 + a = b | a + a = 0 | b + a = 1 |
| 0 + b = b | 1 + b = a | a + b = 1 | b + b = 0 |

TABLE E
Multiplication in GF(4)

| | | | |
|---|---|---|---|
| 0 × 0 = 0 | 1 × 0 = 0 | a × 0 = 0 | b × 0 = 0 |
| 0 × 1 = 0 | 1 × 1 = 1 | a × 1 = a | b × 1 = b |
| 0 × a = 0 | 1 × a = a | a × a = b | b × a = 1 |
| 0 × b = 0 | 1 × b = b | a × b = 1 | b × b = a |

Input Parameters:
poli=equation as a function of the field elements (0, 1, a, b)

Output Parameters:
coef=simplified equation

Program Description:

The program convert.m is used by the following programs: minimalc.m, tabc.m, gxhxc.m, divipoli.m e gxhx2c.m.

### EXAMPLES AND ANALYSIS

The invention is now described by reference to the following examples, which are illustrative only, and are not intended to limit the present invention.

### Example 1

### Generation and Reproduction of DNA Sequences Differing in One Nucleotide without Change of Amino Acid by a Primitive BCH Code Over Ring

In this non-limiting example, we show the generation and reproduction of DNA sequences available in the data bank (NCBI). The DNA sequences shown in Tables 1 and 2 were reproduced by the primitive BCH code using the labelling A. This is the Z₄-linear mapping classifying these sequences as nonlinear sequences. The DNA sequence shown in Table 3 was reproduced by use of the labelling C whose mapping is the Klein mapping, classifying it as a linear sequence. These labellings are related to geometric forms which may be able to provide some indication of the degree of nonlinearity associated with the reproduced sequences.

In Tables 1, 2 and 3 one can verify that the DNA sequences generated and reproduced by the primitive BCH codes are mathematically related with their corresponding complementary strands in the following manner: If a given primitive polynomial p(x) and a generator polynomial g(x) generates and reproduces a specific DNA sequence, then its complementary strand will be reproduced only by the reciprocal polynomial of the former primitive polynomial p(x)′ and by the reciprocal of the generator polynomial g(x)′, always using the same labelling.

### Example 2

### Generation and Reproduction of DNA Sequences Differing in One Nucleotide with Change of Amino Acid within the Same Class by Use of a Primitive BCH Code Over Ring

In this non-limiting example, we show the generation and reproduction of DNA sequences available in the data bank (NCBI). The DNA sequences shown in Tables 4, 5, and 6 were reproduced by the primitive BCH code using the labelling A. This is the Z₄-linear mapping classifying these sequences as nonlinear sequences. The DNA sequence shown in Table 7 was reproduced by the primitive BCH code using the labelling B, whose mapping is the Z₂×Z₂, classifying it as a linear sequence. The DNA sequence shown in Table 8 was reproduced by the primitive BCH code using the labelling C, whose mapping is the Klein mapping, classifying it as a linear sequence. These labellings are related to the geometric forms which provide some indication about the degree of nonlinearity of the reproduced sequences.

In Tables 4, 5, 6, 7, and 8 one can verify that the DNA sequences generated and reproduced by the primitive BCH codes are mathematically related with their corresponding complementary strands in the following manner: If a given primitive polynomial p(x) and a generator polynomial g(x) generates and reproduces a specific DNA sequence, then its complementary strand will be reproduced only by the reciprocal polynomial of the former primitive polynomial p(x)′ and by the reciprocal of the generator polynomial g(x)′, always using the same labelling.

### Example 3

### Generation and Reproduction of DNA Sequences Differing in One Nucleotide with Change of Amino Acid by Use of the Primitive BCH Code Over Ring

In this non-limiting example, we show the generation and reproduction of DNA sequences available in the data bank (NCBI). The DNA sequence shown in Table 9 was reproduced by the primitive BCH code by use of the labelling A. This is the Z₄-linear mapping classifying this sequence as a nonlinear sequence. The DNA sequences shown in Tables 10, 11 and 12 were reproduced by use of the primitive BCH code with the labelling B, whose mapping is the Z₂×Z₂, classifying them as linear sequences. The DNA sequence shown in Table 13 was reproduced by the primitive BCH code with labelling C, whose mapping is the Klein mapping, classifying it as a linear sequence. These labellings are related to geometric forms which provide an indication of the degree of nonlinearity associated with such reproduced sequences.

In Tables 9, 10, 11, 12, and 13 one can check that the DNA sequences generated and reproduced by the primitive BCH codes are mathematically related with their corresponding complementary strands in the following manner: If a given primitive polynomial p(x) and a generator polynomial g(x) generates and reproduces a specific DNA sequence, then its complementary strand will be reproduced only by the reciprocal polynomial of the former primitive polynomial p(x)′ and by the reciprocal of the generator polynomial g(x)′, always using the same labelling.

### Example 4

### Generation and Reproduction of DNA Sequences Differing in Two Nucleotides without Changing Amino Acids by Use of the Primitive BCH Code Over Ring

In this non-limiting example, Tables 14-119 show the generation and reproduction of DNA sequences differing in two nucleotides and without changing of amino acids. These DNA sequences are available in the data bank (NCBI).

### Example 5

### Generation and Reproduction of DNA Sequences Differing in Two Nucleotides with Change of Amino Acids within the Same Class by Use of the Primitive BCH Code Over Ring

In this example, we show in Tables 120-125 the generation and reproduction of DNA sequences differing in two nucleotides with change of amino acids within the same class. These DNA sequences are available in the data bank (NCBI).

### Example 6

### Generation and Reproduction of DNA Sequences Differing in Two Nucleotides without Changing Amino Acids by Use of the Nonprimitive BCH Code Over Ring

In this non-limiting example, Tables 126 and 127 show the generation and reproduction of DNA sequences differing in two nucleotides without changing amino acids. These DNA sequences are available in the data bank (NCBI).

### Example 7

### Generation and Reproduction of DNA Sequences Differing in Two Nucleotides with Change of Amino Acids within the Same Class by Use of the Nonprimitive BCH Code Over Ring

In this non-limiting example, Tables 128, 129 and 130 show the generation and reproduction of DNA sequences differing in two nucleotides with change of amino acids within the same class. These DNA sequences are available in the data bank (NCBI).

### Example 8

### Generation and Reproduction of DNA Sequences by Use Of the Primitive BCH Code Over Ring Differing in Two Nucleotides not Encoding Protein

In this non-limiting example, Tables 131 and 132 show the generation and reproduction of DNA sequences differing in two nucleotides. These DNA sequences are available in the data bank (NCBI).

### Example 9

### Generation and Reproduction of DNA Sequences Differing in One Nucleotide with Change of Amino Acids by Use of the Primitive BCH Code Over Field

In this non-limiting example, Tables 133, 134 and 135 show the generation and reproduction of DNA sequences differing in one nucleotide with change of amino acids. These DNA sequences are available in the data bank (NCBI).

### Example 10

### Generation and Reproduction of Encoded Sequences of The Malate Dehydrogenase of Arabidopsis Thaliana by Use of the Primitive BCH Code Over Ring

The generation of the whole coding sequence of the mitochondrial malate dehydrogenase Arabidopsis thaliana is shown in Table 136a. Note that only one nucleotide differs in the sequence containing 1023 nucleotides (CTT→TTT). This difference leads to a change of amino acid in that triplet (Leu→Phe), although occurring within the same class of amino acid. It is interesting to observe that the non coding sequence is also reproduced by the reciprocal of the generator polynomial (Table 136b).

### Example 11

### Experimental Results Based on Mutation Analysis of Processing of Synthetic Oligopeptides

As a non-limiting example of this method, we employ the following DNA sequence available in the data bank (NCBI): targeting sequence MDH1-21 (mitochondrial malate dehydrogenase), Rattus norvegicus, locus X04240. In [6] laboratorial tests were realized by substituting the arginine residues by alanine and lysine with the purpose of verifying the importance of these arginines for a specific recognition and the correct cleavage of the peptidase extension. To determine the role of arginine residues in the recognition by MPP, three arginine residues at positions 7, 14 and 15 in MDH1-21 were systematically replaced by alanine residues (MDH7A, MDH14A e MDH15A). To examine if arginine residues at positions distal or proximal to the cleavage site of the peptide were replaceable by lysine residues in MDH14A. First of all we reproduced the targeting sequence MDH1-21, by use of an error-correcting code, differing in one nucleotide without changing amino acid with the labelling C, which we define as the MDH1-21* sequence.

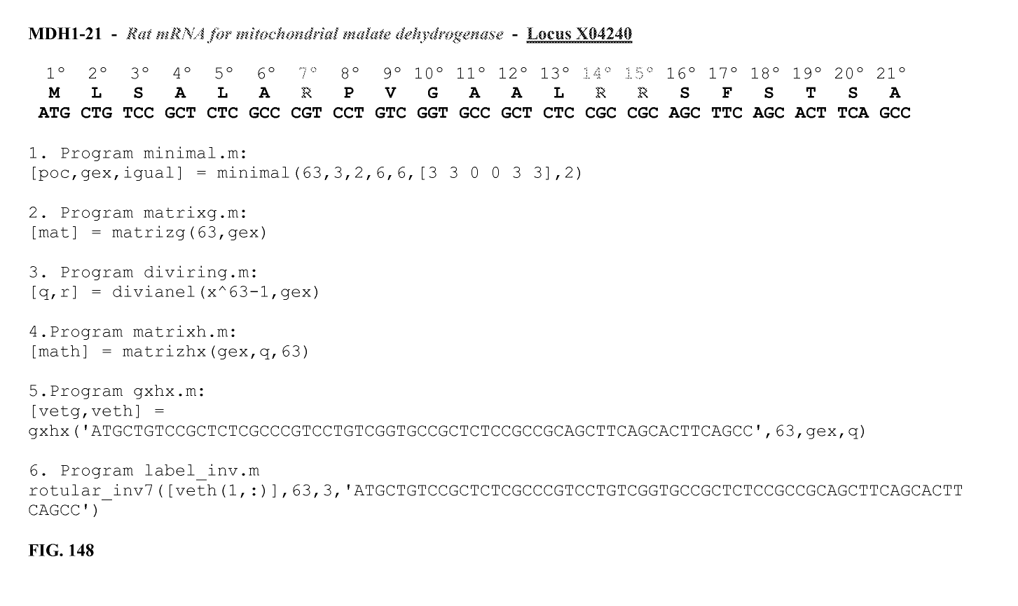

Simulations with Changes in the MDH1-21 Generated by the (63,57,3) BCH Code Over Z₄-Galois Ring GR(4,6) Based on the Paper [6]
Primitive polynomial: x⁶+x⁵+x⁴+x+1—Generator polynomial: x⁶+x⁵+x⁴+2x²+3x+1
MDH1-21—Rat mRNA for Mitochondrial Malate Dehydrogenase—Locus X04240 (see FIG. 148)

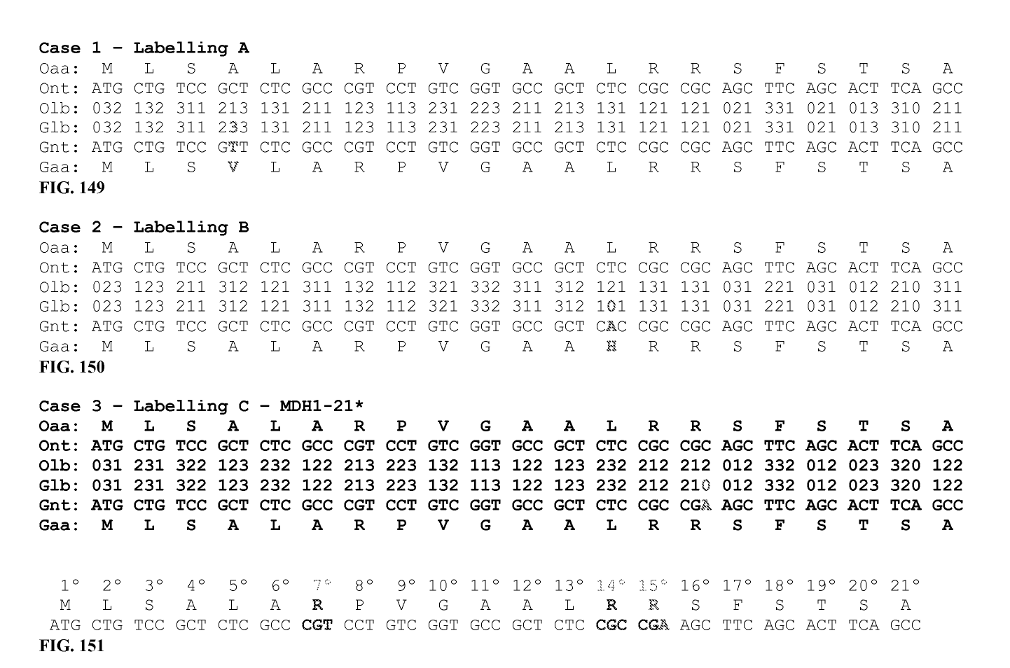

### Result: Case 1—Labelling A—see FIG. 149

### Result: Case 2—Labelling B—see FIG. 150

### Result: Case 3—Labelling C—MDH1-21*—see FIG. 151

### Cases Analyzed According to the Changes Realized in the Paper [6]:

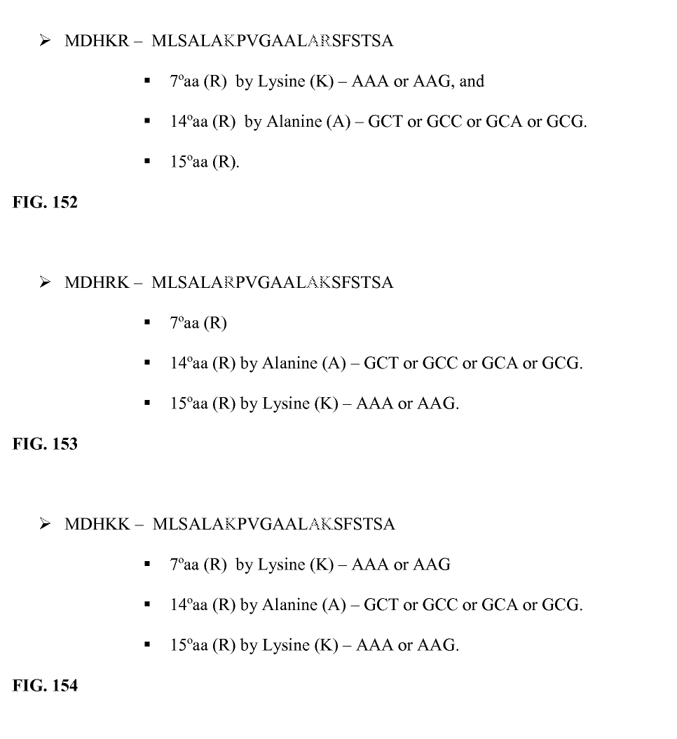

FIG. 152 shows the analysis of the eighth possible combinations between the nucleotides of: K, A and R.
FIG. 153 shows the analysis of the eighth possible combinations between the nucleotides of: R, A and K.
FIG. 154 shows the analysis of the sixteen possible combinations between the nucleotides of: K, A and K.

### Analysis of the Eighth Possible Combinations Between the Nucleotides of: K, A and R.

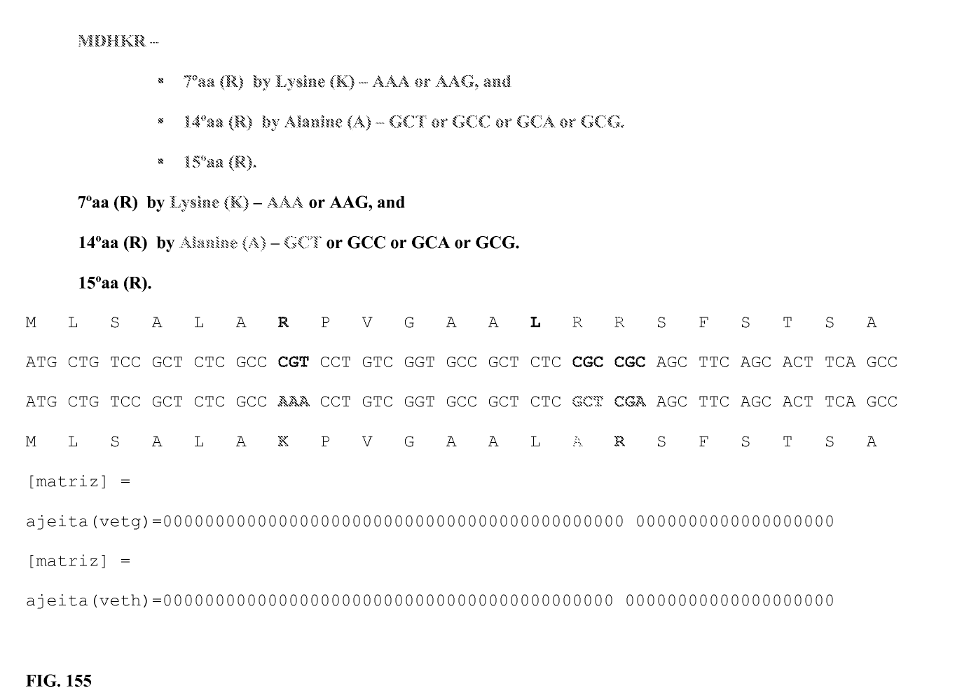

1) MDHKR—analysis of one of the eight possible combinations between the nucleotides of: K, A and R (see FIG. 155)

7° aa (R) by Lysine (K)—AAA or AAG, and

14° aa (R) by Alanine (A)—GCT or GCC or GCA or GCG.

15° aa (R).

Conclusion: The change was not accepted by the code.

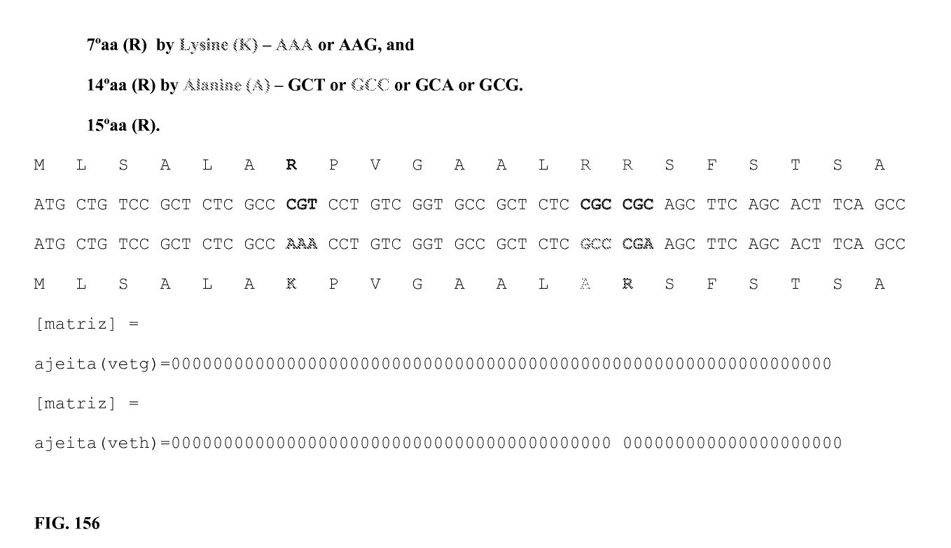

2) MDHKR—analysis of one of the eight possible combinations between the nucleotides of: K, A and R (see FIG. 156)

Conclusion: The change was not accepted by the code.

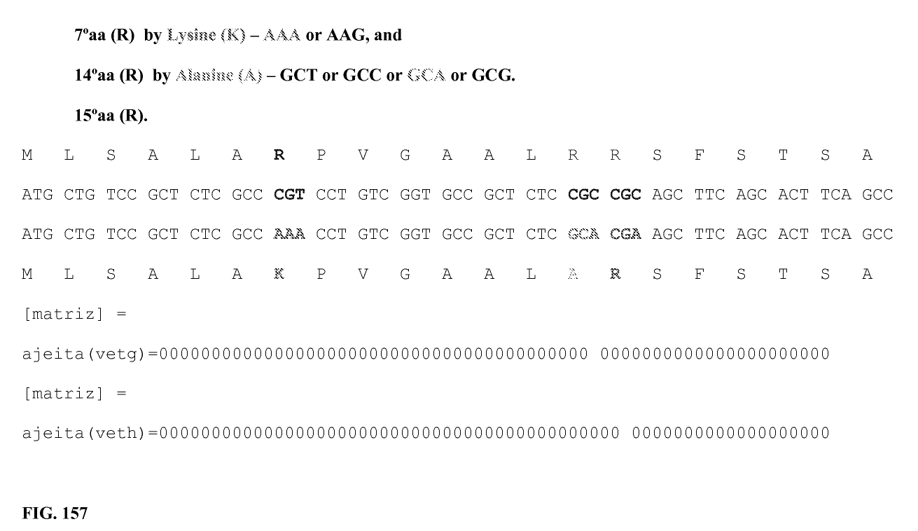

3) MDHKR—analysis of one of the eight possible combinations between the nucleotides of: K, A and R (see FIG. 157)

Conclusion: The change was not accepted by the code.

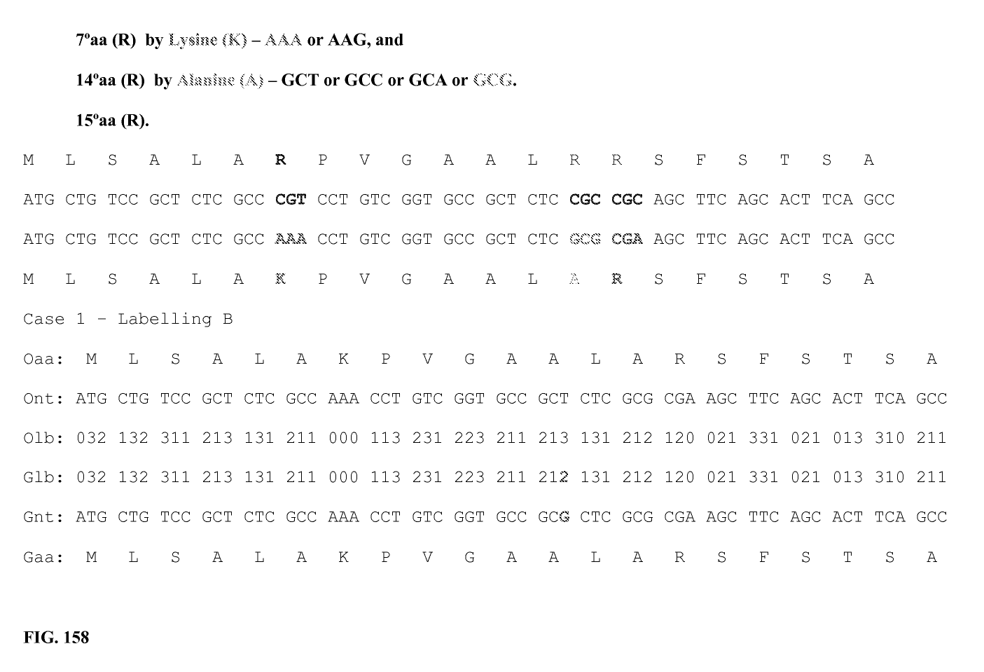

4) MDHKR—analysis of one of the eight possible combinations between the nucleotides of: K, A and R (see FIG. 158)

Conclusion: The code accepted the change of amino acid however by changing the labelling C to the labelling B. On the other hand, biologically we can not assert whether this change will be accepted or not. Hence, its confirmation depends on the realization of experimental tests.

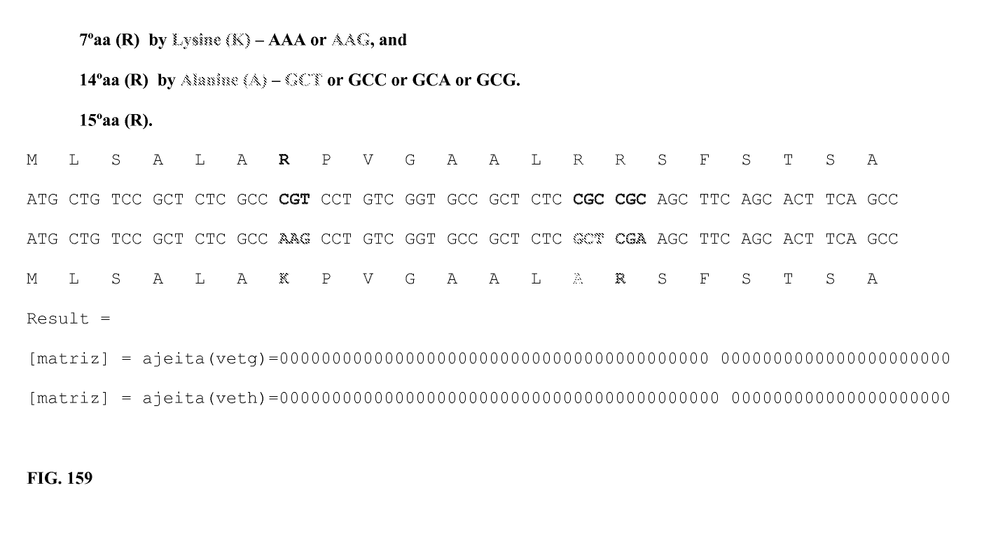

5) MDHKR—analysis of one of the eight possible combinations between the nucleotides of: K, A and R (see FIG. 159)

Conclusion: The change was not accepted by the code.

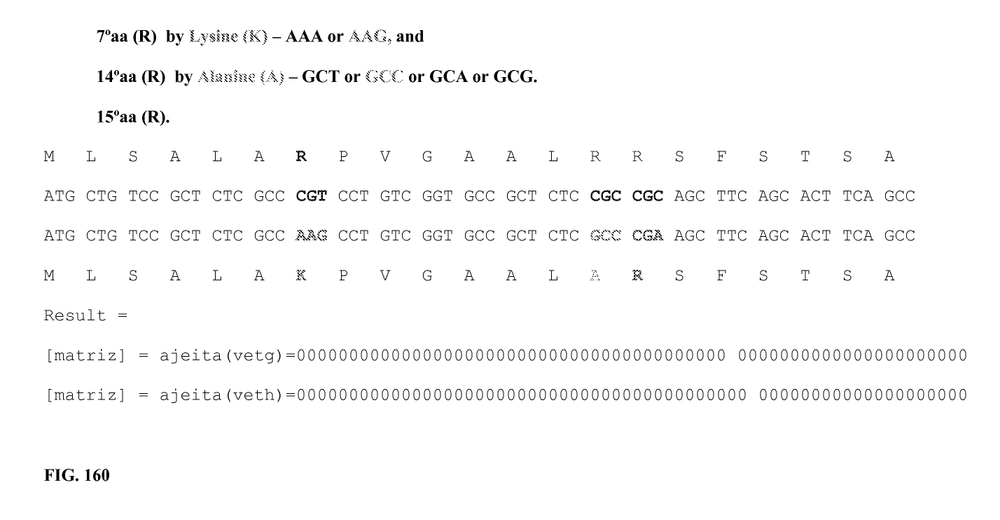

6) MDHKR—analysis of one of the eight possible combinations between the nucleotides of: K, A and R (see FIG. 160)

Conclusion: The change was not accepted by the code.

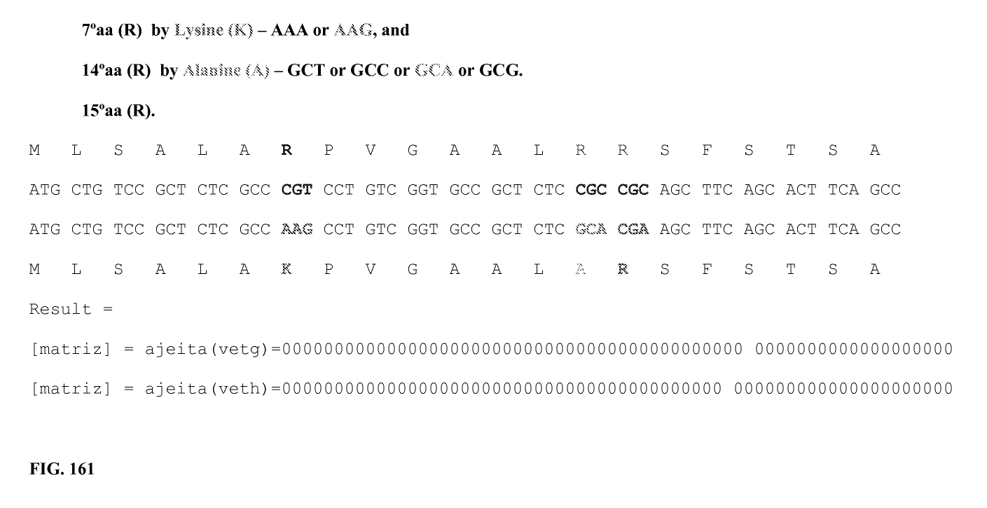

7) MDHKR—analysis of one of the eight possible combinations between the nucleotides of: K, A and R (see FIG. 161)

Conclusion: The change was not accepted by the code.

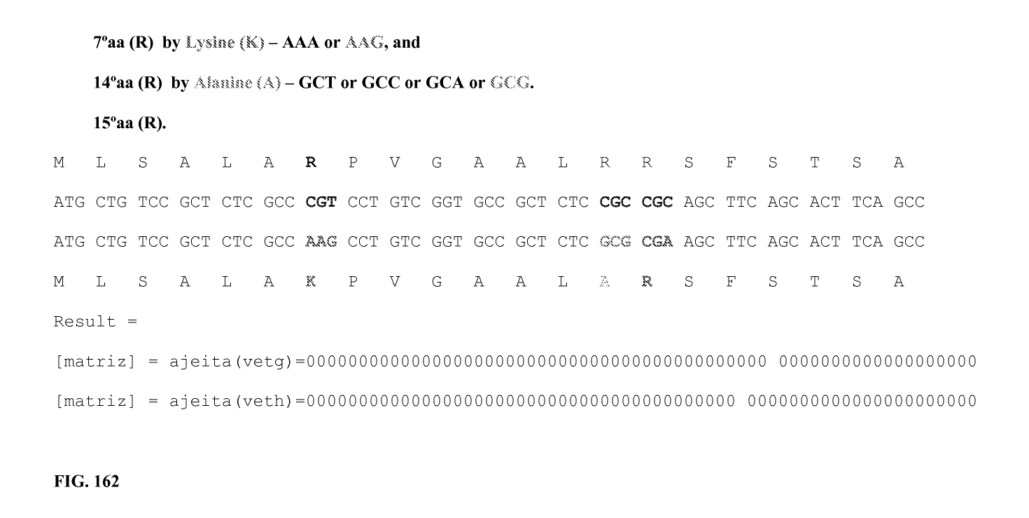

8) MDHKR—analysis of one of the eight possible combinations between the nucleotides of: K, A and R (see FIG. 162)

Conclusion: The change was not accepted by the code.

### Analysis of the Eighth Possible Combinations Between the Nucleotides of: R, A and K.

### MDHRK—

7° aa (R)

14° aa (R) by Alanine (A)—GCT or GCC or GCA or GCG.

15° aa (R) by Lysine (K)—AAA or AAG.

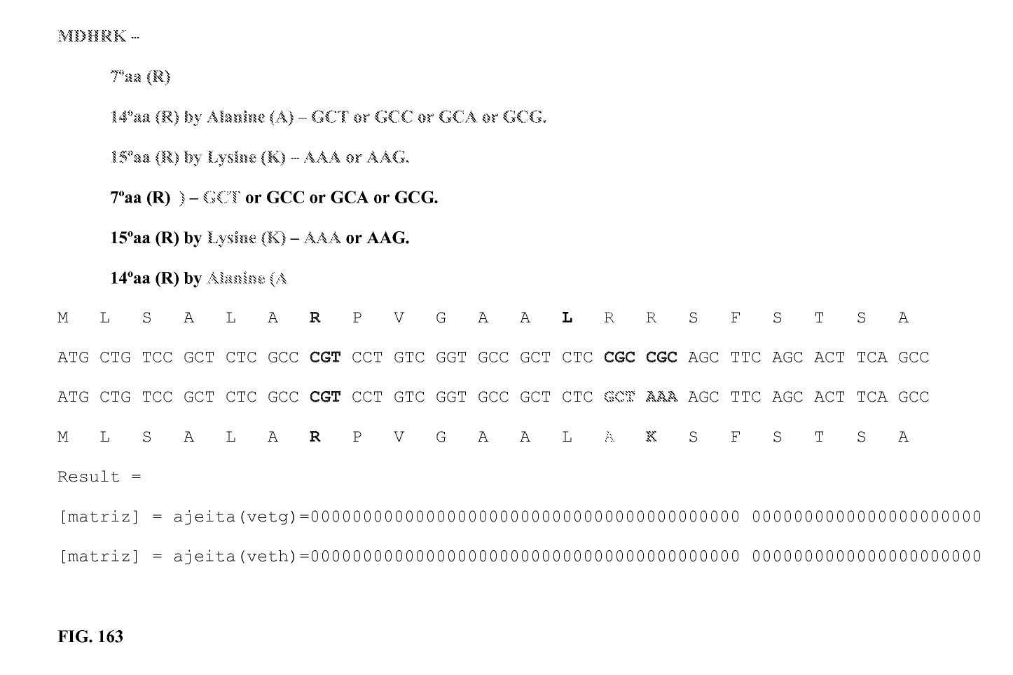

1) MDHRK—the analysis of one of the eight possible combinations between the nucleotides of: R, A and K (see FIG. 163).

Conclusion: The change was not accepted by the code.

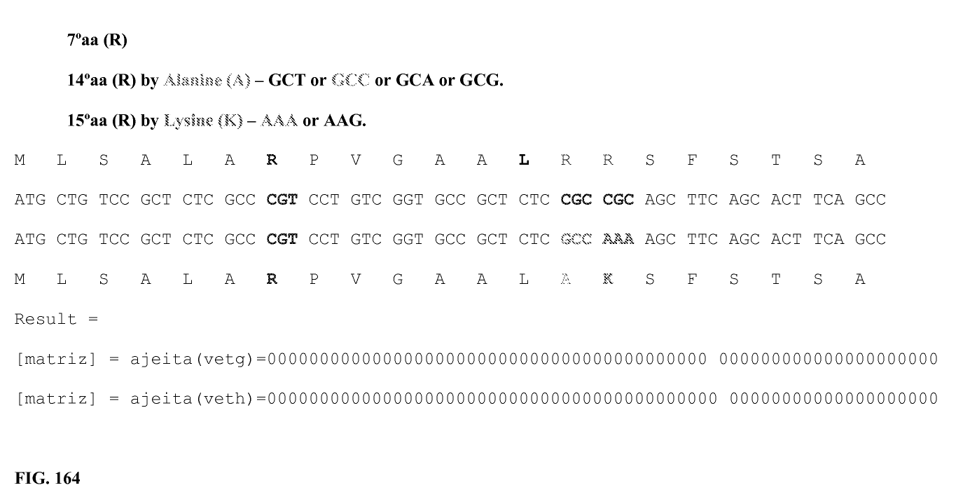

2) MDHRK—the analysis of one of the eight possible combinations between the nucleotides of: R, A and K (see FIG. 164).

Conclusion: The change was not accepted by the code.

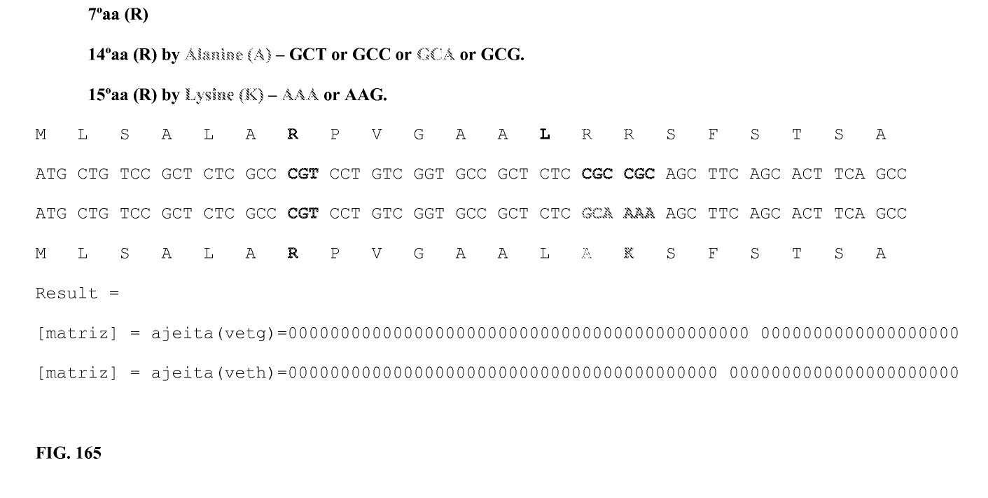

3) MDHRK—the analysis of one of the eight possible combinations between the nucleotides of: R, A and K (see FIG. 165).

Conclusion: The change was not accepted by the code.

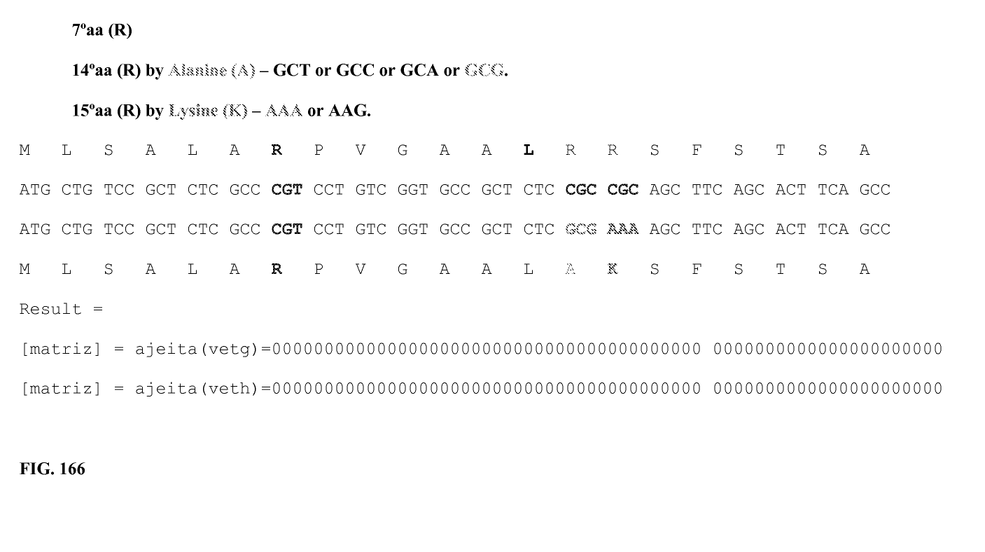

4) MDHRK—the analysis of one of the eight possible combinations between the nucleotides of: R, A and K (see FIG. 166).

Conclusion: The change was not accepted by the code.

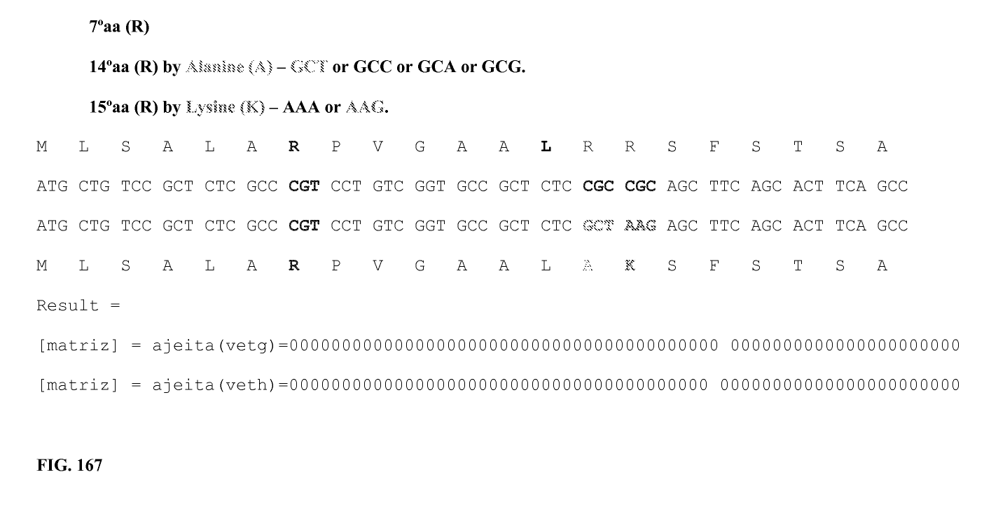

5) MDHRK—the analysis of one of the eight possible combinations between the nucleotides of: R, A and K (see FIG. 167).

Conclusion: The change was not accepted by the code.

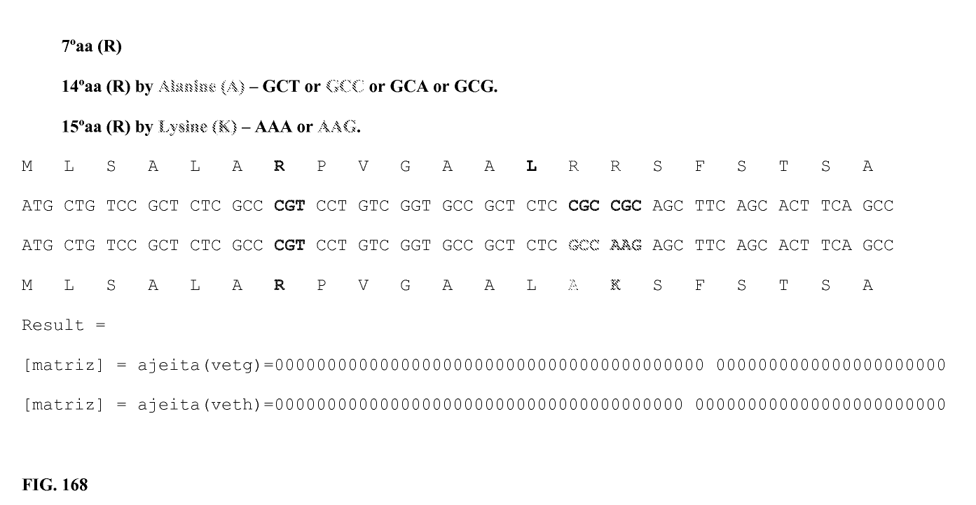

6) MDHRK—the analysis of one of the eight possible combinations between the nucleotides of: R, A and K (see FIG. 168).

Conclusion: The change was not accepted by the code.

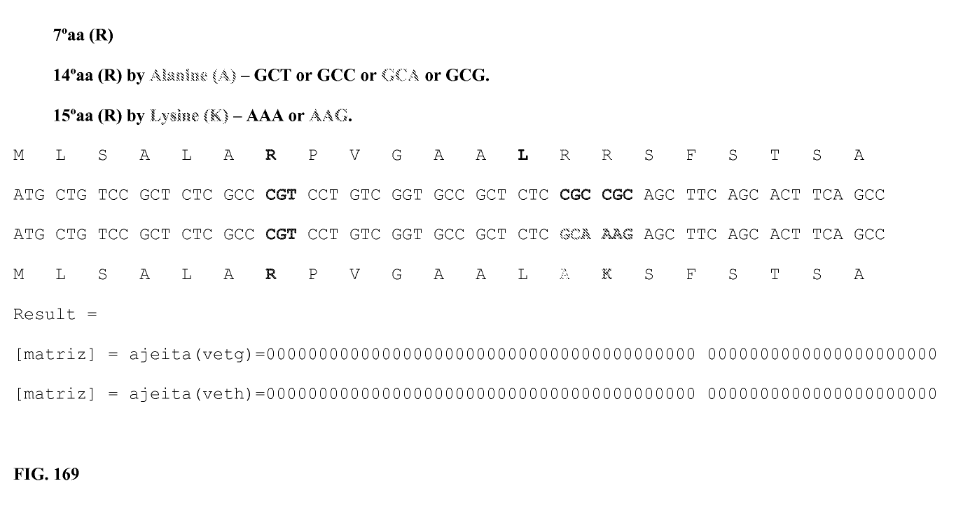

7) MDHRK—the analysis of one of the eight possible combinations between the nucleotides of: R, A and K (see FIG. 169).

Conclusion: The change was not accepted by the code.

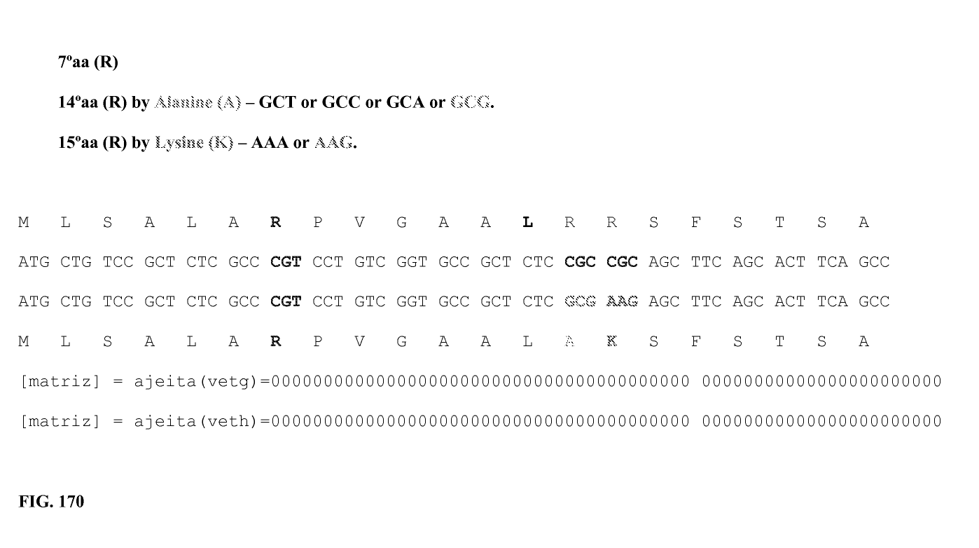

8) MDHRK—the analysis of one of the eight possible combinations between the nucleotides of: R, A and K (see FIG. 170).

Conclusion: The change was not accepted by the code.

### Analysis of the Sixteen Possible Combinations Between the Nucleotides of: K, a and K.

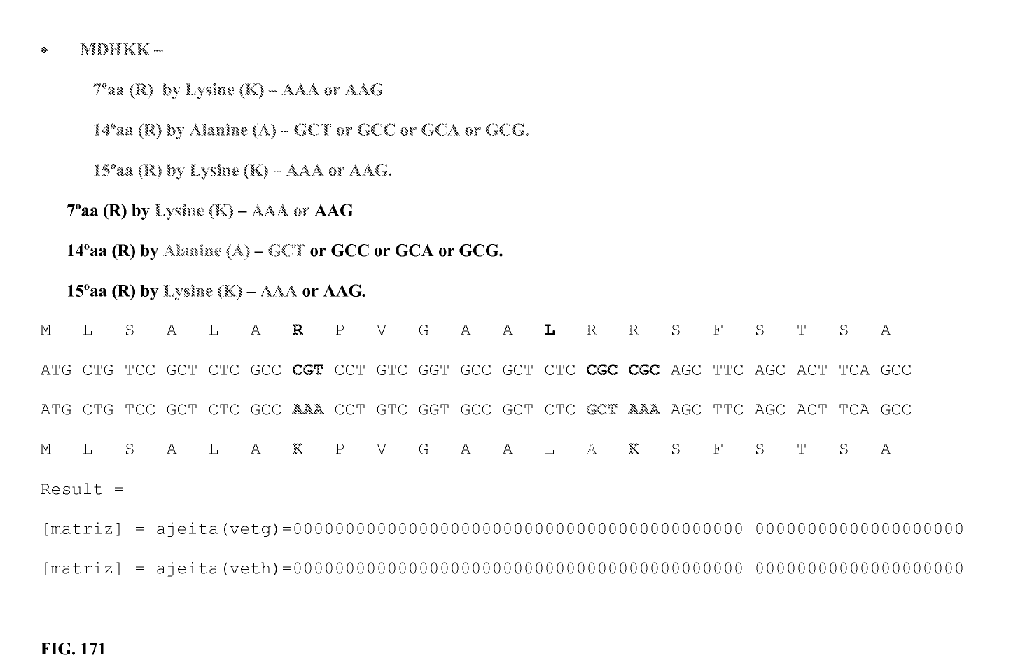

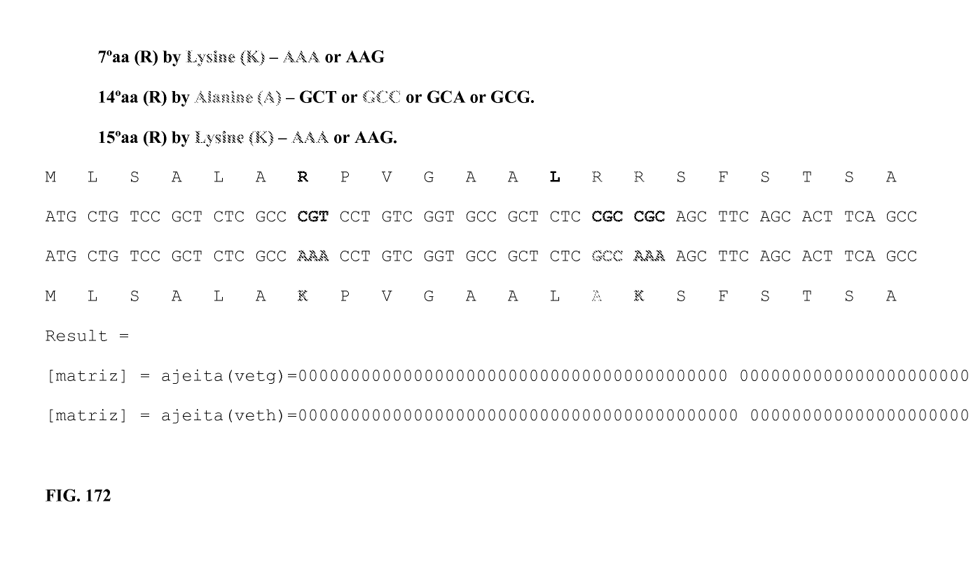

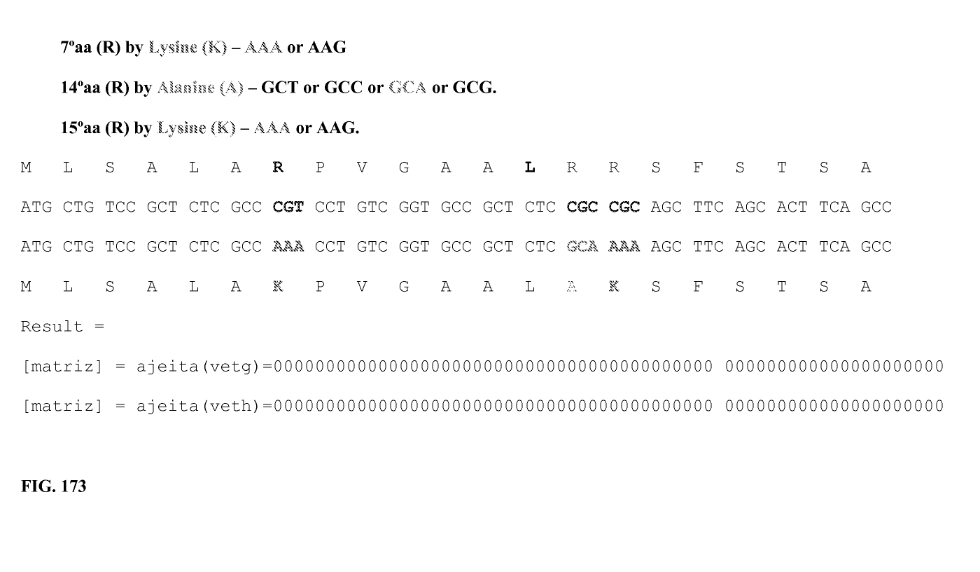

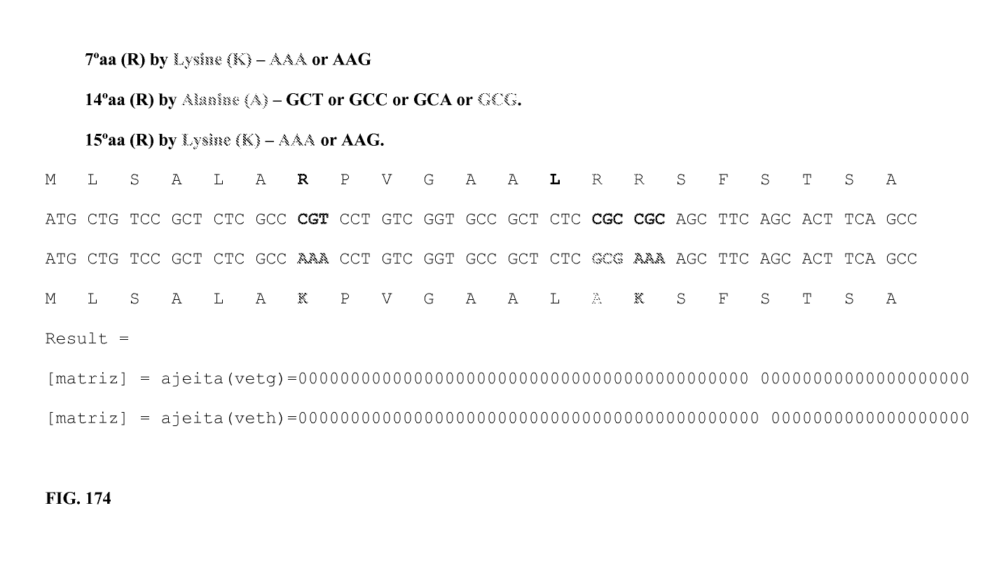

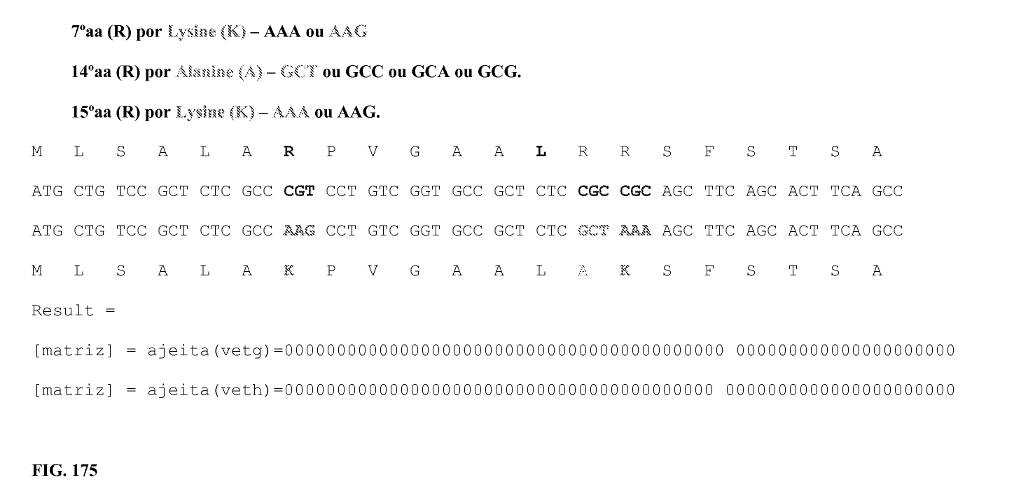

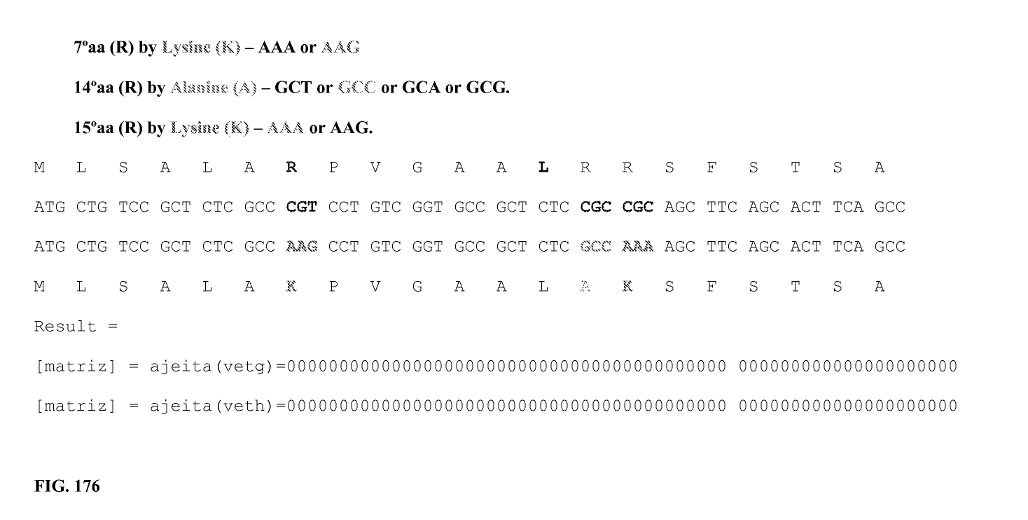

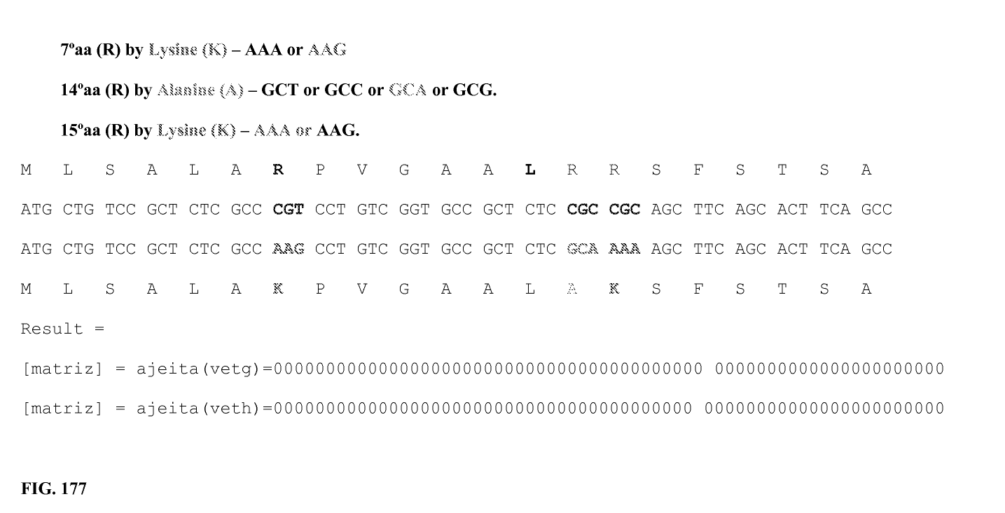

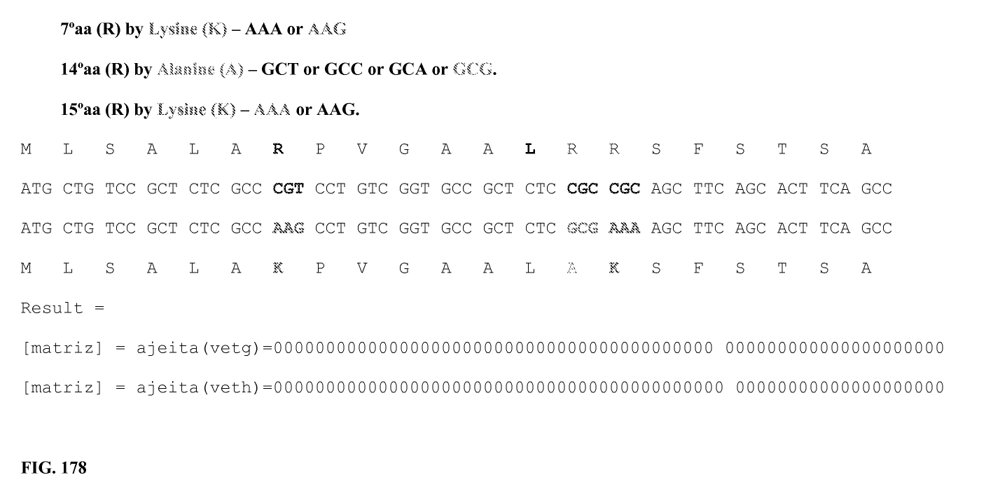

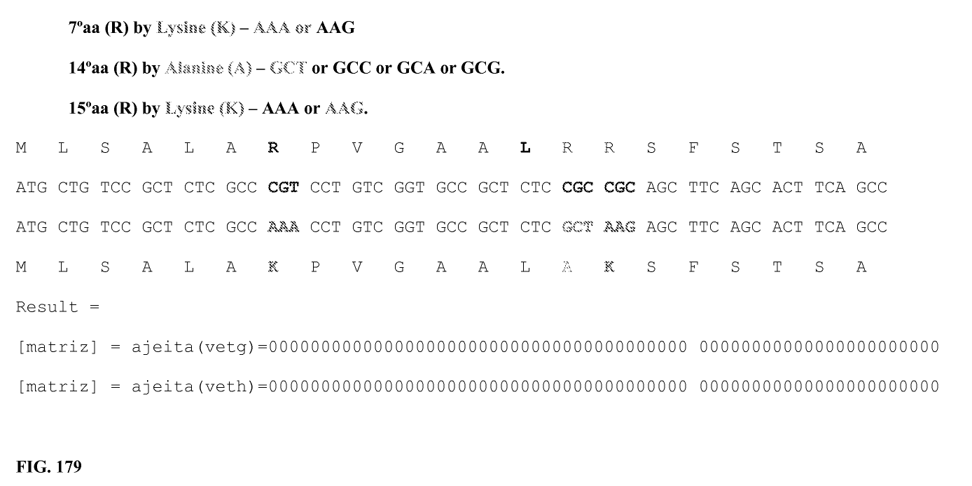

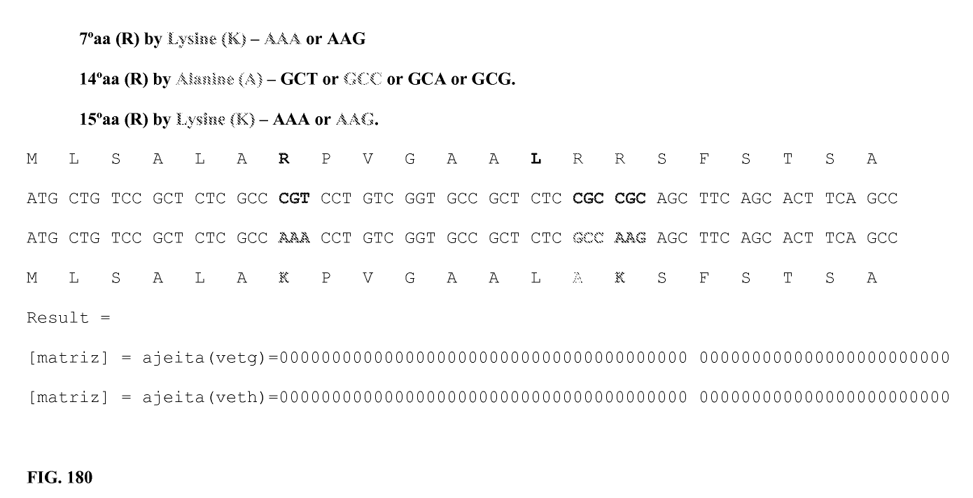

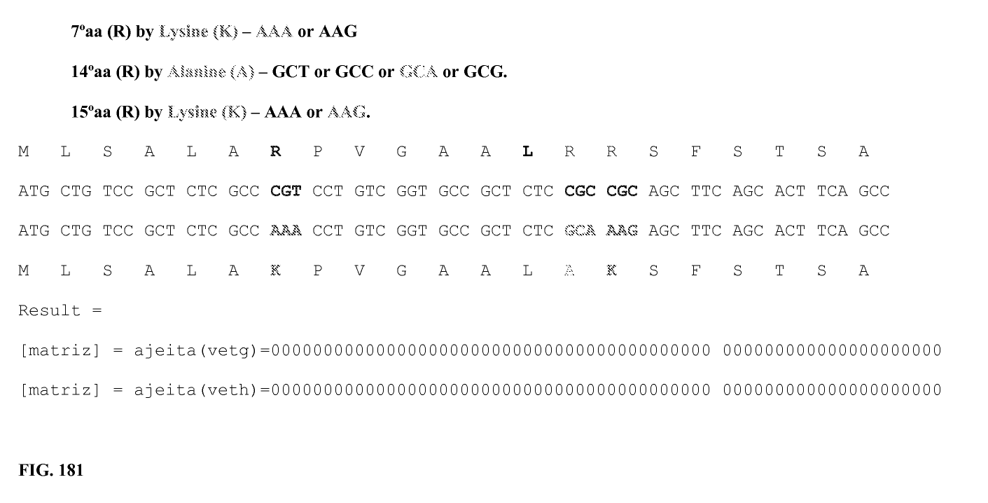

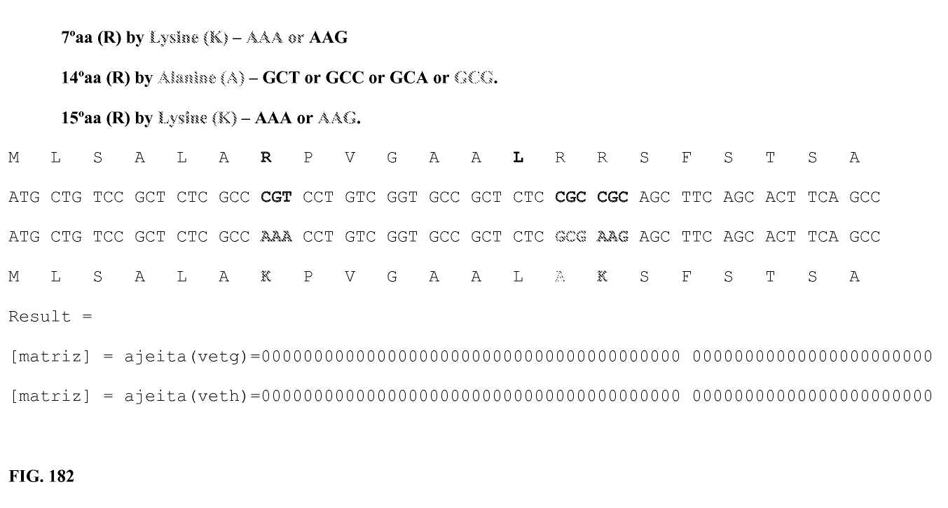

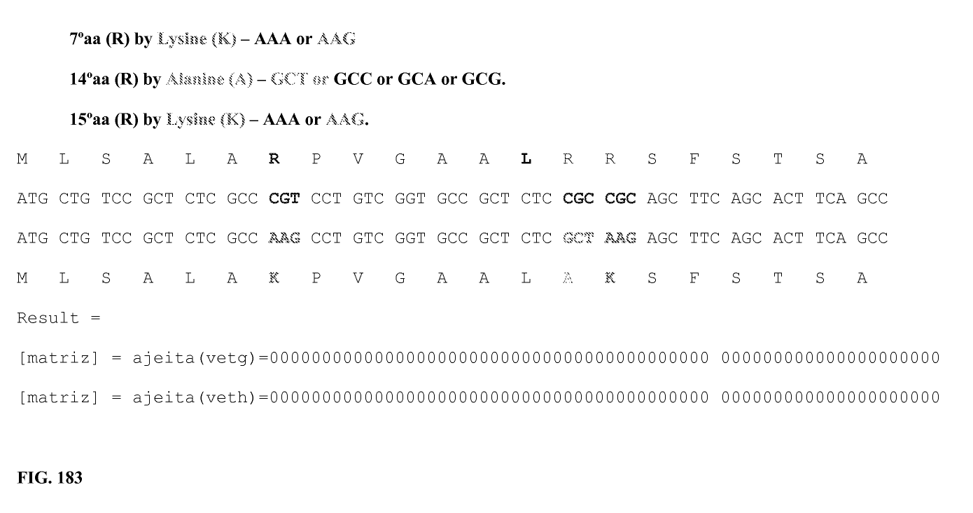

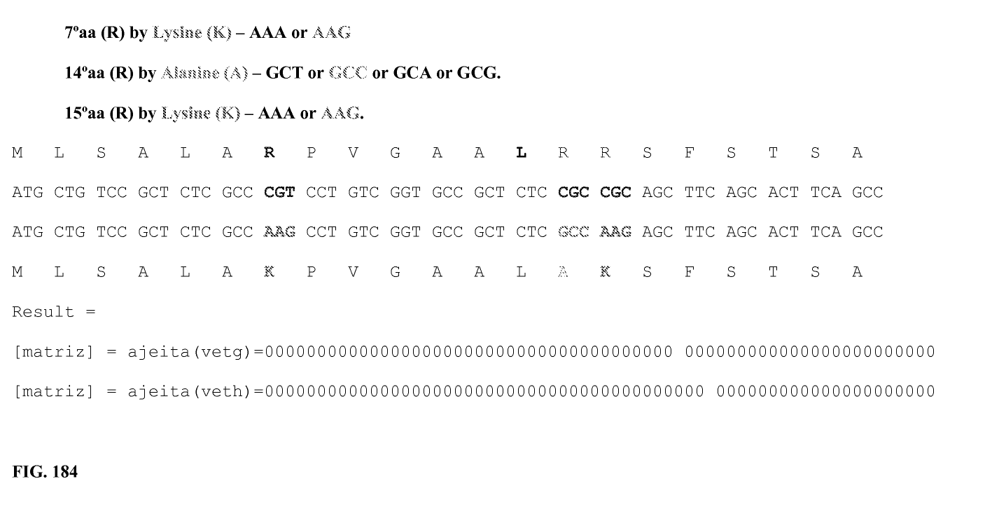

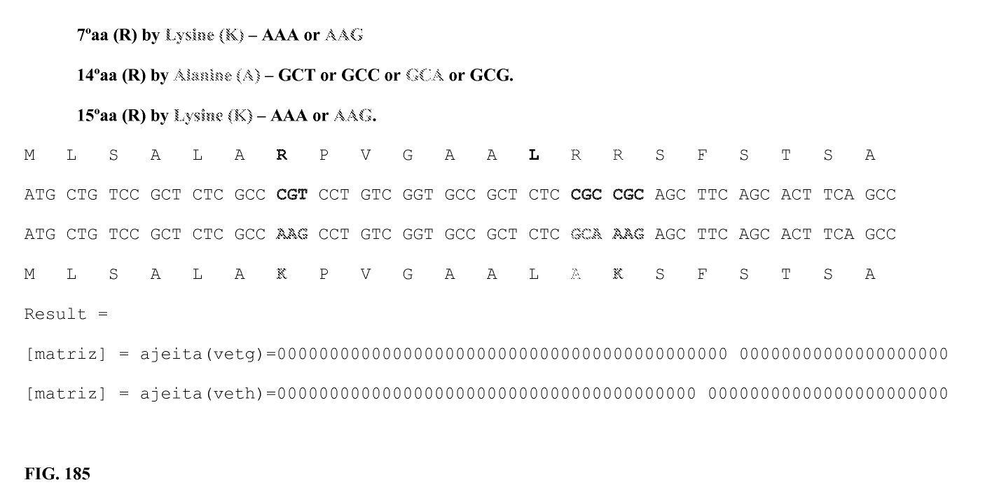

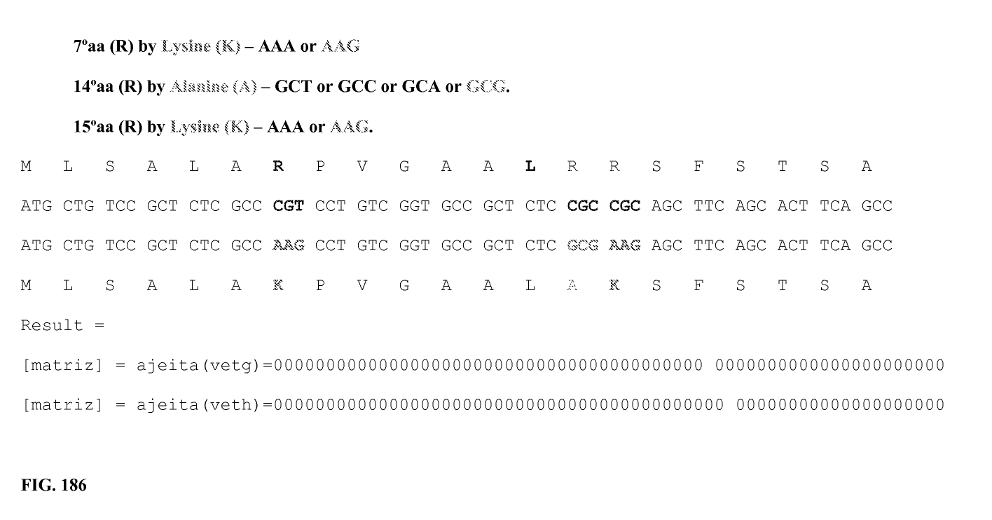

MDHKK—
7° aa (R) by Lysine (K)—AAA or AAG
14° aa (R) by Alanine (A)—GCT or GCC or GCA or GCG.
15° aa (R) by Lysine (K)—AAA or AAG.
1) MDHKK—shows the analysis of one of the sixteen possible combinations between the nucleotides of: K, A and K (see FIG. 171).
Conclusion: The change was not accepted by the code.
2) MDHKK—shows the analysis of one of the sixteen possible combinations between the nucleotides of: K, A and K (see FIG. 172).
Conclusion: The change was not accepted by the code.
3) MDHKK—shows the analysis of one of the sixteen possible combinations between the nucleotides of: K, A and K (see FIG. 173).
Conclusion: The change was not accepted by the code.
4) MDHKK—shows the analysis of one of the sixteen possible combinations between the nucleotides of: K, A and K (see FIG. 174).
Conclusion: The change was not accepted by the code.
5) MDHKK—shows the analysis of one of the sixteen possible combinations between the nucleotides of: K, A and K (see FIG. 175).
Conclusion: The change was not accepted by the code.
6) MDHKK—shows the analysis of one of the sixteen possible combinations between the nucleotides of: K, A and K (see FIG. 176).
Conclusion: The change was not accepted by the code.
7) MDHKK—shows the analysis of one of the sixteen possible combinations between the nucleotides of: K, A and K (see FIG. 177).
Conclusion: The change was not accepted by the code.
8) MDHKK—shows the analysis of one of the sixteen possible combinations between the nucleotides of: K, A and K (see FIG. 178).
Conclusion: The change was not accepted by the code.
9) MDHKK—shows the analysis of one of the sixteen possible combinations between the nucleotides of: K, A and K (see FIG. 179).
Conclusion: The change was not accepted by the code.
10) MDHKK—shows the analysis of one of the sixteen possible combinations between the nucleotides of: K, A and K (see FIG. 180).
Conclusion: The change was not accepted by the code.
11) MDHKK—shows the analysis of one of the sixteen possible combinations between the nucleotides of: K, A and K (see FIG. 181).
Conclusion: The change was not accepted by the code.
12) MDHKK—shows the analysis of one of the sixteen possible combinations between the nucleotides of: K, A and K (see FIG. 182).
Conclusion: The change was not accepted by the code.
13) MDHKK—shows the analysis of one of the sixteen possible combinations between the nucleotides of: K, A and K (see FIG. 183).
Conclusion: The change was not accepted by the code.
14) MDHKK—shows the analysis of one of the sixteen possible combinations between the nucleotides of: K, A and K (see FIG. 184).
Conclusion: The change was not accepted by the code.
15) MDHKK—shows the analysis of one of the sixteen possible combinations between the nucleotides of: K, A and K (see FIG. 185).
Conclusion: The change was not accepted by the code.
16) MDHKK—shows the analysis of one of the sixteen possible combinations between the nucleotides of: K, A and K (see FIG. 186).
Conclusion: The change was not accepted by the code.

According to [6], the drastic substitutions for the specific recognition system and the correct cleavage of the peptidase extension were the substitutions realized in the MDHKR, MDHRK and MDHKK sequences. The analysis resulting from the method being proposed in this invention not only confirmed these substitutions are drastic to the system as well as confirmed that the substitutions of the MDHRK and MDHKK sequences are the most dramatic than the MDHKR sequence. These results are unexpected considering the fact that the results coming from the kinetic parameters might be entirely reproduced by error-correcting codes generated by algebraic structures. These non-limiting findings show that a mathematical approach is systematically applied to protein engineering.

### Example 12

### Analyzing Polymorphisms and Mutations by Amino Acid Changes

This non-limiting example demonstrates that the manipulation of amino acid changes in selected positions in DNA sequences (proteins, organelle targeting sequences, protein motifs, hormones, introns, repetitive DNA, etc), according to the interest of the application, allow either a scientist or a lab technician to analyze the effects of the mutations in the sequences.

The manipulation of the amino acid changes in selected positions in DNA sequences allow to validate or not a mutation, indicating the position and the amino acid that should or should not be modified to guarantee the information content of the sequence.

Another aspect of the present invention is to infer if it will occur or will not the import of organellar protein by the manipulation of the amino acid changes in the targeting sequences.

### Example 13

### Phylogeny

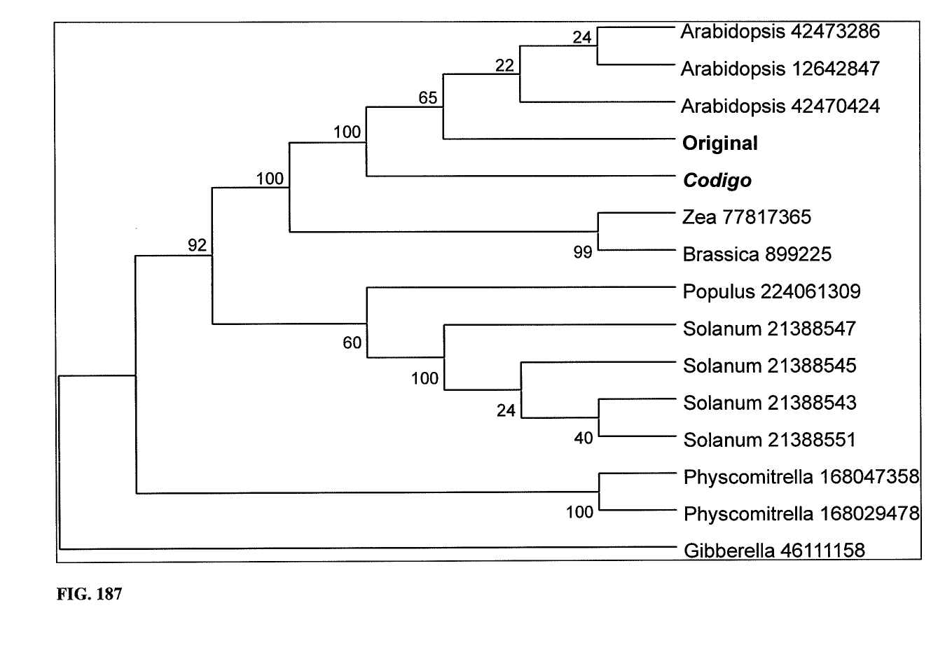

The phylogenetics hypothesis was proposed based on two distinct approaches. First, the Neighbor-Joining method with the evolutionary distances computed using Jukes-Cantor model were performed using MEGA 4.0 [42], the clades consistency were evaluated using the bootstrap non parametric test [36] with 1000 replications. The distance analysis indicates that all Arabidopsis thaliana. sequences are monophyletic with strong bootstrap support. A deeper look, focusing only this group, indicates that the sequence generated by the Mathematical Code (MC) acts as an external group for A. thaliana malate dehydrogenases (FIG. 187).

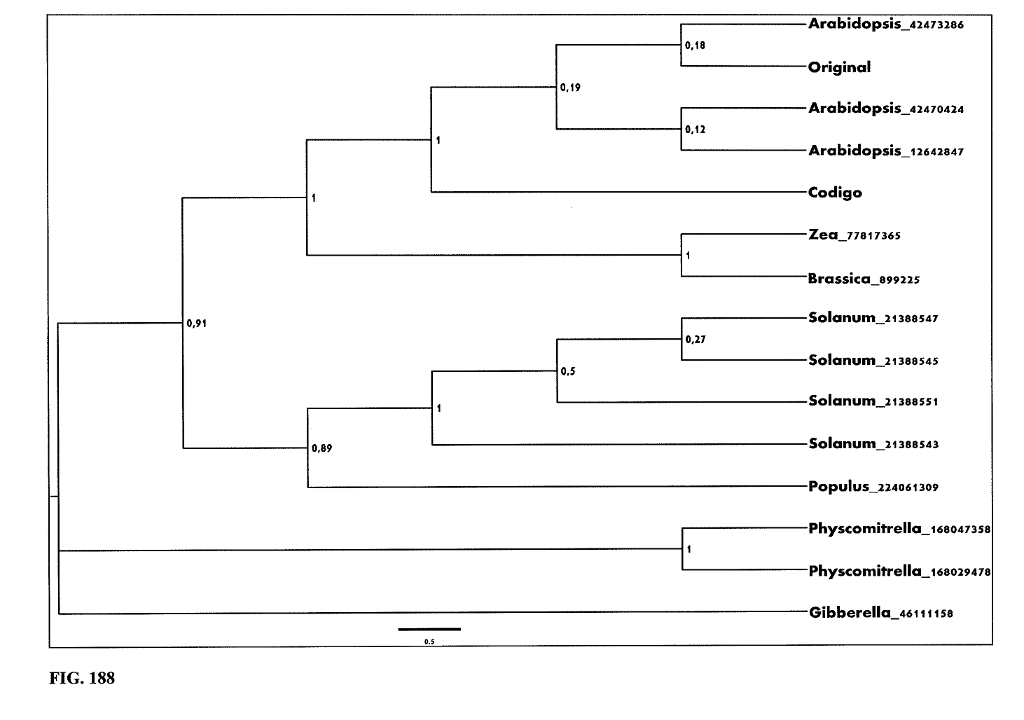

Our second approach was the Bayesian analysis using Mrbayes CVS version [37]. We used the program MODELTEST 3.06 [38] e [39] to determine the available substitution model with the best fit for our data set. Bayesian analyses were carried out for the data set under the model GTR+G+I (General Time-Reversible model [40] e [41], with gamma distribution (F) and with proportion of invariable sites (I)). We conducted six simultaneous chains for 5.0×10⁶ generations, sampling trees every 500 cycles. The 2500 first trees were discarded as “burn in.” For all analysis, Gibberella zeae PH-1 hypothetical protein partial mRNA sequence was used as outgroup to root the tree. Again, A. thaliana form a monophyletic group rooted by the generated sequence from the Code with a strong support (FIG. 188).

The combined analysis of the phenogram and the Bayesian phylogenetic hypothesis points that the small difference present in the sequence outputted from the algorithm is sufficient relevant to outgroup it. It might be premature to avow, but, some evidences shall indicate that the generated sequence may be closer derived from the Arabidopsis t. malate dehydrogenase ancestor than the other paralogs.

### INDUSTRIAL APPLICABILITY

1) Generation and reproduction of DNA sequences of any length by use of trellis codes (convolutional codes), derived from primitive and non primitive linear block codes;

2) Determination of the secondary and terciary structures of DNA sequences from the primary structure, with respect to the topological and geometrical aspects;

3) Predictive analysis with respect to the possibility of developing illness originated by mutations in DNA sequences;

4) To determine the mathematical structure of the DNA sequence and the corresponding polymorphisms (SNPs, InDels, etc) and correlate them with predisposition of developing illness originated by modifications in DNA sequences. This approach will allow mathematical analysis of polymorphisms in populations in order to propose procedures and medical therapies.

5) Another important application is to use this mathematical approach in individual and populational studies in order to verify if the occurrence of mutations/polymorphisms in genes associated to diseases in human beings, animal, plants and microorganisms favors or predisposes to the development of diseases. This methodology may be utilized as a diagnostic test in different organisms to detect in initial phases the predisposition or not for diagnostic and diseases treatment.

The patents and printed publications that have been referred to in the present disclosure, the teachings of which are hereby each incorporated in their respective entireties by reference, are as follows:

### REFERENCES QUOTED IN THE BACKGROUND, SUMMARY AND DETAILED DESCRIPTION

[1] C. E. Shannon, A mathematical theory of communication, Bell Syst. Tech. J., 27 (1948) pp. 379-423 and 623-656. Reprinted in: C. E. Shannon and W. Weaver, eds., A Mathematical Theory of Communication, (Univ. of Illinois Press, Urbana, Ill., 1963).
[2] A. W. Nordstrom and J. P. Robinson, An optimum nonlinear code, Info. and Control, 11 (1967) 613-616.
[3] F. P. Preparata, A class of optimum nonlinear double-error correcting codes, Info. and Control, 13 (1968) 378-400.
[4] F. J. McWillians and N. J. A. Sloane, The Theory of Error Correcting Codes, North-Holland Publishing Company, 1977.
[5] W. W. Peterson and E. J. Weldon, Jr., Error-Correcting Codes, 2^{nd} ed. MIT Press, 1972.
[6] T. Niidome, S. Kitada, K. Shimokata, T. Ogishima, and A. Ito, “Arginine residues in the extension peptide are required for cleavage of a precursor by mitochondrial processing peptidase,” J Biol Chem, vol. 269, pp. 24719-24722, 1994.
[7] J. C. Interlando, R. Palazzo Jr, and M. Elia, “On the decoding of BCH and Reed-Solomon codes over integer residues rings,”IEEE Trans. Inform. Theory, vol. IT43, no. 3, pp. 1013-1021, 1997.
[8] J. R. Gerônimo, R. Palazzo Jr, J. C. Interlando, M. M. S. Alves, and S. I. R. Costa, “The symmetry group of ZqN in the Lee space and the ZqN-linear codes,” Lecture Notes in Computer Science, vol. 1255, pp. 66-77, 1997.
[9] A. A. Andrade, and R. Palazzo Jr, “Construction and decoding of BCH codes over finite commutative rings,” Linear Algebra and Its Applications, vol. 286, pp. 69-85, 1999.
[10] M. Elia, J. C. Interlando, and R. Palazzo Jr, “Computing the reciprocal of units in finite Galois rings,” Journal of Discrete Mathematical Sciences and Cryptography, vol. 3, no. 1-3, pp. 41-55, 2000.
[11] A. A. Andrade, and R. Palazzo Jr, “Alternant and BCH codes over certain local finite rings,” Computational and Applied Mathematics, vol. 22, no. 2, pp. 233-247, 2003.
[12] M. M. S. Alves, J. R. Geronimo, R. Palazzo Jr, S. I. R. Costa, J. C. Interlando, and M. C. Aranjo, “Relating propelinear and binary G-linear codes,” Discrete Mathematics, vol. 243, no. 1-3, pp. 187-194, 2002.
[13] A. R. Hammons, Jr., P. V. Kumar, A. R. Calderbank, N. J. A. Sloane and P. Sole, A Z₄-linearity of Kerdock, Preparata, Goethals, and related codes, IEEE Trans. Inform. Theory, vol. IT-40, No. 2, pp. 301-319, March 1994.
[14] Z. Dawy, P. Hanus, J. Weindl, J. Dingel, and F. Morcos, On genomic coding theory, European Transactions on Telecommunications, vol. 18, pp. 873-879, 2007
[15] H. Yockey, Information Theory and Molecular Biology, Cambridge University Press: Cambridge, 1992.
[16] D. R. Forsdyke, Are introns in-series error detecting sequences?, Intel. J. Theor. Biol., vol. 93, pp. 861-866, 1981.
[17] D. R. Forsdyke, Conservation of stem-loop potential in introns of snake venom phospholipase A₂ genes. An application of FORS-D analysis, Mol. Biol. and Evol., vol. 12, pp. 1157-1165, 1995.
[18] L. S. Liebovitch, Y. Tao, A. T. Todorov, and L. Levine, Is there an error correcting code in the base sequence in DNA?, Biophysical Journal, vol. 71, pp. 1539-1544, 1996.
[19] G. L. Rosen, Examining coding structure and redundancy in DNA, IEEE Engineering in Medicine and Biology, vol. 25, pp. 62-68, 2006.
[20] G. Battail, Information Theory and error correcting codes in genetics and biological evolution, Introduction to Biosemiotics. Springer: New York, USA, 2006.
[21] E. May, M. Vouk, D. Bitzer and D. Rosnick, An error-correcting code framework for genetic sequence analysis, Journal of the Franklin Institute, vol. 34, pp. 89-109, 2004.
[22] P. Hanus, B. Goebel, J. Dingel, J. Weindl, J. Zerch, Z. Dawy, J. Hagenauer, and J. C. Mueller, Information and communication theory in molecular biology, AdeT, vo. X, pp. 1-12, 2007.
[23] Thomas D. Schneider. Information content of individual genetic sequences. Journal of Theoretical Biology, 189:427-441, 1997.
[24] Thomas D. Schneider, Gary D. Stormo, Larry Gold, and Andzej Dhrenfeucht. Information Content of Binding Sites on Nucleotide Sequences. Journal of Molecular Biology, 188:415-431, 1986.
[25] Thomas D. Schneider and R. Michael Stephens. Sequence Logos: a NewWay to Display Consensus Sequences. Nucleic Acids Research, 18 (20):6097-6100, September 1990.
[26] H. A. Loeliger, “Signal sets matched to groups,” IEEE Trans. Inform. Theory, vol. IT37, pp. 1675-1682, 1991
[27] G. D. Formey, “Geometrically uniform codes,” IEEE Trans. Inform. Theory, vol. IT37, pp. 1241-1260, 1991
[28] B. R. McDonald, Finite Rings with Identity, Marcel-Dekker, Inc. New York, 1974.
[29] D. Mac Donnaill, “Why nature chose A, C, G and U/T: an error-coding perspective of nucleotide alphabet composition,” Origins of life and evolution of the Biosphere 2003; 33:433-455.
[30] J. Rzeszowska-Wolny, “Is genetic code error-correting?” J. Theoret. Biol., vol. 104, pp. 701-702, 1983.
[31] R. Sánchez, L. A. Perfetti, R. Grau, E. Morgado, “A new DNA sequences vector space on a genetic code Galois field,” MATCH Commun. Math. Comput. Chem., 54 (2005) 3.
[32] A. Viterbi and J. K. Omura, Principles of digital Communication and Coding. New York: McGraw-Hil, 1979.
[33] Shu Lin and Daniel J. Costello Jr. Error Control Coding: Fundamentals and Applications. Prentice-Hall, Inc., Englewood Clis, N.J., 1983.
[34] P. Shankar, “On BCH Codes over Arbitrary Integer Rings”, IEEE Trans. Inform. Theory, Vol. IT-25, No 4, pp. 480-483, July 1979.
[35] J. C. Interlando. A Contribution to the Encoding and Decoding of Linear Codes over Abelian Groups via Concateation of Codes over IntegerResidue Rings. PhD Thseis, FEEC-UNICAMP, 1994. (in Portuguese).
[36] Felsenstein, J. (1985) Confidence-Limits on Phylogenies—an Approach Using the Bootstrap, Evolution, 39, 783-791.
[37] Huelsenbeck, J. P. and Ronquist, F. (2001) MRBAYES: Bayesian inference of phylogenetic trees, Bioinformatics, 17, 754-755.
[38] Posada, D. (2003) Using MODELTEST and PAUP* to select a model of nucleotide substitution, Current protocols in bioinformatics/editoral board, Andreas D. Baxevanis . . . [et al, Chapter 6, Unit 6 5.
[39] Posada, D. (2006) ModelTest Server: a web-based tool for the statistical selection of models of nucleotide substitution online, Nucleic Acids Res, 34, W700-703.
[40] Rodriguez, F., et al. (1990) The general stochastic model of nucleotide substitution, J Theor Biol, 142, 485-501.
[41] Schoniger, M. and von Haeseler, A. (1995) Simulating efficiently the evolution of DNA sequences, Comput Appl Biosci, 11, 111-115.
[42] Tamura, K., et al. (2007) MEGA4: Molecular Evolutionary Genetics Analysis (MEGA) Software Version 4.0, Mol Biol Evol, 24, 1596-1599.

### REFERENCES QUOTES IN THE TABLES AND IN THE BRIEF DESCRIPTION OF THE FIGURES

[1] Morikami, A., Aiso, K., Asahi, T., & Nakamura, K., The delta'-subunit of higher plant six-subunit mitochondrial F1-ATPase is homologous to the delta-subunit of animal mitochondrial F 1-ATPase. J Biol Chem 267 (1), 72-76 (1992).
[2] Wang, C. G. et al., Molecular characterization of an anti-epilepsy peptide from the scorpion Buthus martensi Karsch. Eur J Biochem 268 (8), 2480-2485 (2001).
[3] Moawad, T. I., Hoffman, D. R., & Zalat, S., Isolation, cloning and characterization of Polistes dominulus venom phospholipase A1 and its isoforms. Acta Biol Hung 56 (3-4), 261-274 (2005).
[4] Kruft, V., Eubel, H., Jansch, L., Werhahn, W., & Braun, H. P., Proteomic approach to identify novel mitochondrial proteins in Arabidopsis. Plant Physiol 127 (4), 1694-1710 (2001).
[5] Grohmann, L. et al., Extended N-terminal sequencing of proteins of the large ribosomal subunit from yeast mitochondria. FEBS Lett 284 (1), 51-56 (1991).
[6] Hochstrasser, D. F. et al., Human liver protein map: a reference database established by microsequencing and gel comparison. Electrophoresis 13 (12), 992-1001 (1992).
[7] Watson, J. D., Beckett-Jones, B., Roy, R.N., Green, N.C., & Flynn, T. G., Genomic sequence, structural organization and evolutionary conservation of the 13.2-kDa subunit of rat NADH:ubiquinone oxidoreductase. Gene 158 (2), 275-280 (1995).
[8] Bonnefoy, N., Chalvet, F., Hamel, P., Slonimski, P. P., & Dujardin, G., OXA1, a Saccharomyces cerevisiae nuclear gene whose sequence is conserved from prokaryotes to eukaryotes controls cytochrome oxidase biogenesis. J Mol Biol 239 (2), 201-212 (1994).
[9] Unseld, M., Marienfeld, J. R., Brandt, P., & Brennicke, A., The mitochondrial genome of Arabidopsis thaliana contains 57 genes in 366,924 nucleotides. Nat Genet 15 (1), 57-61 (1997).
[10] Millar, A. H., Sweetlove, L. J., Giege, P., & Leaver, C. J., Analysis of the Arabidopsis mitochondrial proteome. Plant Physiol 127 (4), 1711-1727 (2001).
[11] Hejgaard, J., Jacobsen, S., Bjorn, S. E., & Kragh, K. M., Antifungal activity of chitin-binding PR-4 type proteins from barley grain and stressed leaf. FEBS Lett 307 (3), 389-392 (1992).
[12] Dreses-Werringloer, U., Fischer, K., Wachter, E., Link, T. A., & Flugge, U. I., cDNA sequence and deduced amino acid sequence of the precursor of the 37-kDa inner envelope membrane polypeptide from spinach chloroplasts. Its transit peptide contains an amphiphilic alpha-helix as the only detectable structural element. Eur J Biochem 195 (2), 361-368 (1991).
[13] Okamura-Ikeda, K., Fujiwara, K., Yamamoto, M., Hiraga, K., & Motokawa, Y., Isolation and sequence determination of cDNA encoding T-protein of the glycine cleavage system. J Biol Chem 266 (8), 4917-4921 (1991).
[14] Walker, J. E., Lutter, R., Dupuis, A., & Runswick, M. J., Identification of the subunits of F1F0-ATPase from bovine heart mitochondria. Biochemistry 30 (22), 5369-5378 (1991).
[15] Song, J., Wurtele, E. S., & Nikolau, B. J., Molecular cloning and characterization of the cDNA coding for the biotin-containing subunit of 3-methylcrotonoyl-CoA carboxylase: identification of the biotin carboxylase and biotin-carrier domains. Proc Natl Acad Sci USA 91 (13), 5779-5783 (1994).
[16] Trebitsh, T., Goldschmidt, E. E., & Riov, J., Ethylene induces de novo synthesis of chlorophyllase, a chlorophyll degrading enzyme, in Citrus fruit peel. Proc Natl Acad Sci USA 90 (20), 9441-9445 (1993).
[17] Heazlewood, J. L. et al., Experimental analysis of the Arabidopsis mitochondrial proteome highlights signaling and regulatory components, provides assessment of targeting prediction programs, and indicates plant-specific mitochondrial proteins. Plant Cell 16 (1), 241-256 (2004).
[18] Meyer, B., Wittig, I., Trifilieff, E., Karas, M., & Schagger, H., Identification of two proteins associated with mammalian ATP synthase. Mol Cell Proteomics 6 (10), 1690-1699 (2007).
[19] Reinders, J. et al., Profiling phosphoproteins of yeast mitochondria reveals a role of phosphorylation in assembly of the ATP synthase. Mol Cell Proteomics 6 (11), 1896-1906 (2007).
[20] Goossens, A., Geremia, R., Bauw, G., Van Montagu, M., & Angenon, G., Isolation and characterisation of arcelin-5 proteins and cDNAs. Eur J Biochem 225 (3), 787-795 (1994).
[21] Winning, B. M., Bourguignon, J., & Leaver, C. J., Plant mitochondrial NAD+-dependent malic enzyme. cDNA cloning, deduced primary structure of the 59- and 62-kDa subunits, import, gene complexity and expression analysis. J Biol Chem 269 (7), 4780-4786 (1994).
[22] Fabbrini, M. S., Valsasina, B., Nitti, G., Benatti, L., & Vitale, A., The signal peptide of human preproendothelin-1. FEBS Lett 286 (1-2), 91-94 (1991).
[23] Gotthardt, D. et al., Proteomics fingerprinting of phagosome maturation and evidence for the role of a Galpha during uptake. Mol Cell Proteomics 5 (12), 2228-2243 (2006).
[24] Bini, L. et al., Two-dimensional gel electrophoresis of Caenorhabditis elegans homogenates and identification of protein spots by microsequencing. Electrophoresis 18 (3-4), 557-562 (1997).
[25] Ghaemmaghami, S. et al., Global analysis of protein expression in yeast. Nature 425 (6959), 737-741 (2003).
[26] Ghrir, R., Lecaer, J. P., Dufresne, C., & Gueride, M., Primary structure of the two variants of Xenopus laevis mtSSB, a mitochondrial DNA binding protein. Arch Biochem Biophys 291 (2), 395-400 (1991).
[27] Grohmann, L. et al., Extended N-terminal sequencing of proteins of the large ribosomal subunit from yeast mitochondria. FEBS Lett 284 (1), 51-56 (1991).
[28] Kopetzki, E., Entian, K. D., Lottspeich, F., & Mecke, D., Purification procedure and N-terminal amino acid sequence of yeast malate dehydrogenase isoenzymes. Biochim Biophys Acta 912 (3), 398-403 (1987).
[29] Van Dyck, E., Foury, F., Stillman, B., & Brill, S. J., A single-stranded DNA binding protein required for mitochondrial DNA replication in S. cerevisiae is homologous to E. coli SSB. EMBO J. 11 (9), 3421-3430 (1992).
[30] Gevaert, K. et al., Exploring proteomes and analyzing protein processing by mass spectrometric identification of sorted N-terminal peptides. Nat Biotechnol 21 (5), 566-569 (2003).
[31] Turner, S.R., Ireland, R., & Rawsthorne, S., Cloning and characterization of the P subunit of glycine decarboxylase from pea (Pisum sativum). J Biol Chem 267 (8), 5355-5360 (1992).
[32] Lenne, C., Block, M. A., Garin, J., & Douce, R., Sequence and expression of the mRNA encoding HSP22, the mitochondrial small heat-shock protein in pea leaves. Biochem J311 (Pt 3), 805-813 (1995). [33] Kopriva, S. & Bauwe, H., Serine hydroxymethyltransferase from Solanum tuberosum. Plant Physiol 107 (1), 271-272 (1995).

1. A method for determining and validating a mutation in a DNA sequence which encodes a polypeptide sequence using a digital communication system comprising:
a. determining a 4-ary alphabet and a code mathematical structure for said DNA sequence;
b. determining the degree of a primitive polynomial to be used in a Galois ring extension for said DNA sequence;
c. selecting from a number of known primitive polynomials, a first primitive polynomial related to said Galois ring extension, wherein said number is based on said degree;
d. determining a Galois field extension from said first primitive polynomial;
e. determining a plurality of elements of said Galois ring extension;
f. determining a primitive element from said plurality of elements;
g. constructing a cyclic code, wherein the length of said code is based on a code minimum distance;
h. determining all possible values for said code minimum distance;
i. determining a first generator polynomial for a first generator matrix using said cyclic code at a first code distance;
j. determining a second generator polynomial for a parity-check matrix;
k. determining said first generator matrix from said first generator polynomial;
l. determining a first transpose matrix from said first generator matrix;
m. determining said parity-check matrix from said second generator polynomial;
n. determining a second transpose matrix from said parity-check matrix;
o. labeling said DNA sequence using said 4-ary alphabet and said code mathematical structure;
p. verifying said DNA sequence as a codeword of said first generator matrix;
q. determining a third generator polynomial using at a second value for said code minimum distance of step (h), wherein said second code distance is different from said first code distance;
r. repeating steps (m) to (p) for said third generator polynomial until all possible values for said code minimum distance are realized;
s. labeling said codeword using said 4-ary alphabet; and
t. comparing said codeword with an original sequence of said DNA sequence,
wherein the comparison identifies a mutation in the DNA sequence.

2. The method of claim 1, wherein the mutation is a single nucleotide polymorphism (SNP).

3. The method of claim 1, wherein the mutation is associated with a human disease.

4. The method of claim 1, wherein the presence of the mutation is predictive of the probability of contracting a disease.

5. The method of claim 1, wherein the presence of the mutation is predictive of the probability of recurrence of a disease after treatment.

6. The method of claim 3, wherein the human disease comprises a neurological disease.

7. The method of claim 6, wherein the neurological disease comprises Alzheimer's or Parkinson's disease.

8. The method of claim 1, wherein the disease comprises cancer, diabetes or cardiovascular disease.

9. The method of claim 1, further comprising:
a. choosing a second primitive polynomial related to said Galois ring extension, wherein said second primitive polynomial is difference from said first primitive polynomial;
b. repeating steps (d) to (r) until said all known primitive polynomials are used.

10. The method of claim 1, wherein the cyclic code is a primitive BCH code over field.

11. The method of claim 1, wherein the cyclic code is a primitive BCH code over ring.

12. The method of claim 1, wherein the DNA sequence encodes malate dehydrogenase of Arabidopsis thaliana.

13. A digital communication system for determining and validating a mutation in a DNA sequence which encodes a polypeptide sequence, comprising software instructions for enabling the computer to perform pre-determined operations, and a tangible computer readable medium bearing the software instructions; the pre-determined operations including the steps of:
a. obtaining a 4-ary alphabet and a code mathematical structure for said DNA sequence;
b. determining a first generator polynomial of a cyclic code;
c. determining a generator matrix;
d. determining a second generator polynomial of a parity check matrix;
e. determining said parity check matrix;
f. generating all possible permutations between said 4-ary alphabet and said code mathematical structure;
g. generating a first subset of DNA sequences from said possible permutations, wherein each DNA sequence from said first subset of DNA sequences differs from said DNA sequence by one nucleotide;
h. generating a second subset of DNA sequences from said possible permutations, wherein each DNA sequence from said second subset of DNA sequences differs from said DNA sequence by two nucleotides;
i. determining a vector from said possible permutations to compare said each DNA sequence from said first subset of DNA sequences and each DNA sequence from said second subset of DNA sequences with said DNA sequence;
j. and outputting the results.

14. A DNA sequence which encodes a polypeptide sequence having a mutation obtained by the digital communication system of claim 13.

15. The DNA sequence of claim 14, wherein the mutation is a single nucleotide polymorphism (SNP).
